(19) United States
(12) Patent Application Publication
(10) Pub. No.: US 2026/0204413 A1
(43) Pub. Date: Jul. 16, 2026

(54) GENERATION OF INSTRUCTIONS FOR TREATMENT PLANNING USING LANGUAGE MODELS

(71) Applicant: Align Technology, Inc., San Jose, CA (US)

(72) Inventors: Yury Slynko, Frankfurt am Main (DE); Mikhail Gorodilov, Cary, NC (US); Georgii Shushuev, Madrid (ES); Mikhail Stroganov, Morrisville, NC (US); Aleksandr Anikin, Cary, NC (US); Sergei Popov, Cary, NC (US); Vasily Paraketsov, Madrid (ES); William Budiatmadjaja, Buettelborn (DE); Danil Pogorelov, Frankfurt am Main (DE); Ekaterina Sakharova, Darmstadt (DE); Dmitrii Shniakin, Frankfurt am Main (DE)

(21) Appl. No.: 19/448,061

(22) Filed: Jan. 13, 2026

Related U.S. Application Data
(60) Provisional application No. 63/745,245, filed on Jan. 14, 2025.
(60) Provisional application No. 63/948,485, filed on Dec. 24, 2025.

(51) Int. Cl. G16H 50/20; A61C 7/00

(52) U.S. Cl. CPC G16H 50/20; A61C 7/002

(57) ABSTRACT

A method includes obtaining treatment provider instructions expressed in natural language. The treatment provider instructions are associated with treatment of a dental patient. The method further includes providing the treatment provider instructions and a prompt including rules for a domain-specific format for dental treatment plans to a trained AI model. The method further includes obtaining, as output from the AI model, machine-readable instructions related to the treatment provider instructions structured according to the domain-specific format. The method further includes causing a treatment planning algorithm to be adjusted based on the machine-readable instructions.

### RELATED APPLICATIONS

This patent application claims the benefit under 35 U.S.C. § 119(e) of U.S. Provisional Application No. 63/745,245, filed Jan. 14, 2025, and further claims the benefit under 35 U.S.C. § 119(e) of U.S. Provisional Application No. 63/948,485, filed Dec. 24, 2025, both of which are incorporated by reference herein.

### TECHNICAL FIELD

Embodiments of the present invention relate to the field of dentistry, and in particular to the generation of treatment protocol instructions by utilizing artificial intelligence models.

### BACKGROUND

When a dentist or orthodontist is engaging with current and/or potential patients, it is often helpful to utilize treatment protocols to assist with treatment planning operations. A treatment protocol may include a set of operations, preferences, or other instructions that enable efficient development of a treatment plan for a particular patient or disorder. For example, in orthodontic treatment, a practitioner may have preferences for making certain adjustments to dentition before others, preferences for aggression of tooth movement, or preferences for treatment of one type of malocclusion before another. These treatment protocols help standardize care delivery while accommodating practitioner-specific clinical approaches.

In conventional systems, generation of a usable treatment protocol may require input by a technician trained to translate a practitioner's protocol notes into a standardized format, such as machine-readable code associated with a particular treatment planning software. Such systems may require significant investment in training and employing technicians to support partnered practitioners, as well as maintenance and management of facilities for the technicians. Further, there may be a loss of efficiency in protocol design related to delays between a practitioner requesting an update to a protocol or a new protocol, the technician receiving the practitioner's notes, and generation of the protocol in the updated format. Such delays may be further exacerbated in cases where additional updates to the protocol are requested by the practitioner, including multiple rounds of communication between the technician and practitioner to update or fine-tune one or more treatment protocols.

Similarly, when designing a specific treatment plan for a patient, a treatment provider may need to communicate case-specific instructions that differ from or supplement an existing protocol. In some systems, designing a treatment plan may include applying a pre-set protocol, determining that one or more portions of the protocol are incorrect or need adjustment for a specific case, requesting a technician update the protocol or treatment plan, and waiting for the updates to be applied. This process may involve multiple steps, multiple rounds of communication, and significant waiting time, reducing the efficiency of treatment planning workflows and potentially delaying patient care. There is a need for improved systems and methods that can automatically translate practitioner instructions expressed in natural language into machine-readable formats suitable for automated treatment planning operations.

### SUMMARY

The following is a simplified summary of the disclosure in order to provide a basic understanding of some aspects of the disclosure. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect of the present disclosure, a method includes obtaining treatment provider instructions associated with a target dental treatment. The instructions may be expressed in natural language. The method further includes providing first input including the instructions to an AI model. The method further includes obtaining output from the AI model including a first treatment protocol in association with the target dental treatment. The method further includes providing an alert to the treatment provider, the alert including the treatment protocol. The alert may be provided in written form, verbal form via audio output, sign language form via visual display, or combinations thereof.

In another aspect of the present disclosure, a method includes obtaining a plurality of treatment provider instructions associated with dental treatments. The method further includes obtaining a plurality of machine-readable instructions corresponding to the treatment provider instructions. The method further includes training a machine learning model to generate a trained machine learning model. Training the model includes providing the plurality of treatment provider instructions as training input and the plurality of machine-readable instructions as target output.

In another aspect of the present disclosure, a method includes obtaining a data model including one or more fields to be filled. The fields may be associated with a dental treatment. The method further includes generating a first prompt in natural language associated with a first one or more of the fields. The method further includes presenting the first prompt. The first prompt may be presented via a GUI. The method further includes obtaining a response to the first prompt. The method further includes providing the response to a trained machine learning model. The method further includes filling the one or more fields using output of the trained machine learning model based on the response.

In another aspect of the present disclosure, a method includes providing a first set of options related to treatment preferences for a first dental conditions. The method further includes obtaining a first selection of one of the first set of options. The method further includes providing the first selection to a model configured to generate machine-readable code to generate a dental treatment plan based on the first selection. The method further includes obtaining form the model the machine-readable code.

In another aspect of the present disclosure, a method includes providing a first set of treatment options related to a first treatment goal in association with a first dental condition. The method further includes providing a second set of treatment options related to a second treatment goal in association with the first dental condition. The method further includes obtaining a first selection from the first set of treatment options and a second selection from the second set of treatment options. The method further includes generating a treatment protocol in a machine-readable format including the first selection and the second selection. The method further includes obtaining and indication that the first treatment goal is to be applied to a patient in association with the first dental condition. The method further includes generating a treatment plan for the patient corresponding to the first treatment goal based on the treatment protocol.

In another aspect of the present disclosure, a method includes providing, via a graphical user interface (GUI), a set of treatment categories in association with operations of a dental treatment. The method further includes providing, for each of the set of treatment categories, a corresponding set of treatment options. The method further includes obtaining, for a first of the set of treatment categories, a selection from the set of treatment options via the GUI. The method further includes providing the selection to a model configured to generate a treatment protocol in a machine-readable format. The method further includes displaying the treatment protocol via the GUI.

### BRIEF DESCRIPTION OF THE DRAWINGS

The present disclosure is illustrated by way of example, and not by way of limitation in the figures of the accompanying drawings.

FIG. 1 is a block diagram illustrating an exemplary system architecture, according to some embodiments.

FIG. 2 illustrates a model training workflow and a model application workflow, according to some embodiments.

FIG. 3A is a diagram depicting a data flow for generation of a treatment plan using an artificial intelligence (AI) model, according to some embodiments.

FIG. 3B depicts a graphical user interface (GUI) for providing a user experience for a practitioner for generating a treatment protocol using an AI model, according to some embodiments.

FIG. 3C is a block diagram of a flow for obtaining practitioner instructions via a chat function, according to some embodiments.

FIG. 3D is a block diagram of a data flow for generating machine-readable instructions based on practitioner natural language instructions, according to some embodiments.

FIG. 3E is a block diagram of a data flow for utilizing a GUI-based system for converting practitioner preferences into machine-readable treatment protocols, according to some embodiments.

FIG. 3F depicts an example GUI for treatment protocol generation based on selections of a practitioner, according to some embodiments.

FIG. 3G depicts an example GUI for treatment protocol generation based on selections of a practitioner, according to some embodiments.

FIG. 3H depicts an example GUI including nested treatment categorization and selection of treatment goals, according to some embodiments.

FIG. 4A is a block diagram of a data flow for generating a treatment plan based on natural language instructions, according to some embodiments.

FIG. 4B is a diagram of a data flow for generating and implementing a machine-generated prompt for an LLM, according to some embodiments.

FIG. 4C is a diagram of a data flow for classifying instructions into categories for further processing, according to some embodiments.

FIG. 4D is an example treatment planning GUI, including an AI assistant, according to some embodiments.

FIG. 4E is an example process flow for using an AI assistant to update one or more stages of treatment planning, according to some embodiments.

FIG. 4F is an example treatment planning GUI including an AI assistant chat interface and dental arch diagram, according to some embodiments.

FIG. 5A is a flow diagram of a method for generating a dataset for a machine learning model, according to some embodiments.

FIG. 5B is a flow diagram of a method for generating treatment protocol data based on natural language input, according to some embodiments.

FIG. 5C is a flow diagram of a method for training a machine learning or AI model for performing operations associated with generating machine-readable treatment protocol instructions, according to some embodiments.

FIG. 5D is a flow diagram of a method for filling fields of a data model associated with dental treatment, according to some embodiments.

FIG. 5E is a flow diagram of a method for generating machine-readable instructions related to dental treatment based on a selection of options, according to some embodiments.

FIG. 5F is a flow diagram of a method for generating a treatment protocol, and using the treatment protocol to generate a treatment plan in accordance with one or more treatment goals, according to some embodiments.

FIG. 5G is a flow diagram of a method for using a GUI to generate a treatment protocol based on a set of provided options, according to some embodiments.

FIG. 6A is a flow diagram of a method for adjusting a treatment planning algorithm based on natural language instructions, according to some embodiments.

FIG. 6B is a flow diagram of a method for updating a treatment planning algorithm, according to some embodiments.

FIG. 6C is a flow diagram of a method for updating a set of machine-readable instructions based on natural language input, according to some embodiments.

FIG. 6D is a flow diagram of a method for generating machine-readable instructions from natural language instructions, according to some embodiments.

FIG. 6E is a flow diagram of a method for indexing ordered objects in a machine-readable format, based on natural language input, according to some embodiments.

FIG. 6F is a flow diagram of a method for adjusting a treatment planning algorithm, according to some embodiments.

FIG. 6G is a flow diagram of a method for translating natural language indexing to machine-readable indexing for updating a treatment planning algorithm, according to some embodiments.

FIG. 7A is a flow diagram of a method for producing a machine- or model-generated prompt for an LLM, according to some embodiments.

FIG. 7B is a flow diagram of a method for producing a model-generated prompt, according to some embodiments.

FIG. 7C is a flow diagram of a method for generating machine-readable instructions from a target category of natural language instructions, according to some embodiments.

FIG. 7D is a flow diagram of a method for obtaining machine-readable instructions corresponding to a target category of natural language instructions in relation to a dental treatment, according to some embodiments.

FIG. 7E is a flow diagram of a method for updating a treatment plan based on a target category of natural language instructions, according to some embodiments.

FIG. 8A is a flow diagram of a method for updating a treatment plan or protocol utilizing an AI assistant, according to some embodiments.

FIG. 8B is a flow diagram of a method for performing dental treatment planning utilizing an LLM, according to some embodiments.

FIG. 8C is a flow diagram of a method for using an LLM via a GUI element to perform treatment planning, according to some embodiments.

FIG. 9A illustrates a tooth repositioning system including a plurality of appliances, in accordance with some embodiments.

FIG. 9B illustrates a method of orthodontic treatment using a plurality of appliances, in accordance with some embodiments.

FIG. 10 illustrates a method for designing an orthodontic appliance to be produced by direct fabrication, in accordance with some embodiments.

FIG. 11 illustrates a method for digitally planning an orthodontic treatment and/or design or fabrication of an appliance, in accordance with some embodiments.

FIG. 12 is a block diagram illustrating a system for dental treatment planning and intraoral scanning, according to some embodiments.

FIG. 13 is a block diagram illustrating a computer system, according to some embodiments.

### DETAILED DESCRIPTION

The present disclosure relates generally to systems and methods for generating treatment instructions, treatment plans, and treatment protocols in healthcare environments and, more particularly, to the use of artificial intelligence models including large language models for translating natural language instructions from treatment providers into machine-readable treatment protocols and treatment plans. While the present disclosure provides detailed examples in the context of dental and orthodontic applications, the systems and methods described herein may be applicable to other healthcare domains including, but not limited to, orthopedics, physical therapy, surgical planning, prosthetics, rehabilitation medicine, dermatology, ophthalmology, cardiology, oncology, and other medical fields where treatment protocols and treatment plans are generated based on practitioner instructions.

In various embodiments, the systems and methods described herein may receive treatment provider instructions expressed in natural language, process those instructions using one or more trained AI models, and generate machine-readable instructions suitable for automated treatment planning operations. The treatment provider instructions may relate to general treatment preferences applicable across multiple patients, or may relate to specific instructions for a particular patient case. Graphical user interfaces may also be utilized to present treatment options to practitioners, with selections being converted to machine-readable treatment protocols through deterministic or rule-based mappings.

The systems and methods disclosed herein may address limitations of conventional approaches that rely on trained technicians to manually translate practitioner instructions into machine-readable formats. By utilizing AI models such as large language models to perform translation operations, the disclosed techniques may reduce turnaround time for treatment protocol generation and updates, reduce reliance on specialized personnel, and enable practitioners to generate or modify treatment protocols without requiring specialized training in machine-readable instruction formats.

Technologies described herein are related to improving processes of healthcare services by inclusion and utilization of modeling techniques, including machine learning models, artificial intelligence (AI) models, natural language processing (NLP) models, large language models (LLMs), etc. In some healthcare environments, treatment may be planned, augmented, monitored, or the like by computing devices, and various portions of treatment may include generating machine-readable code or instructions for performing these tasks. AI models (e.g., LLMs) may be used to assist in treatment planning, treatment protocol generation, or the like, for various health care services including dental (e.g., orthodontic) health care, orthopedic health care, physical therapy, surgical planning, prosthetics design, rehabilitation medicine, and other medical specialties. AI models may be utilized in translating input from a treatment provider (e.g., a doctor who is not trained in producing machine-readable treatment instructions, three-dimensional modeling related to heath care treatments, or the like) in natural language to a more usable or useful form or format. Graphical user interface (GUI) input may also be used in generating treatment protocols or treatment plans. Treatment options may be provided to a healthcare provider via a GUI, and the provider may generate machine-readable treatment instructions based on their selections of the treatment options.

For many operations described in this disclosure, it may be convenient to utilize one or more large language models. However, different language models, including NLPs, small language models, or specialized language models may be utilized in place of one or more LLMs described in operations herein. For example, an operation described as including multiple LLMs may include one large language model, one natural processing model, one small language model, and one specialized language model specifically tuned for a target use case (such as a dental treatment case) and still be within the scope of this disclosure. Language processing models can vary greatly in scope, power, usage requirements, training requirements, etc., and a different combination of model properties may be utilized to target various tasks of interest with respect to healthcare treatment planning, dental treatments, orthodontic treatments, etc. For convenience, this class of language models are all typically referred to as “large language models” herein, unless otherwise specified. In particular, the most generally powerful large language models are often trained on any and all publicly available data, and include many tunable parameters (e.g., on the order of tens of billions to trillions of tunable parameters). Small language models are often more focused, including fewer tunable parameters (e.g., millions or billions of parameters) and may be trained using a subset of publicly available data, such as data from verified sources or data associated with a target field of interest. Specialized language models may be trained using domain specific data, including private data, and may be or include adjustments to general small language models. Utilizing various iterations of these types of language models in any of the language processing operations described herein may be contemplated, and will be recognized to be within the scope of this disclosure.

A treatment provider may generate one or more protocols for treating health care disorders of interest. For example, a number of common disorders or disorders the practitioner commonly encounters may be candidates for generation of treatment protocols. For example, in the case of orthodontics, a practitioner may have a preference for making some adjustments of dentition before others, preference for aggression of movement, preference for treatment of one type of disorder before another, or the like. Similarly, in orthopedic applications, a practitioner may have preferences for surgical approaches, implant selection, rehabilitation timelines, weight-bearing protocols, or sequencing of treatment phases for conditions such as joint replacement, fracture fixation, ligament reconstruction, or spinal procedures.

In some systems, generation of a usable protocol may include input by a technician trained to translate a practitioner's protocol notes to a standardized format, such as machine-readable code (e.g., proprietary code, code associated with a particular treatment planning software, or the like). Such systems may require investment in training and paying a number of technicians to support partnered practitioners, maintenance and management of facilities for the technicians, etc. Further, there may be a loss of efficiency in protocol design related to delays between a practitioner requesting an update to a protocol or a new protocol be developed, the technician receiving the practitioner's notes, and generation of the protocol in the updated format. Such delays may be further exacerbated in cases where additional updates to the protocol are requested by the doctor, e.g., including multiple rounds of communication between the technician and practitioner to update or fine-tune the one or more treatment protocols. Embodiments provide systems and methods to automatically generate a treatment protocol, such as for dental treatment (e.g., orthodontic treatment and/or palatal expansion treatment), that may obviate the need for a trained technician to generate a treatment protocol.

Dental arch data may be utilized in treatment of a dental arch. For example, one or more dental malocclusions (e.g., misalignment of teeth) may be treated using an orthodontic treatment plan, which may include collecting and utilizing jaw pair data of a patient. As a further example, generation of a crown or dental implant may be performed based on dental arch data. Dental arch data may include data of one or more teeth (e.g., including size, shape, positioning, orientation, etc.), a group of teeth, an arch, an upper and lower jaw, etc.

A treatment provider, based on examination of a health care disorder (e.g., malocclusion) and/or data collected in connection with the health care disorder (e.g., dental arch data), may design a specific treatment plan with respect to the patient exhibiting the health care disorder. The treatment plan may be related to an existing protocol, be based on an existing protocol, include one or more adjustments from an existing protocol, or be generated from scratch (e.g., without reliance on a treatment protocol). In some systems, designing of a treatment plan may include applying a pre-set protocol; determining that one or more portions of the protocol are incorrect, not applied correctly to the specific case, or are to be adjusted with respect to a specific case; requesting a technician update the protocol; and waiting for the updates to be applied. Similar to initial generation of a protocol, such a process may take multiple steps, multiple rounds of communication, a significant amount of waiting time, etc. Embodiments provide systems and methods to automatically generate a treatment plan, such as for dental treatment (e.g., orthodontic treatment and/or palatal expansion treatment), that may obviate the need for a trained technician to generate a treatment plan.

Methods and systems of the current disclosure may address one or more shortcomings of conventional solutions. Various tools, systems, and methods are described for streamlining generation of treatment instructions, in particular generation of machine-readable code for enacting treatment. In some embodiments, selectable options presented via a GUI may be used for generation of treatment protocols and/or treatment plans. In some embodiments, an AI model (e.g., such as a language model including large language models, (LLMs) small language models, specialized language models, neural network, etc.) is utilized for generation of one or more treatment protocols. The AI model may further be utilized for validation and/or checking of the treatment protocols. AI models may further be utilized for updating of one or more treatment protocols. The treatment protocols may be related to a variety of healthcare services, including dental, orthodontic, orthopedic, physical therapy, surgical, prosthetic, rehabilitation, or other services for which creation of a treatment protocol may be of interest. In orthopedic contexts, for example, treatment protocols may specify surgical techniques, implant specifications, post-operative care instructions, physical therapy regimens, and recovery milestones for conditions such as hip or knee replacement, rotator cuff repair, ACL reconstruction, spinal fusion, or fracture management.

A treatment protocol may be generated through the use of a GUI. In some embodiments, a GUI may be used to provide some options for a treatment protocol to a provider (e.g., such as a doctor). The provider may select between the options (e.g., via drop-down menus, fillable fields, check boxes, radio buttons, or the like) to generate a treatment protocol or treatment plan based on the provider's preferences. Once the treatment provider's preferences are recorded, a script (e.g., machine-readable code for executing the treatment protocol) may be generated based on the selected options. The machine-readable instructions may be applied to current or future treatment planning operations of the treatment provider.

In some embodiments, a treatment protocol (e.g., a protocol related to a target type of disorder, target treatment type, or the like) may be generated. Generation of the treatment protocol may be performed or assisted by one or more trained AI models. In some embodiments, a treatment provider (e.g., orthodontic practitioner) may provide input in natural language related to treatment protocol for one or more target disorders. The input may be provided by the practitioner via a chat function, a text input, a message (e.g., email), or another input. The input may be provided by the practitioner in natural language. Accordingly, the practitioner may not be required to undergo special training related to treatment protocol generation in order to generate a treatment protocol in embodiments.

In some embodiments, a number of common treatment preferences, common treatment goals, common treatment targets, or the like may be included in options provided via a GUI for generation of treatment protocols. Treatment operations may include more variation than is included in a form outlined by the GUI. For example, healthcare patients may experience disparate, even unique challenges, which may not be captured in a provided form. In some cases, an AI model may be used in situations where relevant options for treatment are not included or have not yet been included in a form-driven treatment protocol application executed via a GUI. In some cases, machine-readable instructions related to treatment planning (which may include proprietary syntax, mechanics, functions, or the like) may be capable of expressing a larger range of treatments than is feasible, cost-effective, or convenient to express via a set of selectable options presented by a GUI. In such cases, LLM- or other AI-based instruction generation may augment the rule-based instruction generation provided by the GUI.

A set of treatment categories may be provided to a practitioner. The set of treatment categories may include general treatment principles relevant to many different treatments (e.g., placing attachments or performing interproximal reduction for orthodontic treatments). The set of treatment categories may include specific conditions for treatment. The set of treatment categories may include options related to treatment appliances.

Under each treatment category, one or more options may be provided for selection by the treatment provider. In some cases, a branching tree of options may be provided, where selections of preferences lead to additional questions or options being presented, which may lead to further selections, etc. The options may include different classifications of a treatment category, different treatment goals, different methods of achieving treatment goals, etc.

In some embodiments, upon filling out a treatment provider's preferences (or utilizing one or more default selections), machine-readable instructions representing a treatment protocol may be generated. Generation of the machine-readable instructions may be deterministic. For example, rule-based generation may be performed in view of the preference selections made via the GUI. The machine-readable protocol may then be applied to any future patient evaluations to generate or update a treatment plan.

In some embodiments, such as in the case of utilizing an AI-based architecture for language processing to generate treatment instructions, natural language input may be provided to one or more AI models. In some embodiments, one or more AI models may be used repeatedly. In an example, output from an AI model may be provided as input to another AI model, which may be configured to perform a different operation than the first AI model. In some embodiments, a single AI model (e.g., a general purpose LLM) may be utilized multiple times, with different inputs, combinations of doctor comments and engineered prompt components, etc., for generation of one or more treatment protocols and/or treatment plans.

In some embodiments, an AI model may be provided doctor or practitioner instructions, which may be accompanied by additional prompt body information. The AI model may be provided with instructions related to protocol generation, protocol editing, treatment planning instructions, additions or changes to a treatment protocol to be used to amend a specific treatment case, or the like. In some embodiments, doctor instructions may be provided to an AI model to perform splitting operations. Splitting operations may split doctor instructions into logical segments. For example, a doctor protocol statement may include a summary of general treatment protocol guidelines for many different disorders, and splitting operations may include separating the instructions into sections each associated with one disorder. In some embodiments, for splitting or other AI/LLM operations, prompt engineering input may be provided in addition to substantive input. For example, a prompt including related instructions, such as “separate the following text into logical sections each related to one dental disorder:” may be provided, with doctor protocol notes appended to it, to perform splitting operations. Any AI model operations may include similar prompt engineering, appended or joined with doctor comments, output of another model, or the like.

In some embodiments, input corresponding to doctor protocol instructions may be provided to an AI model for text formatting. Text formatting instructions may be provided along with a prompt statement, such as instructing an LLM to perform target formatting operations. Text formatting may receive sections splitting via splitting operations. Text formatting may include resolving relevant abbreviations, unifying medical terminology, indexing, labeling, or the like (such as tooth numbering), unifying text formatting, etc.

In some embodiments, input related to doctor protocol instructions may be provided to an AI model for performing title detection. For example, sections split up by an AI model may be provided for title detection operations, which may aid in later operations by identifying key concepts, disorders, treatment categories, or the like associated with a section of doctor instructions. Title detection may assist in assigning disorders or protocol types to sections of doctor input.

In some embodiments, input related to doctor protocol instructions may be provided to one or more transformers. The one or more transformers may generate machine-readable instructions (e.g., coding language statements) related to the doctor protocol instructions. In some embodiments, one statement of the doctor may be translated to one machine-readable instruction, multiple machine-readable instructions, or zero machine-readable instructions, multiple doctor statements may be condensed to a single machine-readable instruction, or the like. The machine-readable instructions may be in a specifically designed or proprietary language, e.g., related to a software used for treatment planning. In some embodiments, different transformers may be configured to provide different interpretations or services. For example, in the case of clear orthodontic aligners, one transformer may be configured to generate code related to appliance attachments, one transformer configured to generate instructions related to final tooth positions, and so on. In some embodiments, the different transformers may be the same AI model (e.g., general purpose LLM), provided with different prompt amendments to adjust operations and/or output of the AI model.

In some embodiments, input related to doctor protocol instructions (e.g., portions of doctor instructions that were not transformed into machine-readable instructions, or left over doctor instructions) may be provided to a default detector AI model. The default detector may determine whether one or more statements are not of clinical interest, not related to a treatment protocol, already included in treatment statements that have been transformed, or the like. For example, routine steps of a treatment may be included in a protocol without specific instructions, and related statements may be removed from consideration by default detection.

In some embodiments, input related to doctor protocol instructions (e.g., the set of machine-readable instructions produced by the one or more transformers) may be provided to an AI model for clinical checking. Clinical checking AI models may be provided with natural language input (e.g., doctor input, a section or portion of doctor input, or the like) and corresponding machine-readable instructions, and instructed to predict whether the instructions match the natural language input. Clinical checking may include one or more safety checks, e.g., to determine whether the protocol is in accordance with one or more clinical threshold conditions associated with a treatment. Further checks may also be performed, such as syntax checks (e.g., correcting syntax for compatalability or the like).

In some embodiments, multiple AI models may be utilized to assess the quality of generated treatment protocols, treatment plans, or machine-readable instructions. Each of the multiple LLMs may be configured to assume a different evaluator role, enabling assessment of the generated protocol from multiple perspectives. The evaluator roles may include, but are not limited to, a clinical advisor role, a marketing specialist role, a software quality assurance (SQA) engineer role, a regulatory compliance reviewer role, a patient safety analyst role, a usability specialist role, or other roles relevant to evaluating treatment protocols. Each role-specific LLM may be configured through specialized prompts that define the evaluation criteria, priorities, and perspective associated with that role. Alternatively or additionally, role-specific LLMs may be configured through fine-tuning on role-specific evaluation data, or by providing role-specific documentation and evaluation criteria via retrieval-augmented generation.

In some embodiments, an LLM configured for a clinical advisor role may evaluate the generated protocol for clinical soundness, adherence to best practices, appropriateness of treatment parameters, and alignment with established clinical guidelines. An LLM configured for a marketing specialist role may evaluate the protocol for clarity of communication, patient-friendliness of explanations, and effectiveness of presenting treatment options to patients. An LLM configured for an SQA engineer role may evaluate the protocol for technical correctness, proper handling of edge cases, consistency of machine-readable instructions, and absence of logical errors or contradictions. An LLM configured for a regulatory compliance reviewer role may evaluate the protocol for compliance with applicable regulatory standards, documentation requirements, and safety regulations. An LLM configured for a patient safety analyst role may evaluate the protocol for potential safety concerns, risk factors, and adherence to safety thresholds.

In some embodiments, the quality assessment using multiple role-specific LLMs may be performed iteratively. Feedback from each of the multiple role-specific LLMs may be aggregated to generate an overall quality score for the generated protocol. The aggregated feedback may identify specific areas of the protocol that may benefit from improvement or revision. In some cases, the feedback from the multiple role-specific LLMs may be provided to a coordinating LLM or aggregation module that synthesizes the individual assessments into a unified quality report. The unified quality report may be provided to a treatment provider for review, or may be used to automatically trigger refinement of the generated protocol. In some embodiments, the quality assessment process may continue iteratively until the generated protocol meets quality thresholds associated with each of the evaluator roles, or until a maximum number of iterations is reached.

In some embodiments, the doctor instructions may be provided based on a statement directed toward one or more treatment protocols provided by the treatment provider. In some embodiments, doctor instructions may be provided via an interactive model. For example, a chat arrangement facilitated by a graphical user interface (GUI) may be utilized in extracting information in natural language from the treatment provider in order to generate one or more treatment protocols. In some embodiments, a data model may include multiple fields to be filled in relation to one or more treatment protocols, and an AI model may be configured to ask natural language questions in a chat setting in order to fill the fields to generate a treatment protocol. In some embodiments, a verification check may be performed to confirm that the machine-readable instructions accurately reflect the intent of the practitioner's natural language input. The AI model may generate machine-readable instructions based on one or more chat responses. A response generator model (which may be AI-based, deterministic, or the like) may then generate a natural language description of the machine-readable instructions for confirmation by the practitioner via the chat. The natural language description may be presented in written form, verbal form via audio or speech synthesis, sign language form via animated visual display, or combinations thereof.

In some embodiments, a machine-readable protocol may be generated. The machine-readable protocol may be applied to a specific patient case (e.g., applied to a three-dimensional scan of patient dentition) to generate a treatment plan, which may also include machine-readable instructions. The treatment plan may include indications of final tooth positions, stages of movement, etc. In some embodiments, the machine-readable protocol may be used to generate a treatment algorithm, which may then be applied to patient data to generate a treatment plan. For example, a first machine-readable language may be used to translate natural language instructions to machine-readable instructions, and may be presented in a machine-readable language that somewhat resembles natural language for ease of generation, ease of review, etc. The protocol expressed in the first machine-readable language may then be used to generate a treatment algorithm, which may be used to generate treatment plans. The treatment algorithm may be in a less intuitive language for human understanding, but more easily applied to the task of generating treatment plans based on patient data.

In some embodiments, doctor instructions related to a specific treatment, specific patient, specific treatment plan, or the like may be provided in natural language to a system. The natural language instructions may be used to augment or supersede treatment operations defined in a treatment protocol. In some embodiments, for a particular dental patient, specific operations may be performed that are different than those expressed in a default treatment plan, default treatment protocol, protocol associated with the treatment provider, or the like. For example, a dental patient may include teeth, implants, pontics, or the like, that are not to be moved during treatment. This may be contrary to a protocol of the treatment provider, but important for proper treatment of this particular patient. Instructions related to treating this patient, including identity of teeth that are not to be moved, may be provided to a treatment planning system.

In some embodiments, updates to a treatment protocol may be provided in natural language. The updates, with some additional prompt language, may be provided to an LLM. The LLM may be configured (e.g., via prompt engineering) to output a machine-readable code related to updates to be made to a treatment protocol, treatment algorithm, treatment plan, or the like.

In some embodiments, machine-readable instructions generated from natural language treatment provider instructions may be organized according to a structured format that categorizes orthodontic instructions into distinct categories, each representing a specific aspect of treatment planning. The structured format may support various methods of teeth representation, accommodating different numbering systems and descriptive approaches used by orthodontists. A standardized schema, such as a JSON schema, may define the structure, allowed values, and relationships between different elements of the format. For instructions that do not fit into predefined categories, the structured format may include a default or “other instructions” category, ensuring that no information from the treatment provider instructions is lost during conversion. The structure of the machine-readable format may be extensible, allowing for the addition of new categories or instruction types as orthodontic practices evolve or as new treatment modalities are introduced.

In some embodiments, the system may distinguish between general treatment preferences and case-specific instructions. General treatment preferences may be applicable to all cases submitted by a treatment provider and may include conditional statements, such as “for cases with missing teeth, perform a specific treatment operation.” Case-specific instructions may be more specific, identifying particular teeth, jaws, or patient-specific details that relate to a particular patient rather than general conditions. The system may handle both types of instructions through different processing pipelines that converge at treatment plan generation. In some cases, case-specific instructions provided in natural language may have higher priority than general preferences when both apply to the same treatment parameter, such that the case-specific instructions override or supplement the general preferences for a particular patient.

In some embodiments, the system may interpret treatment provider instructions contextually, understanding implied meanings based on the surrounding context of the instructions. Ambiguities in the natural language instructions may be resolved based on orthodontic best practices or clinical guidelines. The system may infer missing information based on context or may default to standard treatment approaches when specific details are not provided by the treatment provider. For example, if a treatment provider specifies a treatment goal without specifying particular teeth, the system may infer the relevant teeth based on the treatment goal and clinical conventions.

In some embodiments, a complete automated treatment building workflow may be performed without human intervention for supported instruction types. In such a workflow, an LLM may receive treatment provider instructions and a specially developed prompt, and may return the instructions converted to a machine-readable format. The LLM output may be checked for correctness and compatibility with the treatment planning engine. Upon successful validation, the treatment planning engine may receive the converted instructions along with patient data, such as a three-dimensional model of patient dentition, and may return a treatment plan. This workflow may enable fully automated treatment generation without participation of a CAD designer or treatment planning technician for cases where all instructions are supported and pass validation checks. For cases where instructions are not supported or validation fails, the workflow may fall back to manual treatment planning processes.

In some embodiments, the machine-readable updates to the treatment may be provided for validation operations. Validation operations may be AI-based, may be deterministic, or the like. Treatment validation may include verifying that all commands and syntax included in the LLM output (e.g., generated treatment protocol, treatment plan, etc. expressed in machine-readable format) include allowed functions or forms, etc. Validated machine-readable treatment updates may be provided to an engine for updating a treatment algorithm. For example, machine-readable treatment updates may be added to a treatment algorithm (which may be based on a treatment protocol), may be used to replace portions of a treatment algorithm, may fill spaces in a treatment algorithm intended to have a higher priority than other portions of the algorithm (e.g., specific instructions for a case being given higher priority than general instructions aligning to doctor preferences), or the like.

In some embodiments, validation of machine-readable instructions may include checking that instruction types are supported by the treatment planning engine. Validation may also include checking that parameter values fall within acceptable ranges. For example, when a treatment provider requests a specific number of passive aligners, the system may validate that the requested number falls within protocol limits. If instructions include unsupported instruction types or parameter values outside acceptable ranges, this may indicate conversion errors or instructions that cannot be automatically processed. In such cases, the instructions may be directed to a manual treatment planning process for review by trained personnel. Validation rules may be predefined and may be updated as the treatment planning engine capabilities evolve.

In some embodiments, failure of one or more portions of a procedure designed to use AI models such as LLMs to create or update treatment protocols, treatment algorithms, or treatment plans may cause the operations to be performed in a manual treatment planning process. The manual treatment planning process may include providing the natural language instructions of the doctor to a technician trained to adjust machine-readable language (e.g., protocol generation language, treatment algorithm language, etc.) based on the natural language instructions.

In some embodiments, an AI model (e.g., an LLM) may be instructed to translate natural language instructions of a practitioner related to specific teeth into a machine-readable tooth indexing scheme. A prompt provided to an LLM for generating machine-readable instructions from natural language instructions may include instructions for generating, updating, and/or indexing teeth. In some cases, multiple tooth-indexing schemes (e.g., real-world schemes such as Universal Numbering System (UNS), Palmer, and Fédération dentaire internationale (FID)) may be supported by an LLM.

A prompt provided to an LLM may include instructions for translating various methods for indexing objects (e.g., teeth) into a machine-readable format. For example, the prompt may include descriptions of several popular indexing schemes, may include a target output indexing description, may include descriptions of what teeth belong to various categories (e.g., anteriors, upper left molars, etc.), and/or other examples or instructions for assisting the LLM in correctly identifying teeth from a natural language input.

In some embodiments, validation of instructions may include validating of indexing schemes. In some embodiments, indexing schemes may be applied to patient data to correctly execute doctor instructions. For example, in cases with teeth in unexpected places or orders (e.g., where the patient has missing teeth or extra teeth, large misalignments, etc.), a “true” indexing of the character of teeth may be mismatched from a geometric indexing related to tooth positions. In such cases, instructions including relative positions of teeth or other indexable objects, such as “all teeth between 7 and 10,” or “teeth anterior” to a target tooth, may be misunderstood in an structural indexing. Geometric indexing may be applied to a model of a patient's teeth to match natural language doctor instructions to treatment planning algorithmic instructions.

In some embodiments, natural language instructions from treatment providers may reference teeth in a variety of ways, and the system may be configured to interpret and convert these varied references into a standardized machine-readable format. Teeth may be referenced individually using various numbering systems, such as “UR1”, “#8”, or “1.1”, or as multiple individual teeth, such as “UR1 and UR2”. Teeth may be referenced as anatomical groups, such as “upper left molars” or “anteriors”. Teeth may be referenced as inclusive intervals, such as “between 7 and 10”, indicating a range of teeth along the dental arch. Teeth may be referenced by relative position, such as “mesial of UR3” or “distal to the canine”, indicating a tooth's position relative to another tooth. Teeth may also be referenced by exclusion from a group, such as “all anteriors except centrals”, indicating a set of teeth defined by removing specific teeth from a larger anatomical group. The system may be configured to recognize and convert each of these reference types into a structured machine-readable format suitable for automated treatment planning operations.

In some embodiments, a JSON schema or similar structured data format may be used to represent teeth references in a machine-readable format. The schema may include several components that enable comprehensive representation of teeth references. Individual teeth may be defined as a union of supported numbering systems, such as Palmer notation, Universal Numbering System, and FDI notation, allowing a single tooth to be represented using any of these systems. Tooth groups may represent anatomical groups, such as molars, premolars, canines, incisors, or anteriors, optionally filtered by jaw (upper or lower) and side (left or right). Tooth intervals may define an inclusive range between two teeth, with a begin identifier and an end identifier specifying the boundaries of the interval. Relative positions may specify a tooth relative to another tooth, with a reference tooth identifier and a direction indicator such as mesial or distal. Exclusion logic may represent a group of teeth with specific exclusions, using nested references that include a base set of teeth and an exclude set of teeth to be removed from the base set. This structured representation enables expressive and composable definitions of tooth groups that can be validated programmatically and processed consistently by treatment planning systems.

In some embodiments, to reduce ambiguity in interpreting tooth references, the treatment provider's preferred numbering system may be explicitly included as input to the natural language processing system. This may be particularly important in regions where FDI notation is standard and clinicians often omit dots in tooth numbers. For example, the instruction “more extrusion on 11” may refer to tooth 1.1 in FDI notation, but may refer to a different tooth in Universal Numbering System notation. By capturing the treatment provider's preferred numbering system, the system may ensure correct interpretation of tooth references and accurate mapping to the patient's digital jaw model. The preferred numbering system may be obtained from treatment provider preferences, from a geographic location associated with the treatment provider, from explicit input provided along with the natural language instructions, or from other sources. The preferred numbering system information may be provided to the AI model along with the natural language instructions to guide the conversion process.

In some embodiments, an algorithm for mapping teeth representations to a digital three-dimensional model of patient dentition may include a preprocessing step in which teeth in the digital model are sorted based on their geometric position along the jaw arch. This sorting may ensure consistent ordering even in the presence of irregularities such as unerupted teeth, supernumerary teeth, or pontic teeth. The sorting order may be defined such that teeth in the upper jaw are sorted from right to left along the arch, and teeth in the lower jaw are sorted from left to right along the arch. This sorted array of teeth may be cached and reused across multiple instructions within the same orthodontic case to optimize performance. The geometric sorting may account for actual tooth positions rather than relying solely on tooth numbering, which may not reflect geometric order in cases involving crowded teeth, missing teeth, extra teeth, or other anatomical variations.

In some embodiments, matching logic for mapping teeth representations to digital model teeth may depend on the type of representation. For individual teeth representations, a tooth in the digital model may be selected if its identifier matches any entry in the instruction's teeth array. For tooth group representations, a tooth may be selected if it belongs to the specified anatomical group, such as molars or anteriors, optionally filtered by jaw and side. For interval representations, a tooth may be selected if its position in the sorted array lies between the begin and end identifiers of the interval, inclusive. For relative position representations, a tooth may be selected if it is immediately mesial or distal to the referenced tooth based on the geometric ordering along the arch. For exclusion logic representations, the matching set may be computed by first determining all teeth that match the included group, and then subtracting the teeth that match the excluded set. This matching logic may enable accurate translation of varied natural language tooth references into specific sets of teeth in the patient's digital model.

In some embodiments, for treatment instructions involving interproximal spaces, such as gap closures, interproximal reduction, or black triangle corrections, it may be necessary to derive an array of interteeth intervals from a mapped array of teeth. These intervals may represent the spaces between adjacent teeth and may be used for accurately applying space-related treatment instructions. Given that the array of teeth has been sorted according to anatomical position along the jaw arch, the generation of interteeth intervals may proceed as follows. The mapped array of teeth may first be partitioned into two subarrays, one for the upper jaw and one for the lower jaw, to maintain anatomical coherence. For each jaw-specific subarray, the algorithm may iterate through the list of teeth in order and construct intervals between each pair of adjacent teeth. Each interval may be represented as a pair of tooth identifiers corresponding to the two teeth that bound the interproximal space. This approach may ensure that space-related instructions are accurately and consistently translated into digital treatment plans, even in the presence of anatomical anomalies or incomplete dentition.

In some embodiments, the system may be configured to handle irregular dental cases where teeth may not follow expected numbering order due to crowding, supernumerary teeth, missing teeth, or unusual tooth positions. When treatment providers refer to teeth using relative position terms such as “mesial” or “distal”, they typically refer to geometric position along the dental arch rather than numerical order in a numbering system. The geometric sorting of teeth in the digital model may account for these irregularities, ensuring that relative position references are interpreted correctly. For example, in a case with a supernumerary tooth positioned between two regularly numbered teeth, the geometric sorting may place the supernumerary tooth in its correct position along the arch, enabling accurate interpretation of instructions such as “all teeth mesial to tooth X”. By separating the textual representation of teeth from the digital model mapping, the system may improve the performance of the language model, as the language model may focus on interpreting the natural language instructions without needing to process patient-specific geometric data.

In some embodiments, systems may be in place (e.g., in treatment planning software, protocol generation software, or the like) that are applicable to certain instructions. For example, some categories of instructions may be handled deterministically, some may be handled manually, some may be handled using LLMs or other AI-based models, etc. In some embodiments, natural language instructions may be categorized, and various instructions provided to different systems for resolution. These categorization and routing approaches may be applicable across various medical fields, including dental, orthopedic, surgical, and rehabilitation contexts, where different types of treatment instructions may require different processing approaches based on complexity, safety considerations, or system capabilities.

In some embodiments, natural language comments may be provided to a classification model (e.g., an LLM configured to classify instructions based on training, fine-tuning, prompting strategies, etc.) to classify portions of the comments into relevant categories. Instructions from various categories may be provided to a system suited to execute requested operations or adjustments. In some embodiments, some comments that are not applicable to the target goals (e.g., comments that are not clinically relevant, such as a practitioner thanking an AI assistant) may be excluded from further operations. Comments which are related to instructions that can be deterministically carried out (e.g., comments related to specific updates to clinical practices that are common or well understood) may be provided to a system for deterministically performing the requested updates. Comments which are related to instructions for which a more advanced approach is to be implemented (e.g., where no deterministic system has been generated) may be provided to an LLM for generating machine-readable instructions or the like. In some embodiments, presence or absence of instructions belonging to one or more categories may adjust which systems are utilized in implementing instructions, such as providing instructions which cannot be classified or for which no system is in place to a manual resolution pipeline.

In some embodiments, natural language instructions may be classified to determine whether the natural language instructions include instructions belonging to target categories. The natural language instructions may be classified to determine whether instructions include instructions related to updating dental features. The natural language instructions may be classified to determine whether the instructions include instructions related to dental attachments, such as attachment locations, size, whether to use optimized or custom attachment schemes, stage of treatment for attachments to be utilized, etc. The machine-readable instructions generated from the natural language instructions may comprise updates to one or more treatment plan parameters for a target dental treatment. The treatment plan parameters may comprise dental appliance features for one or more dental appliances to be used for the target dental treatment, such as attachments, bite ramps, modeled appliance features, or mandibular advancement features including buccal blocks or occlusal blocks. The treatment plan parameters may also comprise one or more planning targets, such as intended final positions for one or more teeth, tooth velocities for one or more teeth, target treatment outcome, number of treatment stages, amount of overcorrection, and whether to apply passive aligners. The natural language instructions may be categorized according to the one or more types of treatment plan parameters to be applied for the dental treatment in embodiments.

In some embodiments, upon determining that a target classification of instruction is present, the instructions belonging to the target classification may be provided to a model or system for interpreting or implementing the instructions. In some embodiments, natural language instructions related to the target category (e.g., dental features, attachments, or the like) may be provided to an LLM for interpretation or implementation of the instructions. For example, an attachment model may include a purpose-trained, fine-tuned, or appropriately prompted LLM for generating machine-readable instructions for attachments and/or for other types of treatment categories (e.g., for other treatment plan parameters). Upon determining that instructions related to attachments and/or other specific types of treatment plan parameters are included in the natural language instructions, the instructions may be provided to the attachment model to be integrated into generation or update of treatment plans, treatment protocols, treatment prescriptions, or the like.

In some embodiments, an LLM (which may be a general LLM or an LLM configured to process treatment provider instructions related to dental features and attachments) may be provided with a prompt that defines the role of the AI assistant, specifies action types to be extracted, describes tooth numbering systems, enumerates attachment types, defines stage information formats, and specifies output format requirements. An example prompt for an LLM configured to extract attachment-related information from natural language instructions may include the following components.

The prompt may define the role of the AI assistant as extracting information from doctor instructions related to dental features for use by a technician in preparing a treatment for a patient. The prompt may specify that the response should be provided in a structured format, such as JSON, indicating the type of dental feature, the action required, and the teeth affected by the action.

The prompt may specify a set of action types to be identified from the natural language instructions. The action types may include: an add action for adding specific dental features to particular teeth, where the doctor may use terminology such as “apply attachment,” “schedule attachment,” “start attachment,” or “put attachment”; a forbid_placement action for forbidding placement of new dental features on specific or all teeth; a delay_placement action for delaying placement of an attachment; a remove_existing action for removing dental features from a previous treatment plan; a keep_existing action for keeping particular dental features from a previous treatment plan; a replace_existing action for removing an existing attachment and placing a different one on the same tooth, which may be split into separate remove_existing and add instructions; and a modify_existing action for modifications to already-placed attachments such as resizing, changing position, or changing alignment.

The prompt may include descriptions of multiple tooth numbering systems to enable the LLM to identify which system is being used in the natural language instructions. The tooth numbering systems may include: a Palmer system where teeth identifiers are represented with letters indicating upper (U) or lower (L) and right (R) or left (L) positions followed by a number ranging from 1 to 8, such as “UL2” for upper left 2; an FDI (Fédération Dentaire Internationale) system where teeth identifiers include a first number ranging from 1 to 4, a separator character, and a second number ranging from 1 to 8, such as “1.2” or “2.4”; a Universal system where adult teeth are numbered from 1 to 32 starting from the upper right third molar and moving clockwise; and an unknown classification for cases where the numbering system is unclear or where teeth are referenced by group names such as “molars,” “upper anteriors,” or “centrals.” The prompt may instruct the LLM to identify the numbering system and preserve the original tooth identifiers from the instruction without modification.

The prompt may enumerate attachment types to be identified. Optimized attachments may include specific types such as mesial_distal_root_control (also referred to as MDRC, root control, or distal root tip), multi_plane, extrusion, rotation, retention (also referred to as deep bite), and expansion_support (also referred to as support). The prompt may specify optional variables for optimized attachments including size (regular or largest), MDRC type (single, dual, or free), and extra data (distal, mesial, horizontal, or vertical). Protocol attachments may include predefined protocols such as G4 (corresponding to mesial_distal_root_control attachments applicable to canines or incisors), G5 (corresponding to extrusion attachments applicable to premolars), G7 (corresponding to multi_plane attachments applicable to incisors), and G8 (corresponding to expansion_support attachments applicable to premolars and molars). The prompt may instruct the LLM to verify whether protocol conditions are satisfied based on tooth type.

The prompt may define stage information formats for specifying when actions should be applied during treatment. Stage representations may include: a first_stage indicator for the beginning of treatment (which may be expressed as “stage 0,” “start of treatment,” or “begin stage”); a last_stage indicator for the end of treatment; a passive_aligners indicator for a specific set of stages at the end of treatment; an overcorrection indicator for a specific set of stages; a literal_stage indicator for specific stage numbers; and an all_stages indicator. The prompt may specify optional variables including an offset value representing a number of stages relative to a reference stage, and a time indicator specifying whether an action should be applied before or after a specific stage.

The prompt may specify output format requirements including adherence to a schema (e.g., a JSON schema). The prompt may instruct the LLM to translate instructions not written in English into English in the output. The prompt may include considerations for handling terminology variations and misspellings, such as recognizing “att,” “attach,” “attaches,” or misspellings like “atachemens” as references to attachments. The prompt may include instructions for distinguishing attachment-related instructions from other instruction types, such as interproximal reduction (IPR) instructions, passive aligner instructions, pontics, eruption compensation, eruption tabs, gable bends, or occlusal marks, which should be classified as other instructions rather than dental features.

An example of structured output generated by an LLM based on such a prompt may include an object (e.g., a JSON object) specifying the detected numeric system, an array of dental feature objects each including attachment type information (general type, specific type, and optional parameters), action type, affected teeth, stage information if applicable, and the original instruction text. The output may also include an array for other instructions that are not related to dental features. For example, an instruction such as “add largest dual MDRC to upper anteriors starting at stage 5” may produce output including the numeric system as “unknown,” a dental feature object with general type “optimized_attachments,” specific type “mesial_distal_root_control,” size “largest,” MDRC type “dual,” action “add,” teeth specified as a group “upper_anteriors,” and stage information indicating a literal stage of 5.

In some embodiments, AI models may be utilized in assisting, guiding, and/or integrating various portions of a treatment planning workflow. For example, an AI assistant function may be provided in one or more treatment planning platforms, treatment planning applications, treatment planning programs, or the like. The AI assistant function may be provided via a GUI. The AI assistant function may be implemented via a chat function in some embodiments. The AI assistant function may perform updates to one or more treatment operations, such as performing adjustments to one or more treatment fields (e.g., making selections corresponding to a GUI-based treatment planning or treatment protocol generation program).

In some embodiments, AI models, LLMs, or the like may be provided with information via prompting, contextual information (e.g., previously used prompts or responses), and/or additional documentation. For example, documents may be integrated into LLM responses via retrieval-augmented generation (RAG). In some embodiments, certain responses may be constrained or limited via prompting strategies or RAG. For example, a library of possible machine-readable commands, a library of explanations of various topics, treatments, or disorders, or the like may be provided. The LLM may be constrained to only produce medical explanations that align with provided documentation, only produce machine-readable commands that are included in the library, or the like.

In some embodiments, an AI assistant or guide may be inserted into various steps of a treatment planning process and/or a treatment monitoring process. For example, an AI assistant may be included in generation of a treatment protocol (e.g., a set of guidelines customized to a practitioner to be applied to various patients). The AI assistant may also be used in generating, customizing, or updating a specific treatment plan. In some cases, information may be shared between stages of AI assistance. For example, if a practitioner updates specific treatments multiple times, an AI assistant may recommend updating a treatment protocol to include the change that has been targeted repeatedly by the treatment provider.

In some embodiments, an AI assistant or AI chat function may be used for providing additional information to a treatment provider, or prompting the treatment provider to further clarify instructions. For example, an AI-based (e.g., LLM-based) chat function may provide responses to inquiries about specific treatment options. Treatment options may be provided in a treatment or protocol planning application, browser window, GUI, etc. An AI assistant may be configured to provide additional information about various treatment options responsive to receiving a query. The additional information may be presented in written form, verbal form via audio output or speech synthesis, sign language form via animated visual display, or combinations thereof. An AI assistant may further be configured to ask for additional information upon obtaining a request that is unclear or that does not appear to have any clinical instructions included. In some embodiments, an AI assistant may be provided with various AI tools to, for example, update selectable options of a GUI (e.g., a treatment protocol generation GUI), update a treatment protocol via introduction of machine-readable instructions, update a treatment plan via introduction of machine-readable instructions, or the like.

In some embodiments, AI models may be utilized in developing prompts for efficiently, accurately, and/or reliably causing LLMs or other models to perform any of the tasks discussed herein. In some cases, AI models such as LLMs may be utilized to improve prompts designed to accomplish a target task. For example, a base prompt, along with examples of situations in which the base prompt has performed poorly, may be provided to an LLM, with instructions to rewrite the prompt to generate a machine-generated prompt that correctly handles the example cases.

In some embodiments, an LLM may be used to develop a prompt for performing a task related to an existing task. For example, a base prompt may perform a first task, and an LLM may be used to develop a prompt to perform an extension of the first task, a related task, a task with a somewhat increased or adjusted scope of input data, or the like. In some cases, a categorization task into a set of pre-determined categories may be performed as a first task. A second task may include generating additional categories to assign previously unassigned or otherwise unrelated objects to.

In some embodiments, generating a prompt for an LLM with the assistance of an LLM (which may be the same as the first LLM or a different LLM) may include providing information related to the updated or new prompt. For example, a base prompt intended to solve the same problem or a related problem may be provided. Various additional information may also be provided, such as a description of the intended task, examples of failures of a previous version of the prompt, principles of the target output (such as a machine-readable format for final output), examples of situations in which the new prompt may be expected to perform accurately, etc. Output of the LLM based on these input parameters may be used as a prompt for solving additional problems, or LLM prompt generation may be performed iteratively, and the new prompt may be further provided as a base prompt for further refinement.

Generation of a prompt for a task related to a previous task using an LLM may be applicable to many situations, problems, and industries. In some embodiments, classification or execution of natural language instructions by dental or orthodontic practitioners may benefit from LLM-based prompt generation. Similarly, practitioners in other medical fields such as orthopedics, physical therapy, surgical specialties, and rehabilitation medicine may benefit from LLM-based prompt generation for translating natural language treatment instructions into machine-readable protocols. For example, an orthopedic surgeon may provide natural language instructions regarding joint replacement procedures, fracture fixation approaches, or post-operative rehabilitation protocols, and LLM-based systems may translate these instructions into machine-readable formats for treatment planning systems.

In some embodiments, an AI model may be trained, retrained, adjusted, or the like for one or more operations described herein. In some embodiments, an AI model may be trained for specific portions of the tasks performed by AI models. For example, and AI model may be specifically trained (adjusted, retrained, or the like) for generating machine-readable instructions based on natural language. Training the AI model may include adjusting parameters of an LLM. Training the AI model may include performing parameter-efficient fine-tuning, low-rank adaptations, adjusting adapter layers, or the like. Training the AI model may be based on one or more test cases, feedback data, feedback evaluation data, indirect feedback data (e.g., based on a number of updates provided to one or more AI-generated protocols), or the like.

Aspects of the present disclosure provide technological advantages compared to conventional methods. Aspects of the present disclosure may enable generation of a treatment protocol treatment algorithm, and/or treatment plan without and/or while reducing the role of a technician. Such methods may reduce reliance on a technician, reduce turnaround time for treatment protocol updates or generation, reduce additional delays that may be caused by a technician being in a different time zone or having a different working schedule than a practitioner, or the like. Such methods further improve efficiency by reducing a requirement of training for technicians in parsing doctor comments and generating machine-readable instructions related to protocol generation. In some embodiments, a practitioner could generate one or more treatment protocols essentially immediately without the involvement of any additional personnel. These advantages may be realized across various medical fields, including dental, orthodontic, orthopedic, surgical, physical therapy, and rehabilitation medicine contexts, where treatment protocols guide patient care and may benefit from rapid, accurate translation of practitioner preferences into machine-readable formats.

In some embodiments, options-selection based machine-readable instruction generation may be used along with AI-model based instruction generation. By using these two options together, such as using GUI-provided options for common disorders and AI-based solutions for niche or unusual cases, improvements to efficiency and breadth of options available to a practitioner while reducing or eliminating involvement of a technician trained in generating machine-readable instructions for healthcare treatment may be achieved. For example, dental cases within a scope of options programmed in a GUI-based treatment protocol generation program may be handled deterministically, while dental cases outside a scope of treatment categories may be effectively treated using an AI-based treatment protocol and/or treatment planning solution.

In some aspects, usage of AI- or LLM-based treatment protocol generation, treatment protocol updating, and/or treatment planning may improve a turnaround time of treatment planning operations. Operations including human intervention, which may include waiting due to differing time zones or schedules of a practitioner and a technician, waiting for machine-readable instructions to be generated, waiting for validation from the practitioner, may include multiple rounds of updates, etc., may be avoided by providing instructions to an LLM and generating or updating treatment planning operations based on output of the LLM.

In some embodiments, the systems and methods described herein may be applied to orthopedic treatment planning. Orthopedic treatment protocols may specify treatment approaches for musculoskeletal conditions including joint disorders, fractures, ligament injuries, spinal conditions, and degenerative diseases. Natural language instructions from orthopedic practitioners may be translated into machine-readable protocols that specify surgical approaches, implant types and sizes, fixation methods, rehabilitation timelines, weight-bearing restrictions, and physical therapy regimens. For example, a practitioner may provide natural language instructions such as “use posterior approach for total hip arthroplasty with cementless femoral stem” or “begin partial weight bearing at week 4 post-operatively,” and the AI model may generate corresponding machine-readable instructions for the treatment planning system. Treatment categories in orthopedic applications may include joint replacement parameters, fracture fixation specifications, soft tissue repair techniques, spinal instrumentation preferences, and rehabilitation protocol preferences. The GUI-based treatment option selection systems described herein may present orthopedic practitioners with selectable options for surgical approaches, implant preferences, post-operative protocols, and rehabilitation milestones, with selections being converted to machine-readable treatment protocols through deterministic or AI-based mappings.

In one aspect of the present disclosure, a method includes obtaining treatment provider instructions associated with a target dental treatment. The instructions may be expressed in natural language. The method further includes providing first input including the instructions to an AI model. The method further includes obtaining output from the AI model including a first treatment protocol in association with the target dental treatment. The method further includes providing an alert to the treatment provider, the alert including the treatment protocol.

In another aspect of the present disclosure, a method includes obtaining a plurality of treatment provider instructions associated with dental treatments. The method further includes obtaining a plurality of machine-readable instructions corresponding to the treatment provider instructions. The method further includes training a machine learning model to generate a trained machine learning model. Training the model includes providing the plurality of treatment provider instructions as training input and the plurality of machine-readable instructions as target output.

In another aspect of the present disclosure, a method includes obtaining a data model including one or more fields to be filled. The fields may be associated with a dental treatment. The method further includes generating a first prompt in natural language associated with a first one or more of the fields. The method further includes presenting the first prompt. The first prompt may be presented via a GUI. The method further includes obtaining a response to the first prompt. The method further includes providing the response to a trained machine learning model. The method further includes filling the one or more fields using output of the trained machine learning model based on the response.

In another aspect of the present disclosure, a method includes providing a first set of options related to treatment preferences for a first dental conditions. The method further includes obtaining a first selection of one of the first set of options. The method further includes providing the first selection to a model configured to generate machine-readable code to generate a dental treatment plan based on the first selection. The method further includes obtaining form the model the machine-readable code.

In another aspect of the present disclosure, a method includes providing a first set of treatment options related to a first treatment goal in association with a first dental condition. The method further includes providing a second set of treatment options related to a second treatment goal in association with the first dental condition. The method further includes obtaining a first selection from the first set of treatment options and a second selection from the second set of treatment options. The method further includes generating a treatment protocol in a machine-readable format including the first selection and the second selection. The method further includes obtaining and indication that the first treatment goal is to be applied to a patient in association with the first dental condition. The method further includes generating a treatment plan for the patient corresponding to the first treatment goal based on the treatment protocol.

In another aspect of the present disclosure, a method includes providing, via a graphical user interface (GUI), a set of treatment categories in association with operations of a dental treatment. The method further includes providing, for each of the set of treatment categories, a corresponding set of treatment options. The method further includes obtaining, for a first of the set of treatment categories, a selection from the set of treatment options via the GUI. The method further includes providing the selection to a model configured to generate a treatment protocol in a machine-readable format. The method further includes displaying the treatment protocol via the GUI.

In another aspect of the present disclosure, a method includes obtaining treatment provider instructions in natural language. The treatment provider instructions are related to treatment of a dental patient. The method further includes providing the first treatment provider instructions to a trained AI model. The method further includes obtaining output from the AI model, including machine-readable instructions related to the treatment provider instructions. The method further includes adjusting a treatment planning algorithm based on the first machine-readable instructions.

In another aspect of the present disclosure, a method includes obtaining first instructions in a first machine-readable format configured to generate a treatment plan based on input data. The method further includes obtaining second instructions in natural language corresponding to target adjustments to the first instructions. The method further includes providing the second instructions to a trained AI model. The method further includes obtaining output from the AI model. The output includes third instructions in a second machine-readable format. The method further includes updating the first instructions based on the third instructions.

In another aspect of the present disclosure, a method includes obtaining treatment provider instructions in natural language. The treatment provider instructions are indicative of differences between target dental treatment for a target dental patient and a set of treatment preferences associated with the treatment provider. The method further includes obtaining a prompt. The prompt includes a description of a set of categories of instructions associated with dental treatment. The prompt further includes a set of examples of machine-readable instructions corresponding to natural language instructions within the category of instructions. The method further includes providing the treatment provider instructions and the prompt as input to an AI model. The method further includes obtaining output from the AI model. The method further includes generating a treatment plan based on the output from the AI model.

In another aspect of the present disclosure, a method includes obtaining natural language instructions including a first reference to a set of ordered objects according to a first indexing scheme. The method further includes providing the natural language instructions and an accompanying prompt as input to an AI model. The prompt includes a description of the first indexing scheme. The method further includes obtaining output from the AI model. The output includes machine-readable instructions associated with the natural language instructions. The machine-readable instructions include instructions associated with the set of ordered objects. The machine-readable instructions include a second reference to the set of ordered objects according to a first machine-readable indexing scheme.

In another aspect of the present disclosure, a method includes obtaining treatment provider instructions in natural language associated with treatment of a dental patient. The natural language instructions include a first reference to one or more teeth of the dental patient expressed in accordance with a first indexing scheme. The method further includes providing the natural language instructions and an accompanying prompt as input to an AI model. The prompt includes a description of the first indexing scheme. The method further includes obtaining output from the AI model. The output includes machine-readable instructions related to the treatment provider instructions. The machine-readable instructions include a first reference to a first tooth in accordance with a machine-readable indexing scheme. The method further includes adjusting a treatment planning algorithm based on the machine-readable instructions.

In another aspect of the present disclosure, a method includes obtaining treatment provider instructions in natural language. The treatment provider instructions are indicative of one or more differences between target dental treatment for a target dental patient and a set of treatment preferences associated with the treatment provider. The treatment provider instructions include a first reference to one or more teeth of the dental patient, expressed in accordance with an indexing scheme. The method further includes obtaining a prompt. The prompt includes a description of the indexing scheme. The prompt includes a description of a set of categories of instructions related to dental treatment. The prompt further includes a set of examples of machine-readable instructions corresponding to natural language instructions within the categories of instructions. The method further includes providing the treatment provider instructions and the prompt as input to an artificial intelligence (AI) model. The method further includes obtaining output from the AI model including a second reference to the one or more teeth, expressed in accordance with a machine-readable indexing scheme. The method further includes generating a treatment plan based on the output from the AI model.

In another aspect of the present disclosure, a method includes obtaining a base prompt associated with a first target task for a first LLM. The method further includes providing a first prompt generation request, including the base prompt, as input to a second LLM. The method further includes obtaining, as first output from the second LLM, a model-generated prompt. The method further includes providing the model-generated prompt and input associated with a second target task, different than the first target task, to a third LLM. The method further includes obtaining output from the third LLM based on the model-generated prompt and the second target task.

In another aspect of the present disclosure, a method includes obtaining a base prompt configured to cause a first LLM to assign portions of natural language input to a first set of categories. The method further includes providing a prompt generation request to a second LLM. The prompt generation request includes the base prompt and a description of a target task, which includes generation of additional categories. The method further includes obtaining a model-generated prompt as output from the second LLM. The method further includes providing the model-generated prompt and first input including natural language associated with the third target task to a third LLM. The method further includes obtaining output from the third LLM including assignment of portions of the first input to the second set of categories.

In another aspect of the present disclosure, a method includes obtaining first natural language instructions from a treatment provider associated with a target treatment. The method further includes providing the first natural language instructions to a classification model. The method further includes determining, using the classification model, that a first category of instructions is not present in the first natural language instructions. The method further includes providing at least a portion of the first natural language instructions and a prompt configured to cause an LLM to generate machine-readable instructions corresponding to natural language instructions to the LLM. The method further includes obtaining machine-readable instructions corresponding to the first natural language instructions from the LLM.

In another aspect of the present disclosure, a method includes obtaining first natural language instructions from a treatment provider including target updates to a target dental treatment. The method further includes providing the first natural language instructions to a first LLM. The first LLM is configured to categorize natural language instructions. The method further includes determining that a first category of instructions is not present in the first natural language instructions. The method further includes providing at least a portion of the first natural language instructions and a prompt configured to cause a second LLM to generate machine-readable instructions corresponding to natural language instructions to the second LLM. The method further includes obtaining machine-readable instructions corresponding to the first natural language instructions from the second LLM.

In another aspect of the present disclosure, a method includes obtaining first natural language instructions from a treatment provider associated with a target dental treatment. The method further includes providing the first natural language instructions to a classification model. The method further includes determining that a first category of instructions is not present in the first natural language instructions. The method further includes determining that a second category of instructions is present in the first natural language instructions. The method further includes providing at least a portion of the first natural language instructions and a prompt configured to cause an LLM to generate machine-readable instructions corresponding to natural language instructions to the LLM. The method further includes obtaining machine-readable instructions corresponding to the first natural language instructions from the LLM. The method further includes updating a treatment plan to generate an updated treatment plan associated with the target dental treatment based on the machine-readable instructions. The method further includes providing a representation of the updated treatment plan for treatment provider review.

In another aspect of the present disclosure, a method includes obtaining first natural language comments associated with an update to one of a treatment protocol or a treatment plan. The method further includes providing the first natural language comments to an LLM. The method further includes obtaining, from the LLM, machine-implementable instructions corresponding to the first natural language comments. The method further includes executing the machine-implementable instructions.

In another aspect of the present disclosure, a method includes obtaining natural language instructions associated with an update to one of a dental treatment protocol or a dental treatment plan. The method further includes providing the first natural language instructions to an LLM. The method further includes obtaining, from the LLM, machine-implementable instructions corresponding to the first natural language comments. The method further includes performing dental treatment planning operations based on the machine-implementable instructions.

In another aspect of the present disclosure, a method includes providing, via a GUI, a free text entry element for providing instructions associated with updates to a treatment plan. The method further includes obtaining first natural language instructions associated with updating a target treatment plan. The method further includes providing the natural language instructions to an LLM. The method further includes obtaining, from the LLM, machine-readable instructions including updates to the target treatment plan corresponding to the first natural language instructions. The method further includes implementing the machine-readable instructions by performing treatment planning operations.

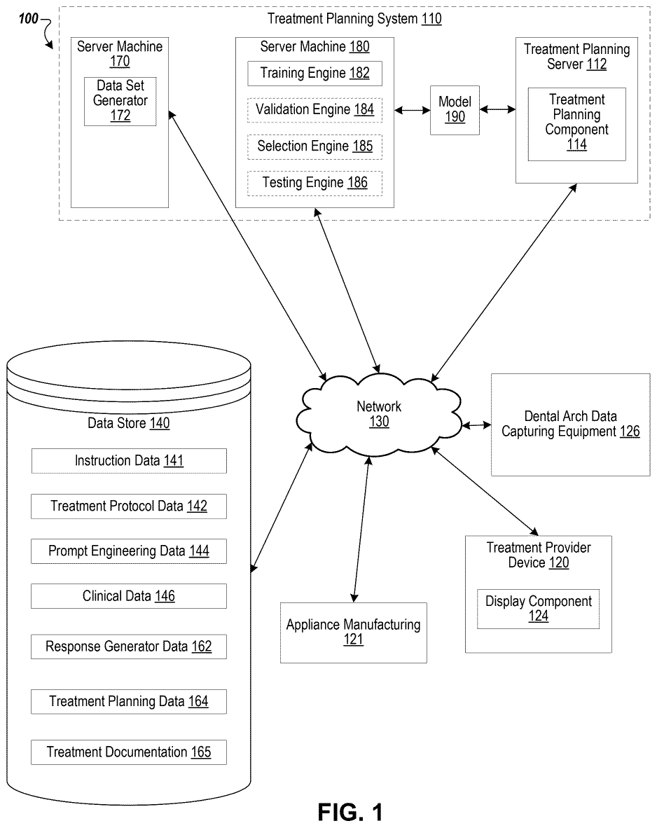

FIG. 1 is a block diagram illustrating an exemplary system 100 (exemplary system architecture), according to some embodiments. The system 100 includes a treatment provider device 120, dental arch data capturing equipment 126, treatment planning server 112, appliance manufacturing 121, and data store 140. The treatment planning server 112 may be part of treatment planning system 110. Treatment planning system 110 may further include server machines 170 and 180. Appliance manufacturing 121 may include any combination of computing devices, hardware (e.g., three-dimensional printers, thermoforming equipment, etc.), software, manufacturing equipment, etc., related to generating or manufacturing appliances (e.g., orthodontic appliances, palatal expanders, retainers, etc.) for providing dental treatment. As used herein, when appropriate, techniques, operations, or components related to dental arches and dental arch data may be extended to include operations related to other oral structures, including upper and/or lower gingiva and palate. For example, dental arch data capturing equipment 126 (e.g., such as intraoral scanning systems) may further be used for performing intraoral scans including generating data of other oral structures in addition to teeth. Some aspects of the present disclosure may be applicable outside a dental context, e.g., to other fields of medicine, other manufacturing fields, other fields including translating natural language instructions to machine-readable instructions, etc. For example, in orthopedic applications, the systems and methods described herein may be used to generate treatment protocols for musculoskeletal conditions, joint replacement planning, fracture treatment, spinal surgery planning, and rehabilitation protocols. In such applications, patient data capturing equipment may include imaging systems such as X-ray machines, MRI scanners, CT scanners, or motion capture systems, and appliance manufacturing may include fabrication of orthopedic implants, prosthetics, braces, splints, or custom orthotics. Aspects related to translating indexing schemes to machine-readable schemes may be applicable to many different technologies, fields, etc. Aspects related to prompt generation by providing a prompt-generation request to an LLM may be applicable to various fields.

Dental arch data capturing equipment 126 may include any combination of equipment for collecting dental arch data, examples of which include intraoral scanning systems (e.g., that includes intraoral scanners and associated computing deices), x-ray machines, cameras, cone beam computed tomography (CBCT) machines, and so on. In some embodiments, dental arch data capturing equipment 126 corresponds to an intraoral scanner as described in U.S. Publication No. 2019/0388193, filed Jun. 19, 2019, entitled “Intraoral 3D Scanner Employing Multiple Miniature Cameras and Multiple Miniature Pattern Projectors,” which is incorporated by reference herein. In some embodiments, dental arch data capturing equipment 126 corresponds to an intraoral scanner as described in U.S. application Ser. No. 16/910,042, filed Jun. 23, 2020 and entitled “Intraoral 3D Scanner Employing Multiple Miniature Cameras and Multiple Miniature Pattern Projectors,” which is incorporated by reference herein. In some embodiments, dental arch data capturing equipment 126 corresponds to an intraoral scanner as described in U.S. Pat. No. 10,835,128, issued Nov. 17, 2020, which is incorporated by reference herein. In some embodiments, dental arch data capturing equipment 126 corresponds to an intraoral scanner as described in U.S. Pat. No. 10,918,286, issued Feb. 21, 2021, which is incorporated by reference herein. Dental arch data may include data of healthy dental arches, dental arches including malocclusion or teeth misalignment, dental arches containing teeth with caries, dental arches containing gingival recession, gingival swelling, tooth wear, tooth cracks, etc.

In some embodiments, treatment provider device 120 may be used to generate or collect doctor instructions related to one or more treatment protocols and/or treatment plans, which may be included in instruction data 141 of data store 140. Doctor instructions stored as instruction data 141 may include doctor preferences, treatment goals, treatment priorities, etc. Doctor instructions may be collected via display component 124 of treatment provider device 120, e.g., via a user interface, graphical user interface (GUI), etc. In some embodiments, a set of options related to treatment may be provided to a treatment provider. The options may be presented as drop boxes, selectable icons, tabs, folders, or the like. Instruction data 141 may be based on selection of treatment preferences via the GUI provided by treatment provider device 120. In some embodiments, doctor instructions may be collected in natural language, e.g., via text fill boxes presented by display component 124. Instruction data 141 may include instructions related to generating a treatment protocol (e.g., an expression of treatment preferences of a practitioner), updating a treatment protocol, generating a treatment plan (e.g., adjustments to a default plan or to a protocol tailored to a specific case or patient), updating a treatment plan, etc.

Instruction data 141 may store doctor comments, notes, and/or instructions related to treatment protocols, treatment plans, etc. Instruction data 141 may relate to or include natural language input by one or more doctors, healthcare professionals, treatment providers, practitioners, etc. Instruction data 141 may be provided via display component 124 of treatment provider device 120. Instruction data 141 may relate to one or more treatment protocols, which may be or include general plans meant to relate to one or more generic, common, repeatedly encountered, or the like healthcare disorders (e.g., dental disorders, malocclusion, misalignment, or the like).

In some embodiments, natural language or machine-readable instructions may be classified. For example, instructions related to particular stages or types of treatment, particular dental disorders, specific appliances or implements, or the like may be grouped together. In some cases, an LLM may be used to perform classification operations, such as determining a category to which natural language instructions or sections of natural language instructions may be assigned. Instruction data 141 may further include classification or categorization data associated with various instructions.

Doctor instructions may be processed (e.g., by the treatment provider device 120 and/or by the treatment planning server 112). Processing of the instruction data 141 may include generating features. In some embodiments, the features are a pattern in the instruction data 141 (e.g., slope, width, height, peak, etc.) or a combination of values from the instruction data 141. Feature generation may include tokenizing the instruction data 141. Instruction data 141 may include features and the features may be used by treatment planning component 114 for performing signal processing and/or for obtaining treatment protocol data 142, e.g., for generation of healthcare treatments. In some embodiments, features may include segmentation data of instruction data 141. Segmentation may be performed (e.g., using a trained machine learning model trained to perform instance segmentation or semantic segmentation), and may include separation of various portions of doctor input into different sections, portions, or categories, such as sections related to different disorders, treatment types, teeth, or the like. Each instance (e.g., set) of instruction data 141 may correspond to an individual (e.g., practitioner), a group of similar dental arches, a group of teeth, or the like.

In some embodiments, treatment planning system 110 may generate treatment protocol data 142 and/or treatment planning data 164 based on instruction data 141. In some embodiments, treatment protocol data 142 may be related to encoding practitioner treatment preferences, and treatment planning data 164 may encode treatment planning operations for a specific treatment, specific patient, specific set of dentition, or the like. Treatment planning system 110 may generate protocol data 142 and/or treatment planning data 164 based on input provided, optionally via a GUI, indicating practitioner or clinician treatment preferences. The options presented by the GUI may be curated, e.g., common preferences may be included. The options presented by the GUI may transfer to a protocol design. For example, there may be a deterministic or rule-based mapping generated that translates options input by the treatment provider to treatment protocols and/or treatment plans. A rule-based mapping may be used to produce machine-readable code that, when executed, applies treatment preferences or a treatment protocol. For example, the machine-readable instructions may be executed in reference to a particular patient, and treatment planning operations in view of doctor selections may be performed based on the machine-readable protocol.

In some embodiments, treatment planning system 110 may generate treatment protocol data 142 and/or treatment planning data 164 using supervised machine learning (e.g., treatment protocol data 142 includes output from a machine learning model that was trained using labeled data, such as incomplete jaw pair data (e.g., data of one or a few teeth) labeled with complete jaw pair data (e.g., jaw pair data including all teeth of the jaw)). In some embodiments, treatment planning system 110 may generate treatment protocol data 142 and/or treatment planning data 164 based on providing doctor instructions to one or more instances of a natural language processing model, an LLM, or the like. In some embodiments, treatment protocol data 142 and/or treatment planning data 164 may be or include machine readable instructions, e.g., related to target positions of one or more teeth to treat orthodontic disorders, related to treatment operations or steps for treating dental disorders, or the like. In some embodiments, treatment planning system 110 may generate treatment protocol data 142 and/or treatment planning data using unsupervised machine learning. For example, treatment protocol data 142 may include output from a machine learning model that was trained using unlabeled data. The output may include clustering results, principle component analysis, anomaly detection, etc. In some embodiments, treatment planning system 110 may generate treatment protocol data 142 using semi-supervised learning (e.g., training data may include a mix of labeled and unlabeled data, etc.). In some embodiments, treatment planning system 110 may generate treatment protocol data 142 and/or treatment planning data using self-supervised learning, e.g., training data may also include target output data, such as in an autoencoder model.

In some embodiments, generation of treatment protocol data 142 and/or treatment planning data 164 (e.g., by providing instruction data 141 to an LLM) may also include providing further data to an AI model, such as instructions to process the input in a target way. Such instructions may be stored as prompt engineering data 144. Prompt engineering data 144 may include one or more prompts, prompt forms, prompt generation algorithms, or the like for generating accompanying data to be provided along with data associated with doctor input to an AI model. Prompt engineering data 144 may include additional text to be provided to an LLM that may be inserted before, in the middle of, after, or otherwise with a selection of doctor input or data based on doctor input (e.g., pre-processed or processed doctor input). Prompt engineering data 144 may cause an LLM to perform a different task based on which prompt is provided. For example, one LLM may be used for many different tasks by providing input data accompanied with various additional prompt information. Prompt engineering data may include information directing the LLM in generating machine-readable code. Prompt engineering data 144 may include information directing the LLM in parsing and generating indexing schemes for ordered objects, e.g., dentition of a patient.

In some embodiments, prompt engineering may include use of an AI model such as an LLM. For example, a description of a target task may be provided to an LLM as a prompt generation request, and the LLM may provide a prompt that is predicted to configure an LLM to perform the target task. The prompt generation request may include further information, such as a related prompt, a description of situations in which the related prompt failed to perform as intended, a description of some examples that the machine-generated prompt may be expected to correctly execute, etc. Information related to LLM prompt generation may also be included in prompt engineering data 142.

In some embodiments, data store 140 may further include clinical data 146. Clinical data 146 may include patient data, such as three-dimensional models (e.g., surface meshes) of patient dentition. Clinical data 146 may include various thresholds, constraints, or the like related to physical realities available for treatment, such as maximum changes available for orthodontic procedures, maximum speed of adjustment, maximum number of treatment stages, maximum treatment time, and/or other constraints. Clinical data 146 may be used to check machine-readable protocol instructions to determine or verify that the instructions are in accordance with one or more rules, principles, or the like. Clinical data 146 may be used for performing validation of machine-readable instructions produced by an AI model such as an LLM. Clinical data 146 may be used to check or adjust treatment protocols generated by one or more AI models, e.g., to maintain safety of the recommended or generated procedures.

Data store 140 may further include response generator data 162. In some embodiments, one or more logical checks may be performed. For example, instructions for a treatment protocol generated by an AI model may be checked by providing a natural language prompt indicating a description of the instructions to a doctor, and asking the doctor whether the description is correct. In some embodiments, such a check response may be generated by a response generator, which may be a deterministic (e.g., rule-based) model. Response generation algorithms, responses, rules, etc., may be stored as response generator data 162. In some embodiments, clinical data 146 and/or treatment planning data 164 may include instructions related to performing treatments, such as models of appliances to be manufactured for treatment, manufacturing instructions for aligners, or the like.

In some embodiments, responses may be generated by an AI model, such as an LLM. For example, a practitioner may provide a query, via a chat function, an AI assistant function, a free text entry function, or the like, related to one or more treatment options, disorders, terminology, best practices, or other aspects relevant to treatment. An LLM may be empowered to generate natural language responses to these queries that clarifies or explains aspects of treatment to the treatment provider. The natural language responses may be presented in written form via a display, verbal form via audio output or speech synthesis, sign language form via animated visual representation, or combinations thereof. In some cases, an LLM may provide a prompt for a practitioner to input additional information. For example, a treatment provider may provide instructions which are not clear (e.g., a confidence in interpretation of the instructions may not meet a target threshold). The LLM, a chat function, a heuristic function, or the like may provide a prompt (e.g., via a chat GUI element) for the practitioner to provide additional clarity that may improve accuracy of further updates, actions, treatments, or the like. The prompt may be presented in written form, verbal form, sign language form, or combinations thereof. These responses, prompting strategies related to the responses, functions for performing deterministic responses, etc., may be included in response generator data 162.

Data store 140 may further include treatment documentation 165. Treatment documentation 165 may include additional documents related to various aspects of treatment that may be available to an LLM, but are not included in a prompt provided to the LLM. Treatment documentation 165 may include documents that are available for external retrieval by the LLM, e.g., in a retrieval-augmented generation (RAG) process. RAG may provide a route for improved accuracy and reliability of output generated by an AI model such as an LLM. RAG may add an additional external retrieval step to processing performed by an LLM. For example, an LLM may search through an external data source (e.g., treatment documentation 165) to gather relevant passages responsive to obtaining a request. The retrieved relevant passages may be provided into the LLM as additional contexts. The LLM may produce a response to the request based on both the query and the retrieved documentation. In some embodiments, certain output of an LLM may be constrained to the documentation. For example, medical questions may be constrained for safety or regulatory reasons to only be answered with information found within treatment documentation 165. Machine-readable or machine-implementable instructions may be constrained to only include functions that exist within an external library, assessable to the LLM via treatment documentation 165. Updating the documentation may enable an LLM to stay up-to-date without additional training or tweaking of prompting strategies, by updating documents accessible to the LLM in treatment documentation 165.

Treatment provider device 120, dental arch data capturing equipment 126, treatment planning server 112, data store 140, server machine 170, and server machine 180 may be coupled to each other via network 130 for generating treatment protocol data 142, e.g., to generate treatment protocol machine-readable instructions based on natural language practitioner input. In some embodiments, network 130 may provide access to cloud-based services. Operations performed by treatment provider device 120, treatment planning system 110, data store 140, etc., may be performed by virtual cloud-based devices.

In some embodiments, network 130 is a public network that provides treatment provider device 120 with access to the treatment planning server 112, data store 140, and other publicly available computing devices. In some embodiments, network 130 is a private network that provides treatment provider device 120 access to dental arch data capturing equipment 126, data store 140, and other privately available computing devices. Network 130 may include one or more Wide Area Networks (WANs), Local Area Networks (LANs), wired networks (e.g., Ethernet network), wireless networks (e.g., an 802.11 network or a Wi-Fi network), cellular networks (e.g., a Long Term Evolution (LTE) network), routers, hubs, switches, server computers, cloud computing networks, and/or a combination thereof.

Treatment provider device 120 may include computing devices such as Personal Computers (PCs), laptops, mobile phones, smart phones, tablet computers, netbook computers, network connected televisions (“smart TV”), network-connected media players (e.g., Blu-ray player), a set-top-box, Over-the-Top (OTT) streaming devices, operator boxes, etc. Treatment provider device 120 may include a display component 124. Display component 124 may receive user input (e.g., via a Graphical User Interface (GUI) displayed via the treatment provider device 120) of an indication associated with dental arch data. In some embodiments, treatment provider device 120 transmits the indication to the treatment planning system 110, receives output (e.g., treatment protocol data 142) from the treatment planning system 110, determines a treatment protocol or update, and causes the protocol to be displayed to the treatment provider via display component 124.

Treatment planning server 112, server machine 170, and server machine 180 may each include one or more computing devices such as a rackmount server, a router computer, a server computer, a personal computer, a mainframe computer, a laptop computer, a tablet computer, a desktop computer, Graphics Processing Unit (GPU), accelerator Application-Specific Integrated Circuit (ASIC) (e.g., Tensor Processing Unit (TPU)), etc. Operations of treatment planning server 112, server machine 170, server machine 180, data store 140, etc., may be performed by a cloud computing service, cloud data storage service, etc.

Treatment planning server 112 may include a treatment planning component 114. In some embodiments, the treatment planning component 114 may receive instruction data 141, (e.g., receive from the treatment provider device 120, retrieve from the data store 140) and generate output (e.g., treatment protocol data 142) based on the input data. In some embodiments, treatment protocol data 142 may include machine-readable instructions (e.g., computer code, code related to a treatment planning software, or the like) for one or more healthcare disorders.

System 100 may include one or more deterministic or rule-based models, e.g., model 190. A rule-based model may be used to convert preferences input by a doctor to machine-readable instructions (e.g., input by selecting from a set of provided options). A rule-based model may be used to convert machine-readable instructions to natural language, e.g., to perform verification steps. System 100 may include one or more machine leaning or AI models, e.g., model 190. AI models may perform many tasks, including mapping dental arch data to a latent space, splitting an input up into related sections, classifying or categorizing natural language instructions, formatting input text, detecting subject matter, transforming natural language instructions into machine-readable instructions, performing clinical checking, or the like. Model 190 may be trained using dental instruction data. Model 190 may be a general purpose LLM, configured via prompt engineering to perform one or more tasks based on treatment provider input.

One type of machine learning model that may be used to perform some or all of the above tasks is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs).

A recurrent neural network (RNN) is another type of machine learning model. A recurrent neural network model is designed to interpret a series of inputs where inputs are intrinsically related to one another, e.g., time trace data, sequential data, etc. Output of a perceptron of an RNN is fed back into the perceptron as input, to generate the next output.

A graph convolutional network (GCN) is a type of machine learning model that is designed to operate on graph-structured data. Graph data includes nodes and edges connecting various nodes. GCNs extend CNNs to be applicable to graph-structured data which captures relationships between various data points. GCNs may be particularly applicable to meshes, such as three-dimensional data.

Many other types and varieties of machine learning models may be utilized for one or more embodiments of the present disclosure. Further types of machine learning models that may be utilized for one or more aspects include transformer-based architectures, generative adversarial networks, volumetric CNNs, etc. Selection of a specific type of machine learning model may be performed responsive to an intended input and/or output data, such as selecting a model adapted to three-dimensional data to perform operations on three-dimensional models of dental arches, a model adapted to two-dimensional image data to perform operations based on images of a patient's teeth, etc.

Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize a scanning role. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

A large language model (LLM) is a type of AI model designed to understand and generate human-like text, natural language, or the like. LLMs are generally built using deep learning techniques and trained on large datasets from diverse sources. LLMs often provide natural language understanding, text generation, contextual learning, instruction following based on nuanced or detailed prompts, and other functions. LLMs have advantages based on a large extent of knowledge mapped by the layers of the models, as well as an ability to make correct connections between concepts to generate relevant output.

Various types of LLMs may be utilized in embodiments. Autoregressive LLMs generate text sequentially by predicting the next token based on preceding tokens, making them well-suited for text generation tasks. Encoder-decoder LLMs process input through an encoder to create contextual representations and then generate output through a decoder, which may be advantageous for translation or transformation tasks such as converting natural language instructions to machine-readable format. Transformer-based LLMs utilize self-attention mechanisms to process relationships between all tokens in an input sequence simultaneously, enabling capture of long-range dependencies in text. Instruction-tuned LLMs are fine-tuned on datasets of instructions and corresponding outputs, improving their ability to follow specific directives. Retrieval-augmented LLMs combine language generation with information retrieval from external knowledge bases, enabling access to domain-specific documentation during inference.

LLMs operate through several key mechanisms. During training, the model learns statistical patterns and relationships between tokens from large corpora of text data. The model architecture typically includes multiple layers of neural network components including attention layers that determine which parts of the input are most relevant for generating each output token, feed-forward layers that transform representations, and normalization layers that stabilize training. During inference, the model receives an input prompt and generates output by iteratively predicting subsequent tokens based on learned probability distributions. Temperature and sampling parameters may control the randomness and diversity of generated outputs.

In some embodiments, specially trained LLMs may be utilized that are trained or fine-tuned specifically for a target environment such as dentistry or orthodontics. Such specially trained LLMs may be trained on domain-specific corpora including dental literature, orthodontic treatment protocols, clinical terminology, tooth numbering systems, and/or examples of machine-readable treatment instructions. Fine-tuning techniques may include full fine-tuning where all model parameters are updated, parameter-efficient fine-tuning such as low-rank adaptation (LoRA) where only a subset of parameters are modified, or adapter-based approaches where additional trainable layers are inserted into a frozen base model. Specially trained LLMs may exhibit improved accuracy in generating machine-readable dental treatment instructions, better understanding of clinical terminology and abbreviations, and/or more reliable adherence to domain-specific output formats in some embodiments.

Alternatively or additionally, general purpose LLMs may be utilized with specialized prompts to achieve domain-specific functionality without requiring model retraining. Specialized prompts may specify parameters and rules for generating machine-readable instructions for a dental treatment plan, treatment algorithm, or treatment protocol. For example, a prompt may include descriptions of valid instruction categories such as tooth movement instructions, attachment instructions, interproximal reduction instructions, or staging instructions. Prompts may specify examples of natural language instructions and corresponding machine-readable output formats, enabling the LLM to learn the desired transformation through in-context learning. Prompts may specify bounds and thresholds for outputs, such as maximum tooth movement velocities, allowable interproximal reduction amounts, and/or valid stage ranges. Prompts may specify types of outputs including required fields, data types, and schema structures for machine-readable instructions. Prompts may further include descriptions of tooth numbering systems such as Universal Numbering System, Palmer notation, or FDI notation, along with mappings to machine-readable indexing schemes. Through careful prompt engineering, general purpose LLMs may be configured to reliably generate machine-readable dental treatment instructions that conform to specified formats and constraints.

In some embodiments, treatment planning component 114 receives doctor protocol descriptions or treatment plan descriptions in natural language, performs signal processing to break down the current data into sets of current data (e.g., via segmentation or paragraph splitting, which may be provided by an LLM such as model 190), provides the sets of current data as input to a trained model 190, and obtains outputs indicative of treatment protocol data 142 from the trained model 190. In some embodiments, model 190 may represent a single general purpose or specifically trained or adjusted LLM, which may be utilized multiple times using different engineered prompts to perform various tasks in association with a set of doctor protocol description, instructions, input, etc.

In some embodiments, the various models discussed in connection with model 190 (e.g., rule-based model, supervised machine learning model, unsupervised machine learning model, etc.) may be combined in one model (e.g., a hierarchical model), or may be separate models.

Data may be passed back and forth between several distinct models included in model 190 and treatment planning component 114, or provided to a single model multiple times. In some embodiments, some or all of these operations may instead be performed by a different device, e.g., treatment provider device 120, server machine 170, server machine 180, etc. It will be understood by one of ordinary skill in the art that variations in data flow, which components perform which processes, which models are provided with which data, and the like are within the scope of this disclosure.

Data store 140 may be a memory (e.g., random access memory), a drive (e.g., a hard drive, a flash drive), a database system, a cloud-accessible memory system, or another type of component or device capable of storing data. Data store 140 may include multiple storage components (e.g., multiple drives or multiple databases) that may span multiple computing devices (e.g., multiple server computers). The data store 140 may store instruction data 141, treatment protocol data 142, prompt engineering data 144, clinical data 146, and response generator data 162.

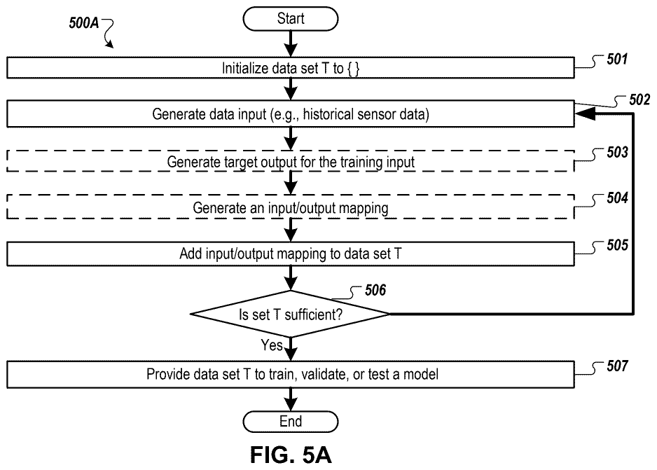

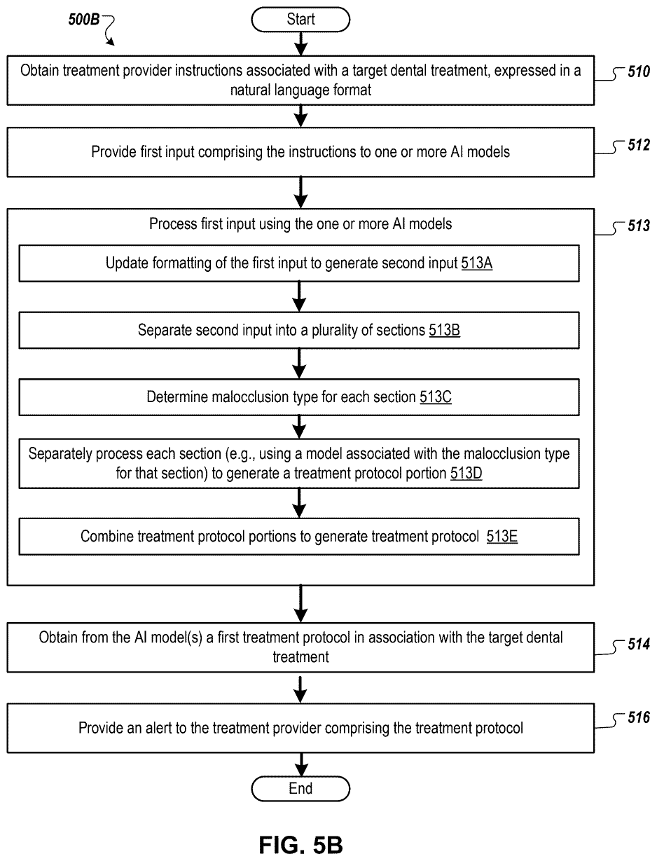

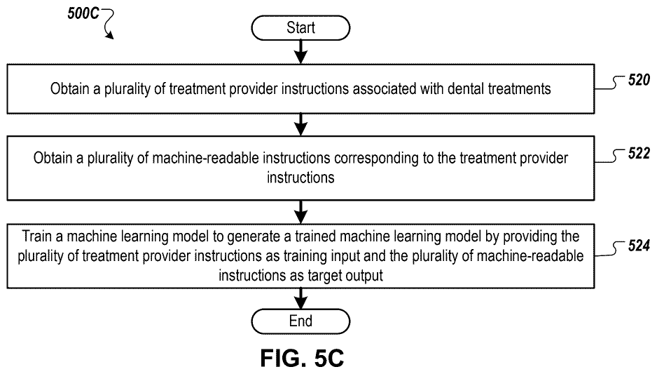

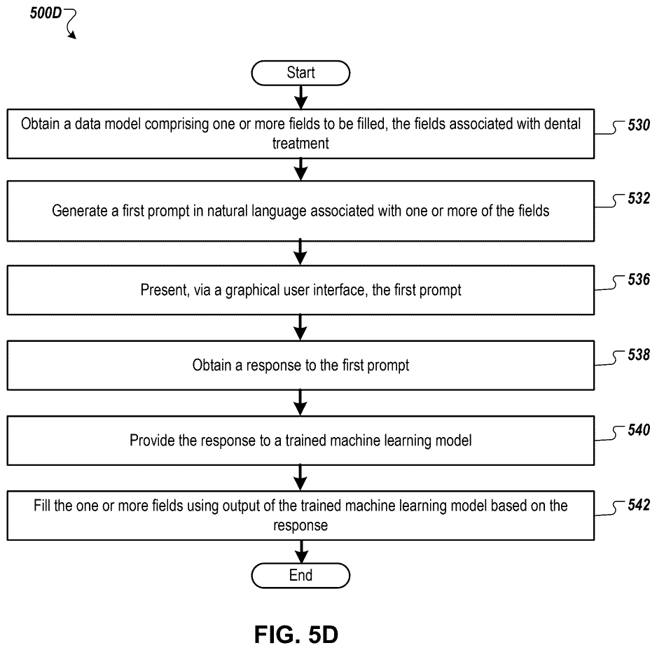

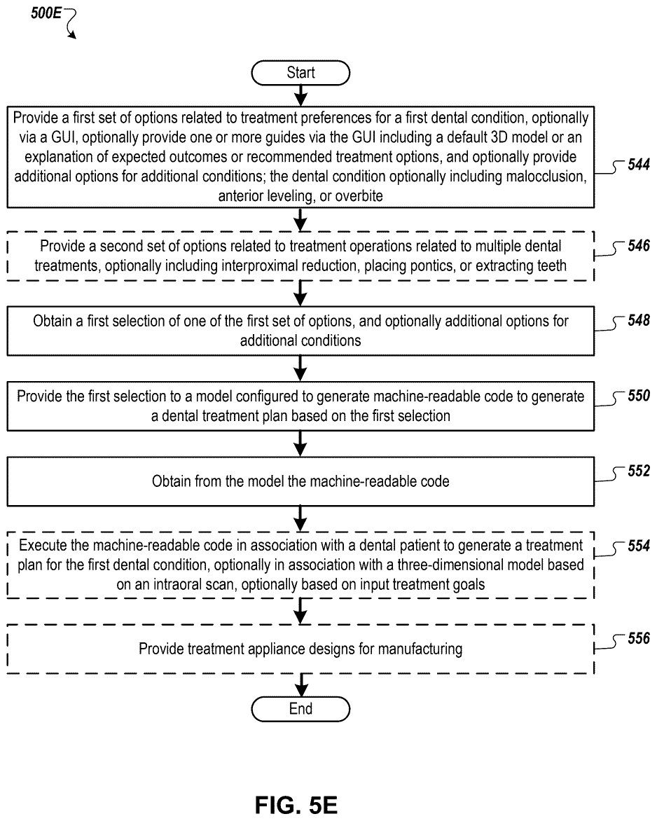

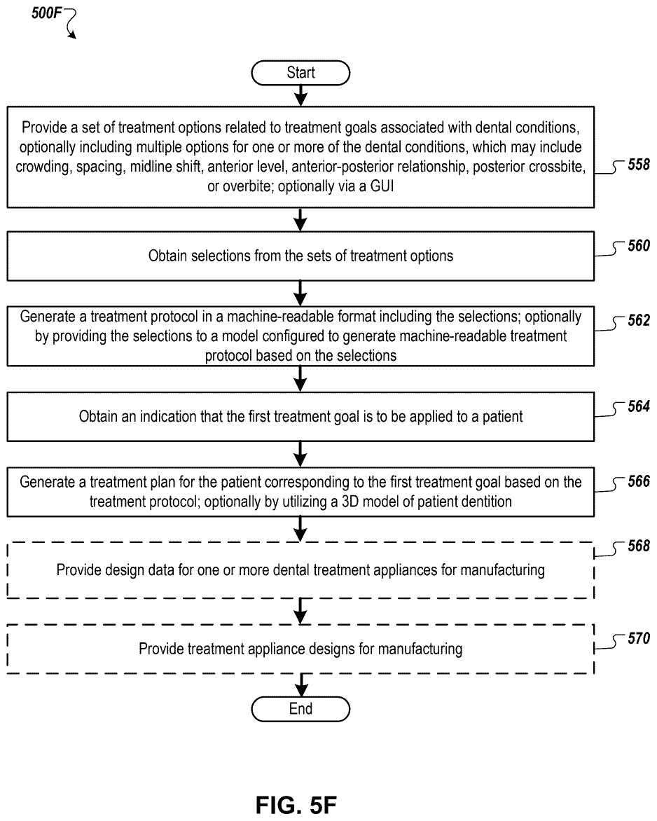

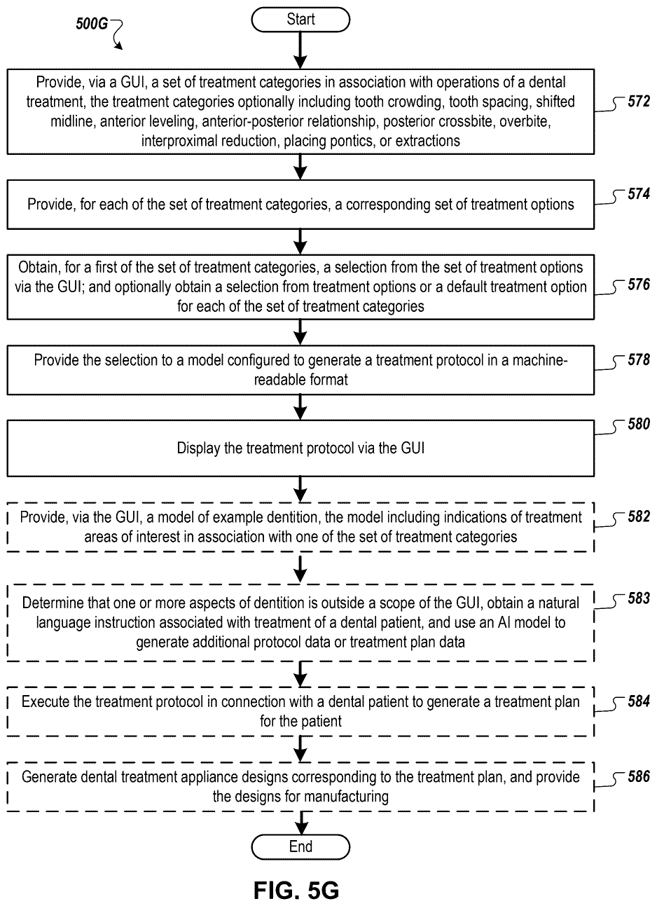

In some embodiments, treatment planning system 110 further includes server machine 170 and server machine 180. Server machine 170 includes a data set generator 172 that is capable of generating data sets (e.g., a set of data inputs and a set of target outputs) to train, validate, and/or test model(s) 190, including one or more machine learning models. Some operations of data set generator 172 are described in detail below with respect to FIG. 5A. In some embodiments, data set generator 172 may partition the historical data (e.g., historical instruction data) into a training set (e.g., sixty percent of the historical data), a validating set (e.g., twenty percent of the historical data), and a testing set (e.g., twenty percent of the historical data).

In some embodiments, treatment planning system 110 (e.g., via treatment planning component 114) generates multiple sets of features. For example a first set of features may correspond to a first subset of instruction data (e.g., data related to a first disorder, first type of treatment, or the like) that correspond to each of the data sets (e.g., training set, validation set, and testing set) and a second set of features may correspond to a second subset of instruction data that correspond to each of the data sets.

In some embodiments, machine learning model 190 is provided historical data as training data. Training data may be used to adjust one or more parameters of an existing model, e.g., to retrain or focus the model for use in a particular application. Various types of retraining schemes may be used, that may be more efficient than fully retraining an AI model, LLM, or the like. The type of data provided will vary depending on the intended use of the machine learning model. For example, a machine learning model may be trained by providing the model with historical doctor inputs as training input. The machine learning model 190 may be configured to generate machine-readable instructions related to doctor instructions. A machine learning model may be provided with corresponding machine-readable instructions, e.g., in a target programming language. Such a machine learning model may be configured to generate mappings (e.g., in a latent space) between natural language instructions and machine-readable instructions.

In one embodiment, server machine 180 includes a training engine 182, a validation engine 184, selection engine 185, and/or a testing engine 186. An engine (e.g., training engine 182, a validation engine 184, selection engine 185, and a testing engine 186) may refer to hardware (e.g., circuitry, dedicated logic, programmable logic, microcode, processing device, etc.), software (such as instructions run on a processing device, a general purpose computer system, or a dedicated machine), firmware, microcode, or a combination thereof. The training engine 182 may be capable of training a model 190 using one or more sets of features associated with the training set from data set generator 172. The training engine 182 may generate multiple trained models 190, where each trained model 190 corresponds to a distinct set of features of the training set (e.g., instruction data from a specific subset of treatment categories, such as disorder types, treatment types, disorder severities, or another categorization). For example, a first trained model may have been trained using all features (e.g., X1-X5), a second trained model may have been trained using a first subset of the features (e.g., X1, X2, X4), and a third trained model may have been trained using a second subset of the features (e.g., X1, X3, X4, and X5) that may partially overlap the first subset of features. Data set generator 172 may receive the output of a trained model (e.g., segmented input data into paragraphs each related with a target topic), collect that data into training, validation, and testing data sets, and use the data sets to train a second model (e.g., a machine learning model configured to perform further operations based on the segmented input data, etc.).

Validation engine 184 may be capable of validating a trained model 190 using a corresponding set of features of the validation set from data set generator 172. For example, a first trained machine learning model 190 that was trained using a first set of features of the training set may be validated using the first set of features of the validation set. The validation engine 184 may determine an accuracy of each of the trained models 190 based on the corresponding sets of features of the validation set. Validation engine 184 may discard trained models 190 that have an accuracy that does not meet a threshold accuracy. In some embodiments, selection engine 185 may be capable of selecting one or more trained models 190 that have an accuracy that meets a threshold accuracy. In some embodiments, selection engine 185 may be capable of selecting the trained model 190 that has the highest accuracy of the trained models 190.

Testing engine 186 may be capable of testing a trained model 190 using a corresponding set of features of a testing set from data set generator 172. For example, a first trained machine learning model 190 that was trained using a first set of features of the training set may be tested using the first set of features of the testing set. Testing engine 186 may determine a trained model 190 that has the highest accuracy of all of the trained models based on the testing sets.

In the case of a machine learning model, model 190 may refer to the model artifact that is created by training engine 182 using a training set that includes data inputs and corresponding target outputs (correct answers for respective training inputs). Patterns in the data sets can be found that map the data input to the target output (the correct answer), and machine learning model 190 is provided mappings that capture these patterns. The machine learning model 190 may use one or more of Support Vector Machine (SVM), Radial Basis Function (RBF), clustering, supervised machine learning, semi-supervised machine learning, unsupervised machine learning, k-Nearest Neighbor algorithm (k-NN), linear regression, random forest, neural network (e.g., artificial neural network, recurrent neural network, CNN, graph neural network, GCN), etc.

Treatment planning component 114 may provide current data to model 190 and may run model 190 on the input to obtain one or more outputs. For example, treatment planning component 114 may provide target instruction data 141 to model 190 and may run model 190 on the input to obtain one or more outputs. Treatment planning component 114 may be capable of determining (e.g., extracting) treatment protocol data 142 from the output of model 190. Treatment planning component 114 may determine (e.g., extract) confidence data from the output that indicates a level of confidence that predictive data (e.g., treatment protocol data 142) is an accurate representation of the natural language instructions provided by the treatment provider. Treatment planning component 114 may use the confidence data to decide whether to cause a corrective action associated with the dental arch, e.g., providing a prompt to the practitioner to provide additional clarity, additional information, or the like.

The confidence data may include or indicate a level of confidence that the treatment protocol data 142 is an accurate prediction associated with the input data. In one example, the level of confidence is a real number between 0 and 1 inclusive, where 0 indicates no confidence that the treatment protocol data 142 is an accurate prediction for the input data and 1 indicates absolute confidence that the treatment protocol data 142 accurately predicts machine-readable instructions associated with the input data. Responsive to the confidence data indicating a level of confidence below a threshold level for a predetermined number of instances (e.g., percentage of instances, frequency of instances, total number of instances, etc.) treatment planning component 114 may cause trained model 190 to be re-trained (e.g., based on an updated pool of training data, etc.). In some embodiments, retraining may include generating one or more data sets (e.g., via data set generator 172) utilizing historical data.

For purpose of illustration, rather than limitation, aspects of the disclosure describe the training of one or more machine learning models 190 using historical data and inputting current data into the one or more trained machine learning models to determine treatment protocol data 142. In other embodiments, a heuristic model, physics-based model, or rule-based model is used to determine treatment protocol data 142 (e.g., without or in addition to using a trained machine learning model. Any of the information described with respect to data inputs to one or more models may be monitored or otherwise used in the heuristic, physics-based, or rule-based model.

In some embodiments, the functions of treatment provider device 120, treatment planning server 112, server machine 170, and server machine 180 may be provided by a fewer number of machines. For example, in some embodiments server machines 170 and 180 may be integrated into a single machine, while in some other embodiments, server machine 170, server machine 180, and treatment planning server 112 may be integrated into a single machine. In some embodiments, treatment provider device 120 and treatment planning server 112 may be integrated into a single machine. In some embodiments, functions of treatment provider device 120, treatment planning server 112, server machine 170, server machine 180, and data store 140 may be performed by a cloud-based service.

In general, functions described in one embodiment as being performed by treatment provider device 120, treatment planning server 112, server machine 170, and server machine 180 can also be performed on treatment planning server 112 in other embodiments, if appropriate. In addition, the functionality attributed to a particular component can be performed by different or multiple components operating together. For example, in some embodiments, the treatment planning server 112 may determine a corrective action based on the treatment protocol data 142. In another example, treatment provider device 120 may determine the treatment protocol data 142 based on output from the trained machine learning model.

The treatment protocol data 142 may be utilized by orthodontic treatment planning software to generate a customized treatment plan for a patient. In some embodiments, the treatment planning software may receive the treatment protocol data 142 and process the data in conjunction with patient-specific dental data (e.g., a three-dimensional model of patient dentition obtained from intraoral scans) to determine a sequence of tooth movements required to achieve a desired final tooth arrangement. The treatment planning process may involve analyzing the patient's initial dental configuration, identifying target positions for each tooth based on the treatment protocol data 142, and calculating intermediate stages between the initial and final configurations. The treatment planning software may generate staging information that defines how teeth are to be repositioned incrementally across multiple treatment stages, with each stage corresponding to a dental appliance configured to move teeth from one arrangement to the next.

In embodiments, a generated treatment protocol may be provided to a treatment planning system and/or treatment management system such as ClinCheck® provided by Align Technology®. A treatment planning system may use digital impressions and/or a treatment protocol or treatment algorithm or pre-generated treatment plan to plan an orthodontic treatment and/or a restorative treatment (e.g., to plan an ortho-restorative treatment). The treatment planning system may plan and simulate orthodontic and/or restorative treatments.

In an example, an orthodontic treatment planning system may use advanced 3D imaging technology to create virtual models of patients' teeth and jaws based on digital impressions or intraoral scans. These digital models may be used to plan and simulate the entire course of orthodontic treatment, including the movement of individual teeth and the progression of treatment over time. Orthodontists can specify the desired tooth movements, treatment duration, and other parameters via a treatment protocol (e.g., by providing such treatment information using natural language instructions and having the natural language instructions converted into machine readable code as described herein), to create personalized treatment plans tailored to each patient's unique anatomy, oral health, and preferences. The orthodontic treatment planning system enables orthodontists to simulate the step-by-step progression of orthodontic treatment virtually, showing patients how their teeth will gradually move and align over the course of treatment. Orthodontists can visualize the planned tooth movements in 3D and make adjustments as needed to optimize treatment outcomes. The orthodontic treatment planning system may provide orthodontists and patients with visualizations of the predicted treatment outcomes, including before-and-after simulations that demonstrate the expected changes in tooth position and alignment, and how those changes might affect the patient's overall oral health. These visualizations help patients understand the proposed treatment plan and make informed decisions about their orthodontic care.

During treatment, updated data may be gathered about a patient's dentition, and such data may be processed, optionally in view of an already generated orthodontic treatment plan, to generate an updated report of the patient's overall oral health and/or to update the treatment plan. This may enable the orthodontic treatment planning/management system to perform informed modifications to the treatment plan.

In some embodiments, the treatment planning process may include receiving a digital representation of a patient's teeth, generating one or more treatment stages based on the digital representation and the treatment protocol data (e.g., which may be automatically generated from natural language instructions provided by a practitioner), and fabricating at least one orthodontic appliance based on the generated treatment stages. The treatment planning software may determine a movement path to move one or more teeth from an initial arrangement to a target arrangement, determine a design for one or more dental appliances shaped to implement the movement path, and generate instructions to fabricate the one or more dental appliances. The treatment protocol data may encode practitioner preferences such as aggression of tooth movement, sequencing of treatment operations, attachment placement preferences, interproximal reduction parameters, and/or other treatment parameters that influence how the treatment plan is generated for a particular patient.

Once a treatment plan has been generated, the treatment plan may be provided to a dental computer aided drafting (CAD) system, such as Exocad® by Align Technology. The dental CAD system may be used for designing dental restorations such as crowns, bridges, inlays, onlays, veneers, and dental implants. The dental CAD system may provide a comprehensive suite of tools and features that enable dental professionals to create precise and customized dental restorations digitally. The dental CAD system may import digital impressions (e.g., 3D digital models of a patient's dental arches) captured using intraoral scanners, and may further import a treatment plan or treatment protocol. The treatment protocol or treatment plan may be used together with a digital impression of a patient's dental arches to develop an appropriate restoration for the patient, for implant planning, for planning of surgery for implant placement, for orthodontic treatment planning, and so on.

In embodiments, the functions of a particular component can be performed by different or multiple components operating together. One or more of the treatment planning server 112, server machine 170, or server machine 180 may be accessed as a service provided to other systems or devices through appropriate application programming interfaces (API).

In embodiments, a “user” may be represented as a single individual. However, other embodiments of the disclosure encompass a “user” being an entity controlled by a plurality of users and/or an automated source. For example, a set of individual users federated as a group of administrators may be considered a “user.”

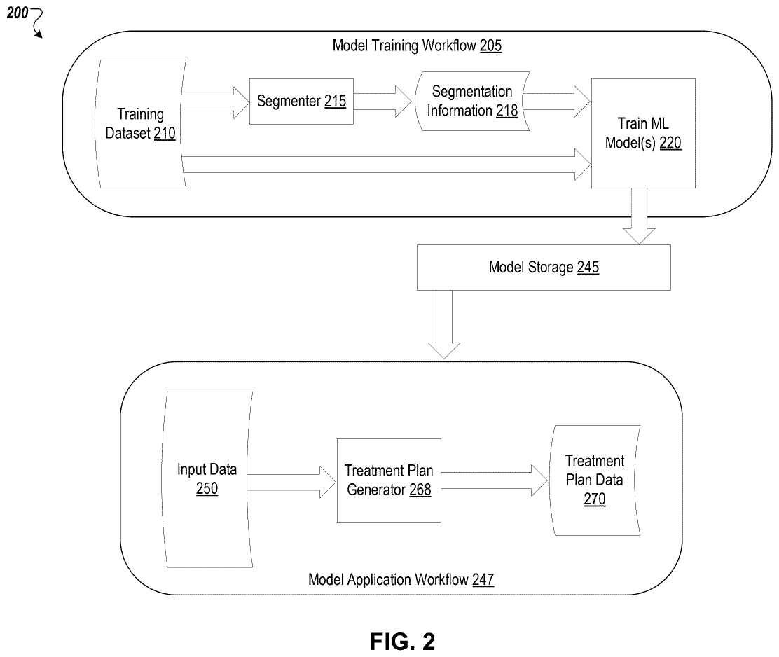

FIG. 2 illustrates workflows 200 for training and implementing one or more machine learning models for performing operations associated with utilizing practitioner instructions to generate treatment protocols treatment algorithms, and/or treatment plans, in accordance with embodiments of the present invention. The illustrated workflows include a model training workflow 205 and a model application workflow 247. The model training workflow 205 is to train or retrain one or more machine learning models (e.g., deep learning models, generative models, LLMS, etc.) to perform one or more data segmentation tasks and/or data generation tasks. The model application workflow 247 is to apply the one or more trained machine learning models to generate treatment protocol data, for instance in the form of machine-readable instructions for protocol generation based on disorder details, based on the input data 250. In some embodiments, the model training workflow may be omitted (e.g., such as where a general purpose LLM is used with specialized prompting focused on dental treatment).

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

The model training workflow 205 and the model application workflow 247 may be performed by processing logic, executed by a processor of a computing device. Workflows 205 and 247 may be implemented, for example, by one or more devices depicted in FIG. 1, such as server machine 170, server machine 180, treatment planning server 112, etc. These methods and/or operations may be implemented by one or more machine learning modules executed on processing devices of devices depicted in FIG. 1.

For the model training workflow 205, a training dataset 210 containing hundreds, thousands, tens of thousands, hundreds of thousands or more examples of input data may be provided. The properties of the input data will correspond to the intended use of the machine learning model(s). For example, a machine learning model for processing natural language instructions to produce machine readable instructions for generating treatment plans may be trained. Training the machine learning model for generating machine readable protocol instructions may include providing a training dataset 210 of natural language input data to be mapped to output machine readable instructions. Training dataset 210 may include additional information, such as contextual information, metadata, etc.

In some embodiments, workflows 200 may be associated with fine-tuning or adjusting a general-purpose artificial intelligence or machine learning model, e.g., for adjusting operations of an LLM to cause the LLM to be more applicable to the target operations of generating treatment protocol instructions based on natural language instruction input. One or more operations as described with respect to model training workflow 205 and/or model application workflow 247 may be performed in a generalized context, e.g., without particular emphasis on input data related to treatment protocol generation. Such a general-purpose AI model (e.g., an LLM) may produce satisfactory results when utilized to generate protocol data in accordance with the present disclosure. In some embodiments, one or more techniques utilizing a training dataset 201 specific to target operations of the model may be used to adjust a generally trained model to perform target tasks with a higher degree of accuracy.

Techniques for adjusting a general-purpose AI model may include parameter-efficient fine-tuning operations. Parameter-efficient fine-tuning is a technique to update AI models in a resource-efficient manner. Parameter-efficient fine-tuning may reduce computational and data volume demands by only updating a subset of a models parameters, e.g., according to methods described with respect to model training workflow 205. Parameter-efficient fine-tuning may include adapter layer tuning, which includes introducing small neural network layers or adapters between existing layers of a pre-trained model. During fine-tuning, only the adapter layers are trained. Parameter-efficient fine-tuning may include lor-rank adaptation techniques, which add low-rank matrices to the weight matrices of the model. During fine-tuning, only these low-rank matrices are updated. Parameter-efficient fine-tuning methods may include bias tuning, where only bias terms are adjusted, prefix-tuning, where learnable vectors are added to input embeddings that are adjusted during fine-tuning, techniques where some parameters are allowed to be updated while others are held static, or other methods for improving performance of a pre-trained AI model.

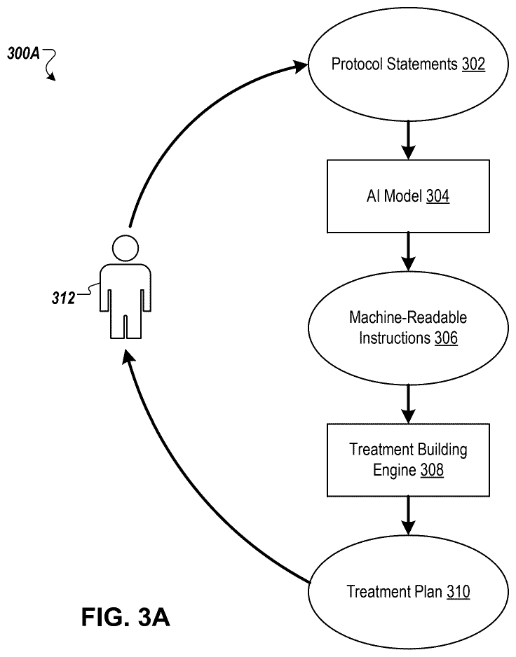

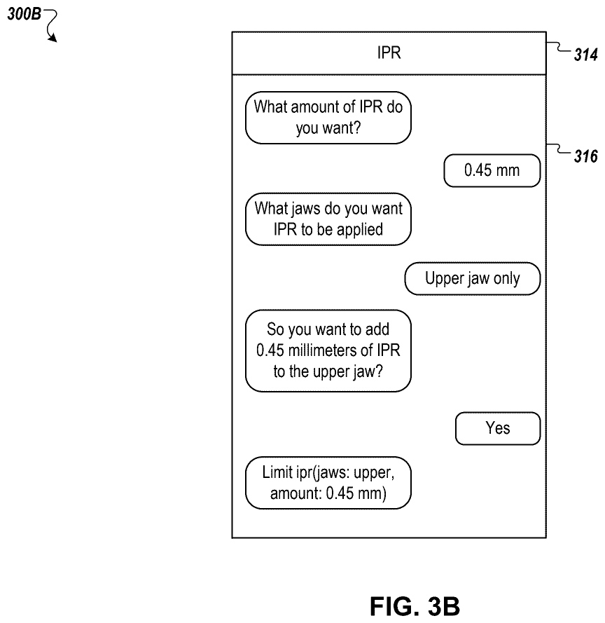

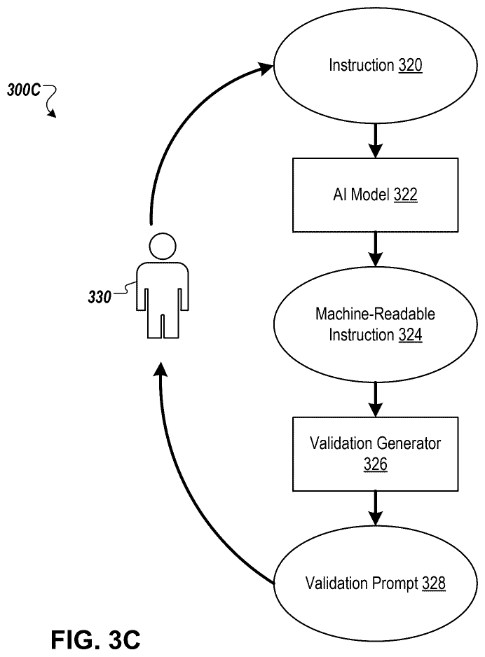

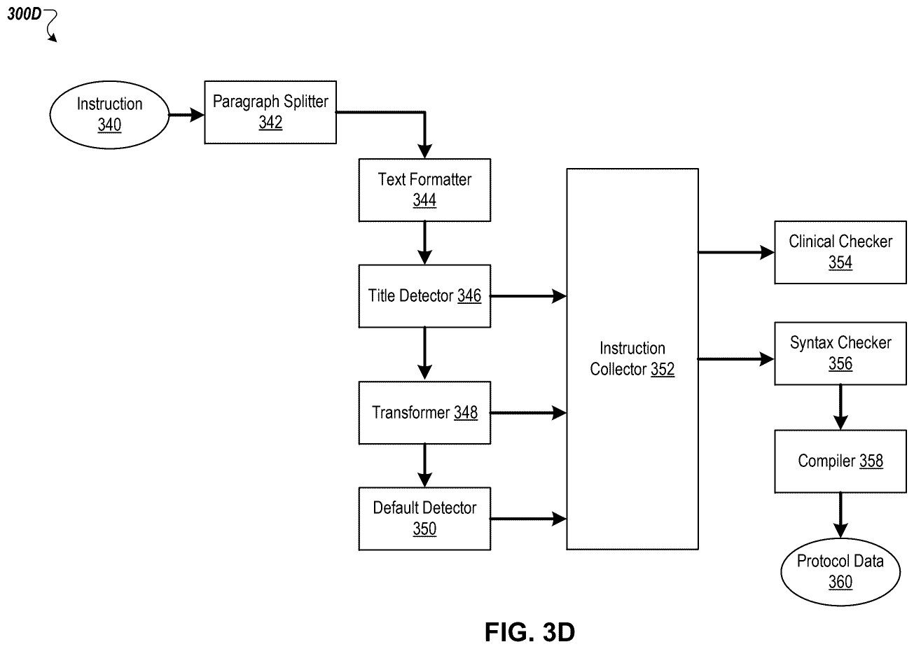

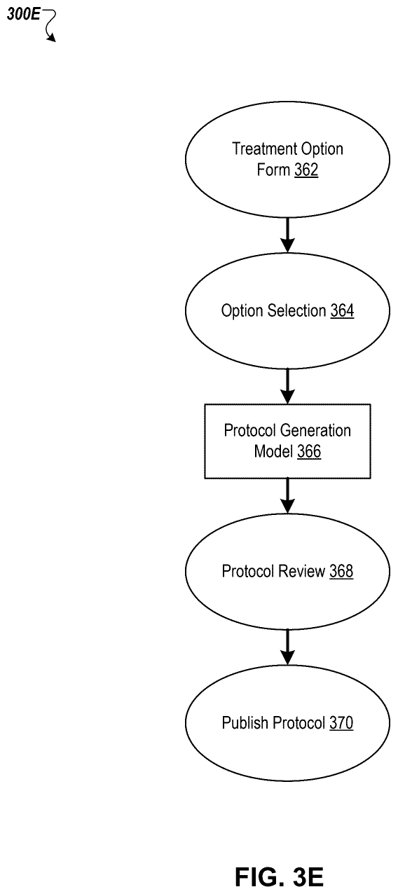

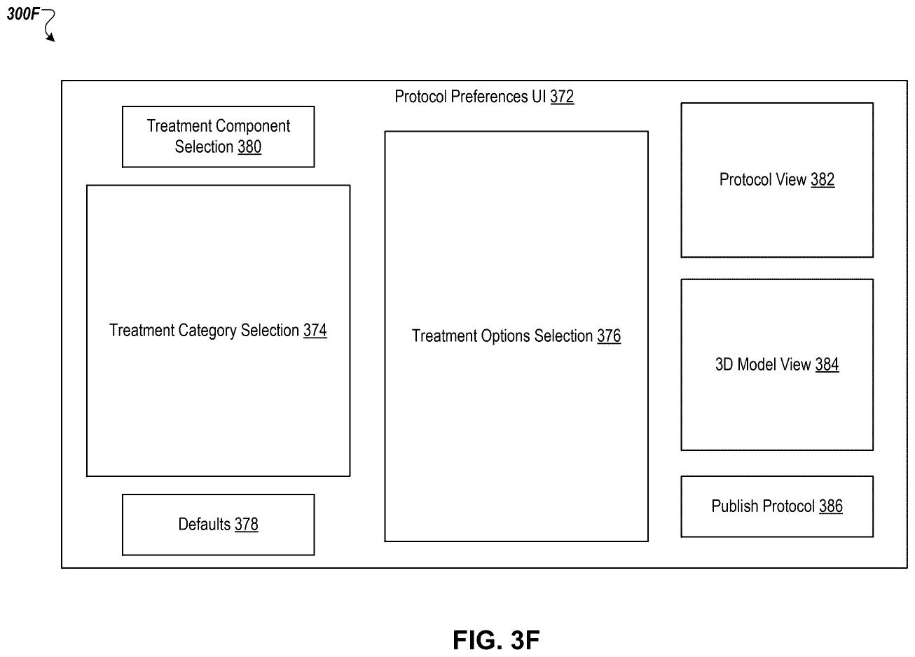

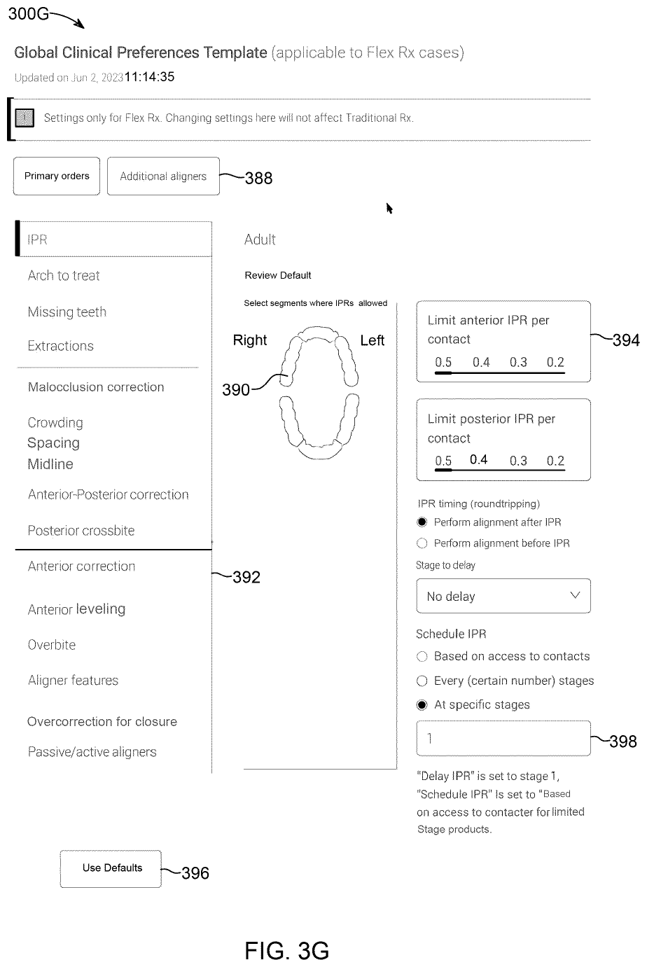

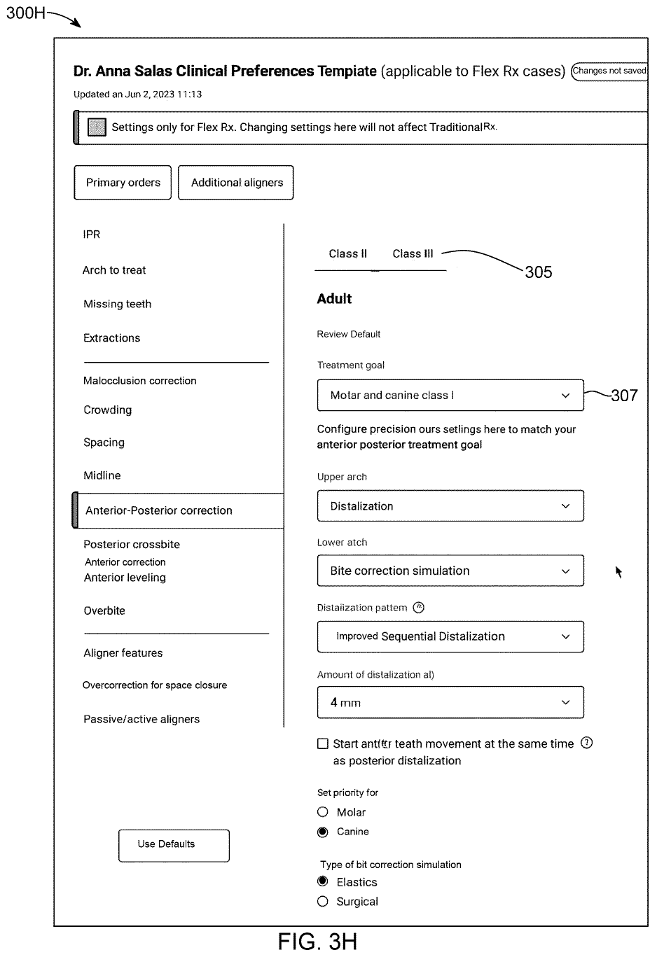

Training dataset 210 may reflect the intended use of the machine learning model. A model may be configured to generate machine-readable protocol instructions. For example, a model may be configured to translate natural language protocol descriptions into machine-readable protocol instructions. The machine learning model configured to predict protocol instructions may be provided with data indicative of one or more natural language and machine-readable instruction pairs as part of training dataset 210. Such a model may be trained to receive a natural language description of a treatment protocol, including practitioner generate treatment guidelines or preferences for a particular healthcare disorder, and generate as output machine-readable instructions that may facilitate generating a specific treatment plan based on disorders of a target patient. In some embodiments, an LLM may be trained (e.g., adjusted, retrained) to perform one or more different tasks with respect to generating machine-readable instructions, such as tasks described with respect to FIG. 3D.

In some embodiments, some or all of the training dataset 210 may be segmented. For example, a model may be trained or configured (e.g., via training adjustments or prompt engineering) to separate input related to multiple topics into sections, each associated with one of the input topics. For example, an input may include descriptions of treatment preferences or protocols related to several disorders, and a segmenter may be used to separate the input into sections related to each target disorder. The segmenter 215 may separate portions of data for training of a machine learning model. For example, individual topics, such as separate disorders or different treatment categories or the like, may be segmented from each other for generating data sets for training a model to generate treatment protocol data.

Data of the training dataset 210 may be processed by segmenter 215 that segments the data of training dataset 210 into multiple different features. The segmenter may then output segmentation information 218. The segmenter 215 may itself be a machine learning model, e.g., a machine learning model configured to separate input into different sections, segments, categories, or the like. Segmenter 215 may be a general purpose LLM, may be an LLM trained specifically to be applicable to dental disorders, an LLM trained specifically to segment natural language instructions related to dental treatment protocols, or the like. In some embodiments, training dataset 210 may not be provided to segmenter 215, e.g., training dataset 210 may be provided to train ML models without segmentation.

In some embodiments, various other pre-processing operations (e.g., in addition to or instead of segmentation) may also be performed before providing input (e.g., training input or inference input) to the machine learning model. Other pre-processing operations may share one or more features with segmenter 215 and/or segmentation information 218, e.g., location in the model training workflow 205. Pre-processing operations may include mesh closing, artifact removal, various text formatters, or other pre-processing that may improve performance of the machine learning models.

Data from training dataset 210 may be provided to train one or more machine learning models at block 220. Training a machine learning model may include first initializing the machine learning model. The machine learning model that is initialized may be a deep learning model such as an artificial neural network. An optimization algorithm, such as back propagation and gradient descent may be utilized in determining parameters of the machine learning model based on processing of data from training dataset 210.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many inputs in the training dataset yields a network that can produce correct output when presented with inputs that are different than the ones present in the training dataset. In high-dimensional settings, such as large images, this generalization is achieved when a sufficiently large and diverse training dataset is made available.

An artificial neural network includes an input layer that consists of values in a data point (e.g., intensity values and/or height values of pixels in a height map). The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer.

Processing logic adjusts weights of one or more nodes in the machine learning model(s) based on an error term. The error term may be based upon a difference between output of the machine learning model and target output provided as part of training dataset 210. For example, the error term may be based upon a difference between output of the machine learning model based on a natural language input, and machine-readable instructions associated with the natural language input (e.g., provided by a subject matter expert). Based on this error, the artificial neural networks adjust one or more of their parameters for one or more of their nodes (the weights for one or more inputs of a node). Parameters may be updated in a back propagation manner, such that nodes at a highest layer are updated first, followed by nodes at a next layer, and so on. An artificial neural network contains multiple layers of “neurons”, where each layer receives as input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

In some embodiments, portions of available training data (e.g., training dataset 210) may be utilized for different operations associated with generating a usable machine learning model. Portions of training dataset 210 may be separated for performing different operations associated with generating a trained machine learning model. Portions of training dataset 210 may be separated for use in training, validating, and testing of machine learning models. For example, 60% of training dataset 210 may be utilized for training, 20% may be utilized for validating, and 20% may be utilized for testing.

In some embodiments, the machine learning model may be trained based on the training portion of training dataset 210. Training the machine learning model may include determining values of one or more parameters as described above to enable a desired output related to an input provided to the model. One or more machine learning models may be trained, e.g., based on different portions of the training data. The machine learning models may then be validated, using the validating portion of the training dataset 210. Validation may include providing data of the validation set to the trained machine learning models and determining an accuracy of the models based on the validation set. Machine learning models that do not meet a target accuracy may be discarded. In some embodiments, only one machine learning model with the highest validation accuracy may be retained, or a target number of machine learning models may be retained. Machine learning models retained through validation may further be tested using the testing portion of training dataset 210. Machine learning models that provide a target level of accuracy in training operations may be retained and utilized for future operations. At any point (e.g., validation, testing), if the number of models that satisfy a target accuracy condition does not satisfy a target number of models, training may be performed again to generate more models for validation and testing.

Once one or more trained machine learning models are generated, they may be stored in model storage 245, and utilized for generating predictive data associated with dental treatment protocols, such as providing predictions of machine-readable instructions associated with natural language protocol descriptions, operations related to a flow to generate the instructions, or the like.

In some embodiments, model application workflow 247 includes utilizing the one or more machine learning models trained at block 220. Machine learning models may be implemented as separate machine learning models or a single combined (e.g., hierarchical, ensemble model, or the like) machine learning model in embodiments.

Processing logic that applies model application workflow 247 may further execute a user interface, such as a graphical user interface. A user may select one or more options using the user interface. Options may include selecting which of the trained machine learning models to use, selecting which of the operations the trained machine learning models are configured to perform to execute, customizing input and/or output of the machine learning models, or the like. For example, a user may only be interested in output of a single or a set of disorders, but may provide doctor-generated natural language input related to a number of disorders or other input that is not relevant to the target output.

Input data 250 is provided to a machine learning model trained in block 220. The input data 250 may correspond to at least a portion or training dataset 210, e.g., be the same type of data, data collected by the same measurement technique, data that resembles data of training dataset 210, or the like. Input data 250 may include new natural language instructions for treatment protocols or treatment plans, e.g., from a new practitioner, a practitioner who wishes to update their standard treatment protocols, etc. Input data 250 may further include ancillary information, metadata, labeling data, etc.

In some embodiments, input data may be preprocessed. For example, preprocessing operations performed on the training dataset 210 may be repeated for at least a portion of input data 250. Input data 250 may include segmented data, data with anomalies or outliers removed, data with manipulated mesh data, or the like.

Input data is provided to treatment plan generator 268. Treatment plan generator 268 generates treatment plan data 270 (e.g., treatment protocol data 142 of FIG. 1, treatment plan data 164 of FIG. 1) based on the input data 250. In some embodiments, treatment plan generator 268 includes a single trained machine learning model. In some embodiments, treatment plan generator 268 includes a combination of multiple trained machine learning models. In some embodiments, treatment plan generator 268 includes multiple applications of input provided to a single trained AI model, e.g., an LLM may be provided various prompts including doctor input to perform multiple operations to facilitate generation of treatment plan data 270. In some embodiments, treatment plan generator 268 may include a combination of machine learning models and other models. For example, a combination of machine learning models and numerical optimization models may be included in treatment plan generator 268. In some embodiments, one or more sets of data may be generated based on protocol data, such as data for generating a treatment plan, data for generating manufacturing plans for one or more appliances for treatment, or the like. In some embodiments, a corrective action may be performed based on the treatment plan data 270. The corrective action may include providing an alert to a user, designing a treatment plan, updating a treatment plan, or the like.

Corrective actions may be associated with design of a treatment protocol, updating of a treatment protocol, providing an alert associated with a treatment protocol to a user, or the like. Corrective actions may be associated with design of a treatment plan, updating of a treatment plan, etc. Corrective actions may be associated with updates to manufacturing data for one or more treatment appliances, updates to manufacturing instructions (e.g., timing of manufacturing) for treatment appliances, etc. Actions performed by system 100 may include generating data for manufacturing of one or more treatment appliances, providing the design data for the treatment appliances to appliance manufacturing 121, manufacturing the appliances, etc.

Various other actions may be performed using a system similar to that depicted and described in connection with FIG. 2, with appropriate adjustments to categories of input data, training data, etc. In some embodiments, model application workflow 247 may include classification of natural language instructions into various categories. For example, some categories of instructions may be provided to a first system for further processing, some categories of instructions may be provided to a second system for further processing, etc. A classification model may take the place of treatment plan generator 268, and the treatment plan data 270 generated may be classification of treatment planning instructions to be used in further actions by the treatment planning system.

In some embodiments, prompt generation may be performed as part of model application workflow 247. For example, descriptions of a target task, related prompting strategies, design principles, examples, etc., may be provided as input data 250 to an AI model such as an LLM. The output treatment plan data 270 may include machine-generated prompts predicted to cause an AI model such as an LLM to perform the target task.

In some embodiments, generation of treatment plan data (e.g., including treatment protocols that express practitioner clinical preferences) may be generated based on a model that is not a machine-learning model, e.g., a rule-based model. For example, a set of options related to dental, orthodontic, or other treatments may be provided via a GUI to the practitioner. The healthcare practitioner may enter, via the GUI, preferences related to various categories of treatment. A model may be used to generate machine-readable instructions expressing a treatment protocol based on the preferences.

FIG. 3A is a diagram depicting a data flow 300A for generation of a treatment plan and/or treatment protocol using an AI model, according to some embodiments. Generation of a treatment plan may include generation of a treatment protocol. For example, generation of a plan for a specific patient may be based on a general protocol for a particular type of disorder. Generation of a treatment plan may be based on one or more statements or instructions provided in natural language by a treatment provider, practitioner, doctor, or the like.

Flow 300A includes protocol statements 302. Protocol statements may be statements made by a practitioner, treatment provider, doctor, or the like with respect to treating one or more disorders. For example, a practitioner associated with orthodontic treatment may generate natural language instructions directed toward methods for treating a number of different disorders that they may regularly encounter, such as a preferred order of operations, a preferred movement or adjustment rate, a preferred technique in association with a target disorder, or the like. The protocol statements 302 may be in natural language and may be related to one or more target disorders, such as multiple dental or orthodontic disorders.

Protocol statements 302 may include a variety of different types of instructions, preferences, and/or directives. Protocol statements 302 may include dental feature instructions, which may specify placement, modification, or removal of attachments, precision cuts, bite ramps, power ridges, or other features applied to teeth or appliances during treatment. Dental feature instructions may specify attachment types such as optimized attachments (including mesial-distal root control attachments, multi-plane attachments, extrusion attachments, rotation attachments, retention attachments, or expansion support attachments), conventional attachments, or protocol-based attachments (such as G4, G5, G7, or G8 protocol attachments). Dental feature instructions may further specify attachment actions including adding attachments, removing existing attachments, keeping existing attachments, replacing existing attachments, modifying existing attachments, forbidding placement of attachments, or delaying placement of attachments. Dental feature instructions may also specify attachment parameters such as size (regular or largest), attachment configuration (single or dual), orientation (distal or mesial), and treatment stages at which attachments are to be placed or removed. Protocol statements 302 may include interproximal reduction (IPR) instructions, which may specify amounts of enamel to be removed between teeth, locations where IPR is to be performed, maximum IPR limits per contact (such as 0.2 mm, 0.3 mm, 0.4 mm, or 0.5 mm for anterior or posterior contacts), staging of IPR operations, and/or whether IPR is to be applied to upper arch, lower arch, or both arches. Protocol statements 302 may include tooth movement instructions, which may specify target positions for individual teeth or groups of teeth, movement sequences, movement rates, movement priorities, and/or constraints on tooth movement. Tooth movement instructions may address specific movement types such as rotation, torque, tip, intrusion, extrusion, translation, and/or root movement. Protocol statements 302 may include arch treatment instructions, which may specify which arches are to be treated (upper only, lower only, or both), coordination between upper and lower arch movements, and/or arch form preferences. Protocol statements 302 may include extraction instructions, which may identify specific teeth to be extracted, timing of extractions relative to treatment stages, and/or space closure protocols following extractions. Protocol statements 302 may include missing teeth instructions, which may identify teeth that are absent and specify how treatment planning should accommodate missing teeth. Protocol statements 302 may include malocclusion correction instructions, which may specify treatment approaches for different malocclusion classes (Class I, Class II, or Class III), treatment goals (such as molar and canine Class I relationships), distalization patterns, amounts of distalization, and/or priorities between molar and canine correction. Protocol statements 302 may include crowding resolution instructions, which may specify methods for resolving crowding such as expansion, IPR, proclination, or extraction, and may indicate preferences for specific teeth or arch regions. Protocol statements 302 may include spacing instructions, which may specify approaches for closing spaces, maintaining spaces, or distributing spaces, and may indicate whether spacing treatment should extend distal to canines or to other landmarks. Protocol statements 302 may include midline instructions, which may specify midline correction goals, methods for achieving midline correction (such as using IPR or tooth movement), and acceptable midline deviation tolerances. Protocol statements 302 may include anterior-posterior correction instructions, which may specify approaches for correcting anterior-posterior discrepancies, including distalization techniques (such as compact sequential distalization or improved sequential distalization), mesialization approaches, and use of elastics or other auxiliaries. Protocol statements 302 may include posterior crossbite instructions, which may specify methods for correcting posterior crossbites, expansion protocols, and coordination between upper and lower arch widths. Protocol statements 302 may include anterior correction instructions, which may address anterior open bite, anterior deep bite, or anterior crossbite correction approaches. Protocol statements 302 may include anterior leveling instructions, which may specify approaches for leveling the curve of Spee, intrusion or extrusion of anterior teeth, and/or coordination with posterior tooth positions. Protocol statements 302 may include overbite instructions, which may specify target overbite values, methods for correcting deep bite or open bite conditions, and use of bite ramps or other features. Protocol statements 302 may include aligner feature instructions, which may specify preferences for passive aligners, active aligners, overcorrection aligners, or retention aligners, and may indicate staging or sequencing of different aligner types. Protocol statements 302 may include overcorrection instructions, which may specify amounts of overcorrection (e.g., for space closure), tooth positions, or other treatment parameters. Protocol statements 302 may include staging instructions, which may specify the number of treatment stages, stage duration, sequencing of movements across stages, and coordination of different treatment operations across the treatment timeline. Protocol statements 302 may include pontic instructions, which may specify placement of pontics in edentulous spaces, pontic designs, and coordination with adjacent tooth movements. Protocol statements 302 may include treatment length instructions, which may specify target treatment duration, maximum number of aligners, or constraints on treatment timeline. Protocol statements 302 may include patient-specific instructions, which may address unique characteristics of individual patients such as teeth that are crowns, implants, or pontics that should not be moved, teeth with root resorption concerns, teeth with periodontal considerations, or other patient-specific factors that may affect treatment planning. Protocol statements 302 may include polite expressions or non-clinical comments, which may include greetings, expressions of gratitude, or other communications that do not contain clinical instructions but may be included in treatment provider communications.

Protocol statements 302 are provided to an AI model 304. The AI model 304 may be an NLP model, an LLM, or the like. The AI model 304 may be a general purpose model (e.g., a general purpose LLM), specifically trained to perform operations related to protocol statements 302, fine-tuned to perform operations related to protocol statements 302, or the like. In some embodiments, additional data may be provided to AI model 304, e.g., via prompt engineering. For example, a prompt may instruct an LLM how to behave, what desired output is, or the like. A prompt may include one or more features of the following example: “you are an AI assistant that helps to convert text description into a C-like programming language for generating treatment protocols.” Prompt engineering may further include a description of target settings or parameters (e.g., maximum response length, temperature, etc.). Prompt engineering may further include examples (e.g., few-shot examples, such as “user input: allow pontics placement with no mesial and distal spaces around them. Response: allow pontics(md_space: 0m)” or other examples which may assist the AI model in generating machine-readable instructions related to treatment protocols. Additional prompt examples may include action-related prompts such as: “user input: add optimized attachments to teeth 3, 4, and 5. Response: add_attachment(teeth: [3, 4, 5], type: optimized)”; “user input: remove existing attachments from upper anteriors. Response: remove_attachment(teeth: upper_anteriors, action: remove_existing)”; “user input: keep attachments on canines from previous treatment. Response: modify_attachment(teeth: canines, action: keep_existing)”; and “user input: delay attachment placement until stage 5. Response: schedule_attachment(stage: 5, action: delay_placement)”. Prompt examples related to attachment types may include: “user input: add largest dual MDRC attachments to tooth 12. Response: add_attachment(teeth: [12], type: optimized, specific_type: mesial_distal_root_control, size: largest, mdrc_type: dual)”; “user input: place rotation attachments on lower incisors. Response: add_attachment(teeth: lower_incisors, type: optimized, specific_type: rotation)”; “user input: add extrusion attachments to premolars. Response: add_attachment(teeth: premolars, type: optimized, specific_type: extrusion)”; and “user input: use G4 protocol attachments on 1.2 and 1.3. Response: add_attachment(teeth: [1.2, 1.3], type: protocol, protocol: G4, specific_type: mesial_distal_root_control)”. Prompt examples related to tooth numbering systems may include: “user input: extract UR4 and UL4. Response: extract(teeth: [UR4, UL4], numbering_system: palmer)”; “user input: no IPR on teeth 2.1 through 2.5. Response: forbid_ipr(teeth: [2.1, 2.2, 2.3, 2.4, 2.5], numbering_system: fdi)”; and “user input: place attachments on 8, 9, 24, and 25. Response: add_attachment(teeth: [8, 9, 24, 25], numbering_system: universal)”. Prompt examples related to interproximal reduction may include: “user input: limit IPR to 0.3 mm per contact on anteriors. Response: limit_ipr(region: anteriors, amount: 0.3 mm)”; “user input: schedule IPR at stage 10. Response: schedule_ipr(stage: 10)”; “user input: allow IPR only distal to canines. Response: allow_ipr(region: distal_to_canines)”; and “user input: no IPR on lower arch. Response: forbid_ipr(arch: lower)”. Prompt examples related to treatment staging may include: “user input: start posterior distalization before anterior movement. Response: set_staging(posterior_distalization: first, anterior_movement: after)”; “user input: add passive aligners at the end of treatment. Response: add_aligners(type: passive, stage: final)”; and “user input: begin attachments at stage 3. Response: schedule_attachment(stage: 3, action: add)”. Prompt examples related to tooth movement restrictions may include: “user input: do not move tooth 14, it is an implant. Response: restrict_movement(teeth: [14], reason: implant)”; “user input: teeth 18 and 19 are pontics, do not include in treatment. Response: exclude_teeth(teeth: [18, 19], type: pontic)”; and “user input: limit movement on crowned teeth 30 and 31. Response: restrict_movement(teeth: [30, 31], reason: crown)”. Prompt examples related to malocclusion correction may include: “user input: achieve Class I molar relationship. Response: set_goal(type: molar_relationship, target: class_I)”; “user input: correct anterior crossbite using elastics. Response: set_correction(type: anterior_crossbite, method: elastics)”; and “user input: distalize upper arch 4 mm. Response: set_distalization(arch: upper, amount: 4 mm)”. Prompt examples related to spacing and crowding may include: “user input: close all spaces using IPR. Response: close_spaces(method: ipr)”; “user input: maintain 2 mm space for future implant at site 19. Response: maintain_space(site: 19, amount: 2 mm, reason: future_implant)”; and “user input: resolve crowding by expansion. Response: resolve_crowding(method: expansion)”. Prompt examples related to overbite and overjet may include: “user input: reduce overbite to 2 mm. Response: set_overbite(target: 2 mm)”; “user input: correct deep bite using bite ramps. Response: correct_deepbite(method: bite_ramps)”; and “user input: reduce overjet by retracting upper anteriors. Response: reduce_overjet(method: retract_upper_anteriors)”. Prompt examples related to midline correction may include: “user input: shift upper midline 1 mm to the right. Response: adjust_midline(arch: upper, direction: right, amount: 1 mm)”; and “user input: achieve coincident midlines using IPR on lower left. Response: adjust_midline(method: ipr, region: lower_left, goal: coincident)”. Prompt examples related to polite or non-clinical expressions may include: “user input: thank you for your help. Response: polite_expression(action: none)”; and “user input: please proceed with the treatment. Response: polite_expression(action: confirm_proceed)”. In some embodiments, multiple prompts related to protocol statements 302 may be provided to one or more AI models to perform various operations as a part of generating machine-readable instructions related to protocol statements 302.

Output of AI model 304 may include machine-readable instructions 306. Machine-readable instructions 306 may be based on treatment protocols, e.g., may be used to generate treatment plans for specific patients or disorders, may be based on general preferences or include instructions to generate a treatment plan when provided data related to a specific patient or disorder, or the like. In various embodiments, machine-readable instructions 306 may comprise sequences of commands, directives, parameters, or data structures that can be interpreted and executed by a computing device, treatment planning engine, or other processing component. Examples of machine-readable instructions include, but are not limited to, executable code segments, configuration files, structured data payloads, treatment parameter specifications, tooth movement directives, attachment placement instructions, interproximal reduction specifications, staging information, and treatment action commands such as add, remove, keep, replace, modify, delay placement, or forbid placement operations associated with dental features or treatment elements. In some embodiments, machine-readable instructions are or represent a treatment protocol.

In some embodiments, the machine-readable instructions 306 may be formatted according to a JSON schema. A JSON schema defines the structure, required properties, allowed values, data types, and relationships between different elements of the instructions, enabling validation and consistent interpretation across different system components. The JSON schema may constrain the output of AI models to ensure that generated instructions conform to expected formats and include only valid, supported operations. For example, a JSON schema may specify allowed values for attachment types, tooth numbering systems, treatment actions, stage specifications, and other treatment parameters, thereby ensuring robustness and correctness of the machine-readable instructions.

In some embodiments, machine-readable instructions 306 may be expressed in a protocol language designed for dental treatment planning. One such protocol language is SPICE-L (Special Instruction Conversion Engine Language), which is a machine-readable JSON format configured to structure free-form and varied instructions from treatment providers into a standardized format suitable for automatic generation of treatment plans. SPICE-L organizes orthodontic instructions into distinct categories, each representing a specific aspect of treatment planning, such as dental features, attachments, tooth movements, staging, and other treatment parameters. The format supports various methods of teeth representation, accommodating different numbering systems (e.g., Universal, Palmer, FDI) and descriptive approaches used by orthodontists. SPICE-L may include an extensible structure allowing for the addition of new categories or instruction types as orthodontic practices evolve. For instructions that do not fit into predefined categories, SPICE-L may include an “other instructions” category to ensure no information is lost. The system may interpret instructions contextually, understanding implied meanings and resolving ambiguities based on orthodontic best practices. Other protocol languages described herein may include IPL (Invisalign Planning Language), which may be designed to represent treatment provider preferences in a machine-readable form for consideration during treatment generation processes. Such protocol languages enable translation of natural language treatment instructions into structured formats that can be processed by treatment planning engines to generate treatment plans automatically.

Machine-readable instructions 306 may be provided to a treatment building engine 308. Treatment building engine 308 may be configured to determine treatment plan 310 based on machine-readable instructions (e.g., a treatment protocol) and data related to a specific patient or disorder. Treatment building engine 308 may be deterministic, e.g., based on machine-readable instructions 306 and a set of rules or commands, rather than being AI or ML-based, in some embodiments. Treatment building engine 308 may generate one or more treatment plans 310, which may be provided to a practitioner 312 for approval. Upon approval, the treatment plans 310 may be enacted, in some embodiments, e.g., data may be provided to a manufacturing facility to generate one or more appliances for treatment of a disorder. Upon practitioner 312 not accepting the treatment plan 310, additional protocol statements may be generated to adjust treatment protocols, adjust machine-readable instructions 306, adjust treatment plan 310, etc., until an acceptable treatment protocol is developed for generating treatment plans for treating patient disorders. In some embodiments, treatment building engine 308 corresponds to, includes, or is in communication with, a treatment planning system and/or treatment management system, such as ExoCAD or ClinCheck, offered by Align Technologies.

In embodiments, a chat interface may be provided to a user for development of a treatment plan and/or a treatment protocol. The chat interface may be an interface to one or more AI models (e.g., one or more LLMs), and may enable a chat agent to interact with a practitioner to develop a dental treatment protocol and/or a dental treatment plan. The chat agent may be implemented as a conversational assistant that receives natural language input from the treatment provider and generates contextually appropriate responses, recommendations, or actions based on the input. The chat agent may utilize one or more LLMs as a foundation for understanding and generating natural language. LLMs are AI models trained on large corpora of text data that learn statistical patterns and relationships between words, phrases, and concepts. When a user provides input to the chat agent, the input text may be tokenized and processed by the LLM, which generates a probability distribution over possible next tokens or responses based on the input context and the model's learned parameters. The LLM may be configured with specific prompts, system instructions, or fine-tuning that constrain or guide its responses toward treatment planning tasks. The chat agent may maintain session context data comprising a history of prior exchanges between the treatment provider and the chat agent, enabling the LLM to generate responses that account for previous instructions, clarifications, and/or preferences expressed during the conversation. The chat agent may be configured to perform multiple functions, including answering questions about treatment options or clinical terminology, generating proposed modifications to treatment protocols or treatment plans based on natural language requests, requesting clarification when instructions are ambiguous or incomplete, and providing explanations of proposed changes in human-readable format. Responses, explanations, and prompts generated by the chat agent may be presented in written form, verbal form via audio output or speech synthesis, sign language form via animated visual representation, or combinations thereof. The chat agent may be constrained to operate within predefined protocol rules, such that output generated by the underlying LLM is limited to modifications that are valid within a predefined set of protocol commands or treatment options. The chat agent may be provided with access to a library of protocol documentation, clinical explanations, or machine-implementable instructions via retrieval-augmented generation, enabling the chat agent to provide accurate and contextually relevant responses based on authoritative documentation rather than solely relying on the LLM's training data.

FIG. 3B depicts a graphical user interface 300B for providing a user experience for a practitioner for generating a treatment protocol using an AI model, according to some embodiments. Graphical user interface (GUI) 300B optionally includes a title section 314 and a chat section 316. In some embodiments, practitioner input may be provided in one or more sections, e.g., a text input field may be provided for a practitioner to input descriptions of preferences for a set of treatment types, a text input field may be provided for each treatment type (e.g., disorder type), or the like. In some embodiments, a chat function may be utilized for incorporating practitioner instructions into protocol instructions. In some embodiments, a data model including a number of fields may be generated. The fields may be filled via methods of the present disclosure to generate machine-readable instructions for treatment protocol generation, e.g., via AI translation of natural language input into machine-readable instructions.

In some embodiments, GUI 300B may be provided in a chat-like configuration, as depicted in FIG. 3B. In such a configuration, different chat elements may be presented for different tasks (e.g., determining protocol for IPR, as indicated in title section 314). In some embodiments, a chat element may be used to determine multiple treatment protocols (e.g., for multiple disorder types). A title element may still be used to assist a practitioner in accurately indicating correct instructions via the chat section 316. In some embodiments, a chat function such as that included in GUI 300B may be utilized as a means of obtaining doctor input for treatment protocol, obtaining AI model input from a practitioner, parsing AI output for generating machine-readable instructions, for filling fields of a data model directed at generating treatment protocol instructions (e.g., a data model may include a number of fields, and a chat function may operate along with one or more AI models to generate prompts and obtain practitioner responses until all fields, target fields, or the like of the data model are filled), or the like.

In some embodiments, GUI 300B may provide machine readable instructions to a practitioner, as indicated in FIG. 3B. In some embodiments, GUI 300B may additionally or alternatively provide the machine-readable instructions to a treatment planning platform (e.g., treatment building engine 308 of FIG. 3A), optionally without providing the machine-readable instructions to the practitioner.

FIG. 3C is a block diagram of a flow 300C for obtaining practitioner instructions via a chat function, according to some embodiments. Flow 300C depicts an example flow of data for generating machine-readable protocol instructions, e.g., via an LLM and a chat function presented to a practitioner by a GUI. Practitioner 330 may provide an instruction 320. Instruction 320 may be or include a description of a treatment protocol, a correction to an existing treatment protocol, or the like. Instruction 320 may be provided based on a prompt, question, or field presented to the practitioner. Instruction 320 may be in natural language.

In some embodiments, instruction 320 may be provided to AI model 322, e.g., an LLM. AI model 322 may be configured (e.g., by training, by prompt engineering, or the like) to generate machine-readable instruction 324 based on the input instruction 320. The machine-readable instructions 324 may be a treatment protocol in some embodiments.

Machine-readable instruction 324 generated by AI model 322 may be provided to validation generator 326. Validation generator 326 may generate one or more validation prompts 328 based on the machine-readable instructions 324. Validation generator 326 may provide a natural language description of an addition or update to the treatment protocol associated with instruction 320. Validation generator 326 may be a deterministic model, e.g., may be rule-based to generate a description of code related to treatment protocol. A validation prompt 328 may comprise a human-readable summary or explanation of the operations encoded in the machine-readable instruction 324, enabling a user to verify that the AI model 322 correctly interpreted the original natural language instruction 320. The validation prompt 328 may describe specific treatment parameters, tooth identifiers, treatment stages, or other clinical details extracted from the instruction 320 and encoded in the machine-readable instruction 324. The validation prompt 328 may be output along with the machine-readable instruction 324 for concurrent review, or may be output separately for sequential verification. In some embodiments, the validation prompt 328 may be output via a graphical user interface, such as within a chat interface or a dedicated validation panel, allowing the user 330 to confirm accuracy or provide corrections before the machine-readable instruction 324 is applied to treatment planning operations.

Validation prompt 328 may be provided to practitioner 330 to obtain a validation response. The validation response may comprise an indication from the practitioner 330 confirming or rejecting the proposed machine-readable instruction 324. The validation response may include an affirmative response indicating that the machine-readable instruction 324 correctly captures the intent of the original natural language instruction 320, a negative response indicating that the machine-readable instruction 324 does not accurately reflect the practitioner's intent, a modification request specifying adjustments to be made to the machine-readable instruction 324, or a clarification providing additional context or details to refine the instruction. The validation response may be provided via the chat interface, through selection of confirmation or rejection options presented in a GUI, or through free-text entry allowing the practitioner 330 to elaborate on desired changes. The validation prompt 328 may be presented in written form, verbal form via audio output or speech synthesis, sign language form via animated visual display, or combinations thereof. The validation response may be utilized as instruction 320 to further update the protocol, update the machine-readable instruction 324, or the like. For example, an affirmative validation response may cause the machine-readable instruction 324 to be enacted, included in the protocol, added to the data model, or the like, and a further field to be investigated by the chat function. A negative validation response may cause the AI model to generate further updates, to provide a prompt to practitioner 330 for clarification, to present alternative interpretations of the original instruction 320, or to request additional information to resolve ambiguities in the treatment protocol specification.

As an example, a practitioner 330 may choose to update a treatment protocol using a chat function. The practitioner 330 may provide as instruction 320 a target update in natural language related to a treatment protocol, for example, an instruction to not place attachments on target teeth for an orthodontic treatment. The AI model may generate an update or command based on the instruction, for example, a command to disable attachments on target teeth. The response generator may, based on the machine-readable command, generate a validation prompt in natural language which may be provided to the practitioner for validation. For example, in response to a machine-readable command to not use attachments on target teeth, a validation prompt such as “did you mean to disable attachments on all second molars?” may be provided to the practitioner. The practitioner may determine whether the description of the proposed addition to the protocol is appropriate, respond accordingly, and continue exchanging messages with the chat function until a target set of data has been obtained (e.g., until target fields of a data model related to treatment protocols have been filled and validated).

FIG. 3D is a block diagram of a data flow 300D for generating machine-readable instructions based on practitioner natural language instructions, according to some embodiments. In some embodiments, one or more operations depicted in FIG. 3D may not be utilized, multiple operations may be combined, etc. In some embodiments, one or more operations of flow 300D may be performed by purpose-trained AI models, multiple operations may be performed by the same model (e.g., an LLM configured to perform multiple different operations by prompt engineering), one or more operations may be performed by a general purpose LLM that is provided sufficient context (e.g., prompt) to perform the operations, or the like.

Instructions 340 are provided to an AI model. The instructions 340 may be doctor or practitioner instructions related to a treatment protocol, presented in natural language. In some embodiments, the instructions may be entered via a GUI, via a text entry field, via a chat function, via a data model, or the like. Instructions 340 may be provided to paragraph splitter 342, which may be or include an AI model (e.g., LLM). In some embodiments, paragraph splitter 342, text formatter 344, title detector 346, transformer(s) 348, default detector 350, clinical checker 354, and/or other operative functions may be performed by one or more agents that provide input to an AI model (e.g., LLM) and obtains output from the AI model for further operations. In some embodiments, e.g., if multiple protocols are expected to be included in instructions 340, instructions 340 may be provided to paragraph splitter 342. The paragraph splitter 342 may analyze the natural language instructions to identify logical boundaries between distinct topics, treatment categories, and/or clinical directives. The paragraph splitter 342 may utilize semantic analysis to detect transitions between different subject matters within the instructions, such as identifying when a practitioner's notes shift from discussing one dental condition to another, one tooth or set of teeth to another, and so on. In some embodiments, the paragraph splitter 342 may identify section boundaries based on linguistic cues including transitional phrases, changes in referenced anatomical structures (e.g., shifting from upper arch to lower arch discussions), changes in treatment modalities (e.g., from attachment placement to interproximal reduction), or explicit section headers or numbering provided by the practitioner. The paragraph splitter 342 may be configured via prompt engineering to recognize domain-specific patterns in orthodontic or dental instructions, such as identifying when instructions transition between different malocclusion types, different treatment phases, or different patient populations (e.g., adult versus teen cases). In some embodiments, the paragraph splitter 342 may output structured data indicating the boundaries of each identified section along with preliminary categorization information that assists downstream processing components. Sections of the input data may be separated into topics, e.g., based on prompt engineering and provided natural language instructions. In some embodiments, each section may be associated with a disorder, e.g., one section for deep bite correction, one for open bite correction, etc. Each section (e.g., paragraph) may be processed separately for subsequent operations, e.g., to generate a protocol for each disorder separated by the paragraph splitter 342.

Input is provided to text formatter 344. Input may include one section separated by paragraph splitter 342, as well as prompt engineering input configured for an LLM to perform target operations. Input to each module (e.g., by an agent for interfacing with an LLM) of flow 300D may include prompt engineering to configure an LLM to perform target operations. Text formatter 344 may be configured to improve text processability, e.g., by resolving abbreviations, attempting to resolve typographical errors, unifying units, scale, indexing, or other metrics, etc.

In some embodiments, text formatter 344 performs normalization operations on dental terminology and tooth numbering systems. Treatment providers may express tooth identifiers using different numbering conventions, such as Universal Numbering System (UNS), Palmer notation, or Fédération dentaire internationale (FDI) notation. Text formatter 344 may be configured to detect which numbering system is being used in the input text and normalize tooth references to a standardized format for downstream processing. For example, a treatment provider instruction referencing “tooth #3” in Universal notation, “UR6” in Palmer notation, or “1.6” in FDI notation may all refer to the same tooth, and text formatter 344 may convert these references to a unified representation. Text formatter 344 may also resolve variations in dental terminology, such as recognizing that “MDRC,” “mesial distal root control,” and “root control attachment” refer to the same type of optimized attachment. Additionally, text formatter 344 may handle variations in how treatment providers express attachment-related terms, including misspellings, abbreviations such as “att” for “attachment,” or colloquial expressions commonly used in clinical practice.

Text formatter 344 may further be configured to handle multilingual input and standardize formatting conventions across different languages. In some embodiments, treatment provider instructions may be provided in languages other than English, and text formatter 344 may translate or transliterate such instructions to a standardized language format for subsequent processing by downstream components. Text formatter 344 may also normalize stage references and temporal expressions used by treatment providers. For example, instructions may reference treatment stages using various formats such as literal stage numbers (e.g., “stage 5”), relative references (e.g., “first stage,” “last stage,” “final aligner”), or offset expressions (e.g., “3 stages before the end”). Text formatter 344 may convert these varied expressions into a consistent machine-readable format. Furthermore, text formatter 344 may identify and separate polite expressions, greetings, or other non-clinical content from substantive treatment instructions, flagging such content for exclusion from clinical processing while preserving the clinically relevant portions of the input for further analysis by subsequent components in flow 300D.

Input may be provided to title detector 346. Title detector 346 may be configured to predict a topic of input text, e.g., a topic of a section split by paragraph splitter 342. Title detector 346 may generate an understanding of a disorder or topic of a paragraph or section of input text. Detecting and applying a topic title may improve performance of further operations of flow 300D for generating protocol data 360.

In some embodiments, title detector 346 may be implemented as an AI model, such as an LLM, configured to analyze the semantic content of input text and assign appropriate category labels or topic identifiers. Title detector 346 may identify key concepts, dental conditions, treatment categories, malocclusion types, or other clinically relevant subject matter associated with a section of treatment provider instructions. For example, title detector 346 may determine that a particular section of input text relates to interproximal reduction (IPR) parameters, attachment placement preferences, spacing treatment goals, overbite correction, or anterior-posterior correction. The identified title or topic may be used to route the section to an appropriate transformer 348 or other downstream processing component that is specialized for handling instructions of that particular category. Title detector 346 may utilize pattern recognition, keyword analysis, contextual understanding, or combinations thereof to accurately classify input text sections.

Title detector 346 may be configured to handle ambiguous or multi-topic sections of input text. In some cases, a section of treatment provider instructions may relate to multiple treatment categories or may include instructions that span different aspects of dental treatment. Title detector 346 may assign multiple topic labels to such sections, or may flag sections for further analysis when the topic cannot be determined with sufficient confidence. In some embodiments, title detector 346 may generate confidence scores associated with topic predictions, enabling downstream components to handle low-confidence predictions differently than high-confidence predictions. Title detector 346 may also be configured to identify sections that do not correspond to any predefined treatment category, which may indicate novel instruction types, non-clinical content, or instructions requiring manual review. The output of title detector 346 may include structured metadata that accompanies the input text through subsequent processing stages, enabling transformer 348 and other components to leverage the topic information when generating machine-readable instructions.

Input may be provided to one or more transformers 348. Transformer(s) 348 may include multiple transformers, multiple AI models, multiple iterations of providing input data and/or prompt engineering input to an LLM, etc. In some embodiments, different transformers included in transformer(s) 348 may be configured to be applied to different topics, e.g., attachments or other categories of commands. Transformers 348 may be configured to transform one or more natural language instructions to machine-readable instructions, e.g., of a proprietary language related to a treatment planning software. Different transformers may be configured to enable transformation of different types of instructions. In some embodiments, an updated instruction may be generated based on removing statements from the provided instructions associated with machine-readable instructions generated by the one or more transformers 348.

The output of transformer(s) 348 may include machine-readable instructions structured in a standardized format, such as a JSON schema or other text-based schema. The structured output may organize orthodontic instructions into distinct categories, each representing a specific aspect of treatment planning. For example, the output may include fields specifying an action to be performed (e.g., add, remove, keep, replace, modify, forbid placement, or delay placement), teeth identifiers indicating which teeth are affected by the action, attachment types specifying the type of dental feature to be applied, treatment stages at which the action is to be performed, and additional parameters such as attachment size, position, or orientation. The output format may support various methods of teeth representation, accommodating different numbering systems such as Universal, Palmer, and FDI numbering systems used by orthodontists. A standardized schema may define the structure, allowed values, and relationships between different elements of the format, constraining the transformer output to only include modifications that are valid within a predefined set of protocol commands.

In some embodiments, transformer(s) 348 may be configured to extract specific parameters from natural language instructions based on the category of instruction being processed. For attachment-related instructions, the transformer may extract attachment type information including general types (e.g., optimized attachments, conventional attachments, protocol attachments) and specific types (e.g., mesial distal root control, multi-plane, extrusion, rotation, retention, or expansion support attachments). The transformer may further extract optional parameters such as attachment size (e.g., regular or largest), attachment configuration (e.g., single or dual for root control attachments), and directional information (e.g., distal, mesial, horizontal, or vertical). For instructions that do not fit into predefined categories, the transformer output may include an “other instructions” category to ensure no information is lost during the transformation process. The transformer may interpret instructions contextually, understanding implied meanings and resolving ambiguities based on orthodontic best practices. The output structure may be extensible, allowing for the addition of new categories or instruction types as orthodontic practices evolve or as additional treatment parameters are supported by the treatment planning system.

In some embodiments, different transformers may be configured to handle different types of treatment planning operations or parameters. Each transformer may be specialized to process a particular category of natural language instructions and generate corresponding machine-readable instructions optimized for that category. The system may include multiple transformers operating in parallel or in sequence, with each transformer receiving instructions that have been classified as belonging to its designated category. The transformers may include an attachment transformer configured to process instructions related to dental attachments, including placement, removal, modification, and configuration of optimized attachments, conventional attachments, and protocol-based attachments such as G4, G5, G7, and G8 protocol attachments. The transformers may further include an interproximal reduction (IPR) transformer configured to process instructions related to IPR operations, including IPR amounts, locations, timing, and scheduling across treatment stages. The transformers may include a tooth movement transformer configured to process instructions related to tooth repositioning, including translation, rotation, torque, tip, intrusion, extrusion, and other movement parameters. The transformers may include a staging transformer configured to process instructions related to treatment staging, including stage sequencing, passive aligners, active aligners, and timing of treatment operations across stages. The transformers may include an extraction transformer configured to process instructions related to tooth extractions, including identification of teeth to be extracted and timing of extractions relative to treatment stages. The transformers may include a spacing transformer configured to process instructions related to space management, including space closure, space maintenance, and distribution of spacing across the dental arch. The transformers may include a midline transformer configured to process instructions related to midline correction, including midline goals, correction methods, and prioritization of upper versus lower midline alignment. The transformers may include an anterior-posterior correction transformer configured to process instructions related to Class II and Class III malocclusion correction, including distalization, mesialization, and bite correction parameters. The transformers may include an overbite transformer configured to process instructions related to overbite and deep bite correction, including intrusion and extrusion parameters for anterior teeth. The transformers may include a crossbite transformer configured to process instructions related to posterior crossbite correction, including expansion and constriction parameters. The transformers may include a crowding transformer configured to process instructions related to crowding resolution, including methods for gaining space such as expansion, proclination, IPR, and extraction. The transformers may include an overcorrection transformer configured to process instructions related to overcorrection parameters for various tooth movements to account for relapse tendencies. The transformers may include a precision cuts transformer configured to process instructions related to precision cuts in aligners, including cut locations and configurations. The transformers may include a bite ramps transformer configured to process instructions related to bite ramp placement and configuration. The transformers may include a power ridge transformer configured to process instructions related to power ridge features in aligners. The transformers may include a finishing transformer configured to process instructions related to treatment finishing, including final tooth positions and refinement parameters.

In some embodiments, the different transformers may be implemented as distinct specially trained AI models, such as distinct large language models (LLMs) that have been fine-tuned or trained specifically for their designated category of instructions. Each specially trained AI model may be trained on a dataset comprising natural language instructions within its designated category paired with corresponding machine-readable instructions, enabling the model to learn the specific vocabulary, patterns, and output formats associated with that category. For example, an attachment transformer may be implemented as an LLM that has been fine-tuned on a dataset of attachment-related instructions and corresponding JSON-formatted attachment specifications, while a staging transformer may be implemented as a separate LLM that has been fine-tuned on staging-related instructions and corresponding stage configuration outputs. The specially trained AI models may be smaller, more efficient models that are optimized for their specific tasks, enabling faster inference and reduced computational requirements compared to general-purpose models. In some embodiments, the specially trained AI models may be based on architectures such as encoder-decoder models, text-to-text models, or other transformer-based architectures that are well-suited for the task of converting natural language to structured output formats.

In some embodiments, the different transformers may be implemented using one or more general-purpose AI models, such as one or more LLMs, that are provided with different specialized prompts to configure their behavior for different categories of instructions. Each specialized prompt may include a system message or instruction set that defines the task, specifies the expected input format, describes the target output schema, provides examples of input-output pairs, and includes rules for handling edge cases and ambiguities specific to that category. For example, an attachment transformer may be implemented by providing a general-purpose LLM with a specialized attachment prompt that describes the types of attachment actions (add, remove, keep, replace, modify, forbid placement, delay placement), the attachment type taxonomy (optimized attachments, conventional attachments, protocol attachments), the specific attachment subtypes (mesial distal root control, multi-plane, extrusion, rotation, retention, expansion support), and the expected JSON output schema for attachment instructions. Similarly, an IPR transformer may be implemented by providing the same or a different general-purpose LLM with a specialized IPR prompt that describes IPR parameters, scheduling options, and the expected output format for IPR instructions. The specialized prompts may be developed through iterative refinement processes, where initial prompts are tested against representative datasets and refined based on analysis of false positives, false negatives, and edge cases.

In some embodiments, the different transformers may be implemented using one or more AI models that are provided with different specialized context or access to specific information via retrieval-augmented generation (RAG) or similar techniques. Each transformer may be configured to retrieve and incorporate relevant documentation, reference materials, or domain-specific knowledge bases when processing instructions within its designated category. For example, an attachment transformer may be provided with access to a library of attachment specifications, including detailed descriptions of each attachment type, clinical indications for each attachment, placement guidelines, and examples of properly formatted attachment instructions. A staging transformer may be provided with access to staging guidelines, movement rate limits, and sequencing rules that inform how treatment stages should be configured. A protocol transformer may be provided with access to protocol documentation describing the specific requirements and constraints of various treatment protocols, such as G4, G5, G7, and G8 protocols, including which teeth are eligible for each protocol and what attachment configurations are required. The RAG-based approach may enable transformers to access up-to-date information without requiring retraining, as the retrieved documentation can be updated independently of the underlying AI model. The retrieved context may be incorporated into the prompt provided to the AI model, enabling the model to generate outputs that are consistent with the retrieved documentation and constrained to valid options defined in the documentation. In some embodiments, the AI model may be configured to limit its output to instructions and parameters that are explicitly defined in the retrieved documentation, preventing the generation of invalid or unsupported instructions.

In some embodiments, input may be provided to default detector 350. Input to default detector 350 may include natural language input. Default detector 350 may be configured to identify statements that do not correspond to or require machine-readable equivalents. In some embodiments, only a portion of input may be provided to default detector 350, e.g., only inputs that are not associated with machine-readable statements that were generated by transformer 348. In some embodiments, additional input statements may be provided (e.g., all input) and tracking may be applied to determine whether every statement was either flagged as a default (e.g., not relevant to machine-readable protocol instruction) or transformed to an instruction. For example, a practitioner may provide a statement that is not clinically relevant (e.g., a “thank you” statement), or a statement that has clinical relevance but is not relevant for machine-readable protocol instructions (e.g., attachments for orthodontic care may be automatically included based on various other commands, a statement by a practitioner such as “add attachments as necessary” may be disregarded by default detector 350). If any statements are not accounted for (e.g., either transformed to machine-readable format or identified by default detector as not relevant to machine-readable instructions), further operations may be performed, e.g., the statements may be provided to an LLM configured to generate a prompt asking a practitioner for clarity, the statements may be provided to a sequence of LLM operations to attempt to generate machine-readable instructions again, the entire instruction 340 may be reprocessed, a fault or error may be indicated, or the like.

Output of one or more AI models (e.g., title detector 346, transformer(s) 348, and/or default detector 350) may be provided to instruction collector 352. Instruction collector 352 may be configured to collect and/or arrange instructions from various sources to generate a treatment protocol associated with a particular topic or set of topics, e.g., labeled in accordance with output of title detector 346.

Output of instruction collector 352 may be provided for one or more checks. Checkers may determine whether the instructions correlate to the input instructions (e.g., an LLM may be provided with a prompt to predict whether the machine-readable instructions are an accurate representation of the natural language instructions they are based on). Checkers may determine if the code is compilable. Clinical checker 354 may be or include an AI model. Clinical checker 354 may increase reliability of the process by configuring an LLM to predict whether the instructions provided by instruction collector 352 correspond to the instructions 340. Clinical checker 354 may further ensure that various safety provisions are adhered to, e.g., maximum safe treatment times, distances, procedures, or the like.

Clinical checker 354 may perform semantic validation to verify that the machine-readable instructions accurately capture the clinical intent expressed in the original natural language instructions. In some embodiments, clinical checker 354 may receive both the original natural language instructions 340 and the corresponding machine-readable instructions generated by transformer(s) 348, and may utilize an AI model to assess whether the machine-readable instructions faithfully represent the treatment provider's intent. Clinical checker 354 may identify discrepancies between the natural language input and the generated machine-readable output, such as incorrect tooth identifications, misinterpreted treatment actions, or omitted instructions. In some embodiments, clinical checker 354 may generate a confidence score indicating the likelihood that the machine-readable instructions correctly represent the natural language instructions. Instructions that fall below a confidence threshold may be flagged for manual review or may be routed to manual treatment planning 418. Clinical checker 354 may further validate that the generated instructions are compatible with the treatment planning engine and conform to expected schemas or formats required for downstream processing.

Clinical checker 354 may apply a set of clinical safety heuristics to the machine-readable instructions to ensure that proposed treatment operations do not violate established clinical guidelines or safety constraints. The clinical safety heuristics may include constraints related to maximum tooth movement distances per treatment stage, maximum interproximal reduction amounts per contact, restrictions on attachment placement for certain tooth types, and limitations on treatment duration. Clinical checker 354 may access clinical data 146 to retrieve applicable safety thresholds and treatment constraints. In some embodiments, clinical checker 354 may determine whether proposed attachment placements are appropriate for the specified teeth, such as verifying that protocol-based attachments are applied only to eligible tooth types (e.g., G4 protocol attachments applied only to canines or incisors, G5 protocol attachments applied only to premolars). Clinical checker 354 may further validate that treatment actions specified in the machine-readable instructions are supported by the treatment planning system and that all required parameters are present and within acceptable ranges. Instructions that fail clinical validation may be rejected, flagged for review, or routed to manual treatment planning for resolution by a trained technician.

Syntax checker 356 may be used to determine that machine-readable instructions are formatted correctly, may determine that the instructions are compilable, may recommend one or more updates based on predicted target instructions, or the like. The syntax checker 356 may validate that the machine-readable instructions conform to a predefined schema or grammar associated with the treatment planning system. The syntax checker 356 may parse the machine-readable instructions to identify structural errors, missing required fields, invalid parameter values, or malformed expressions. In some embodiments, the syntax checker 356 may compare the machine-readable instructions against a library of valid instruction formats and flag any instructions that do not match expected patterns. The syntax checker 356 may verify that tooth identifiers conform to supported numbering systems, that stage references are within valid ranges, that attachment types correspond to recognized categories, and that action keywords match predefined operations such as add, remove, keep, replace, or modify. The syntax checker 356 may also perform type checking to ensure that parameter values are of expected data types, such as verifying that numerical values are provided where numbers are expected and that string values conform to enumerated options. In some embodiments, the syntax checker 356 may attempt to automatically correct minor syntax errors, such as normalizing terminology variations or correcting common misspellings of attachment-related terms. The syntax checker 356 may generate error reports identifying specific locations and types of syntax violations, enabling targeted correction of the machine-readable instructions before they are provided to subsequent processing stages such as the compiler 358.

After various checks, the machine-readable instructions may be provided to compiler 358 to compile code based on the machine-readable instructions. The compiler 358 processes the validated machine-readable instructions and transforms them into executable protocol code. In some embodiments, the compiler 358 may perform additional optimization operations on the machine-readable instructions, such as consolidating redundant instructions, ordering instructions for efficient execution, or resolving dependencies between different instruction components. The compiler 358 may generate treatment protocol code in a format compatible with a treatment building engine or treatment planning system. In some embodiments, the compiler 358 may generate code in a domain-specific language designed for orthodontic treatment planning operations. The compiler 358 may further perform linking operations to integrate the compiled instructions with existing treatment protocol libraries or frameworks. In some embodiments, the output of the compiler 358 is protocol data 360, which may be used for generating treatment plans based on practitioner instructions 340. The protocol data 360 may include compiled treatment algorithms, parameter configurations, and/or execution sequences that can be applied to patient-specific dental data to generate customized treatment plans. In some embodiments, the output of compiler 358 is a treatment algorithm or executable treatment plan, or portion thereof.

FIG. 3E is a block diagram of a data flow 300E for utilizing a GUI-based system for converting practitioner preferences into machine-readable treatment protocols, according to some embodiments. At the beginning of data flow 300E, treatment option form 362 is provided to a practitioner. Treatment option form 362 may be provided on a provider's device, e.g., via a GUI. Treatment option form 362 may include a plurality of treatment categories, each associated with one or more selectable options. For example, treatment option form 362 may include categories such as interproximal reduction (IPR), arch to treat, missing teeth, extractions, malocclusion correction, crowding, spacing, midline, anterior-posterior correction, posterior crossbite, anterior correction, anterior leveling, overbite, aligner features, overcorrection for space closure, passive/active aligners, attachments, precision cuts, bite ramps, and/or power ridge features. Within each category, treatment option form 362 may present specific options for selection by the practitioner. For example, an IPR category may include options to limit anterior IPR per contact with selectable values such as 0.5 mm, 0.4 mm, 0.3 mm, or 0.2 mm, as well as options to limit posterior IPR per contact with similar selectable values. Treatment option form 362 may further include options for scheduling IPR at specific stages, such as based on access to contacts, every certain number of stages, or at specific stages specified by the practitioner. Treatment options may be presented via various GUI elements including drop-down menus, fillable fields, check boxes, radio buttons, sliders, and/or text entry areas. The practitioner may interact with treatment option form 362 by clicking on selectable elements, entering values into text fields, selecting options from drop-down menus, and/or toggling check boxes to indicate preferences. In some embodiments, treatment option form 362 may include a dental arch diagram that allows the practitioner to select specific teeth or segments of the dental arch where particular treatment operations are to be applied or restricted. Treatment option form 362 may further include a button or option to reset settings to default values, such as a “use align defaults” button. In some embodiments, treatment option form 362 may include tabs for different case types, such as primary orders and additional aligners, allowing the practitioner to configure preferences for different treatment scenarios. In some embodiments, treatment option form 362 may be accessed via a web browser, and operations related to doctor instruction collection, treatment protocol generation, etc., may be performed by a server device accessed by the practitioner via the internet. In some embodiments, various operations including doctor preference collection and machine-readable protocol generation may be performed locally.

In some embodiments, treatment categories may be divided based on patient age groups. For example, treatment options may be divided between adult patients and teen patients, with different treatment options or parameters available for each age group. In some cases, if no division is specified for a particular treatment category, the options may apply to both adult and teen age groups. The age-based division may enable treatment providers to specify different clinical preferences for patients of different ages, reflecting differences in treatment approaches, tooth movement rates, compliance expectations, or other age-related factors.

In some embodiments, treatment categories may be further divided based on malocclusion classification. For example, options for anterior-posterior correction may be divided between Class II malocclusion and Class III malocclusion, with similar but distinct treatment options available for each class. The treatment trees for Class II and Class III malocclusions may include overlapping options but may differ in specific parameters, treatment sequences, or available treatment goals based on the clinical characteristics of each malocclusion class.

In some embodiments, treatment categories provided may include specific options for interproximal reduction (IPR). IPR options may include selection of jaw sections where IPR is allowed, such as anterior segments, posterior segments, or specific inter-tooth intervals. IPR options may include per-contact limits specifying maximum IPR amounts, such as 0.2 mm, 0.3 mm, 0.4 mm, or 0.5 mm per contact. IPR options may include stage scheduling options specifying when IPR should be performed, such as based on access to contacts, at specific stages, or at regular intervals every certain number of stages.

In some embodiments, treatment categories may include options for managing spacing and tooth size discrepancy. Spacing options may include equal spacing distribution, spacing concentrated around lateral incisors, spacing distal to canines, or spacing distal to lateral incisors. These options may enable treatment providers to specify how residual spaces should be distributed when tooth size discrepancies exist.

In some embodiments, treatment categories may include options for managing crowding. Crowding options may include arch expansion limits between specific teeth, such as limiting expansion between upper first molars to a specified measurement in millimeters. These limits may constrain the treatment planning algorithm to avoid excessive arch expansion while resolving crowding.

In some embodiments, treatment categories may include options for overbite correction. Overbite options may be divided between anterior open bite cases and deep bite cases. For open bite cases, options may include selection of a target final overbite value and selection of treatment approach such as extrusion of anterior teeth, intrusion of posterior teeth, or combinations thereof. For deep bite cases, similar options may be provided with intrusion of anterior teeth as an additional or alternative approach. The treatment provider may select the desired final overbite measurement and the biomechanical approach to achieve that outcome.

In some embodiments, treatment categories may include options for anterior leveling. Anterior leveling options may relate to vertical control of anterior teeth, enabling treatment providers to specify preferences for how anterior teeth should be vertically positioned relative to one another during treatment.

In some embodiments, treatment categories may include options for distalization patterns in anterior-posterior correction. Distalization pattern options may include compact sequential distalization, improved sequential distalization, and ordinary distalization, each representing different approaches to moving posterior teeth distally. The GUI may provide explanations of what each distalization pattern means and how they differ from one another. The treatment provider may select an amount of distalization within an allowed range, such as a specific number of millimeters. Additional options may include whether to start anterior teeth movement at the same time as posterior distalization or to perform these movements sequentially. Priority options may also be provided for cases where both molar and canine correction cannot be fully achieved, allowing the treatment provider to specify which should be prioritized.

Option selection 364 may include a practitioner utilizing treatment option form 362 to input their preferences, protocols, etc., via a GUI. Option selection 364 may include options related to various types of treatments (e.g., different dental disorders, different categories of malocclusions or other conditions, etc.). Option selection 364 may include providing selection of different categories that may apply to several different treatments. For example, a practitioner's preferences on timing of interproximal reduction (removing enamel from between teeth to create more space) may be applied to many different conditions and treatments.

Option selection 364 may include obtaining selections of practitioner preferences within one or more treatment categories, treatment preference categories, etc. Option selection 364 may include obtaining various options based on conditions, condition severity, presence of interrelated conditions, etc. Option selection 364 may include different flows for different treatment goals. For example, option selection 364 may provide a different set of treatment preference selections based on a patient's desired outcome, such as a dental patient desiring realignment of their front teeth (e.g., the “social six”), a dental patient desiring improved chewing or bite alignment, or the like. Option selection 364 may include a branching tree of options that may be provided, e.g., selection of one option may open or close additional sets of selectable options. Option selection 364 may present a user (e.g., a treatment provider) with a curated, pre-selected list of choices. The practitioner may select from between the options via the GUI, using any input method of relevance to that option, including a drop-down menu, checkbox, radio buttons, selectable icons indicating shape or positions of effected teeth, etc. Additional information with respect to treatment option form 362 and option selection 364 may be found in the discussion in connection with FIG. 3F.

After doctor preferences have been collected, the preferences may be provided to protocol generation model 366. Protocol generation model 366 may be configured to generate machine-readable protocol instructions based on the practitioner preferences provided in option selection 364. The protocol generation model 366 may be a deterministic model, e.g., for a given set of options selected it may always output the same instructions. The protocol generation model 366 may be a rule-based model, configured to output machine-readable protocol instructions by following a set of instructions that convert practitioner preferences into instructions that express those preferences, and can be executed to generate treatment plans and facilitate treatment that is aligned with the practitioner preferences.

Output of protocol generation model 366 may be provided for protocol review 368. In some embodiments, code (e.g., machine-readable instructions) may be provided via the GUI. The practitioner may review the code. In some embodiments, multiple versions may be accessible. For example, versions of the machine-readable code based on settings selected by the practitioner before and after a change may be made available for review.

The practitioner may publish protocol 370, e.g., based on confirming via review that the protocol was generated as expected based on the practitioner preferences. Publishing of the protocol may include posting the protocol to be applied to all future patients of the practitioner, reevaluating treatment plans associated with current patients of the practitioner in view of the new protocol, or the like. In some embodiments, treatment planning operations may be accessed via the same application or portal as the protocol generation. For example, a web browser portal may include a section dedicated to treatment option form 362, and a second section dedicated to generating patient treatment plans based on protocols created via the treatment option form 362.

In some embodiments, a flow based on obtaining practitioner preferences via a user interface, an embodiment of which is described in connection with FIG. 3E, and a flow based on obtaining natural language practitioner comments, an embodiment of which is described in connection with FIG. 3A, may be combined or used together. For example, the treatment option form 362 may provide options that are commonly used or commonly preferred by practitioners, e.g., due to limited development time of the treatment option form 362 or protocol generation model 366, to avoid excessive visual or information clutter in treatment option form 362, or the like. For cases where the provided options don't apply to a specific case, don't meet the providers needs, or are otherwise inadequate, LLM- or AI-based methods may be utilized in protocol generation to augment the option selection methods.

FIG. 3F depicts an example GUI 300F for treatment protocol generation based on selections of a practitioner, according to some embodiments. GUI 300F may be provided via an application on a provider's device, via a web portal, or the like. Operations related to GUI 300F may be performed locally, by a server device via the internet, or the like.

GUI 300F includes protocol preferences user interface (UI) 372. GUI 300F may include additional elements, e.g., tabs for UI elements related to active patients, treatment planning operations, appliance manufacturing or shipping information, or other categories of information that may be of relevance to the provider. Protocol preferences UI 372 may include various selections, options, treatment categories, treatment goals, etc., to be chosen by the healthcare provider for generation of treatment protocols, e.g., generation of machine-readable code capturing the treatment provider's treatment preferences as a treatment protocol.

Protocol preferences UI 372 may include treatment component selection 380. Treatment component selection 380 may include a selection of a stage or component of a treatment plan. For example, a dental or orthodontic treatment may include a number of appliances for rearranging or otherwise adjusting patient dentition. Broad categories may be selectable under treatment component selection 380. For example, in some cases additional appliances may be provided, which are not part of a primary treatment. For example, appliances to be worn to maintain tooth placement (e.g., to be worn at night), rather than to enact change to a dental arch, may be accessed by selecting a treatment component. Various options related to additional appliances, such as how and when to perform interproximal reductions, extractions, and features of the appliances including attachments and hooks, may be selected in accordance with practitioner preferences. Main appliances, for a primary component of the treatment, may have additional available options related to various conditions, various treatments, etc.

Protocol preferences UI 372 may include treatment category selection 374. Treatment category selection 374 may include various aspects on which a practitioner may express a preference that will adjust how treatment is performed, adjust treatment planning operations, adjust treatment appliance design and manufacturing, etc. Treatment category selection 374 may include selection of specific conditions or treatments. Treatment category selection 374 may include selection of aspects of treatment that affect treatment of multiple conditions, that affect design or manufacture of treatment appliances generally, or the like. Treatment category selection 374 may be separated or segmented by various categories, such as a section for malocclusion correction, a section for anterior correction, a section for general treatment preferences, etc.

Treatment category selection 374 may provide a category selection. Options within the category may be presented by the treatment options selection 376 element, upon selection of the category in treatment category selection 374.

As an example, treatment category selection 374 may include a number of malocclusion categories, anterior correction categories, and general treatment categories. Selectable categories available in treatment category selection 374 may include any treatment categories of relevance (e.g., that may assist in organizing the treatment preferences) and protocol generation questions for a set of treatment providers (e.g., treatment providers providing a specific treatment, treatment providers in a target industry, or the like).

Treatment category selection 374 may include a number of categories related to treatment of dental and orthodontic conditions with aligner appliances. Treatment category selection 374 may include interproximal reduction. Treatment category selection 374 may include approaches related to single dental arch treatments. Treatment category selection may include an approach to missing teeth. Treatment category selection may include an approach to extractions during treatment. Treatment category selection may include preferences for treating crowding. Treatment category selection may include preferences for treating tooth spacing. Treatment category selection may include treating midline misalignment. Treatment category selection may include anterior-posterior correction. Treatment category selection may include preferences for treating posterior crossbite. Treatment category selection may include treating anterior leveling. Treatment category selection may include treating overbite. Treatment category selection may include appliance features that may be applicable to many treatments. Treatment category selection may include providing preferences related to overcorrection. Treatment category selection may include preferences related to timing and use of passive and active treatment appliances.

Protocol preferences UI 372 may include defaults 378. In some embodiments, a UI element may be provided to view or set one or more categories or options to a default, such as default treatment options provided by the treatment planning application or program.

Protocol preferences UI 372 includes treatment options selection 376. A user selection of a treatment category via treatment category selection 374 may cause options related to the selection treatment category to be presented via treatment options selection 376. Treatment options selection 376 may present one or more sets of selectable options. In some cases, treatment options selection 376 may provide treatment goals, which may each be relevant for different treatment cases. For example, generating a treatment plan for a new patient may include selecting one or more treatment goals, which may cause the protocol script or machine-readable instructions to implement treatment planning operations based on the preferences reflected with respect to those treatment goals. Treatment options selection 376 may provide a branching tree of options, where selection of some goals or preferences may cause additional categories, options, or selections to be presented based on previous choices.

In some embodiments, treatment options selection 376 may include separated tabs, types, categories, groupings, or the like. For example, different preferences may be expressed by a practitioner for different classifications of disorder (e.g., class II or class III malocclusion, related to one jaw or the other protruding too far, may be treated differently by a practitioner). Different preferences may be expressed for different severities of disorder. Different preferences may be expressed for different types of patients, e.g., developmentally (for example, teens and adults may be treated differently), with respect to patient preferences, or other patient categories. These classifications or categorizations may be presented via treatment options selection 376, e.g., via tabs, as sections or groupings of the presented options, or the like. In some cases, one of the choices for one or classifications may be treated as a default, and if no different preferences are expressed by the practitioner, the system may generate machine-readable treatment protocol instructions that reflect the default selections for all groups or all groups without contradicting instructions.

Treatment options selection 376 may include options related to interproximal reduction. Interproximal reduction is a process of removing some material or enamel from teeth in the interproximal region, or the region between adjacent teeth. Final positions of teeth after treatment may accommodate different sizes of teeth, and interproximal reduction may be performed in various stages and with respect to various conditions to improve treatment and improve the final fit of the dentition. Different doctors, clinicians, practitioners, etc., may have different preferences of when or how to perform interproximal reduction. In some cases, a picture or model may be presented, for example, presenting zones or dentition or teeth that may be selected for different preferences. In the case of interproximal reduction, for example, segments of teeth where interproximal reduction is allowed may be selected. A maximum amount of interproximal reduction allowed may be selected, e.g., a posterior and anterior limit to interproximal reduction per contact may be selected. Timing of interproximal reduction may be selected, e.g., before or after alignment, delayed by a target number of treatment stages, or the like. Scheduling preferences may be presented, such as basing scheduling of interproximal reductions based on access to the regions of the teeth to be reduced, performing the interproximal reduction on a regular schedule with respect to treatment stage, performing interproximal reduction at specific target treatment stages, or the like.

In some embodiments, treatment options selection 376 may include options related to operations of single arch treatment, such as whether to simulate treatment on the opposite arch, perform no movement on the opposite arch, manufacture passive aligners for the opposite arch, etc.

In some embodiments, treatment options selection 376 may include options related to missing teeth during treatment. Options for placing pontics (an artificial tooth that replaces a missing natural tooth on a fixed dental prosthesis) may be presented for selection. Pontics may be allowed for anterior missing teeth, posterior missing teeth, both, or neither. Any other options related to pontics that may be expressed as a treatment preference or protocol may be included.

In some embodiments, treatment options selection 376 may include options related to tooth extractions to be performed during treatment. Options may include scheduling extractions, e.g., delay extractions to a target stage of treatment. Treatment options selection 376 may provide options for pontics following extractions, e.g., whether pontics are to be placed for extracted teeth, extracted posterior teeth, extracted anterior teeth, etc.

In some embodiments, treatment options selection 376 may include treatment options for one or more malocclusions, or bite misalignments. Malocclusion treated with options for treatment presented under treatment options selection 376 may include crowding. Crowding treatment preferences may include a limit to arch expansions between upper first molars. Crowding treatment preferences may provide an options for a maximum measurement of expansions between upper first molars, for example, up to 1 mm of expansions.

Malocclusion treatment options presented in treatment options selection 376 may include tooth spacing. Spacing may include options for how spaces and tooth size discrepancy are managed in treatment. Options for space management for tooth size discrepancy may include distal to canines, distal to laterals, equally around laterals, or no spaces to account for tooth size discrepancy, for example.

Malocclusion treatment options may include options for midline correction. Midline correction options may include a treatment goal, such as improving midline with interproximal reduction, or showing a resulting midline after alignment. A general approach to achieving the goal may be selected, such as moving the upper arch, moving the lower arch, or moving both arches to match the opposing arch.

Malocclusion treatment options may include options for anterior-posterior correction, or treatment of misalignment between the upper and lower jaws in the front-to-back direction. A treatment goal may be selected, related to which tooth alignments to prioritize. Specific treatments or corrections may be applied to the upper arch and/or lower arch. For example lower arch options may include bite correction simulation, mesialization (pulling teeth forward toward the midline), or no correction; upper arch options may include bite correction, distalization (pushing teeth backward away from the midline), or no correction, etc. A distalization pattern may be selected, such as improved sequential distalization (moving teeth in a temporally overlapping sequential pattern), compact sequential distalization (distalizing many teeth at the same time, which may cause additional stress), sequential distalization (moving a second tooth after the first has arrived in its target placement), or any other preference that may be expressed by a treatment provider for a pattern of distalization. Similar options may be provided for mesialization. An allowed maximum distance of distalization may be selected. A type of bite correction may be selected, e.g., using elastics or surgical intervention. A timing of a surgical intervention and/or elastics use may be selected, e.g., at the beginning or end of treatment, during or between one or more stages of treatment, etc. Options related to timing of treatment may be selected, such as whether or not to begin adjusting positions of anterior teeth while still performing posterior distalization, or after posterior distalization. A priority may be set, e.g., if only alignment of molars or canines can be achieved, a selection of which is more important to the treatment provider for a treatment protocol may be selected.

Malocclusion treatment options may include options for treatment of posterior crossbite, or a misalignment where the upper back teeth bite inside the lower back teeth. Options selected in treatment options selection 376 may include whether or not to perform correction, whether to correct molars and premolars, molars only, premolars only, etc.

Categories presented by treatment category selection 374 may include anterior leveling, or performing vertical adjustments to anterior teeth. Options presented in treatment options selection 376 may include a general approach to the upper and/or lower arch, e.g., providing the lateral teeth 0.5 mm more gingival than central teeth, leveling incisal edges, leveling gingival margins, or the like.

Categories presented by treatment category selection 374 may include overbite treatment, with options for overbite treatment presented via treatment options selection 376. Separate sections for anterior open bite and deep bite may be presented. For practitioner preferences in generation of a treatment protocol, whether to extrude or intrude (depending on whether the bite is open or deep, for example) anterior teeth only, extrude or intrude anterior and posterior teeth, and a desired final position (e.g., final overbite) may be selected.

Treatment category selection 374 may include features of treatment appliances, that may apply to multiple conditions or treatments. Features of treatment appliances may be applicable to multiple dental treatments, multiple treatment goals, multiple types of treatment interventions, etc. Treatment options selection 376 may include options related to attachment size, e.g., attachments for treatment appliances that are applied to a patient's teeth. Attachment size may be selected (e.g., selecting an attachment size, a standard size, an indication to use the largest attachments that fit, etc.). Options may be selected differently for different teeth or different zones of teeth, e.g., upper teeth, lower teeth, posterior or anterior teeth, etc. A delay to a target treatment stage for attachments may be provided.

Options related to additional appliance features for additional treatments may be provided, such as elastic attachments for anterior-posterior correction. Elastic attachments may be disabled, a delay to a target treatment stage for applying elastic attachments may be specified, an interaction between attachments and elastics may be indicated, elastic attachment style may be selected, etc. In some embodiments, a general preference may be expressed by the practitioner, which the treatment protocol may be configured to apply to any case where possible, with different options being implemented when the practitioner preference is unavailable.

In some embodiments, aligner features available for selection via the GUI may include precision cuts. Precision cuts may be cutouts or hooks on aligners configured to engage elastics for orthodontic correction, such as Class II or Class III elastic correction. Options for precision cuts may include enabling or disabling precision cuts, delaying placement of precision cuts to specific stages of treatment, and prioritizing precision cuts relative to other aligner features such as attachments. The GUI may provide separate options for precision cuts based on malocclusion class (Class II or Class III) and patient age group (adult or teen).

In some embodiments, aligner features may include bite ramps. Bite ramps may be placed automatically for deep bite cases to assist in bite opening. Options for bite ramp placement may include selection of placement locations such as canines, lateral incisors, central incisors, or combinations thereof. The treatment provider may select any combination of placement locations or may choose not to place bite ramps. Options related to bite ramps may be presented via treatment options selection 376. A selection of bite ramp type (e.g., which teeth it is permissible to utilize bite ramps), whether or not to place bite ramps, whether to include the bite ramps automatically or allow the practitioner to customize bite ramp placement, etc., may be included in treatment options selection 376.

In some embodiments, treatment options may include overcorrection settings. Overcorrection options may include whether to add additional aligners for overcorrection at the end of treatment. Overcorrection type options may include canine-to-canine overcorrection or molar-to-molar overcorrection. The treatment provider may also select which arch or arches to apply overcorrection to, such as upper arch only, lower arch only, or both arches. Options related to overcorrection may be provided by treatment options selection 376. A number of overcorrection appliances may be selected to add to each treatment plan generated with respect to the treatment protocol. A type of overcorrection may be selected, which dental arch to overcorrect may be selected, etc.

In some embodiments, treatment options may include passive aligner settings. Passive aligner options may include whether to use passive aligners at the end of active treatment. Options for finishing active aligners may include starting and finishing both arches at the same time, or starting both arches at the same time and finishing each arch when it is ready. These coordination options may enable treatment providers to specify how the transition from active treatment to passive retention should be managed across the upper and lower arches. Options related to passive appliances may be presented via treatment options selection 376. Passive appliances may be used if the upper and lower dentition require a different amount of time, different number of treatment stages, different number of treatment appliances, or the like for completion of treatment. Whether or not to schedule passive aligners along with active aligners, whether both arches are to start treatment or end treatment at the same time, etc., may be selected by the practitioner.

In some embodiments, one or more treatment options presented in treatment options selection 376 may include a description or information related to the option, selection, treatment goal, or the like. For example, while selecting options for midline correction, information may be displayed that explains how midline correction may be affected or may be ineffective based on other option selections. A description may indicate a combination of options, which may include options in the current treatment category and/or other treatment categories, which are predicted to result in the closest adherence to one or more treatment goals. In the case of midline correction, a hint or description may indicate that the best results for midline correction may be achieved by setting anterior-posterior correction to canine class I (in the anterior-posterior correction category of treatment category selection 374, for instance), allowing interproximal reduction, and to not close anterior residual spaces. In some embodiments, selecting a hint icon or hovering a cursor over an option may cause the UI to provide additional information. For example, while selecting a distalization pattern in anterior-posterior correction, a short description of each of the options may be provided to ease decision-making of the treatment provider.

In some cases, related treatments may include links or connections to enable ease of filling the option selection process. For example, in a category for anterior-posterior correction, configuring attachments for elastics or other corrective tools may be particularly relevant to the treatment goals of anterior-posterior correction, and a link to these settings or a copy of the options for those settings may be provided in the anterior-posterior correction section.

In some embodiments, for each treatment category or treatment option presented via the GUI, a default configuration may be displayed. The default configuration may represent recommended or standard treatment parameters. A 3D model visualization may be provided showing how the default treatment would appear on example dentition, allowing the treatment provider to visualize the expected treatment outcome associated with the default settings. The treatment provider may compare their own selections against the recommended defaults by viewing both the default 3D model and a 3D model reflecting their selected preferences. This comparison capability may assist treatment providers in understanding the implications of their selections and in making informed decisions about treatment parameters

Upon filling selections in treatment options selection 376, a practitioner may view the treatment protocol in protocol view 382. In some embodiments, protocol view may be in a separate tab or page than other elements of protocol preferences UI 372, or may be presented together as shown in FIG. 3F. Protocol view 382 may show the machine-readable code or instructions related to the protocol based on input of the practitioner preferences. Generation of protocol view 382 may be based on an indication from a user that they have finished inputting parameters, e.g., a “generate protocol” button may be included in protocol preferences UI 372. For generating treatment plans, the machine-readable protocol instructions may be used along with additional input, such as a doctor indicating treatment or patient categories (e.g., whether the patient is a teenager or adult), the doctor indicating treatment goals (e.g., complete dental alignment, social six alignment, etc.), the doctor providing any specific case-by-case updates to perform treatment in a different manner than the protocol, etc.

In some embodiments, protocol preferences UI 372 may include a 3D model view 384. The 3D model view may be accessed on a different page or tab. The 3D model view 384 may display a model of an effected region in connection with the treatment protocol, with a specific treatment category or treatment option, etc. The 3D model view may provide for inspection a three-dimensional model of dentition in relation to a treatment, treatment category, treatment category options, or the like. In some embodiments, a default option may be presented, e.g., a model highlighting final tooth locations, attachment locations, or the like of the default treatment selections for a particular treatment category. In some embodiments, treatment provider selection provided in treatment options selection 376 and/or expressed in protocol view 382 may be presented. In some cases, example dentition may be used for the 3D model view 384, or a practitioner may select from a list of dentition, which may include former and/or current patient dentition, in some embodiments. Protocol preferences UI 372 may include an option to publish protocol 386, which may apply the updated protocol based on updated treatment provider preferences to future treatment planning options, re-assess current treatment plans, etc.

In some embodiments, each time a treatment provider updates their protocol via the GUI, a new version of the machine-readable code may be generated and stored and/or an updated 3D model may be generated and/or output to a display. The system may maintain a version history comprising multiple treatment protocol versions, with each version associated with a version identifier such as version 1, version 2, version 3, and so on. The GUI may provide an interface for viewing different versions of the treatment protocol, comparing versions, or reverting to a prior treatment protocol version. This version history may enable treatment providers to track changes to their clinical preferences over time and to restore previous configurations if desired.

In some embodiments, upon updating a protocol via the GUI, such as by clicking an “update template” button or similar control, the new protocol version may be immediately applied. Immediate application may mean that any new treatment plans entered after the update may use the updated protocol. This immediate application may enable treatment providers to quickly implement changes to their clinical preferences without delay or additional confirmation steps beyond the initial update action

In some embodiments, machine-readable code generation based on GUI selections may be performed locally on a treatment provider's device or by a server accessed via the internet. The GUI-based protocol generation application may be accessed via a web browser, with operations related to preference collection and protocol generation performed by a remote server. Alternatively, various operations including preference collection and machine-readable protocol generation may be performed locally on the treatment provider's device. However, execution of the generated machine-readable code to perform treatment planning operations may require proprietary treatment planning engines that may be hosted on remote servers. Accordingly, while the machine-readable protocol code may be generated locally, the treatment provider may transmit the generated code to a server for execution in connection with patient data to generate treatment plans.

FIG. 3G depicts an example global clinical preferences template GUI 300G for treatment protocol generation based on selections of a practitioner, according to some embodiments. GUI 300G may share one or more features with GUI 300F of FIG. 3F. GUI 300G may be configured for Flex Rx cases and may include settings applicable to treatment protocol generation for such cases.

GUI 300G includes additional aligners tab 388. Additional aligners tab 388 may share one or more features with treatment component selection 380. Additional aligners tab 388 may be presented as selectable buttons, tabs, options in a drop-down menu, or in another manner. In the case of GUI 300G, the additional aligners tab 388 is selected, indicating that the current view pertains to additional aligner settings. GUI 300G may also include a primary orders tab that, when selected, displays settings associated with primary treatment orders.

GUI 300G includes treatment category menu 392. Treatment category menu 392 may share one or more features with treatment category selection 374. Treatment category menu 392 may include a set of treatment categories that are to be used in generating a treatment protocol. Treatment categories displayed in treatment category menu 392 may include IPR, Arch to treat, Missing teeth, Extractions, Malocclusion correction, Crowding, Spacing, Midline, Anterior-Posterior correction, Posterior crossbite, Anterior correction, Anterior leveling, Overbite, Aligner features, Overcorrection for space closure, and/or Passive/active aligners. Treatment categories may be separated into sections or displayed as a scrollable list.

GUI 300G includes use align defaults button 396. In some embodiments, use align defaults button 396 may act on all treatment components, a single treatment component, a single treatment category, a single selection within a treatment category, or the like. In some embodiments, selecting use align defaults button 396 may cause treatment settings to be reset to default values provided by the system. Use align defaults button 396 may be positioned at the bottom of the GUI 300G to allow the user to reset settings after reviewing current configurations.

GUI 300G includes dental arch diagram 390. For some treatment options, it may be helpful to provide a graphical guide to one or more selections. Dental arch diagram 390 displays a representation of upper and lower dental arches, allowing selection of segments where treatment operations such as interproximal reduction (IPR) are allowed. Various segments of dentition may be selected for one or more treatment operations via the dental arch diagram 390, enabling practitioners to visually specify which areas of the dental arches should receive particular treatments.

GUI 300G includes IPR limit selection fields 394 and/or other numerical selection fields. For some treatment options, it may be convenient to provide a limited list of potential numerical values that may be selected. For other treatment options, it may be useful to provide more or fewer options, or a free entry option, such as free entry element 398. IPR limit selection fields 394 provide options to limit anterior IPR per contact with selectable values such as 0.5, 0.4, 0.3, and 0.2 millimeters, as well as options to limit posterior IPR per contact with the same selectable values. The IPR limit selection fields 394 allow practitioners to specify maximum amounts of interproximal reduction to be performed at each contact point.

GUI 300G includes stage specification field 398. Stage specification field 398 provides options for scheduling IPR at specific stages of treatment. Stage specification field 398 may include options such as scheduling IPR based on access to contacts, scheduling IPR every certain number of stages, or scheduling IPR at specific stages. Stage specification field 398 may include a text entry area for specifying particular stage numbers at which IPR should be performed. For other treatment options, it may be useful to provide more or fewer options, or a free entry option similar to stage specification field 398.

FIG. 3H depicts an example clinical preferences template GUI 300H including nested treatment categorization and selection of treatment goals, according to some embodiments. GUI 300H includes malocclusion class tabs 305, which provide class selection such as between different malocclusion categories such as Class II and Class III, with an Adult designation shown below the tabs. In some cases, different severities of disorder, directionality of disorder (e.g., overbite vs underbite), or the like may be treated differently, and further options and selections may be entirely dependent on class selection. In some cases, the same set of selections and options may be provided for each class, while in some cases one or more selections or options may be different between classes. GUI 300H may share one or more features with GUIs 300F and 300G.

GUI 300H includes a treatment goal configuration panel 307 for treatment goal selection. Treatment goal configuration panel 307 may enable a practitioner to address multiple cases that may arise within a single treatment category. The treatment goal configuration panel 307 contains multiple configuration options including a treatment goal dropdown menu (e.g., set to “Molar and canine class I”), upper arch and lower arch selection fields, a distalization pattern dropdown, an amount of distalization specification field (e.g., set to “4 mm”), a checkbox option for starting anterior teeth movement at the same time as posterior distalization, priority selection radio buttons for Molar and Canine, and/or type of bite correction simulation options including Elastics and Surgical. For example, different targets may be applicable, appropriate, or selected for different patients, such as prioritizing aligning particular dental structures over others, prioritizing molar alignment or anterior tooth alignment, or the like. In some cases, options may be selected for multiple treatment goals, and during treatment planning operations for a particular patient, a treatment goal may be selected. Further operations of treatment planning may be performed based on the selected goal, and the options selected in reference to that treatment goal via GUI 300H. The left side of GUI 300H displays a navigation menu listing various treatment categories including IPR, Arch to treat, Missing teeth, Extractions, Malocclusion correction, Crowding, Spacing, Midline, Anterior-Posterior correction, Posterior crossbite, Anterior correction, Anterior leveling, Overbite, Aligner features, Overcorrection for space closure, and Passive/active aligners. The interface includes tabs for Primary orders and Additional aligners at the top, along with a notification indicating that settings apply only for Flex Rx cases and that changing settings will not affect Traditional Rx. A “Use Align Defaults” button and a “Review Align Default” link are provided for accessing default configuration options.

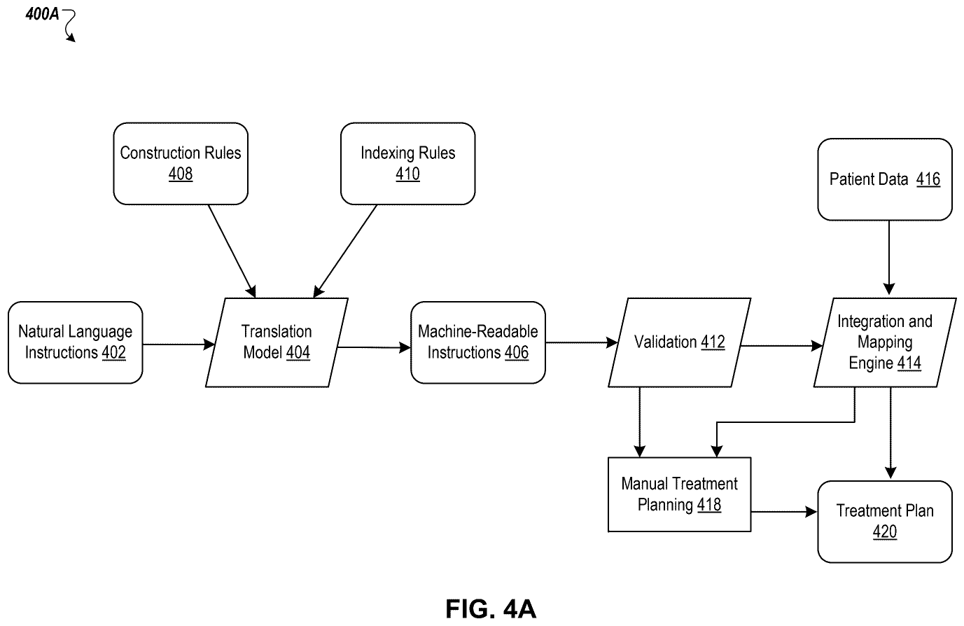

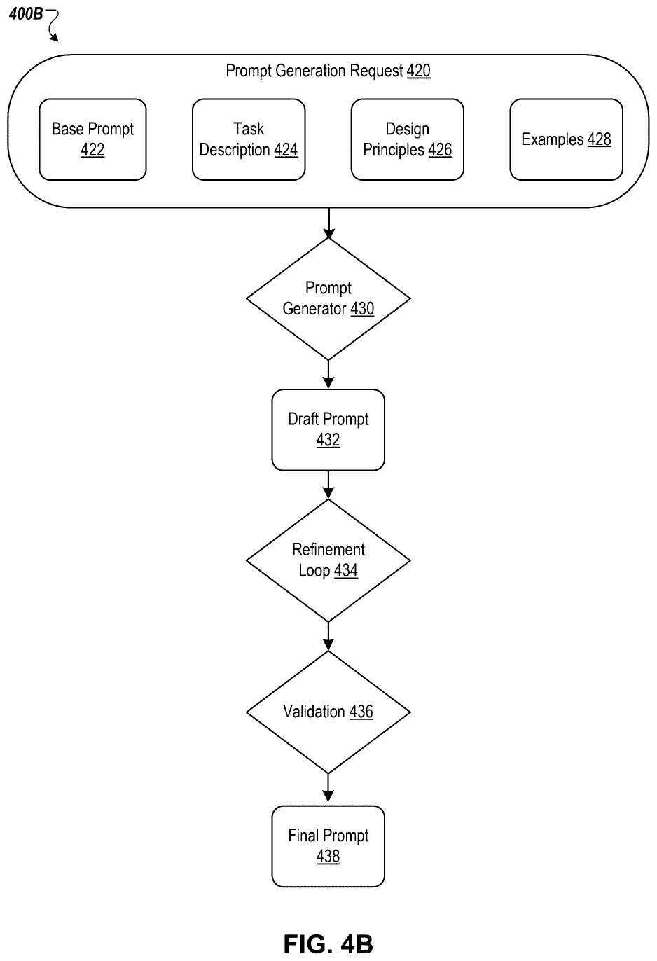

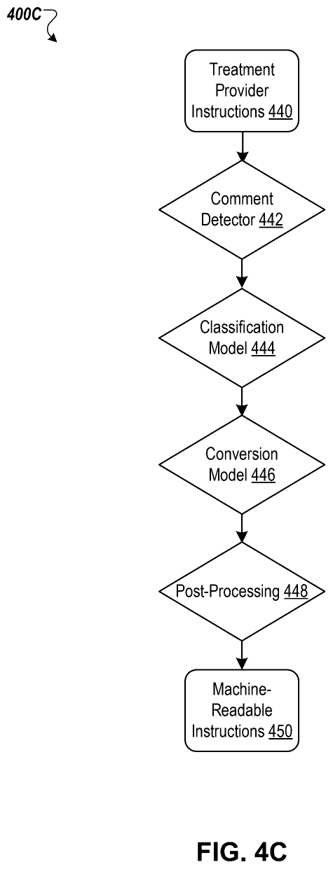

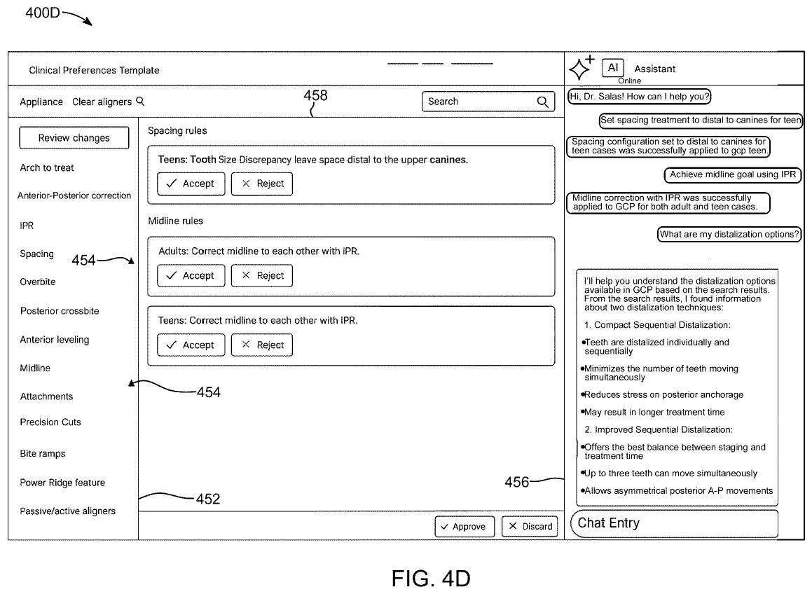

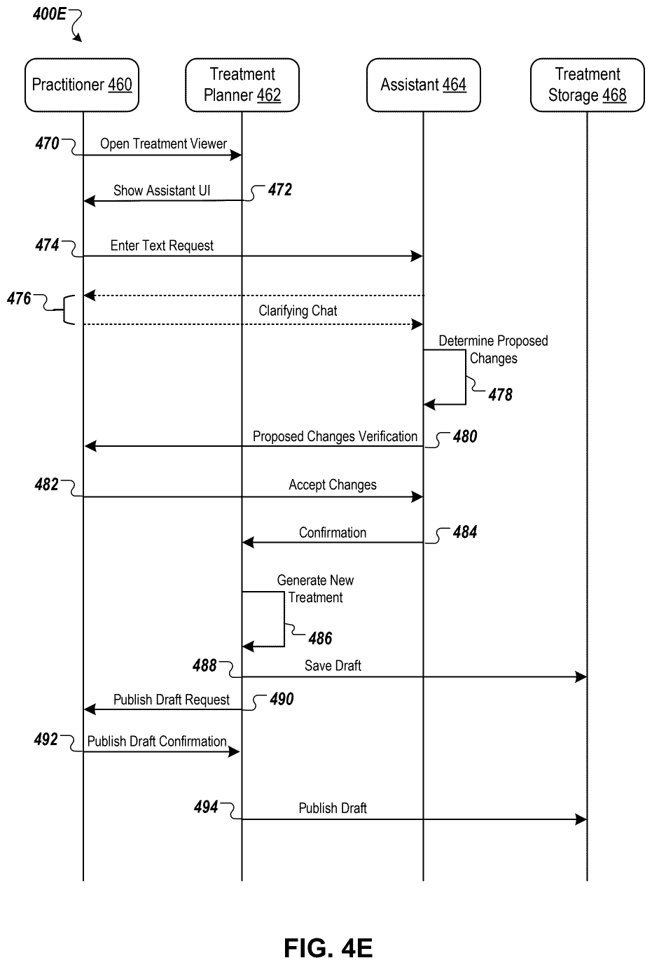

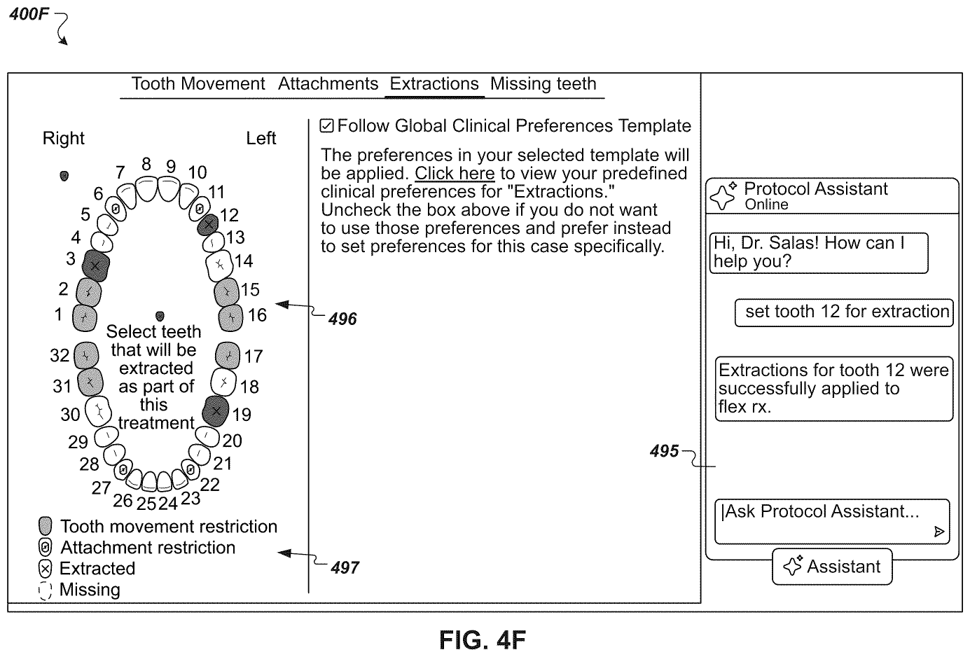

FIG. 4A is a block diagram of a data flow 400A for generating a treatment plan 420 based on natural language instructions 402, according to some embodiments. At block 402, natural language instructions are obtained by a treatment planning system. The natural language instructions may be related to differences between a treatment protocol (e.g., a default protocol or a protocol based on practitioner preferences) and specific treatment goals for a patient. Alternatively, the natural language instructions may be instructions to generate a new treatment plan from scratch. The natural language instructions 402 may be provided via a text input GUI element of a treatment planning GUI. Alternatively, the natural language input may be received as spoken audio, which may be transformed into text using a speech to text engine (e.g., one or more AI models trained to convert speech into text). The natural language instructions 402 may include one or more references to teeth of the dental patient. The references to teeth may include references to specific teeth. The references may include references to groups of teeth, including named groups of teeth (e.g., upper left molars, anteriors, etc.), ranges of teeth (e.g., all teeth between 7 and 10), teeth referenced by relative position (e.g., mesial of UR3), teeth referenced by by exclusion (e.g., all anteriors except centrals), or the like. The teeth or groups of teeth may be referred to by an indexing system related to dentition, such as UNS, Palmer, FDI, etc. In some embodiments, an indication of a preferred teeth numbering or indexing system of the practitioner may also be provided as part of natural language instructions 402, or part of construction rules 408 and/or indexing rules 410.

Various prompt engineering information may also be provided along with natural language instructions 402 to an LLM or other AI model for processing. Prompt information may include construction rules 408 and/or indexing rules 410. Construction rules 408 may include an outline for generating machine-readable instructions related to patient treatment. Construction rules 408 may include examples of machine-readable instructions corresponding to natural language instructions. Construction rules 408 may indicate a similarity of structure between a target machine-readable format and a known format, e.g., “in a machine-readable format similar to JSON” may be included in construction rules 408. Categories of potential instructions may be described and defined, with examples of instructions corresponding to each category provided. Construction rules 408 may specify a set of classifications of treatment instruction categories, such as dental features, attachments, interproximal reduction, passive aligners, overcorrection, treatment length, and other treatment-related categories. For each classification, construction rules 408 may provide one or more examples of machine-readable instructions that correspond to natural language instructions within that category. Construction rules 408 may further specify allowed values, actions, and relationships between different elements of the machine-readable format. For example, construction rules 408 may define a set of permissible actions such as add, remove, keep, replace, modify, forbid placement, or delay placement in connection with dental features. Construction rules 408 may also specify attachment types including optimized attachments, conventional attachments, and protocol attachments, along with their associated parameters such as size, position, and specific type. Construction rules 408 may include a schema definition that constrains the output of the AI model to only include modifications that are valid within a predefined set of protocol commands, ensuring that generated machine-readable instructions conform to required formatting and structural requirements.

Indexing rules 410 may include similar information, related to various indexing schemes. The indexing rules 410 may include input schemes (e.g., description of various common teeth indexing rule sets, such as Palmer, FDI, Universal, etc.). Indexing rules 410 may provide detailed descriptions of each numbering system to enable the AI model to recognize and interpret tooth references expressed in different formats. For example, indexing rules 410 may specify that in the Palmer system, teeth identifiers are represented with at least one letter at the beginning indicating upper or lower and right or left quadrants, followed by a number ranging from one to eight. Indexing rules 410 may specify that in the FDI system, teeth identifiers are represented with a first number ranging from one to four indicating the quadrant, a separator character, and a second number ranging from one to eight indicating the tooth position. Indexing rules 410 may specify that in the Universal system, adult teeth are numbered from one to thirty-two starting from the upper right third molar and moving clockwise. Indexing rules 410 may further include mappings between tooth types and their corresponding identifiers in each numbering system, such as central incisors, lateral incisors, canines, premolars, and molars. Indexing rules 410 may include an output scheme, e.g., instructions to number objects such as teeth based on positions, instructions to number teeth in order of current position, or the like. Indexing rules 410 may specify a preferred machine-readable indexing scheme for output, enabling consistent representation of tooth references regardless of the input numbering system used by the treatment provider. In some cases, geometric ordering may be applicable to natural language instructions (e.g., a first tooth anterior to a second tooth is a geometric instruction), while standard indexing schemes may use identity of an object without respect to positioning (e.g., in cases where one or more objects of a set are missing, when objects are ordered in a non-standard way, when there are extra teeth in a dental arch, or the like). Indexing rules 410 may include instructions for handling ambiguous or mixed numbering system references, where the treatment provider may use terminology from multiple systems within a single instruction set. Indexing rules 410 may also specify rules for converting natural language tooth group references, such as anteriors, posteriors, molars, or incisors, into specific tooth identifiers in the machine-readable format. Prompt information may also include examples, references to documents to be accessed via RAG, and/or any other prompt information described herein.

Prompt data and the natural language instructions 402 may be provided to translation model 404. The translation model 404 may be an LLM in embodiments. In some embodiments, the translation model 404 is a general purpose LLM. In some embodiments, the translation model 404 is a specifically trained AI model trained to perform the task of translating natural language instructions 402 into machine-readable instructions 406 in a specific medical field, such as dentistry or orthodontics. In some embodiments, the translation model 404 may be adjusted (e.g., through machine learning tuning techniques, such as parameter efficient fine tuning) to be more likely to produce machine-readable instruction 406 that satisfy one or more quality thresholds.

Machine-readable instructions 406 may be generated by translation model 404 based on processing of the input prompts. The machine-readable instructions 406 may represent an intermediate representation that encodes the treatment provider's natural language instructions in a structured, machine-interpretable format. In some embodiments, these machine-readable instructions 406 are distinct from both the treatment algorithm and the treatment plan. Alternatively, the machine-readable instructions, treatment algorithm, and/or treatment plan may be merged into a single construct. In some embodiments, the treatment algorithm comprises the computational logic and rules that govern how treatment planning operations are performed, and the machine-readable instructions 406 may be used to adjust or configure this treatment algorithm by adding to it, replacing portions of it, or otherwise modifying its behavior. In some embodiments, the treatment plan represents the final patient-specific output that is generated when the treatment algorithm (as configured by the machine-readable instructions 406) is applied to patient data such as a three-dimensional model of the patient's dentition. Thus, the machine-readable instructions 406 may serve as a bridge between the treatment provider's natural language input and the treatment algorithm, which in turn may generate the treatment plan when executed with patient-specific data. In some embodiments, the machine-readable instructions 406 may be configured to resemble an existing programming language, or may be formatted in a new language tuned to specific challenges to be addressed. A few examples of natural language instructions and corresponding machine-readable instructions, intended to make clear differences between the natural language input to translation model 404 and machine-readable instructions output by translation model 404 follow.

As a first example, treatment provider instructions may include an instruction to “add attachment UL2.” An output of the translation model 404 may be or include:
{
   “dental_features”: {
      “add”: [
         {
         “feature”: “attachments”,
         “teeth”: [“UL2” ]
         }
      ]
   }
}

As a second example, treatment provider instruction may include an instruction to add bite ramps to U2-2 for the upper jaw. An output of the translation model 404 may be or include:
{
   “dental_features”: {
      “add”: [
         {
         “feature”: “bite_ramps”,
         “teeth”: {“begin”: “UL2”, “end”: “UR2”}
         }
      ]
   }
}

As a third example, treatment provider instructions may include instructions to add 2 passive aligners at the end of treatment for the upper jaw, and 3 passive aligners at the end of treatment for the lower jaw. An output of the translation model 404 may be or include:
{
   “passive_aligners_placement”:
   {
      “jaw”: “upper
      “amount”: 2,
      “position”: “last_stage”
   },
   “passive_aligners_placement”:
   {
      “jaw”: “lower”,
      “amount”: 3,
      “position”: “last_stage”
   },
}

As a fourth example, doctor instructions may include instructions to add attachments for extrusion to the upper 1's. Particularly relevant in cases such as this, indexing schemes may be included in instruction parsing, according to indexing rules 410 provided as input to the translation model 404. Teeth may be classified in various ways according to an indexing scheme, which may then be mapped to patient data geometrically. Output of translation model 404 may be or include:
{
   “dental_features”: {
      “add”: [
         {
         “feature”: “attachments”,
         “teeth”: {
         “group”: “central incisors”,
         “jaw”: “upper”
         },
         “purpose”: “extrusion”
         }
      ]
   }
}

As a fifth example, tooth indexing in the machine-readable instructions 406 may include exclusion. For example, a instruction related to upper anteriors except central incisors may produce machine-readable instructions that include a tooth indexing such as:
{
   “teeth”: [
      {
         “include”: {
         “group”: “anteriors”,
         “jaw”: “upper”
         },
         “exclude”: [“UL1”, “UR1” ]
      }
   ]
}

As a sixth example, indexing of teeth may include references of one or more teeth relative to other teeth. This may be of particular interest in cases where indexed objects are out of order, expected objects are missing, extra objects are present, or the like. An example of a tooth indexing in machine-readable instructions 406 including relative tooth specification may include:
{
   “teeth”: [
      {
         “tooth”: “UL3”,
         “direction”: “mesial”
      }
   ]
}

Results such as these may be obtained by providing, included in a prompt to the translation model 404, multiple features including definitions of supported instructions and representations of teeth, examples illustrating how to convert natural language phrases into a target machine-readable format, and guidelines for addressing edge cases.

Machine readable instructions 406 may then provided for validation 412. Validation 412 may be AI-based in some embodiments. Validation 412 may be heuristic or rules-based validation in some embodiments. Validation 412 may include determining whether the machine-readable instructions 406 are reasonable, whether the machine-readable instructions 406 correlate to best practices in treatment, determining whether machine-readable instructions 406 comply with one or more treatment requirements, etc. Examples of machine-readable instructions 406 that may not comply with validation 412 may include instructions not supported by a treatment planning engine, violations of treatment best practices such as speed of alignment for orthodontic treatments, a target length of time for treatment, or the like. Validation 412 may be performed using multiple validation techniques applied sequentially or in parallel. In some embodiments, validation 412 includes syntax validation to confirm that the machine-readable instructions 406 conform to a required schema or format specification, ensuring that the instructions are parseable and structurally correct for processing by downstream components such as the integration and mapping engine 414. Syntax validation may verify that required fields are present, that data types are correct, that values fall within permitted ranges, and/or that the overall structure of the machine-readable instructions 406 adheres to a predefined schema (e.g., JSON schema) or other format specification.

In some embodiments, validation 412 includes clinical validation to determine whether the machine-readable instructions 406 adhere to clinical safety constraints and treatment heuristics. Clinical validation may include checking that proposed tooth movements do not exceed maximum safe movement thresholds per treatment stage, that interproximal reduction amounts do not exceed safe limits per contact point, that attachment placements are compatible with the specified tooth movements, and/or that the overall treatment duration falls within acceptable ranges. Clinical validation may compare the machine-readable instructions 406 against a database of clinical rules, safety thresholds, and/or best practice guidelines to identify potential violations.

In some embodiments, validation 412 includes semantic validation to confirm that the machine-readable instructions 406 accurately reflect the intent of the original natural language instructions 402. Semantic validation may involve providing both the natural language instructions 402 and the machine-readable instructions 406 to an AI model configured to predict whether the machine-readable instructions 406 correctly capture the meaning of the natural language instructions 402. The AI model may generate a confidence score indicating the likelihood that the translation is accurate.

In some embodiments, validation 412 includes compatibility validation to determine whether the machine-readable instructions 406 are compatible with the treatment building engine or treatment planning engine that will execute the instructions. Compatibility validation may verify that all instruction types, parameters, and values in the machine-readable instructions 406 are supported by the target engine, and that no unsupported or deprecated instruction formats are present.

Validation 412 may produce multiple possible outcomes based on the results of the validation operations. In a first outcome, validation 412 determines that the machine-readable instructions 406 pass all validation checks, and the machine-readable instructions 406 are provided to the integration and mapping engine 414 for further processing and treatment plan generation. In a second outcome, validation 412 identifies one or more validation failures that can be automatically corrected, and validation 412 applies corrections to the machine-readable instructions 406 to generate corrected instructions that are then re-validated or provided to the integration and mapping engine 414. Automatic corrections may include adjusting values that slightly exceed thresholds to fall within acceptable ranges, correcting minor syntax errors, or resolving ambiguities based on default values. In a third outcome, validation 412 identifies one or more validation failures that cannot be automatically corrected, and the machine-readable instructions 406 are directed to manual treatment planning 418 for review and correction by a trained technician. Instructions directed to manual treatment planning 418 may include instructions that violate clinical safety constraints, instructions that contain unsupported operations, instructions that have low semantic validation confidence scores, or instructions that contain ambiguities that require human judgment to resolve.

In some embodiments, validation 412 generates a validation report that identifies specific validation failures, provides explanations for why each failure occurred, and suggests potential corrections or modifications. The validation report may be provided to a treatment provider or technician to facilitate manual review and correction of the machine-readable instructions 406. In some embodiments, validation 412 maintains a log of validation results for auditing and quality assurance purposes, enabling analysis of common validation failures and identification of opportunities to improve the translation model 404 or the validation rules.

Upon failure of validation 412, flow may continue to a manual treatment planning 418. Manual treatment planning 418 may include providing instructions (e.g., natural language instructions 402) to a technician trained to generate a treatment planning algorithm based on practitioner instructions. Other points of data flow 400A may also cause manual treatment planning 418 to be performed, e.g., failure of any earlier steps may also cause flow to be diverted to manual treatment planning 418.

Upon machine-readable instructions 406 passing validation 412, the instructions may be provided to integration and mapping engine 414. Integration and mapping engine 414 may update or create a treatment algorithm based on the machine-readable instructions 406. The integration and mapping engine 414 serves as a bridge between the validated machine-readable instructions 406 and the patient-specific dental data, translating abstract treatment directives into concrete treatment operations applicable to a particular patient's dentition. In some embodiments, the integration and mapping engine 414 receives the machine-readable instructions 406 along with patient data 416, which may include a three-dimensional model of the patient's dentition obtained from intraoral scan data or other dental arch data capturing equipment. In embodiments, the integration and mapping engine 414 processes these inputs to generate treatment plan 420, which incorporates the updates encoded in natural language instructions 402. In some embodiments, e.g., if integration fails due to incompatibility between the instructions and the patient data, data flow 400A may be directed to manual treatment planning 418 at this stage.

In some embodiments, integration and mapping engine 414 may perform operations mapping tooth identifiers from the machine-readable instructions 406 to patient data 416. The mapping operations may include resolving references to teeth expressed in various numbering systems (such as Universal, FDI, or Palmer notation) to the specific teeth present in the patient's three-dimensional dental model. In some embodiments, a digital model's teeth (e.g., patient data 416) may be sorted based on geometric position along a jaw arch. Geometric sorting may ensure consistent ordering even in the presence of irregularities such as unerupted, supernumerary, or pontic teeth. The sorting may be cached and reused across multiple instructions within the same case, e.g., to optimize performance. The integration and mapping engine 414 may segment the three-dimensional model to identify individual teeth, determine their positions relative to one another, and establish correspondences between the tooth references in the machine-readable instructions 406 and the actual teeth represented in the patient data 416.

In some embodiments, instructions may be mapped to the array of teeth. Selection may include iterating through the sorted array and selecting only those teeth that match the description included in machine-readable instructions 406 for any treatment instruction. Matching logic may include individual teeth, tooth groups, intervals, relative positions, exclusion, etc. The integration and mapping engine 414 may apply the treatment operations specified in the machine-readable instructions 406 to the selected teeth, generating staging information that defines how teeth should move from their initial positions to target positions over a series of treatment stages. The engine may also determine placement of dental features such as attachments, precision cuts, and/or bite ramps based on the instructions.

For some instruction types, one or more interteeth intervals may be utilized. For example, instructions involving interproximal spaces, such as gap closures or interproximal reduction (IPR), may include references for spaces between teeth. Parsing instructions may include deriving an array of interteeth intervals from a mapped array of teeth. Geometrical sorting of teeth (or other ordered objects with relevant spaces between) facilitates generation of interteeth intervals. For example, an interval i may be defined as (tooth i, tooth i+1) or interval j may be defined as (tooth j, tooth j+1). These interproximal intervals may be utilized by integration and mapping engine 414 for generation of treatment plan 420.

The final output of integration and mapping engine 414 is treatment plan 420 in some embodiments, which may include a comprehensive specification for the dental treatment. Treatment plan 420 may be an orthodontic treatment plan including staging information defining the sequence of tooth movements across multiple treatment stages, final tooth positions representing the target arrangement of the patient's dentition, specifications for dental features to be applied at various stages, and/or design parameters for treatment appliances. In some embodiments, treatment plan 420 may be stored in a treatment planning data format that encodes the three-dimensional model of the teeth along with the treatment planning information. Treatment plan 420 may be provided to a treatment provider for review and approval via a graphical user interface. Upon approval, treatment plan 420 may be used to generate manufacturing data for one or more dental treatment appliances, such as orthodontic aligners, which may then be fabricated using direct fabrication techniques or other manufacturing processes. The treatment appliances may be provided to the patient for use in implementing the treatment plan to progressively reposition the patient's teeth from an initial arrangement toward the target arrangement specified in treatment plan 420.

Machine-generated prompts may provide significant advantages over manually crafted prompts in dental treatment planning systems. Conventional prompt engineering typically requires substantial human expertise and iterative refinement to develop prompts that reliably cause an LLM to generate accurate, well-formatted output. This manual process can be time-consuming, may not scale well across different treatment categories or instruction types, and may fail to account for edge cases or unusual input patterns that arise in practice. Furthermore, as treatment planning systems evolve and new categories of instructions or treatment options are introduced, manually updating prompts to accommodate these changes can introduce errors or inconsistencies.

Machine-generated prompts may address these limitations by leveraging the capabilities of LLMs to analyze existing prompts, identify deficiencies, and generate improved or extended prompts that better capture the semantics of target tasks. By providing an LLM with a base prompt, task descriptions, design principles, and representative examples, the system can automatically generate prompts that handle a broader range of input scenarios, maintain consistency with output schema requirements, and adapt to new categories or instruction types without requiring extensive manual intervention. This approach may reduce the time and expertise required to develop effective prompts, improve the reliability of LLM-based treatment planning operations, and enable more rapid iteration when prompt improvements are needed.

FIG. 4B is a diagram of a data flow 400B for generating and implementing a machine-generated prompt for an LLM, according to some embodiments. Data flow 400B begins with generation of a prompt generation request 420. Prompt generation request 420 may be based on a previous prompting strategy for an LLM. Prompt generation request 420 may be configured to generate a prompt that performs more effectively than a previous version, for example. Prompt generation request 420 may be configured to generate a prompt that extends utility or addresses a related query to a previous LLM prompt in some embodiments. The prompt generation request 420 serves as the foundational input that defines the objectives, constraints, and context for the machine-generated prompt.

Prompt generation request 420 may include a base prompt 422. The base prompt 422 may be a previously developed or previously used prompt for an LLM to configure the LLM to perform a base task. The base task may be the same task as a target task related to prompt generation request 420, or a different task. In some embodiments, the base prompt 422 may have been utilized in one or more situations in which the base prompt 422 did not perform adequately, e.g., an LLM prompted with base prompt 422 may have generated erroneous output, may have classified one or more instructions incorrectly, may have generated pseudo machine-readable output that was not compatible with a target system, or the like. In some embodiments, a target task may be related to a base task of base prompt 422. For example, a target task may be an extension of a base task, such as a base task including categorizing input into a first set of categories, and a target task including assigning all input into a second set of categories, which may include additional categories not accounted for in base prompt 422. The base prompt 422 provides a starting point that captures existing knowledge about how to structure prompts for the domain (e.g., dental treatment domain), enabling the prompt generator to build upon proven approaches rather than generating prompts from scratch. In some embodiments, the base prompt 422 may be derived from a prompt used in a related treatment planning context, such as a prompt adapted for use in processing digital detailing (DDT)-related instructions or other categories of treatment provider input.

Prompt generation request 420 may include task description 424. Task description 424 may include a description of problems or deficiencies of the base prompt 422. Task description 424 may include a description of the extended utility of a target task, compared to a base task of base prompt 422. Task description 424 may indicate that the machine-generated prompt is to perform actions included in the base prompt 422, as well as additional actions. Task description 424 may include one or more examples where base prompt 422 did not perform as intended, did not perform to an adequate degree of confidence or accuracy, or the like. The task description 424 may specify the semantic requirements of the target task, including what types of natural language input the prompt should handle, what categories or classifications should be recognized, and what format the output should take. In some embodiments, task description 424 may identify specific false positive cases that occurred when using base prompt 422, enabling the prompt generator to understand patterns of errors and generate prompts that avoid similar mistakes. Task description 424 may further specify performance metrics or accuracy thresholds that the machine-generated prompt should achieve, providing quantitative targets for the prompt generation process.

Prompt generation request 420 may further include design principles 426. Design principles 426 may include a description of schema of the intended output of an LLM based on the machine-generated prompt. Design principles 426 may include references to one or more machine-readable languages that output is to emulate. Design principles 426 may include references to external documents to reference for output generation, e.g., in a RAG system. Design principles 426 may provide one or more examples of output in a target format, of input/output pairing including output exhibiting target output properties (e.g., format, syntax, etc.), or the like. The design principles 426 may specify schema (e.g., JSON schema) definitions that constrain the structure and allowed values of LLM output, ensuring that generated machine-readable instructions conform to formats expected by downstream treatment planning systems. Design principles 426 may include specifications for how teeth should be referenced using various numbering systems (e.g., Universal, FDI, Palmer), how treatment stages should be represented, and/or how different categories of dental features or treatment operations should be encoded. In some embodiments, design principles 426 may specify that the machine-generated prompt should cause the LLM to limit its output to instructions from a predefined library of machine-implementable instructions, preventing the LLM from generating invalid or unsupported instruction types.

Prompt generation request 420 may include examples 428. Examples 428 may include examples of scenarios that the machine-generated prompt generated in response to prompt generation request 420 may be expected to correctly classify, translate, describe, or otherwise manage. In some embodiments, examples 428 may include example input, but not include target output. For example, in a case where a classification scheme is to be extended to include additional categories, some input that is either expected to cause a system to operate incorrectly, or historical examples of inaccurate or insufficient classification, may be provided as part of examples 428. The LLM may be provided with a task of determining a prompt that can correctly manage the examples 428, without providing target output for the classification with respect to the examples 428. Examples 428 may be selected to represent a diverse range of input patterns, including common cases, edge cases, and cases that have historically caused errors. In some embodiments, examples 428 may include representative samples of false positives identified from processing historical treatment provider instructions, enabling the prompt generator to understand patterns of misclassification and generate prompts that correctly handle similar cases. Examples 428 may further include examples in multiple languages, as treatment providers may submit instructions in various languages, and the machine-generated prompt may need to handle multilingual input appropriately.

Prompt generation request 420 is provided to prompt generator 430. Prompt generator 430 may be an AI model. Prompt generator 430 may be an LLM. Prompt generator 430 may be the same LLM that is used to accomplish the base task of base prompt 422, the same LLM to which the machine-generated prompt is eventually to be provided as input, or the like. Prompt generator 430 may be a different LLM than one or more other LLMs used in a prompting system, e.g., to enable higher degrees of robustness in prompt generation by excluding some intrinsic patterns or biases that may be present in a single LLM. The prompt generator 430 analyzes the components of prompt generation request 420 to understand the requirements of the target task and synthesizes a prompt that addresses those requirements. Prompt generator 430 may leverage its understanding of natural language, prompt engineering patterns, and domain-specific terminology to generate prompts that effectively communicate task requirements to downstream LLMs. In some embodiments, prompt generator 430 may be a state-of-the-art LLM with strong reasoning capabilities, enabling it to identify subtle patterns in the examples 428 and generate prompts that capture those patterns. Prompt generator 430 may generate prompts that include detailed instructions, category definitions, output format specifications, and examples, structured in a manner that maximizes the likelihood of correct LLM behavior on the target task.

Prompt generator 430 generates a draft prompt 432. Draft prompt 432 may be configured to cause an LLM, with potential additional input, to perform a target task. For example, the draft prompt 432 may accompany natural language instructions, and may cause the LLM to classify instructions, generate machine-readable instructions related to the input natural language instructions, or the like. Draft prompt 432 represents an initial version of the machine-generated prompt that captures the prompt generator's interpretation of the requirements specified in prompt generation request 420. Draft prompt 432 may include multiple sections, such as a system message section that establishes the role and context for the LLM, a task description section that specifies what the LLM should accomplish, a category definition section that describes the categories or classifications relevant to the task, an output format section that specifies the schema and structure of expected output, and/or an examples section that provides representative input/output pairs to guide LLM behavior. In some embodiments, draft prompt 432 may include instructions for handling ambiguous or unclear input, specifying how the LLM should respond when input does not clearly match any defined category or when multiple interpretations are possible.

Draft prompt 432 may optionally be provided to refinement loop 434. Refinement loop 434 may include machine-supervised and/or human-supervised refinement tasks. Refinement loop 434 may include validating some portions of a prompt. For example, an entire prompt may be quite long, e.g., difficult to proofread or correct in its entirety. However, draft prompt 432 may include a schema, which indicates for example design principles 426 in a format that is predicted to be managed well by an LLM. These schema may be more easily inspected, either by a subject matter expert or electronically (e.g., by another instance of an LLM, to determine whether the schema correctly reflect the intended design principles of the machine-generated prompt). In some embodiments, one or more changes may be made to the draft prompt, and/or one or more changes may be made to prompt generation request 420, until a draft prompt that appears to satisfy one or more target conditions is generated. Refinement loop 434 may involve iteratively evaluating the draft prompt 432 against the examples 428 to assess how well the prompt captures the semantics of the task description 424. During each iteration, the refinement loop 434 may identify weaknesses in the draft prompt, such as edge cases that are not handled correctly, ambiguities in category definitions, or inconsistencies between the prompt instructions and the design principles 426. The refinement loop 434 may suggest improvements to address identified issues, such as clarification of edge cases, addition of rules to handle specific patterns, or addition of examples to illustrate correct behavior. In some embodiments, refinement loop 434 may be performed automatically by providing the draft prompt 432 and evaluation results to an LLM configured to analyze prompt performance and suggest improvements. An output of a cycle of the refinement loop 434 may be an updated version of the machine-generated prompt. The updated version may be processed in a similar manner to further refine the prompt to generate still a further iteration of the prompt. The refinement loop 434 may continue until stopping criteria are satisfied, such as achieving a target accuracy threshold on the examples 428, completing a maximum number of iterations, or determining that no further improvements are being made.

After refinement loop 434, data flow continues to validation 436. Validation 436 may include testing performance of draft prompt 432. Validation 436 may include providing draft prompt 432 and test input data (e.g., natural language instructions not used in training or generation of the draft prompt 432) to an LLM to gauge performance of draft prompt 432. In some embodiments, validation 436 may cause a return to refinement loop 434, including updating one or more portions of prompt generation request 420 (e.g., additional examples 428 may be provided corresponding to situations which were addressed incorrectly in validation 436). In some embodiments, validation 436 may be performed based on labeled data, e.g., input/output pairs may be used for testing draft prompt 432. Upon passing validation, draft prompt 432 is output as final prompt 438 (e.g., a machine-generated LLM prompt), for use in further applications related to the target task associated with prompt generation request 420. Validation 436 may include multiple types of tests, such as accuracy tests that measure the percentage of test cases correctly handled, format validation tests that confirm output conforms to the specified schema, and safety tests that verify the prompt does not cause the LLM to generate instructions that violate treatment heuristics or safety constraints. Validation 436 may further include testing the prompt on a held-out dataset of historical treatment provider instructions to assess real-world performance. In some embodiments, validation 436 may include human review of a sample of LLM outputs generated using the draft prompt 432, enabling subject matter experts to identify subtle errors or issues that automated tests may not detect. The validation 436 process ensures that the final prompt 438 meets quality standards before being deployed for use in production treatment planning systems.

The final prompt 438 represents the completed machine-generated prompt that has been refined and validated for use in downstream tasks. Final prompt 438 may be stored in a prompt registry or database for retrieval when processing treatment provider instructions. In some embodiments, final prompt 438 may be versioned, enabling tracking of prompt changes over time and rollback to previous versions if issues are identified. Final prompt 438 may be used in pipelines for automated text understanding, including generating machine-readable instructions for dental treatment planning operations. The final prompt 438 may be provided along with natural language treatment provider instructions to an LLM, causing the LLM to generate machine-readable instructions that can be validated and applied to treatment planning algorithms. By utilizing machine-generated prompts, the treatment planning system may achieve more consistent and reliable translation of natural language instructions into machine-readable format, reducing errors and improving the efficiency of automated treatment planning operations.

In conventional dental treatment planning workflows, treatment providers may submit case modification or design requests that include free-text comments or instructions describing desired changes to a treatment plan or details for new treatment plan. These comments may relate to various aspects of treatment, such as adding or removing attachments, adjusting treatment stages, modifying tooth movements, or other treatment parameters. Processing such free-text instructions has traditionally required manual intervention by trained technicians who interpret the treatment provider's intent and translate the instructions into machine-readable format that can be applied to the treatment planning system. This manual process introduces delays, increases costs, and may result in inconsistent interpretation of treatment provider instructions across different technicians.

The method of FIG. 4C addresses these limitations by providing an automated pipeline for processing treatment provider instructions. By classifying natural language instructions into categories and converting those instructions into machine-readable format using AI models, the method enables automated processing of case modifications without requiring manual technician intervention for supported instruction categories. This approach may reduce turnaround time for case modifications, improve consistency in instruction interpretation, and allow treatment providers to receive updated treatment plans more quickly. The method also provides a mechanism for routing instructions that cannot be automatically processed to manual processing pathways, thereby ensuring that complex or unsupported instructions are still handled appropriately while enabling automation for routine instruction categories.

FIG. 4C is a diagram of a data flow 400C for classifying instructions into categories for further processing, according to some embodiments. The data flow 400C illustrates a multi-stage pipeline for processing treatment provider instructions, where natural language input is progressively analyzed, categorized, and converted into machine-readable format suitable for automated treatment planning operations. At block 440, treatment provider instructions are provided to a comment or instruction management system. The treatment provider instructions may include natural language instructions. The treatment provider instructions may additionally or alternatively include instructions that are not natural language instructions, e.g., selections in a GUI-guided treatment protocol process may be provided, optionally along with free text comments provided in a free text entry GUI element of the treatment protocol GUI. The treatment provider instructions may be provided through free-text, speech, and/or other instruction providing methods. The treatment provider instructions may be provided via a comment element in a treatment planning and/or protocol generation GUI in some instances. For example, a treatment provider may enter instructions such as “add optimized attachments to teeth 12 and 13” or “remove all attachments from the last 4 stages” in a free text field associated with a case modification request. The treatment provider instructions may be provided to a comment detection 442 component, e.g., for determining whether the instructions include natural language comments.

At comment detection 442, the system determines whether manual comments are present in the treatment provider instructions 440. If a comment is detected, the comment detection 442 component may identify and extract relevant portions of the natural language input for further processing. For example, comment detection 442 may be performed to distinguish between structured data fields that have been populated through GUI selections and free-text comments that require natural language processing. In some embodiments, if no manual comments are detected, the treatment provider instructions may bypass subsequent natural language processing stages and proceed directly to automated processing based on the structured data. Responsive to a determination that the treatment provider instructions 440 include natural language comments, treatment provider instructions 440 may be provided to a classification model 444. The classification model 444 may be an AI-based model. The classification model 444 may be an LLM. The classification model 444 may also be provided with a prompt, e.g., a prompt to configure an LLM to classify incoming natural language instructions into one or more categories.

In some embodiments, classification model 444 may be configured to separate treatment provider instructions 440 (e.g., natural language portions of treatment provider instructions 440) into categories which are to be managed differently by the treatment planning system. The classification model 444 may categorize instructions into a plurality of predefined categories, such as dental features, passive aligners, overcorrection, treatment length, interproximal reduction, and/or other treatment-related categories. For example, an instruction such as “add G4 attachments to teeth 1.2 and 1.3” may be classified into a dental features category, while an instruction such as “add 4 passive trays at the end” may be classified into a passive aligners category. Some categories of instructions may be managed deterministically by a heuristic or rule-based system, such as adjusting tooth numbering to a standard tooth numbering scheme. Some categories of instructions may be managed by an AI model such as an LLM, which may convert some types of free-text instructions to machine-implemented instructions (e.g., adjusting selections that may be made in a drop-down menu or selections made on a GUI) or machine-readable code. Some categories of instruction may be provided for manual processing by a technician (e.g., instructions that cannot be understood or classified, instructions that do not provide a high level of confidence that an AI model will manage them correctly, etc.). In some embodiments, the classification model 444 may determine that instructions belong to multiple categories, and the instructions may be processed by multiple downstream systems accordingly. For example, instructions may be divided into sections, and each section may be processed using a different downstream system.

In some embodiments, classification model 444 may classify or categorize instructions into categories, and the categories may later be assigned to appropriate systems for further processing. In some embodiments, classification of instructions may include classifying all instructions, e.g., to ensure that no clinically relevant instructions are skipped. In some embodiments, instructions may be classified into categories based on clinical relevance. For example, instructions related to treatment may be classified into a first set of categories, and non-clinical comments such as polite comments (e.g., “thanks for your help” or “please let me know if you have questions”) may be classified as non-clinical or as polite expressions. In some embodiments, non-clinical comments may be excluded from further processing. In some embodiments, one or more classifications of instructions may be provided for further AI processing. For example, specific types of instructions which have an associated AI prompt that is understood to perform well may be provided for LLM-based processing. In some embodiments, instructions related to dental features (e.g., attachments) may be provided for further processing.

In some embodiments, the machine-readable instructions generated from the natural language instructions may comprise updates to one or more treatment plan parameters for the target dental treatment. The treatment plan parameters may encompass various aspects of the treatment planning process that can be adjusted based on treatment provider instructions.

In some embodiments, the one or more treatment plan parameters may comprise dental appliance features for one or more dental appliances to be used for the target dental treatment. Dental appliance features may include physical characteristics or functional elements that are incorporated into orthodontic appliances such as aligners. For example, a treatment provider instruction such as “add retention attachments to the upper premolars” may be processed to generate machine-readable instructions that update the dental appliance features for the corresponding treatment stages.

In some embodiments, the dental appliance features may comprise at least one of attachments, bite ramps, or modeled appliance features. Attachments may include optimized attachments configured for specific tooth movements such as rotation, extrusion, or root control. Bite ramps may be features incorporated into aligners to address overbite or facilitate specific occlusal relationships. Modeled appliance features may include precision cuts, power ridges, or other geometric modifications to the appliance design. For example, an instruction such as “add bite ramps to the upper anterior teeth” may be classified and processed to generate machine-readable instructions specifying the placement and configuration of bite ramp features.

In some embodiments, the dental appliance features may comprise mandibular advancement features selected from at least one of buccal blocks or occlusal blocks. Mandibular advancement features may be used in treatments addressing Class II malocclusions or other conditions where repositioning of the mandible is desired. Buccal blocks may be positioned on the buccal surfaces of posterior teeth, while occlusal blocks may be positioned on occlusal surfaces. Instructions related to mandibular advancement features may be classified and processed to generate corresponding machine-readable instructions for appliance design.

In some embodiments, the one or more treatment plan parameters may comprise one or more planning targets. Planning targets may define the goals or endpoints of the treatment planning process, guiding how the treatment planning algorithm generates tooth movements and staging.

In some embodiments, the one or more planning targets may comprise at least one of intended final positions for one or more teeth, tooth velocities for one or more teeth, target treatment outcome, number of treatment stages, amount of overcorrection, or whether to apply passive aligners. For example, an instruction such as “limit treatment to 20 stages” may be processed to generate machine-readable instructions that constrain the number of treatment stages. An instruction such as “add 3 mm of overcorrection for the upper anterior spacing” may be processed to update the amount of overcorrection parameter. An instruction such as “add 4 passive aligners at the end of treatment” may be processed to update parameters related to passive aligner application. Instructions specifying final tooth positions or movement velocities may similarly be classified and converted to machine-readable format for integration into the treatment planning algorithm.

In an example, the classification model 444 may determine that a case includes only instructions related to optimized attachments and polite expressions, and such a case may be eligible for fully automated processing. In some embodiments, instructions related to other treatment parameters may similarly be classified and processed. For example, instructions related to interproximal reduction (IPR) may be classified into a category for automated processing, where the classification model 444 identifies requests to add, limit, or schedule IPR at specific stages or contacts. Instructions related to passive aligners may be classified into a category indicating requests for additional passive stages at the end of treatment. Instructions related to overcorrection may be classified into a category for processing requests to add overcorrection stages or specify overcorrection parameters. Instructions related to treatment length may be classified into a category for processing requests to extend or shorten the overall treatment duration or number of stages. Instructions related to residual spaces may be classified into a category for processing requests to preserve or close gaps between specific teeth. Instructions related to bite ramps may be classified into a category for processing requests to add or modify bite ramp features. Instructions related to precision cuts may be classified into a category for processing requests to add precision cuts to specific teeth or stages. Instructions related to pontics may be classified into a category for processing requests to place pontics in areas of missing teeth. Instructions related to extractions may be classified into a category for processing requests to mark specific teeth for extraction. Instructions related to tooth movement restrictions may be classified into a category for processing requests to limit or prevent movement of specific teeth. Instructions related to attachment placement may be classified into a category for processing requests to add attachments to specific teeth, where the classification model 444 identifies the action as adding attachments and extracts relevant parameters such as teeth identifiers, attachment types, and staging information. Instructions related to attachment removal may be classified into a category for processing requests to remove existing attachments from specific teeth. Instructions related to attachment retention may be classified into a category for processing requests to keep or retain existing attachments on specific teeth. Instructions related to attachment replacement may be classified into a category for processing requests to replace existing attachments with different attachments on specific teeth. Instructions related to attachment modification may be classified into a category for processing requests to modify properties of existing attachments such as size, position, or alignment. Instructions related to forbidding attachment placement may be classified into a category for processing requests to prevent placement of attachments on specific teeth. Instructions related to delaying attachment placement may be classified into a category for processing requests to delay placement of attachments to later stages of treatment. In some embodiments, the classification model 444 may determine that a case includes only instructions belonging to one or more of these supported categories along with polite expressions, and such a case may be eligible for fully automated processing. Conversely, if the classification model 444 identifies instructions belonging to categories that are not yet supported for automated processing, the case may be routed to manual processing by a technician.

One or more categories of input classified by classification model 444 may be provided to conversion model 446. Conversion model 446 may be heuristic, may be rules-based, may include one or more AI models, etc. Conversion model 446 may perform additional processing techniques on input instruction data. Conversion model 446 may perform conversion from natural language instructions to machine-readable instructions, e.g., instructions in or resembling a programming language for implementation in further treatment planning operations. The conversion model 446 may be a specialized AI model configured to understand the meaning and intent of treatment provider instructions and extract specific parameters therefrom. For example, the conversion model 446 may extract information such as teeth identifiers, treatment stages, treatment actions (e.g., add, remove, keep, modify), attachment types (e.g., optimized attachments, conventional attachments), and/or other parameters from natural language instructions. The conversion model 446 may be provided with a prompt that includes a schema defining the structure and allowed values for the output, thereby constraining the model to produce output that conforms to a predefined format. For example, an instruction such as “add largest dual MDRC attachments to upper anteriors starting at stage 5” may be converted to a JSON object specifying the attachment type as “mesial_distal_root_control,” the size as “largest,” the MDRC type as “dual,” the teeth as upper anterior teeth, and the starting stage as stage 5. The conversion model 446 may also handle different tooth numbering systems used by treatment providers, such as Universal, FDI, and Palmer numbering systems, and may identify which numbering system is being used based on the format of the tooth identifiers in the instructions.

In some embodiments, output of conversion model 446 may be provided for post-processing 448. Post-processing 448 may include adjustments to formatting, features, syntax, or the like of output of conversion model 446 to enable use of the output in further operations related to treatment planning. Post-processing 448 may include renumbering of indices, such as tooth numbering, to a standard scheme. For example, if the treatment provider used FDI numbering (e.g., “1.2” for upper right lateral incisor) but the treatment planning system uses Universal numbering, post-processing 448 may convert the tooth identifiers to the Universal numbering scheme (e.g., tooth 7). Post-processing 448 may include validation to confirm that the converted instructions are compatible with the treatment planning engine and that the specified parameters are supported. For example, post-processing 448 may verify that the specified attachment type is a valid attachment type, that the specified teeth exist in the patient's dentition, and that the specified stages are within the range of stages in the treatment plan. Post-processing 448 may include removing portions of a response accompanying machine-readable instructions, such as LLM-generated descriptions of the machine-readable instructions. Post-processing 448 may also include checking for inconsistencies between the detected numbering system and the numbering system known to be used by the treatment provider, and resolving such inconsistencies. After post-processing 448, machine-readable instructions 450 related to the target instructions categories may be generated. The machine-readable instructions 450 may be in a structured format such as JSON that can be directly applied to update a treatment plan or treatment protocol. In some embodiments, if post-processing 448 determines that the converted instructions are not compatible with the treatment planning engine or that certain instructions are not supported, the case may be routed to manual processing rather than proceeding with automated treatment planning.

FIG. 4D is an example treatment planning GUI 400D, including an AI assistant, according to some embodiments. Treatment planning GUI 400D may share one or more features with other GUIs discussed herein, such as those discussed in connection with FIG. 3F, 3G, or 3H. A GUI such as treatment planning GUI 400D may be applicable to protocol generation, treatment plan or treatment prescription generation, or other aspects of treatment planning.

GUI 400D includes a treatment category menu 452 positioned on the left side of the interface. Treatment category menu 452 may share one or more features with treatment category selection 374 of FIG. 3F. Treatment category menu 452 may include various aspects of treatment within which a treatment provider may provide instructions, exhibit treatment preferences, update treatment plans, or the like. Treatment category menu 452 lists various treatment categories including arch to treat, anterior-posterior correction, interproximal reduction (IPR), spacing, overbite, posterior crossbite, anterior leveling, midline, attachments, precision cuts, bite ramps, power ridge feature, and passive/active aligners. The treatment category menu 452 enables a treatment provider to navigate between different categories of treatment parameters and preferences.

In some embodiments, treatment category menu 452 may include one or more alert indicators 454. The alert indicators 454 appear adjacent to certain treatment categories in the treatment category menu 452, providing a visual flag indicating areas of the treatment planning that are to be updated, that may have been updated, that may be targeted for updating, or the like. The alert indicators 454 may indicate that changes or updates are available for review in the associated treatment category. In some embodiments, an AI assistant chat panel 456 may be used to make or recommend one or more adjustments to a treatment planning template. Changes made or recommended via the AI assistant chat panel 456 may be marked for review with alert indicators 454.

GUI 400D includes a search field. The search field enables a treatment provider to search within the clinical preferences template for specific treatment categories, parameters, or settings. The search field may facilitate rapid navigation to desired treatment options without requiring manual navigation through the treatment category menu 452.

In some embodiments, a category of recommended changes may be included on the GUI, e.g., in treatment category menu 452. For example, a “review changes” category may be added to the treatment category menu 452. Selecting the recommended changes (e.g., “review changes”) may cause a recommended changes window 458 to be displayed. In some embodiments, options to accept or reject all recommended changes, or options to accept or reject recommended changes individually, may be provided. In the illustrated example, the recommended changes window 458 displays spacing rules and midline rules with accept and reject options for each rule, allowing the treatment provider to review and approve proposed changes.

In some embodiments, GUI 400D includes AI assistant chat panel 456, displayed on the right side of the interface in the illustrated example. AI assistant chat panel 456 enables a user (e.g., treatment provider) to chat with an AI assistant function through a conversational interface. The AI assistant chat panel 456 displays a chat exchange including treatment provider requests and corresponding AI assistant responses. For example, the chat exchange may include treatment provider requests such as requests for setting spacing treatment to distal to canines for teen cases, achieving a midline goal using IPR, and inquiring about distalization options. In the illustrated example, the AI assistant chat panel 456 provides responses confirming that spacing configuration has been successfully applied, that midline correction with IPR was successfully applied to both adult and teen cases, and detailed information about distalization techniques including compact sequential distalization and improved sequential distalization with their respective characteristics.

The AI assistant function may be executed by an LLM, provided with prompting specific to the context of the AI assistant in embodiments. For example, the AI assistant may be provided additional prompting information related to healthcare, orthodontic or dental care, a relevant stage of care (e.g., treatment protocol generation for an assistant tied to a protocol tool, treatment plan generation for an assistant associated with a treatment planning tool, etc.), and the like. The AI assistant may be coupled to data storage, e.g., the AI assistant may store one or more previous requests, request results, patterns in previous requests, or the like as additional context, additional prompting information, or the like. In some embodiments, AI assistants across various stages of treatment planning may exchange information, e.g., an update to a protocol that has not been requested in previous treatment planning operations may prompt an AI assistant to ask a clarifying question, such as whether the treatment provider is sure they would like to make the indicated protocol adjustment. In another example, after multiple instances of a particular change being made in a treatment planning stage, an AI assistant may provide a query to determine whether the treatment provider would like to update a treatment protocol to include the change, to be automatically applied to all treatment plans to which the change is applicable (e.g., cases where an associated dental disorder is present).

FIG. 4E is an example process flow 400E for using an AI assistant to update one or more stages of treatment planning, according to some embodiments. Process flow 400E depicts an exchange of information between four entities, practitioner 460 (e.g., a doctor or treatment provider), treatment planner 462 (e.g., a GUI-based application or web portal for performing one or more treatment planning activities), assistant 464 (e.g., an LLM-based assistant, including an agent for interacting with the LLM, potentially an API or other system for accessing the LLM, and/or the LLM itself), and treatment storage 468 (e.g., data store 140 of FIG. 1). The entities depicted in process flow 400E may be co-located at a single physical location or may be distributed across different geographic locations. For example, the practitioner 460 may interact with the system via a client device, such as a desktop computer, laptop computer, tablet, or other computing device located at the practitioner's office or clinic. In some embodiments, a treatment planning application executes on the client device of the practitioner 460. The treatment planning application may comprise a native application installed on the client device that provides a graphical user interface for the practitioner 460 to interact with the treatment planner 462, assistant 464, and treatment storage 468. The treatment planning application may communicate with the treatment planner 462, assistant 464, and treatment storage 468 via network communications, such as HTTP requests, WebSocket connections, or other network protocols. The treatment planning application may transmit natural language instructions from the practitioner 460 to the assistant 464 and receive machine-readable instructions, treatment plan updates, and other data in response. In some embodiments, a web browser executes on the client device of the practitioner 460. The web browser may load a web-based treatment planning interface from one or more servers that host the treatment planner 462. The web browser may communicate with the servers via HTTP or HTTPS requests to transmit practitioner instructions and receive treatment planning data, treatment protocol updates, and responses from the assistant 464. The web browser may render the graphical user interface elements, including chat interfaces for interacting with the assistant 464, treatment category menus, and treatment plan visualizations. The servers may process requests from the web browser, invoke the assistant 464 to generate machine-readable instructions from natural language input, and return responses to the web browser for display to the practitioner 460. The treatment planner 462, assistant 464, and/or treatment storage 468 may execute on one or more remote servers that communicate with the client device of the practitioner 460 over a network, such as network 130 of FIG. 1. In some embodiments, the treatment planner 462, assistant 464, and/or treatment storage 468 may execute on computing devices that are virtual machines or containerized instances running in a cloud computing environment. The cloud computing environment may provide scalable computing resources, enabling the treatment planner 462 and assistant 464 to handle varying workloads and multiple concurrent practitioner sessions. In other embodiments, some or all of the treatment planner 462, assistant 464, and/or treatment storage 468 may execute locally on the client device of the practitioner 460 or on computing devices within a local network at the practitioner's facility.

Practitioner 460 may perform an action to open treatment viewer 470. Opening treatment viewer may include launching an application, opening a web page, logging in to a web portal, or another means of launching a GUI for viewing and editing treatment options. The treatment planner 462 may be configured to edit treatment protocols. Treatment planner 462 may additionally or alternatively be configured to edit treatment plans. In some embodiments, treatment planner 462 may include GUI-based selections, e.g., check boxes, drop-down menus, etc., for selecting treatment options. In some embodiments, treatment planner 462 may include free text entry, including natural language comment entry or elements for entering machine-readable instructions, e.g., for coding operations related to generating treatment plans. In some embodiments, treatment planner 462 may include an AI assistant element, e.g., a chat element for accessing an LLM-based AI assistant 464. Treatment planner 462 may show assistant UI 472 to practitioner 460 in embodiments.

Flow continues with practitioner 460 entering text request 474 via the AI assistant. The text request may include one or more instructions, one or more queries (e.g., directed toward treatment options presented in treatment planner 462), or the like. In some embodiments, one or more messages may be passed back and forth between assistant 464 and practitioner 460, e.g., as assistant 464 determines whether instructions provided have clinical relevance, as proposed adjustments to treatment are recommended or confirmed, as information responding to practitioner queries are provided, or the like.

Assistant 464 determines proposed changes 478 based on text request of the practitioner 460. Determining proposed changes may include utilizing one or more models, one or more functions, one or more LLMs, one or more prompting strategies, etc. For example, a classification model (e.g., an LLM prompted to classify instructions into categories) may be used to separate the instructions into different groups, one or more translation models (e.g., LLM models prompted to generate machine-readable instructions from natural language instructions) may be used to generate machine-readable instructions, etc.

Assistant 464 may provide the proposed changes for proposed changes verification 480. Proposed changes verification may include providing the proposed changes for review by practitioner 460 by transmitting the proposed changes to a client device of the practitioner. Proposed changes verification may include providing a description of changes in an AI assistant GUI element. Proposed changes verification may include providing a description of proposed changes in a proposed changes GUI element, e.g., recommended changes window 458 of FIG. 4D. Proposed changes verification may include providing an indicator to a setting that has been adjusted, e.g., on a GUI-based treatment planner. Proposed changes verification may include providing instructions to implement changes requested by practitioner 460, e.g., guiding a practitioner to a setting within a GUI-based treatment planner that corresponds to the requested update. Practitioner 460 may accept changes 482 by interacting with any of these or other methods for verification or review of proposed changes. This may cause a confirmation that the changes are accepted to be transmitted from the client device of the practitioner to the server computing device of the treatment planner in embodiments.

In some embodiments, confirmation 484 of the proposed changes may be provided from the assistant 464 (e.g., entered via the AI chat function) to treatment planner 462. In some embodiments, acceptance of changes may bypass the AI chat function, e.g., selecting “accept changes” provided via the treatment planner 462 outside the context of the AI assistant. Treatment planner 462 may perform one or more operations to generate new treatment 486. Generating new treatment may include generating or editing a treatment protocol, generating or editing a treatment plan or prescription, generating or editing manufacturing data for one or more treatment appliances, or other actions associated with generating new treatment data based on the instructions provided by practitioner 460. Treatment planner 462 may save draft 488 to treatment storage 468, which may be accessed locally, over the internet, over a private network, via an API, or in another manner known in the art.

In some embodiments, treatment planner 462 may provide a publish draft request 490 associated with the new treatment. Publishing the draft may include implementing the draft in future treatment planning operations. Publishing a draft of a treatment protocol may include making the draft the default protocol for generation of treatment plans. Practitioner 460 may provide a publish draft confirmation 492, upon which treatment planner 462 may publish draft 494 for use in future treatment planning operations.

FIG. 4F depicts a treatment planning GUI 400F including an AI assistant chat interface 495 and dental arch diagram 496 for managing dental treatment parameters, according to some embodiments. The treatment planning GUI 400F provides an interface for practitioners to configure treatment parameters while receiving assistance from an AI-powered protocol assistant.

The treatment planning GUI 400F includes multiple tabs for accessing different categories of treatment parameters. The illustrated tabs include Tooth Movement, Attachments, Extractions, and Missing teeth. In the depicted view, the Extractions tab is currently selected.

When the Tooth Movement tab is selected, the treatment planning GUI 400F may display options for configuring tooth movement parameters. The tooth movement view may include settings for movement velocity limits, staging preferences, and movement sequencing options. The dental arch diagram 496 may display indicators showing which teeth have movement restrictions applied, movement directions, or staging information associated with planned tooth movements.

When the Attachments tab is selected, the treatment planning GUI 400F may display options for configuring attachment placement and parameters. The attachments view may include settings for attachment types such as optimized attachments, conventional attachments, or protocol-based attachments. The view may include options for specifying attachment placement stages, attachment sizes, and/or teeth to receive attachments. The dental arch diagram 496 may display indicators showing teeth with attachment restrictions, teeth scheduled to receive attachments, or teeth with existing attachments.

When the Extractions tab is selected, as shown in the depicted view, the treatment planning GUI 400F displays options for configuring tooth extractions. The dental arch diagram 496 displays a schematic representation of upper and lower dental arches with numbered teeth positions ranging from 1 to 32. Various tooth status indicators are displayed on the dental arch diagram 496, including teeth marked with X symbols indicating teeth selected for extraction, teeth marked with circle symbols indicating attachment restrictions, and teeth marked with crossed circle symbols indicating extracted teeth. A treatment status legend 497 is positioned below the dental arch diagram 496 and provides definitions for the status indicators including Tooth movement restriction, Attachment restriction, Extracted, and Missing.

When the Missing teeth tab is selected, the treatment planning GUI 400F may display options for configuring treatment parameters related to missing teeth. The missing teeth view may include settings for pontic placement, space closure options, and treatment approaches for gaps in the dental arch. The dental arch diagram 496 may display indicators showing locations of missing teeth and any associated treatment parameters.

The treatment planning GUI 400F includes a checkbox option to follow a global clinical preferences template with explanatory text indicating that preferences in the selected template will be applied. This option allows practitioners to apply previously configured treatment preferences and/or a generated treatment protocol to the current treatment plan.

In the illustrated example, the AI assistant chat interface 495 is positioned on the right side of the treatment planning GUI 400F and displays a protocol assistant that is shown as online. The AI assistant chat interface 495 shows a chat exchange where the assistant greets the user with a message asking how it can help, followed by a user request to set tooth 12 for extraction, and a confirmation response indicating that extractions for tooth 12 were successfully applied to flex rx. The AI assistant chat interface 495 includes a text entry field for entering additional requests and an assistant button at the bottom. The AI assistant chat interface 495 enables practitioners to make treatment parameter adjustments using natural language instructions, which are then converted to machine-readable instructions and applied to the treatment plan.

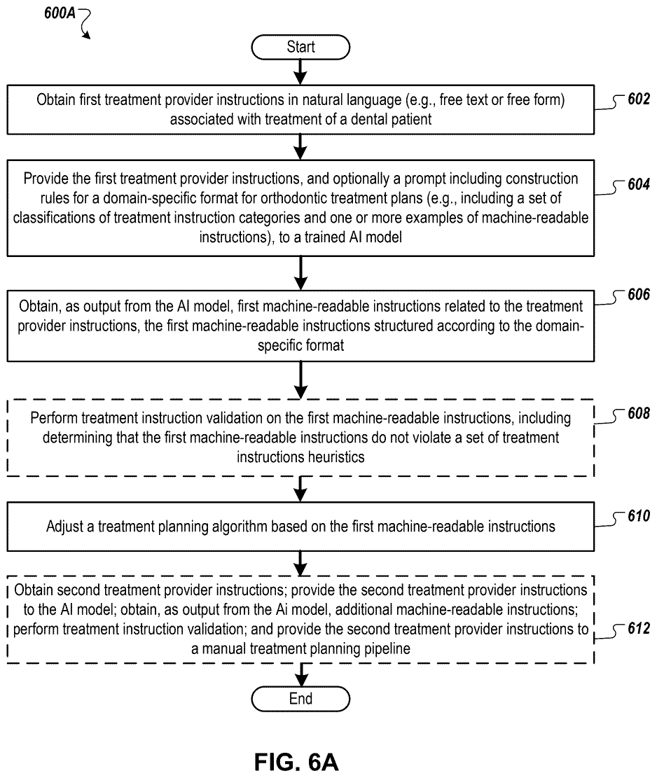

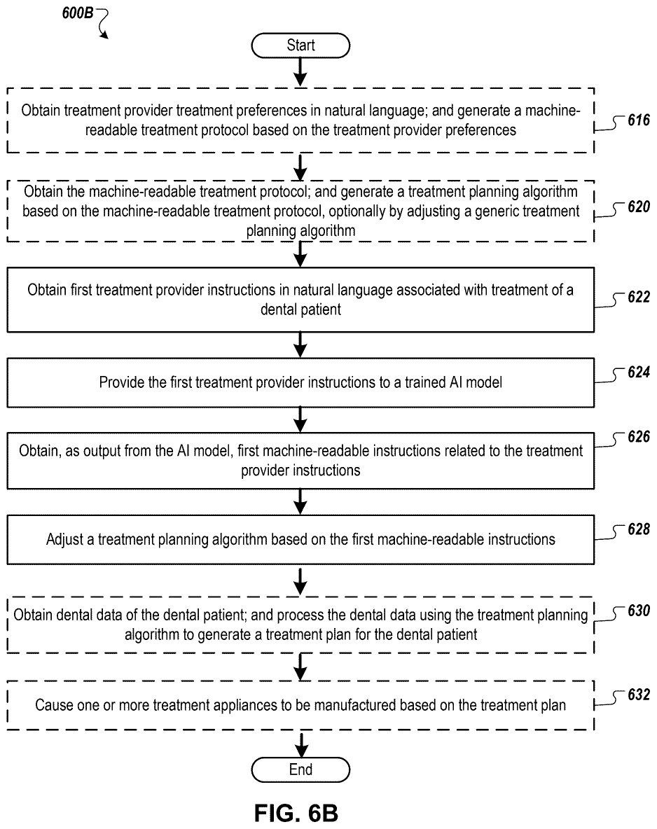

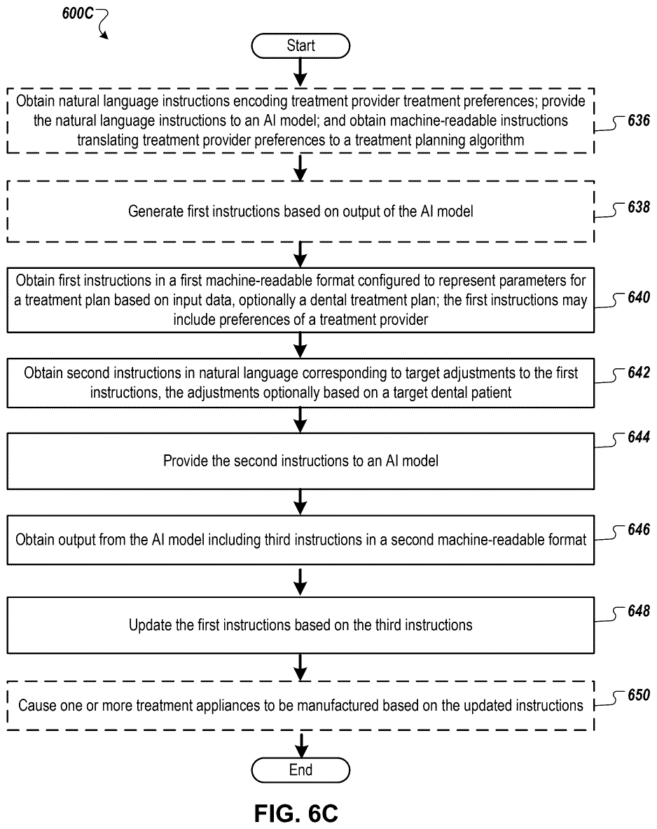

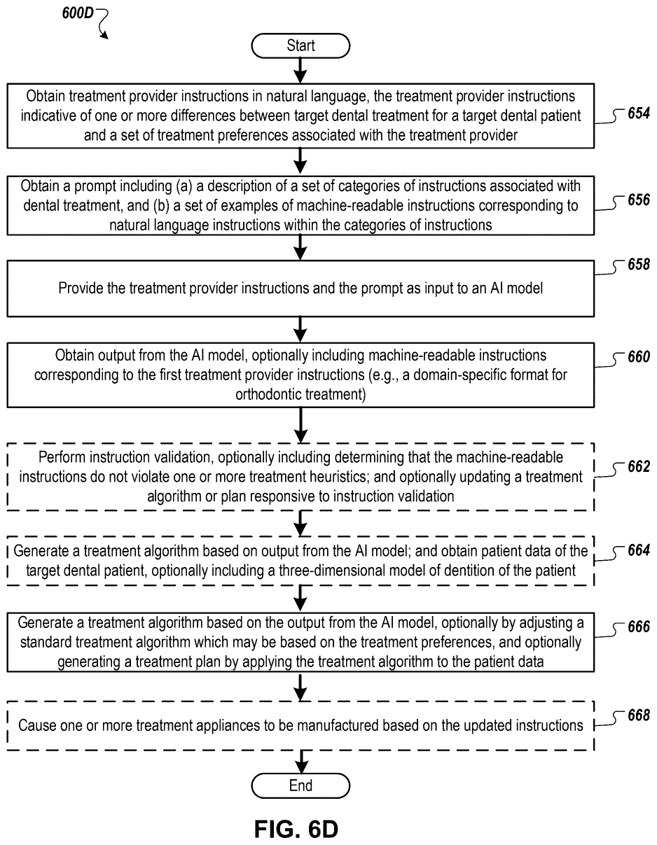

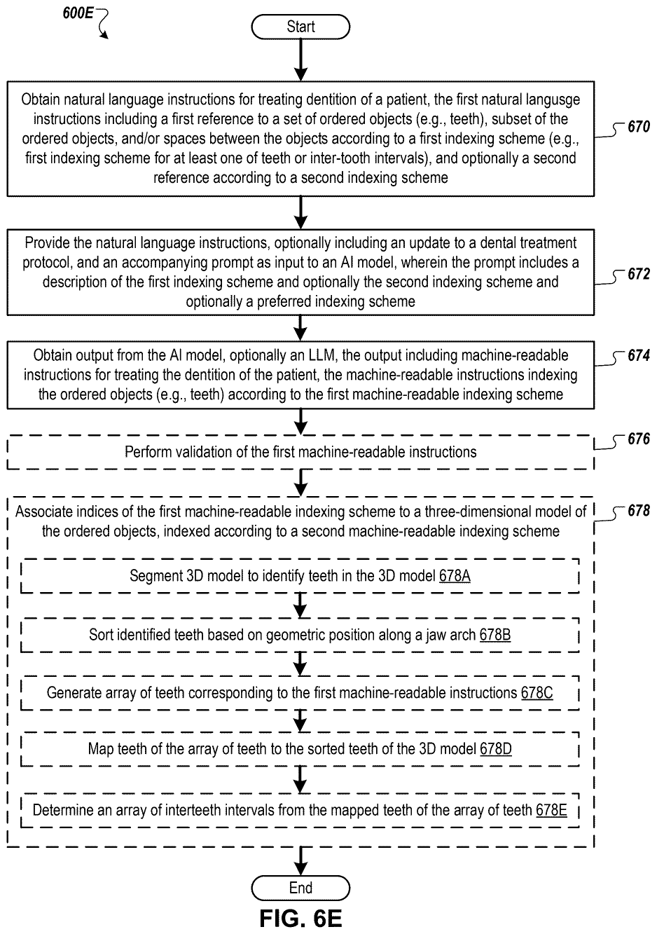

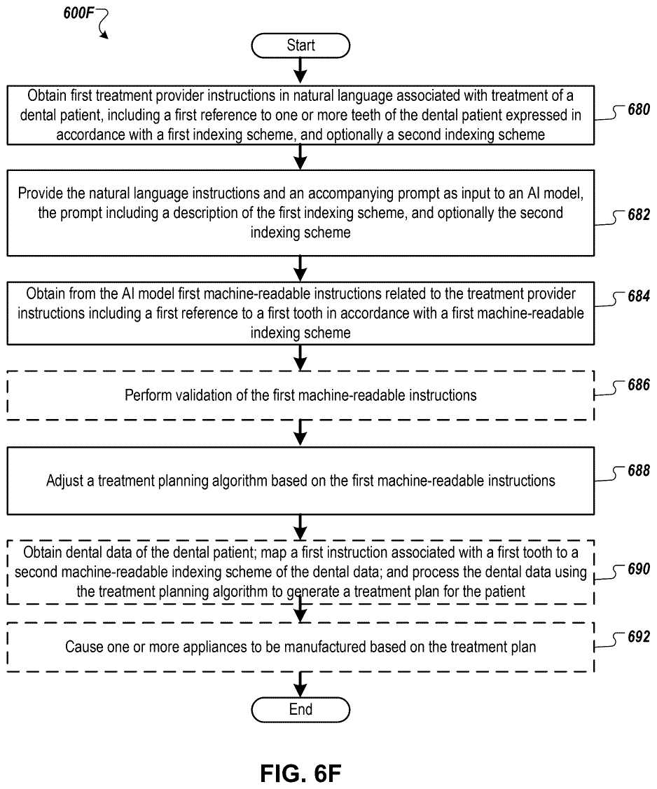

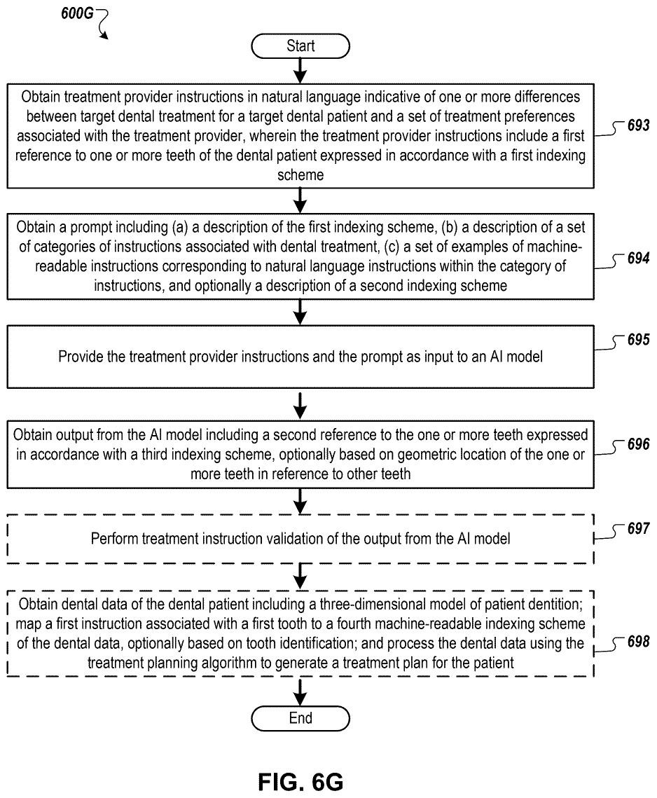

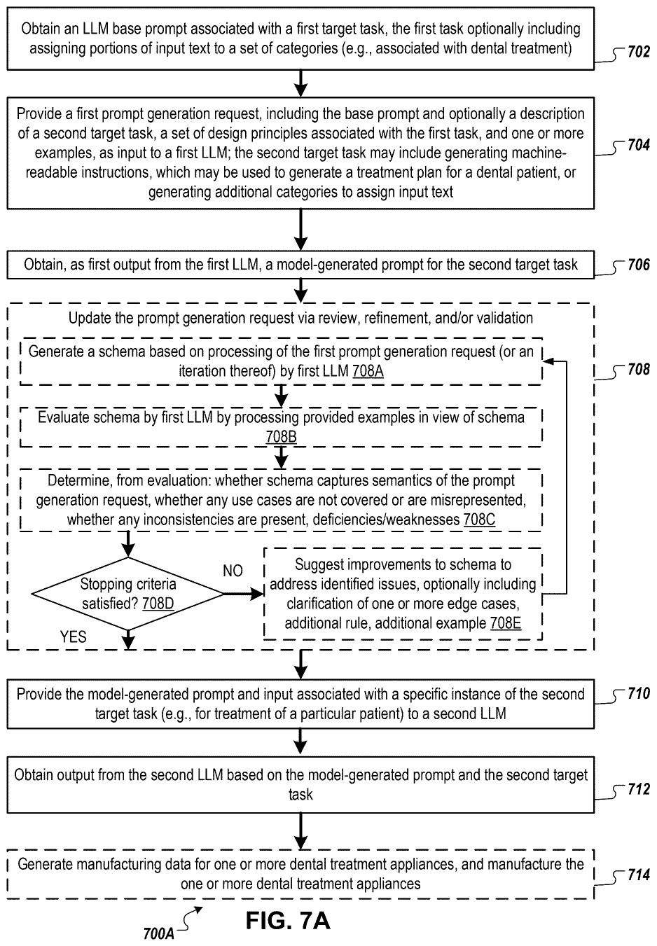

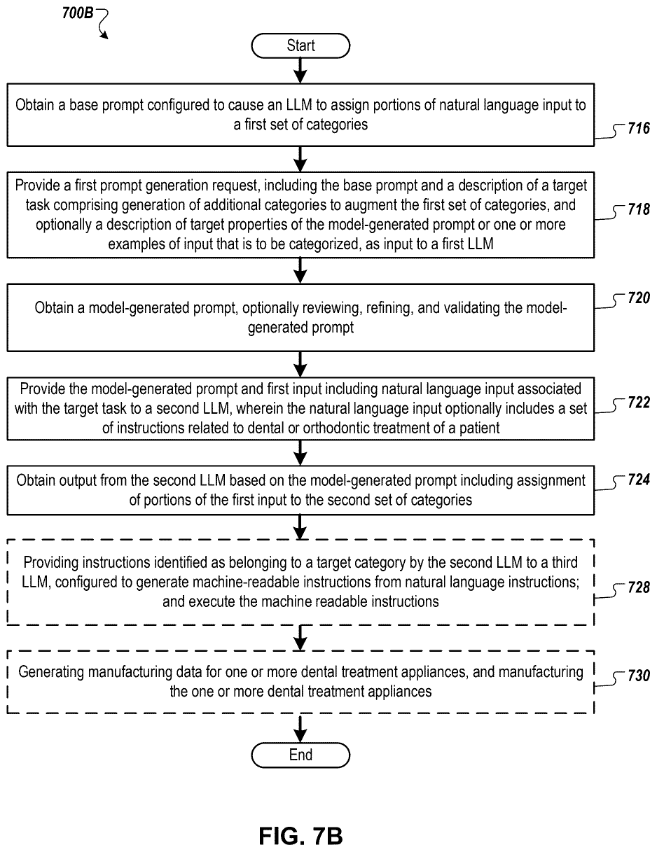

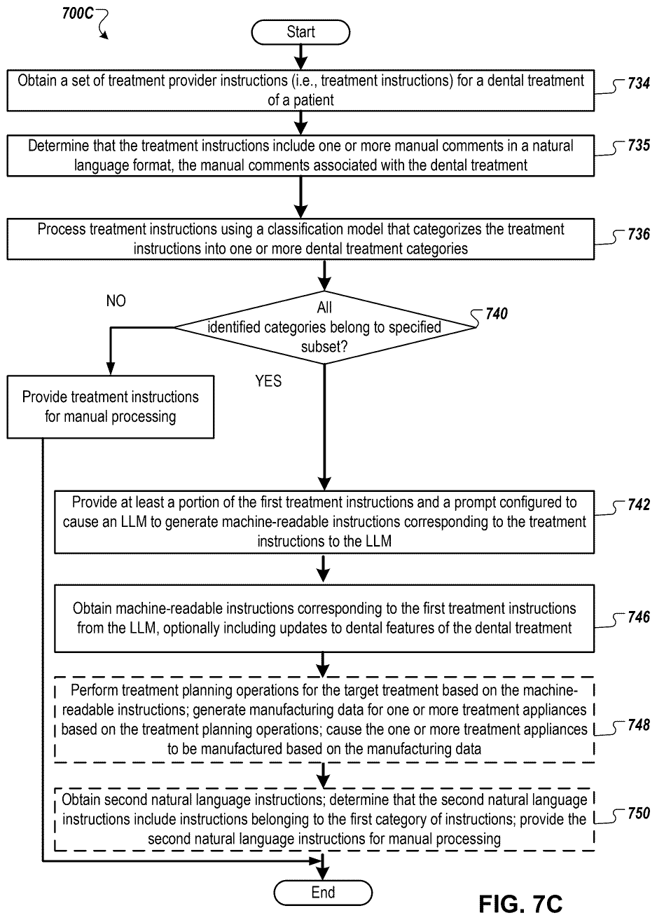

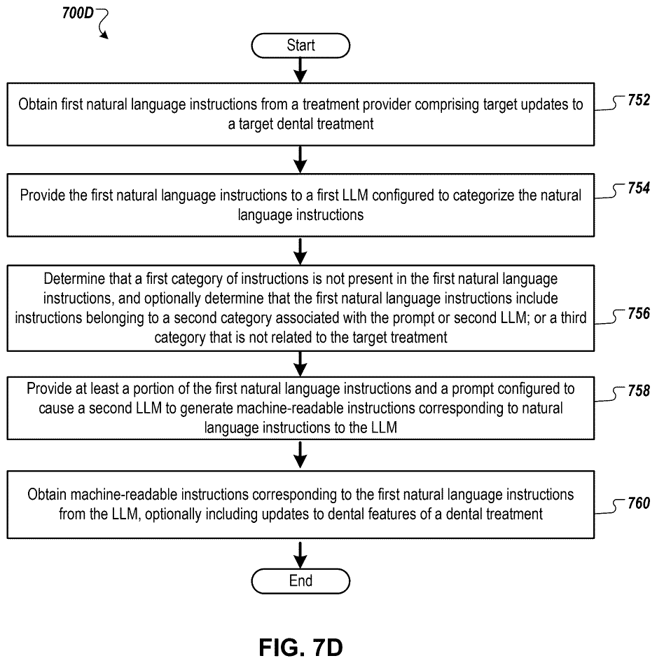

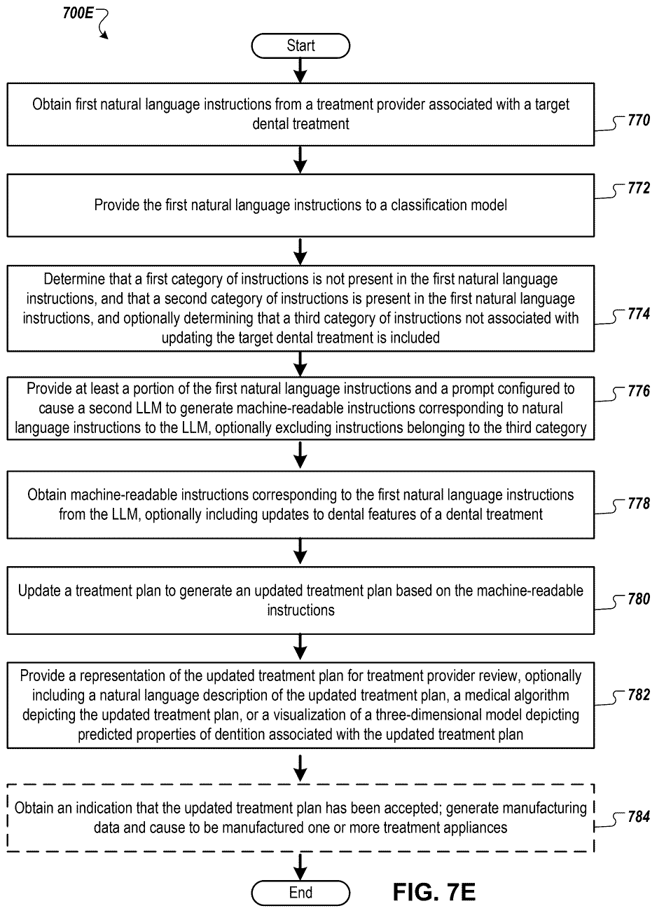

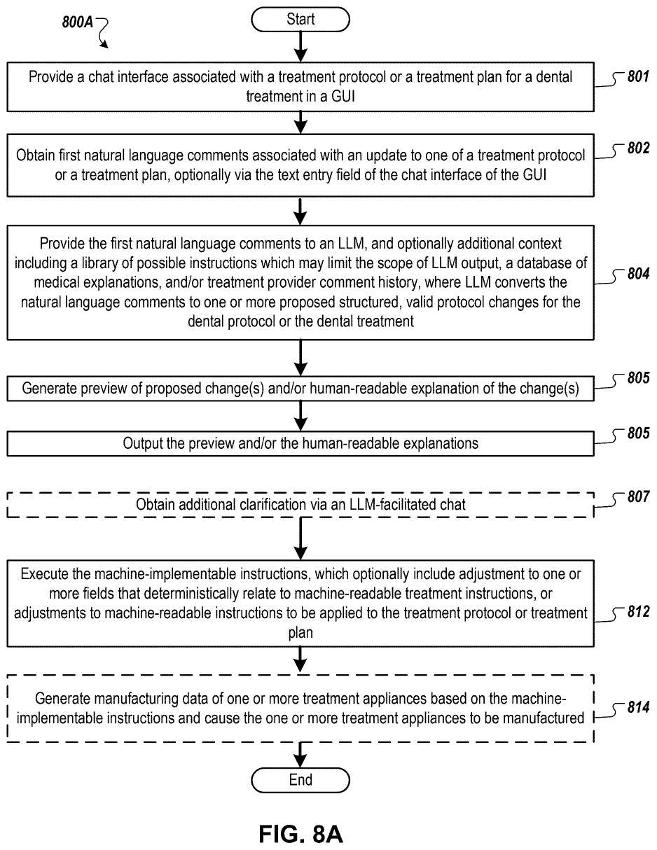

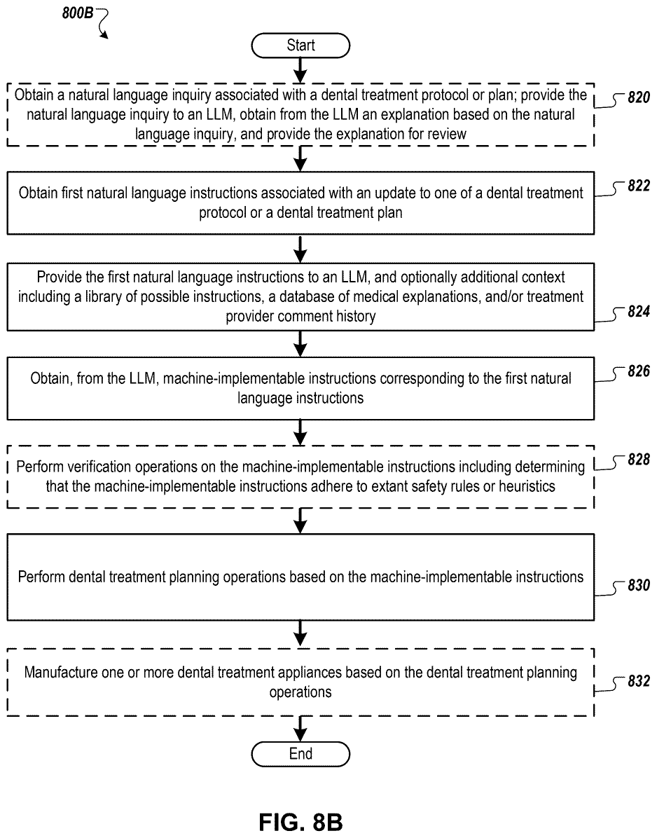

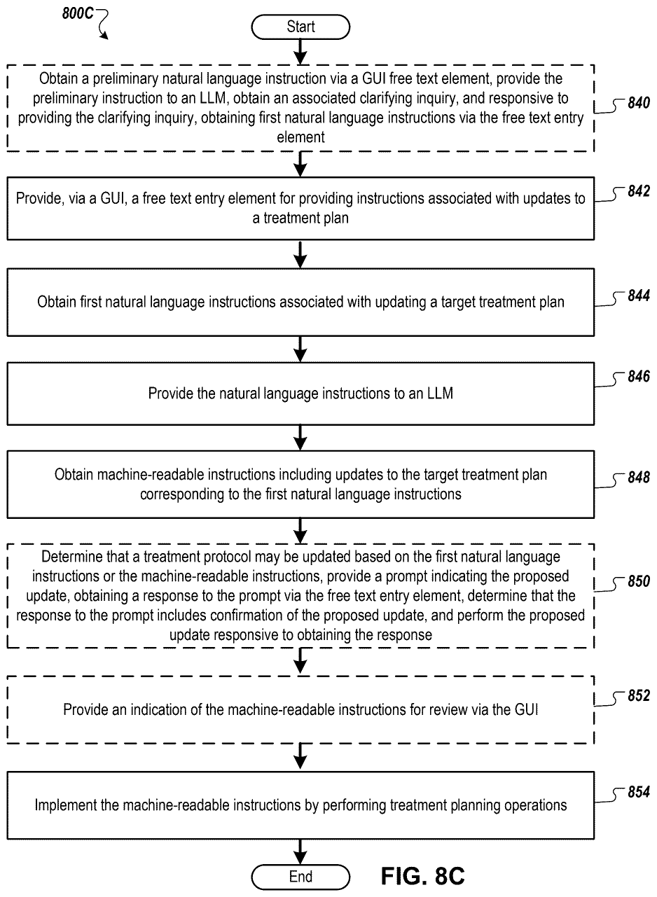

FIGS. 5A-G, FIGS. 6A-G, FIGS. 7A-E, and FIGS. 8A-C are flow diagrams of methods 500A-G, methods 600A-G, methods 700A-E, and methods 800A-C associated with training and utilizing models for generating treatment protocols and/or treatment plans, according to certain embodiments. Methods 500A-G, 600 A-G, 700A-E, and 800A-C may be performed by processing logic that may include hardware (e.g., circuitry, dedicated logic, programmable logic, microcode, processing device, etc.), software (such as instructions run on a processing device, a general purpose computer system, or a dedicated machine), firmware, microcode, or a combination thereof. In some embodiment, these methods may be performed, at least in part, by treatment planning system 110 of FIG. 1. Method 500A may be performed, at least in part, by treatment planning system 110 (e.g., server machine 170 and data set generator 172 of FIG. 1). Treatment planning system 110 may use method 500A to generate a data set to at least one of train, validate, or test a machine learning model, in accordance with embodiments of the disclosure. Methods 500B-G, 600A-G, 700A-E, and 800A-C may be performed by treatment planning server 112 (e.g., treatment planning component 114), treatment provider device 120, and/or server machine 180 (e.g., training, validating, and testing operations may be performed by server machine 180). In some embodiments, a non-transitory machine-readable storage medium stores instructions that when executed by a processing device (e.g., of treatment planning system 110, of server machine 180, of treatment planning server 112, etc.) cause the processing device to perform one or more of these methods.

For simplicity of explanation, each of the below methods are depicted and described as a series of operations. However, operations in accordance with this disclosure can occur in various orders and/or concurrently and with other operations not presented and described herein. Furthermore, not all illustrated operations may be performed to implement these methods in accordance with the disclosed subject matter. In addition, those skilled in the art will understand and appreciate that these methods could alternatively be represented as a series of interrelated states via a state diagram or events.

FIG. 5A is a flow diagram of a method 500A for generating a data set for a machine learning model, according to some embodiments. Referring to FIG. 5A, in some embodiments, at block 501 the processing logic implementing method 500A initializes a training set T to an empty set.

At block 502, processing logic generates first data input (e.g., first training input, first validating input). The first data input may include data types related to an intended use of the machine learning model. The first data input may include natural language instructions related to a treatment of a dental disorder. The first data input may include data appropriate for other operations, such as data of several topics to be split into sections, statements to be converted to machine-readable instructions, statements to be detected as default statements, or any of the operations described with respect to FIG. 3D. Input data may include output data of another model. For example, a machine learning model configured to determine a topic of a selection of text may take as input output of a paragraph splitter AI model.

In some embodiments, at block 503, processing logic optionally generates a first target output for one or more of the data inputs (e.g., first data input). In some embodiments, target output may represent an intended output space for the model. For example, a machine learning model configured to generate machine-readable instructions may be provided natural language instructions and machine-readable instructions as training input and corresponding target output.

At block 504, processing logic optionally generates mapping data that is indicative of an input/output mapping. The input/output mapping (or mapping data) may refer to the data input (e.g., one or more of the data inputs described herein), the target output for the data input, and an association between the data input(s) and the target output. In some embodiments, data segmentation may also be performed. For example, various sections related to different disorders or treatments may be separated for further operations. In some embodiments, such as in association with machine learning models where no target output is provided, block 504 may not be executed.

At block 505, processing logic adds the mapping data generated at block 504 to data set T, in some embodiments.

At block 506, processing logic branches based on whether data set T is sufficient for at least one of training, validating, and/or testing a machine learning model, such as model 190 of FIG. 1. If so, execution proceeds to block 507, otherwise, execution continues back at block 502. It should be noted that in some embodiments, the sufficiency of data set T may be determined based simply on the number of inputs, mapped in some embodiments to outputs, in the data set, while in some other embodiments, the sufficiency of data set T may be determined based on one or more other criteria (e.g., a measure of diversity of the data examples, accuracy, etc.) in addition to, or instead of, the number of inputs.

At block 507, processing logic provides data set T (e.g., to server machine 180) to train, validate, and/or test machine learning model 190. In some embodiments, data set T is a training set and is provided to training engine 182 of server machine 180 to perform the training. In some embodiments, data set T is a validation set and is provided to validation engine 184 of server machine 180 to perform the validating. In some embodiments, data set T is a testing set and is provided to testing engine 186 of server machine 180 to perform the testing. In the case of a neural network, for example, input values of a given input/output mapping (e.g., numerical values associated with data inputs) are input to the neural network, and output values (e.g., numerical values associated with target outputs) of the input/output mapping are stored in the output nodes of the neural network. The connection weights in the neural network are then adjusted in accordance with a learning algorithm (e.g., back propagation, etc.), and the procedure is repeated for the other input/output mappings in data set T. After block 507, a model (e.g., model 190) can be at least one of trained using training engine 182 of server machine 180, validated using validating engine 184 of server machine 180, or tested using testing engine 186 of server machine 180. The trained model may be implemented by treatment planning component 114 (of treatment planning server 112) to generate treatment protocol data 142 for performing signal processing, or for performing a corrective action.

FIG. 5B is a flow diagram of a method 500B for generating treatment protocol data based on natural language input, according to some embodiments.

At block 510, processing logic obtains treatment provider instructions associated with a target dental treatment, where the instructions are expressed in a natural language format. The treatment provider instructions may comprise free-text instructions, clinical preferences, and/or specific directives related to dental treatment planning provided by a treatment provider such as a dentist or orthodontist.

At block 512, processing logic provides first input comprising the instructions to one or more AI models. The AI model(s) may comprise a large language model (LLM), a combination of models, or a model that is referenced multiple times by a number of agents each associated with a target operation. This may include transmitting treatment provider instructions to a remote computing device for processing by the AI model. The AI model may be provided with input including at least a portion of the natural language instructions, as well as a prompt that configures the AI model to provide a target response type, format, or the like. For example, the prompt may include instructions to the AI model to perform one or more operations related to obtaining treatment protocol based on natural language treatment instructions. In some embodiments, inputs may be provided to the AI model, outputs received from the AI model, at least a portion of the output provided back to the AI model as input, and so forth.

At block 513, processing logic processes the first input using the one or more AI models (e.g., one or more LLMs). The processing may involve multiple operations performed by the AI model or models to transform the natural language instructions into a structured format (e.g., machine-readable format) suitable for treatment protocol generation and/or treatment plan generation. The AI model may analyze the content of the treatment provider instructions, identify relevant clinical concepts and treatment parameters, and organize the information according to categories associated with dental treatment. The processing may include formatting operations, segmentation of the instructions into logical portions, classification of instruction types, and transformation of natural language statements into corresponding machine-readable representations. In some embodiments, the processing may involve iterative interactions with the AI model, where intermediate outputs are refined through additional processing steps. The output of the processing at block 513 comprises structured data (e.g., machine-readable instructions) that represents the treatment provider's instructions in a format that can be used to generate a treatment protocol and/or treatment plan.

In some embodiments, block 513 can include several sub-operations for transforming the natural language instructions into machine-readable format.

At block 513A, processing logic may update formatting of the first input to generate second input. The formatting update may include resolving abbreviations, unifying medical terminology, standardizing indexing or labeling such as tooth numbering systems, and unifying text formatting. In some embodiments, the formatting operations may be configured to normalize variations in how treatment providers express instructions, such as converting different tooth numbering conventions (e.g., Universal, FDI, or Palmer systems) into a standardized format. The formatting operations at block 513A may address the substantial variability in how treatment providers express clinical instructions. Treatment providers may use different abbreviations for common terms (e.g., “att” or “attach” for attachments, “IPR” for interproximal reduction), different numbering systems for identifying teeth, and varying levels of formality or specificity in their instructions. The text formatter may resolve these variations by expanding abbreviations to their full forms, converting tooth references to a unified numbering scheme, and standardizing terminology to match expected input formats for downstream processing. In some embodiments, the formatting operations may also handle multilingual input by detecting non-English instructions and translating them to English for consistent processing. The formatting operations may further include removing extraneous content such as polite expressions (e.g., “thank you” or “please”) that do not contribute to the clinical substance of the instructions, while preserving all clinically relevant information for subsequent processing stages.

At block 513B, processing logic may separate the second input into a plurality of sections. The AI model may be configured to separate the treatment provider instructions into sections, where each of the sections may be associated with a different dental disorder or treatment, with one of the sections associated with the target dental treatment. In some embodiments, the separation may be performed based on detected subject matter, paragraph boundaries, or semantic analysis of the instruction content. The separation operations at block 513B may utilize a paragraph splitter or similar component to identify logical boundaries within the treatment provider instructions. Treatment providers often include instructions relating to multiple aspects of treatment within a single communication, such as instructions for attachments, interproximal reduction, staging preferences, and final tooth positions. The separation operation may analyze the semantic content of the instructions to identify distinct topics or treatment categories, grouping related instructions together while separating instructions that pertain to different aspects of treatment. In some embodiments, the separation may be performed using natural language processing techniques that identify topic shifts, conjunctions indicating new subjects, or explicit section markers provided by the treatment provider. The separation operation may also identify instructions that span multiple categories and appropriately duplicate or cross-reference such instructions across relevant sections. This segmentation enables subsequent processing stages to apply specialized models or processing logic tailored to each category of instructions, improving the accuracy and relevance of the generated machine-readable output.

At block 513C, processing logic may determine a malocclusion type, treatment parameter(s), and/or dental condition(s) for each section. The malocclusion type may include classifications such as Class I, Class II, or Class III malocclusion, or other dental condition categories. In some embodiments, the determination may be performed using a classification model that is configured to identify relevant dental conditions based on the content of each section. The classification operations at block 513C may employ a title detector or classification model to assign each section to one or more dental treatment categories. The categories may include malocclusion classifications (Class I, Class II, Class III), treatment operation categories (such as dental features, interproximal reduction, passive aligners, overcorrection, treatment length), and/or specific clinical conditions (such as crowding, spacing, midline shift, overbite, crossbite). The classification model may be trained on historical treatment provider instructions and their corresponding category assignments to accurately identify the subject matter of each section. In some embodiments, the classification may be performed using a large language model configured with a prompt that describes the available categories and provides examples of instructions belonging to each category. The classification operation may assign multiple categories to a single section when the instructions address multiple treatment aspects, enabling appropriate processing by multiple specialized downstream components. The determined categories may be used to route each section to appropriate specialized processing models and to validate that the generated machine-readable instructions are consistent with the identified treatment categories.

At block 513D, processing logic may separately process each section using a model associated with the malocclusion type, treatment parameter(s), and/or dental condition(s) for that section to generate a treatment protocol portion. Different models or model configurations may be applied based on the determined malocclusion type, treatment parameter(s), and/or dental condition(s) to generate appropriate machine-readable instructions for each section. In some embodiments, specialized prompts or fine-tuned models may be utilized for different malocclusion types to improve accuracy of the generated machine-readable instructions. The specialized processing at block 513D may involve applying category-specific transformation logic to convert natural language instructions into structured, machine-readable format. For each identified category, a corresponding transformer or conversion model may be applied that is optimized for that category of instructions. For example, instructions classified as relating to dental features (such as attachments) may be processed by a specialized model configured to extract attachment type, action (add, remove, keep, modify), affected teeth, and staging information. Instructions relating to interproximal reduction may be processed by a model configured to extract IPR amounts, affected contacts, and scheduling preferences. The specialized models may be implemented as large language models with category-specific prompts that include detailed descriptions of the expected output format, examples of input-output pairs for that category, and schemas that constrain the model output to valid values. In some embodiments, the specialized models may be fine-tuned smaller language models that have been trained specifically on instructions within their assigned category, providing faster inference and reduced computational requirements compared to general-purpose large language models. The output of each specialized model may be a structured representation (such as JSON) that captures the clinical intent of the natural language instructions in a format suitable for automated treatment planning systems.

At block 513E, processing logic may combine the treatment protocol portions to generate a treatment protocol. The combined treatment protocol can represent the aggregated machine-readable instructions derived from processing each section of the treatment provider instructions. In some embodiments, the combination operation may include validation checks to ensure consistency across the combined protocol portions and to resolve any conflicts between instructions from different sections. The combination operations at block 513E may aggregate the machine-readable instructions generated from each section into a unified treatment protocol. An instruction collector or similar component may receive the outputs from the specialized processing models and merge them into a coherent protocol structure. The combination operation may include post-processing steps to validate the combined instructions, such as clinical checking to verify that the instructions do not violate safety heuristics or clinical constraints, and syntax checking to ensure that the combined output conforms to the required schema and format specifications. In some embodiments, the combination operation may detect and resolve conflicts between instructions from different sections, such as contradictory staging preferences or incompatible treatment operations. The combination operation may also identify instructions that were not successfully processed by the specialized models and flag such instructions for manual review or alternative processing. The resulting combined treatment protocol may be compiled into a final machine-readable format suitable for execution by a treatment building engine, which may apply the protocol to patient-specific data to generate individualized treatment plans. In some embodiments, the combination operation may generate both the machine-readable protocol and a human-readable summary or explanation of the protocol for treatment provider review and confirmation prior to application.

At block 514, processing logic obtains, from the AI model or models, machine-readable instructions that represent and/or can be used to generate a first treatment protocol, treatment plan, and/or treatment algorithm in association with the target dental treatment. The machine-readable instructions may be structured according to a domain-specific format suitable for automated treatment planning operations. In some embodiments, the AI model (e.g., one of the models included in the AI model workflow) may perform safety checks to determine whether the first treatment protocol is in accordance with one or more clinical threshold conditions associated with the target dental treatment.

At block 516, processing logic provides an alert to the treatment provider comprising the treatment protocol.

In some embodiments, one or more operations may be facilitated by a GUI. For example, a GUI may be provided to the treatment provider to obtain the treatment provider instructions. The GUI may include a fillable text field, a chat function element, or another method for inputting natural language instructions. A prompt may be provided to the practitioner via the GUI to obtain the treatment provider instructions. In some embodiments, a validation prompt in natural language may be provided in association with the first treatment protocol, which the practitioner may respond to in order to verify that the treatment protocol is correct, or to trigger additional clarification, changes, updates, or the like to the treatment protocol. In some embodiments, a response generator model may generate a natural language validation prompt in a deterministic manner based on machine-readable instructions associated with the first treatment protocol.

FIG. 5C is a flow diagram of a method 500C for training a machine learning or AI model for performing operations associated with generating machine-readable treatment protocol instructions, according to some embodiments. The method 500C enables the creation of trained models capable of translating natural language treatment provider instructions into structured, machine-readable formats suitable for automated treatment planning operations. At block 520, process logic obtains a plurality of treatment provider instructions associated with dental treatments. The treatment provider instructions may be expressed in natural language format and may include free-text instructions, clinical preferences, treatment directives, and/or other communications from orthodontists or other dental practitioners. The treatment provider instructions may relate to various aspects of dental treatment including tooth movement preferences, attachment placement, interproximal reduction parameters, staging preferences, or treatment goals for specific dental conditions such as malocclusions, crowding, spacing, or overbite correction.

At block 522, process logic obtains a plurality of machine-readable instructions corresponding to the treatment provider instructions. The machine-readable instructions may be structured in a domain-specific format suitable for orthodontic treatment planning, such as JSON format or another structured data representation. The machine-readable instructions may encode treatment parameters, tooth identifiers according to standardized numbering systems, treatment actions, attachment types, staging information, and/or other treatment-related data in a format that can be processed by treatment planning engines or other automated systems. The correspondence between the treatment provider instructions and the machine-readable instructions establishes input-output pairs that serve as training examples for the machine learning model.

At block 524, process logic trains a machine learning model by providing the plurality of treatment provider instructions as training input and the plurality of machine-readable instructions as target output. The training process generates a trained machine learning model configured to convert natural language instructions into machine-readable format. Training the machine learning model may include adjusting one or more parameters of an LLM (e.g., a general purpose, pre-trained LLM). Training the model may include performing parameter-efficient fine-tuning operations. Training the model may include performing low-rank adaptation operations, which enable efficient adaptation of large models by training low-rank decomposition matrices rather than full model weights. Training the machine learning model may include introducing and adjusting adapter layers between existing layers of the machine learning model. The use of adapter layers allows for task-specific customization while preserving the general capabilities of the underlying model. In some embodiments, the trained model may be a smaller, specialized model compared to commercial large language models, enabling deployment on internal resources with reduced computational requirements and latency. The trained model may be configured to output structured data conforming to a predefined schema, such as a JSON schema that defines allowed values, data types, and relationships between treatment parameters. In some embodiments, data for training may be or include feedback data, e.g., data indicative of correctness of previously performed operations of the machine learning model. The feedback data may include evaluation data submitted by a user. The feedback data may be indirect feedback data, e.g., data collected indicative of accuracy of previous operations of the machine learning model. For example, a number of updates performed on a treatment protocol, an amount of time spent refining a protocol or one or more dental treatments based on the protocol, a level of confidence produced by the AI model that the protocol is accurate, or the like may be used as indirect feedback data, and may be used to determine whether retraining operations are to be performed, to focus retraining operations on particular aspects, subjects, or areas, may be used in determining what to use as training data, or the like. The trained machine learning model may be stored in model storage and subsequently used in treatment planning workflows to process treatment provider instructions and generate corresponding machine-readable instructions for automated treatment plan generation.

FIG. 5D is a flow diagram of a method 500D for filling fields of a data model associated with dental treatment, according to some embodiments. The method 500D provides a structured approach for utilizing conversational or prompt-based interactions to populate treatment-related data fields, where natural language prompts are generated to elicit responses from a user, and a trained machine learning model processes those responses to automatically fill the corresponding fields of the data model. At block 530, process logic obtains a data model comprising one or more fields to be filled, the fields associated with dental treatment. The data model may include fields related to treatment protocols, treatment plans, patient-specific treatment parameters, clinical preferences, or other dental treatment-related information. The fields may correspond to various aspects of dental treatment such as interproximal reduction parameters, attachment specifications, tooth movement preferences, staging information, or treatment goals. The data model may be structured according to a predefined schema that defines the types of values expected for each field and the relationships between different fields.

At block 532, process logic generates a first prompt in natural language associated with one or more of the fields. The prompt may, for example, ask a practitioner a question related to treatment preferences, treatment protocols, one or more dental disorders, or other prompts related to generating treatment protocol data. The prompt generation may be performed by a deterministic or rule-based system that maps fields of the data model to corresponding natural language questions, or by an AI model configured to generate contextually appropriate prompts based on the field type and any previously provided information. The prompt may be tailored to the specific field being populated, such as asking about preferred IPR amounts, attachment placement preferences, staging preferences, or treatment goals for specific malocclusion types. The prompt may also include contextual information to help the practitioner understand what information is being requested and how it will be used in the treatment planning process.

At block 536, process logic presents, via a GUI, the first prompt to a user, e.g., a dental healthcare practitioner. The GUI may include a chat interface, a dialog box, or another interactive element configured to display the prompt and receive user input. The presentation of the prompt may be accompanied by visual aids such as dental arch diagrams, 3D model views, and/or explanatory text to assist the practitioner in understanding the context of the question. The GUI may also display previously provided responses or current field values to provide context for the current prompt. At block 538, process logic obtains a response to the first prompt. The prompt and the response may be presented in natural language, allowing the practitioner to provide input without requiring specialized training in machine-readable instruction formats. The response may be obtained via a text entry field, voice input, or other input mechanisms supported by the GUI. The response may include specific values, preferences, conditions, or instructions expressed in the practitioner's own words.

At block 540, process logic provides the response to a trained machine learning model (e.g., an LLM). The trained machine learning model may be configured to interpret natural language responses and extract structured information suitable for populating the fields of the data model. The model may be provided with additional context such as the original prompt, the field schema, previously filled fields, and/or a library of valid field values to constrain the model output to appropriate values. In some embodiments, process logic further provides a second prompt to the practitioner, e.g., a verification or validation prompt that summarizes the understanding of the model, and/or asks the practitioner whether the adjustments, inclusions, or updates to the data model is accurate, acceptable, correct, or the like. This verification step enables the practitioner to confirm that the model correctly interpreted the natural language response before the field values are committed to the data model. The verification prompt may present the proposed field values in a human-readable format along with an explanation of how the response was interpreted.

At block 542, process logic fills the one or more fields using output of the trained machine learning model based on the response to the first prompt. The output from the machine learning model may include structured data in a machine-readable format that corresponds to the schema of the data model fields. Process logic may perform validation operations on the output to confirm that the values conform to expected formats, fall within acceptable ranges, and do not violate clinical constraints or safety heuristics before populating the fields. The filled fields may be stored in association with the data model and made available for subsequent treatment planning operations.

In some embodiments, operations of method 500D may be repeated, e.g., to fulfill all fields or a target number or selection of fields of the data model. The iterative application of the method 500D enables progressive population of the data model through a series of conversational exchanges, where each iteration addresses one or more fields until the data model is sufficiently complete for its intended purpose. The method 500D may also be applied to update previously filled fields based on new practitioner input or changed treatment requirements.

FIG. 5E is a flow diagram of a method 500E for generating machine-readable instructions related to dental treatment based on a selection of options, according to some embodiments. At block 544, process logic provides a first set of options related to treatment preferences for a first dental condition. The first set of options may be presented via a GUI, such as the GUI depicted in FIG. 3F. The GUI may include dental conditions, treatment protocol categories, etc. In some embodiments, one or more guides may be provided via the GUI that provide additional information related to a treatment protocol or treatment plan. For example, additional information indicating relationships between treatment options may be presented, a three-dimensional model of dentition depicting example dentition and reflecting default or selected treatment options may be available for inspection via the GUI, and/or an indication of an expected outcome or recommended treatment option with respect to a selection may be presented via the GUI. In some embodiments, the GUI may provide additional sets of options for additional dental conditions, additional treatment categories, or the like.

The dental conditions may include malocclusion (including Class I, Class II, and Class III malocclusions), anterior leveling, underbite, posterior crossbite, anterior crossbite, overbite, open bite, deep bite, overjet, crowding, spacing, diastema, midline shift or deviation, anterior-posterior discrepancy, rotated teeth, tipped or tilted teeth, impacted teeth, supernumerary teeth, missing teeth, ectopic eruption, tooth intrusion, tooth extrusion, arch width discrepancy, arch length discrepancy, asymmetric arches, bimaxillary protrusion, retrognathia, prognathia, dental protrusion, dental retrusion, gummy smile, canted occlusal plane, scissor bite, edge-to-edge bite, end-on relationship, and/or any combination thereof.

In some embodiments, the first set of options may comprise a hierarchical tree structure, and obtaining a selection may cause one or more additional options to be displayed or hidden based on the selection. For example, a high-level selection of a malocclusion correction category may expose sub-choices including treatment goal options (e.g., molar and canine class I, molar class I only), distalization pattern options, amount of distalization specification fields, and/or priority selection options between molar and canine correction. As another example, a high-level selection of an IPR category may expose sub-choices including anterior IPR limit per contact options (e.g., 0.5 mm, 0.4 mm, 0.3 mm, 0.2 mm), posterior IPR limit per contact options, jaw selection options (e.g., upper jaw, lower jaw, both), and/or stage scheduling options (e.g., based on access to contacts, every certain number of stages, at specific stages). As a further example, a high-level selection of an attachment type category may expose sub-choices including action options (e.g., add, remove, keep, replace, modify), specific attachment type options (e.g., mesial distal root control, multi-plane, extrusion, rotation, retention, expansion support), size options (e.g., regular, largest), and/or stage specification options for when the attachment action should be applied.

At block 546, process logic optionally provides a second set of options, related to treatment operations applicable to multiple dental treatments. These options may be related to appliances, general treatment preferences, or the like. For example, options related to interproximal reduction, placing pontics, and extracting teeth may further be presented via a GUI for selection by a treatment provider. In some embodiments, process logic may provide a second set of options related to treatment preferences for a second dental condition, and may obtain a second selection of one of the second set of options. The second selection may be further processed using the model or an additional model to generate the machine-readable code in the domain-specific language for orthodontic treatment, wherein the machine-readable code is associated with the first dental condition and the second dental condition.

At block 548, process logic obtains a first selection of one of the first set of options. The selection may be made by checkbox, radio button, drop-down menu, or any other suitable method for selecting from a list of options. Selections for additional conditions, or additional treatment categories, may also be obtained. The first selection may be obtained via the GUI.

At block 550, process logic provides the first selection to a model configured to generate machine-readable code or instructions, and the model generates the machine-readable code in a domain-specific language for orthodontic treatment. The machine-readable code constitutes a clinical protocol for generating orthodontic treatment plans. The machine-readable instructions may define or enact a treatment protocol, e.g., a set of treatment preferences of a treatment provider that guide treatment planning. The machine-readable instructions may be used to generate a dental treatment plan based on the first selection.

At block 552, process logic obtains, from the model, the machine-readable code. In some cases, the model may be deterministic. For example, a set of selections made via the GUI may always produce the same set of machine-readable instructions by following a rule-based model to translate the selections to the instructions.

At block 554, process logic optionally executes the machine-readable code in association with a dental patient to generate a treatment plan for the first dental condition. Executing the machine-readable code may be performed via a web application or portal, e.g., the same web portal that provides a GUI for treatment protocol generation. Generating the treatment plan may comprise obtaining a three-dimensional model of the patient's dentition (e.g., as generated based on intraoral scan data of the dentition), optionally obtaining a second selection of one or more treatment goals, and executing the machine-readable code in view of the intraoral scan and the one or more treatment goals. Executing the machine-readable code may include inputting various information specific to the patient, such as treatment goals, developmental stage (e.g., teenager or adult), differences for the treatment from one or more general preferences of the protocol, patient dentition data (e.g., intraoral scan data, three-dimensional dentition model, or other dental data), or the like.

At block 556, process logic optionally provides treatment appliance designs for manufacturing. Generating the treatment plan may include generating designs for one or more treatment appliances for one or more stages of treatment, and the method may further comprise providing the designs for manufacturing of the appliances. In some cases, treatment may begin once the appliances have been manufactured and obtained by the dental patient.

In some embodiments, process logic may generate a version identifier associated with the machine-readable code, store the machine-readable code in association with the version identifier, and maintain a history of prior versions of the machine-readable code. This versioning capability enables treatment providers to track changes to treatment protocols over time and revert to prior versions if needed.

FIG. 5F is a flow diagram of a method 500F for generating a treatment protocol and using the treatment protocol to generate a treatment plan in accordance with one or more treatment goals, according to some embodiments. The method 500F enables treatment providers to configure treatment preferences through selection of options associated with various dental conditions and treatment categories, and subsequently apply those preferences to generate patient-specific treatment plans. At block 558, process logic provides a set of treatment options. The set of treatment options may be associated with one or more dental conditions. The dental conditions may include, for example, dental crowding, spacing, midline shift, anterior level conditions, anterior-posterior relationship conditions, posterior crossbite, or overbite. The options may be presented via a GUI. In some embodiments, the treatment options may be organized hierarchically, with treatment categories presented at a first level and specific treatment options presented at a second level within each category. The treatment options may include options for treatment goals, such as achieving molar and canine class I occlusion, as well as options for treatment operations, such as distalization patterns, amounts of distalization, and priority settings for molar versus canine correction. The GUI may include visual aids such as dental arch diagrams, three-dimensional model views, or explanations of expected outcomes to assist the treatment provider in making informed selections.

At block 560, process logic obtains selections from the sets of treatment options. One or more options may be selected for each condition and/or treatment category. In embodiments, multiple topics may be presented with multiple options for a single treatment category or condition. Selections from multiple treatment categories or dental conditions may be obtained. The selections may be obtained via drop-down menus, fillable fields, check boxes, radio buttons, or similar GUI elements. In some embodiments, the treatment provider may select options for malocclusion correction, including specifying treatment goals such as molar and canine class I, selecting upper arch and lower arch configurations, choosing distalization patterns, specifying amounts of distalization in millimeters, and indicating whether anterior teeth movement should start at the same time as posterior distalization. The treatment provider may also specify priority settings indicating whether molar or canine correction should take precedence, and may select types of bite correction simulation such as elastics or surgical approaches. Default options may be provided for each treatment category, and the treatment provider may choose to accept the defaults or override them with custom selections.

At block 562, process logic generates a treatment protocol in a machine-readable format including the selections. Generating the treatment protocol may include providing the selections to a model. The model may be configured to generate the treatment protocol based on the selections. The model may be a rule-based or deterministic model that maps the treatment provider's selections to corresponding machine-readable instructions according to predefined mappings. Alternatively, the model may be an AI model (e.g., an LLM). In some embodiments, the machine-readable format may comprise a domain-specific language (DSL) for orthodontic treatment planning, such as a JSON-based format that structures the treatment provider's preferences into standardized categories and parameters. The treatment protocol may encode instructions for various aspects of treatment, including interproximal reduction limits, attachment placement preferences, staging parameters, and movement sequencing. The generated treatment protocol may be stored in association with a version identifier and may be associated with the treatment provider's account for application to current and future patient cases. In some embodiments, the treatment protocol may be maintained in a draft state prior to publication, allowing the treatment provider to review and refine the protocol before it becomes active.

At block 564, process logic obtains an indication that the first treatment goal is to be applied to a patient, e.g., a dental patient. A treatment provider, in consultation with the dental patient, may determine that a treatment goal is appropriate and provide the indication (e.g., via a treatment planning software or treatment planning web portal) to a treatment planning model. The indication may specify which of the treatment goals defined in the treatment protocol should be applied to the particular patient case. In some embodiments, the treatment provider may select from among multiple treatment goals that were configured during protocol generation, such as selecting between different malocclusion correction approaches or different levels of treatment aggressiveness. The indication may be provided in response to reviewing patient-specific data, such as intraoral scan data, diagnostic images, or clinical examination findings. The treatment planning system may present the available treatment goals along with descriptions of expected outcomes to assist the treatment provider in selecting an appropriate goal for the patient. Process logic may also receive patient data for a patient to be treated. The patient data may include, for example, a 3D model of the patient's current dentition and/or a 3D model of the final target dentition of the patient.

At block 566, process logic generates a treatment plan for the patient. Generating the treatment plan for the patient may be based on the machine-readable treatment protocol, e.g., it may include executing the machine-readable treatment protocol with respect to the patient. In some embodiments, additional information may be used for generating the treatment plan, such as patient biographical information, models (e.g., three-dimensional models) of patient dentition, intraoral scan data, etc. The treatment plan may specify a sequence of tooth movements from an initial tooth arrangement to a target tooth arrangement, with intermediate stages defining incremental repositioning of teeth. The treatment plan may include staging information indicating the number of treatment stages, the duration of each stage, and the specific tooth movements to be achieved at each stage. The treatment plan may further include specifications for dental features such as attachments, precision cuts, bite ramps, and/or power ridges to be applied at various stages of treatment. In some embodiments, a treatment building engine may receive the machine-readable instructions from the treatment protocol along with the patient data and generate the treatment plan by applying the protocol instructions to the patient-specific dental configuration. The generated treatment plan may be presented to the treatment provider for review, optionally including visualizations of predicted tooth positions at various stages of treatment.

At block 568, process logic optionally provides design data for one or more dental treatment appliances. The design data may be based on treatment planning data, e.g., the treatment plan. The design data may indicate a shape, size, material, etc., of one or more treatment appliances for carrying out a treatment in accordance with the treatment goals. The design data may specify the geometry of orthodontic aligners configured to reposition teeth according to the staging information in the treatment plan. In some embodiments, the design data may include specifications for attachment templates, precision cut locations, or other appliance features that correspond to the dental features specified in the treatment plan. The design data may be generated by processing the treatment plan through an appliance design algorithm that translates tooth movement specifications into appliance geometries.

At block 570, process logic optionally provides the treatment appliance design data to a facility for manufacturing the treatment appliances in accordance with the treatment plan and design data. The manufacturing facility may receive the design data via a network connection and may utilize direct fabrication techniques such as additive manufacturing or thermoforming to produce the treatment appliances. The manufactured appliances may be shipped to the treatment provider for delivery to the patient, enabling implementation of the treatment plan generated based on the treatment protocol.

FIG. 5G is a flow diagram of a method 500G for using a GUI to generate a treatment protocol based on a set of provided options, according to some embodiments. At block 572, process logic provides, via a GUI, a set of treatment categories in association with operations of a dental treatment. The treatment categories may include tooth crowding, tooth spacing, shifted midline, anterior leveling, anterior-posterior relationship, posterior crossbite, overbite, interproximal reduction, pontics, and/or extractions.

At block 574, process logic provides, for each of the set of treatment categories, a corresponding set of treatment options. In some embodiments, the set of treatment categories may be selectable, and upon selection of a treatment category the relevant treatment options may be displayed via the GUI.

At block 576, process logic obtains, for a first of the set of treatment categories, a selection from the set of treatment options via the GUI. Optionally, process logic may obtain a user selection for one or more options, and/or a default selection for one or more options for the treatment categories. In some embodiments, each selection may include a default option, which may be accepted if no user selection is provided.

At block 578, process logic provides the selection to a model configured to generate a treatment protocol in a machine-readable format. The model may be a rule-based, deterministic model. The model may be configured to express the selections, provided via the GUI, in a machine-readable format that is of relevance to the treatment, disorders, treatment categories, etc.

At block 580, process logic displays the treatment protocol via the GUI. Displaying the treatment protocol may include displaying machine-readable code, e.g., for review. Displaying the treatment protocol may include displaying a medical algorithm, e.g., in a block form that may be reviewed by a practitioner who is not trained in the use of machine-readable code related to the treatments.

At block 582, process logic optionally provides, via the GUI, a model of dentition. The model may be of example dentition. The model may be of specific dentition, e.g., previous or current patient dentition. The model may exhibit aspects of a default treatment option. The model may exhibit aspects of a target treatment option, e.g., a selection made by a user via the GUI. The model may include indications of treatment areas of interest in association with one of the set of treatment categories.

At block 583, process logic optionally determines that one or more aspects of dentition of a dental patient is outside the scope of treatment options or treatment categories provided by the GUI. Treatment protocol generation operations or treatment planning operations may be augmented, for example when one or more aspects of dental data, of a patient, of a category of treatment, or the like with outside a scope of treatment options, treatment goals, treatment categories, or the like included in the GUI. Based on determining that one or more aspects of a treatment, patient, or the like are outside a scope of the options provided by the GUI, a natural language instruction associated with the condition or treatment (e.g., a natural language instruction associated with treatment of a patient) may be obtained. The natural language instruction may be provided to an AI model, in accordance with methods described herein such as in connection with FIG. 5B, to obtain treatment protocol data (e.g., additional treatment protocol data to augment the machine-readable treatment protocol generated via the GUI), treatment plan data, or the like for improving treatment of one or more dental patients, dental conditions, to improve a treatment response to one or more treatment goals, or the like.

At block 584, process logic optionally generates a treatment plan for the patient. Generating the treatment plan for the patient may include executing the machine-readable instructions of the treatment protocol. Generating the treatment plan may include generating design data for manufacturing of one or more appliances for the treatment. At block 586, process logic optionally generates dental treatment appliance designs corresponding to the treatment plan, and provides the designs to a facility for manufacturing.

FIG. 6A is a flow diagram of a method 600A for adjusting a treatment planning algorithm based on natural language instructions, according to some embodiments. At block 602, process logic obtains first treatment provider instructions in natural language. The first treatment provider instructions are associated with treatment of a dental patient. The first treatment provider instructions may include free-text treatment instructions from a treatment provider associated with a specific dental patient. The first treatment provider instructions may include updates or differences between target dental treatment for the dental patient and a set of treatment preferences or treatment protocol associated with the treatment provider. The first treatment provider instructions may be indicative of one or more differences between target dental treatment for a target dental patient and a set of treatment preferences or treatment protocol associated with the treatment provider.

At block 604, process logic provides the first treatment provider instructions and a prompt comprising rules for a domain-specific format for orthodontic treatment plans to a trained artificial intelligence (AI) model. The trained AI model may be an LLM. The prompt may include a description of a plurality of categories of instructions associated with dental treatment. The prompt may include a set of classifications of treatment instruction categories. The prompt may include a plurality of examples of machine-readable instructions corresponding to natural language instructions within the categories of instructions. The prompt may include one or more examples of natural language instructions corresponding to the example machine-readable instructions. In some embodiments, providing the first treatment provider instructions and the prompt to the trained AI model comprises transmitting at least one of the first treatment provider instructions or the prompt to a remote computing device that executes the trained AI model.

At block 606, process logic obtains, as output from the trained AI model, first machine-readable instructions related to the treatment provider instructions structured according to the domain-specific format. The first machine-readable instructions may be formatted in a target style, e.g., prompt information provided to the trained AI model may include instructions to format output according to a target style. The output from the trained AI model may comprise a machine-readable format that is a domain-specific format for orthodontic treatment. The first machine-readable instructions may be structured according to a language-independent data-interchange format schema that organizes the treatment provider instructions into distinct categories, each category representing a specific aspect of treatment planning. The schema may include a default category for instructions that do not fit into predefined instruction categories. In some embodiments, obtaining the first machine-readable instructions comprises receiving the first machine-readable instructions from the remote computing device.

At block 608, process logic optionally performs treatment instruction validation on the first machine-readable instructions to determine compatibility with a treatment planning engine. Validation may include performing one or more checks to ensure the machine-readable instructions include one or more target attributes. Validation may include performing checks to ensure the machine-readable instructions are compliant with standards related to treatment. Validation may include determining that the first machine-readable instructions do not violate a set of treatment instruction heuristics or rules. Performing validation of the first machine-readable instructions may comprise determining whether each instruction type in the first machine-readable instructions is supported by the treatment planning engine, determining whether parameter values in the first machine-readable instructions fall within predefined acceptable ranges, and responsive to determining that an instruction type is unsupported or a parameter value falls outside an acceptable range, directing the first treatment provider instructions to a manual treatment planning process.

At block 610, process logic causes a treatment planning algorithm to be adjusted based on the first machine-readable instructions. The treatment planning algorithm may be configured to act on patient data to generate a treatment plan, including final tooth positions, intermediate tooth positions (e.g., treatment stages), plans for treatment appliances, or the like. In some embodiments, causing the treatment planning algorithm to be adjusted comprises receiving confirmation to apply the first machine-readable instructions and transmitting the first machine-readable instructions to the remote computing device or a second remote computing device executing an orthodontic treatment planning application. Process logic may generate a treatment algorithm based on output from the trained AI model. Process logic may obtain a standard treatment algorithm, wherein generating the treatment algorithm comprises adjusting the standard treatment algorithm. The standard treatment algorithm may be associated with the treatment preferences. Responsive to instruction validation, process logic may update a treatment algorithm based on the output from the trained AI model.

In some embodiments, process logic obtains second machine-readable instructions encoding treatment preferences of the treatment provider and generates the treatment planning algorithm based on the second machine-readable instructions. The second machine-readable instructions may encode general treatment preferences of the treatment provider applicable to multiple patients. Process logic may obtain treatment provider preferences in natural language and generate the second machine-readable instructions based on the treatment provider preferences. Process logic may obtain a generic treatment planning algorithm, wherein generating the treatment planning algorithm comprises adjusting the generic treatment planning algorithm based on the second machine-readable instructions. Process logic may determine a priority relationship between the first machine-readable instructions and the second machine-readable instructions and adjust the treatment planning algorithm such that the first machine-readable instructions override corresponding portions of the second machine-readable instructions. The first machine-readable instructions may be case-specific machine-readable instructions that have a higher priority than general treatment protocol instructions such that the case-specific machine-readable instructions override corresponding portions of the general treatment protocol instructions in some embodiments.

In some embodiments, process logic obtains dental data of the dental patient and processes the dental data using the adjusted treatment planning algorithm to generate a treatment plan for the dental patient. The patient data may comprise a three-dimensional model of dentition of the patient. Responsive to determining that the first machine-readable instructions are compatible with the treatment planning engine, process logic may provide the first machine-readable instructions and patient data to the treatment planning engine, and the treatment planning engine may generate a treatment plan for the specific dental patient based on the first machine-readable instructions. Process logic may generate a treatment plan based on the output from the trained AI model. Process logic may cause one or more treatment appliances to be manufactured based on the treatment plan.

In some embodiments, the first treatment provider instructions include a reference to one or more teeth expressed according to a first indexing scheme. Process logic may obtain a three-dimensional model of dentition of the specific dental patient, sort teeth of the three-dimensional model based on geometric position along a jaw arc, and map the reference to the one or more teeth to corresponding teeth in the sorted three-dimensional model based on the geometric position. The reference to the one or more teeth may comprise one or more of an interval specification identifying teeth between two specified teeth, a group specification identifying teeth belonging to an anatomical category, a relative position specification identifying teeth based on position relative to a reference tooth, or an exclusion specification identifying teeth by excluding specified teeth from a group. The three-dimensional model may include one or more of a supernumerary tooth or a missing tooth, and mapping the reference to the one or more teeth may account for the supernumerary tooth or the missing tooth based on the geometric position along the jaw arc.

At block 612, process logic optionally obtains second treatment provider instructions in natural language. The second treatment provider instructions may further be provided to the trained AI model. Process logic may optionally further obtain, as output from the trained AI model, second machine-readable instructions. Process logic may further optionally perform treatment instruction validation on the second machine-readable instructions. Responsive to determining that the second machine-readable instructions violate one or more of a set of treatment instruction heuristics, process logic may provide the second treatment provider instructions to a manual treatment planning pipeline. For example, responsive to determining that the second machine-readable instructions do not conform to one or more standards included in the instruction validation, process logic may provide the second treatment provider instructions to a technician for manual adjustment of the treatment planning algorithm based on the second treatment provider instructions.

In some embodiments, the method 600A may be applied to update existing machine-readable instructions. Process logic may obtain first instructions in a first machine-readable format configured to represent parameters for a treatment plan based on input data. The first instructions may encode preferences of a treatment provider. Process logic may obtain second instructions in natural language corresponding to target adjustments to the first instructions. The target adjustments to the first instructions may be based on a target dental patient. The second instructions may comprise one or more deviations from the preferences of the treatment provider. Process logic may provide the second instructions to the trained AI model and obtain output from the trained AI model based on the second instructions, the output comprising third instructions in a second machine-readable format corresponding to the second instructions. Process logic may update the first instructions based on the third instructions. The treatment plan may comprise a dental treatment plan. Process logic may obtain fourth instructions in a third machine-readable format associated with treatment preferences of a treatment provider, wherein the first instructions are based on the fourth instructions. Process logic may obtain fifth instructions in natural language, wherein the fifth instructions comprise the treatment preferences of the treatment provider, provide the fifth instructions to the trained AI model, and obtain, as output from the trained AI model, the fourth instructions.

FIG. 6B is a flow diagram of a method 600B for updating a treatment planning algorithm based on natural language instructions and treatment provider preferences, according to some embodiments. The method 600B illustrates a comprehensive workflow that begins with obtaining treatment provider preferences, converts those preferences into machine-readable formats, and ultimately generates treatment plans and treatment appliances for dental patients. The method 600B demonstrates how natural language input from treatment providers can be processed through AI models to adjust treatment planning algorithms for specific patient cases while maintaining consistency with the treatment provider's general clinical preferences.

At block 616, process logic optionally obtains treatment provider treatment preferences in natural language. The treatment provider preferences may represent general clinical preferences that the treatment provider wishes to apply across multiple patients or patient categories, rather than instructions specific to a single patient case. For example, a treatment provider may specify preferences regarding the aggressiveness of tooth movement, preferred sequencing of treatment operations, maximum amounts of interproximal reduction per contact, or preferred approaches for treating specific types of malocclusions. The treatment provider preferences may be expressed in free-text natural language format, allowing the treatment provider to articulate their clinical philosophy and standard practices without requiring knowledge of machine-readable instruction formats. Process logic may use these treatment provider preferences to generate a treatment protocol for the treatment provider. The treatment protocol serves as a template that encodes the treatment provider's standard approach to dental treatment, which can then be applied as a baseline when generating treatment plans for individual patients. The treatment protocol may act as a default treatment planning template, establishing the treatment provider's preferred parameters and approaches that will be applied unless specifically overridden by patient-specific instructions. In some embodiments, a machine-readable treatment protocol may be generated based on the treatment provider preferences. The generation of the machine-readable treatment protocol may be performed by an AI model, such as a large language model (LLM), that is trained to translate natural language clinical preferences into structured, machine-readable formats. In some embodiments, the machine-readable treatment protocol may be in a programming language intended to bridge a gap between human instruction and a treatment planning algorithm. This intermediate programming language may be a domain-specific language (DSL) designed specifically for orthodontic or dental treatment planning, enabling precise specification of treatment parameters while remaining more accessible than general-purpose programming languages. Alternatively, the protocol may be incorporated directly into a treatment planning algorithm as executable code or configuration parameters. In some cases, an intermediate machine-readable protocol may be used to generate a treatment planning algorithm, where the protocol serves as a specification from which algorithm components are derived or configured. In some embodiments, a treatment protocol may have previously been generated for a practitioner, and the treatment protocol may be accessed.

At block 620, process logic optionally obtains the machine-readable treatment protocol and generates a treatment planning algorithm based on the machine-readable treatment protocol. The treatment planning algorithm represents executable logic that can process patient dental data to generate treatment plans in accordance with the treatment provider's preferences. In some embodiments, a generic treatment planning algorithm may be obtained as a starting point. The generic treatment planning algorithm may represent a standard or default approach to dental treatment planning that incorporates generally accepted clinical practices. Process logic may then modify the generic treatment planning algorithm by adding or replacing one or more portions based on the treatment provider preferences or treatment protocol to generate a customized treatment planning algorithm. For example, if the treatment provider's preferences specify a particular approach to treating Class II malocclusions, the corresponding portion of the generic algorithm may be replaced with logic implementing the treatment provider's preferred approach. This customization process enables each treatment provider to have a personalized treatment planning algorithm that reflects their individual clinical philosophy while building upon a foundation of established treatment planning practices.

At block 622, process logic obtains first treatment provider instructions in natural language associated with treatment of a dental patient. Unlike the treatment provider preferences obtained at block 616, which represent general preferences applicable across multiple patients, the first treatment provider instructions obtained at block 622 are specific to a particular dental patient and may indicate desired modifications or adjustments to the standard treatment approach for that patient's case. The first treatment provider instructions may be expressed as free-text comments, notes, or directives that the treatment provider enters when reviewing a patient case. For example, the treatment provider may specify that a particular tooth should be extracted, that interproximal reduction should be limited in a specific region, or that a particular treatment goal should be prioritized for the patient. Operations of block 622 may share one or more features with operations of block 602 of FIG. 6A, including obtaining instructions via a graphical user interface, receiving instructions through a chat interface, or obtaining instructions from stored treatment records.

At block 624, process logic provides the first treatment provider instructions to a trained AI model. The trained AI model may comprise a large language model (LLM) or other natural language processing model that has been trained or configured to translate natural language treatment instructions into machine-readable formats suitable for treatment planning operations. The AI model may be provided with the first treatment provider instructions along with the treatment protocol and/or an accompanying prompt that includes construction rules for a domain-specific format, descriptions of instruction categories, examples of machine-readable instructions corresponding to various types of natural language instructions, and indexing rules for tooth numbering systems. The AI model processes the natural language instructions and generates corresponding machine-readable instructions as output. Operations of block 624 may share one or more features with operations of block 604 of FIG. 6A, including providing prompts with the instructions, utilizing retrieval-augmented generation to access relevant documentation, or providing context from the treatment provider's preference history.

At block 626, process logic obtains output from the AI model. The output from the AI model may be based on the first treatment provider instructions provided at block 624. The output from the AI model may include first machine-readable instructions that are related to and correspond to the treatment provider instructions. The first machine-readable instructions may be structured according to a domain-specific format for orthodontic treatment planning, such as a JSON schema or other structured data format that can be parsed and executed by treatment planning systems. The machine-readable instructions may specify treatment parameters, tooth movements, staging information, attachment placements, interproximal reduction amounts, and/or other treatment-related data in a format that can be directly applied to adjust treatment planning operations. Operations of block 626 may share one or more features with operations of block 606 of FIG. 6A, including obtaining instructions in a standardized machine-readable format, receiving instructions that have been validated against a schema, or obtaining instructions that include parameterized treatment specifications.

At block 628, process logic adjusts a treatment planning algorithm based on the first machine-readable instructions. The treatment planning algorithm that is adjusted may be the algorithm generated at block 620 based on the treatment provider's general preferences, or may be a standard treatment planning algorithm. The adjustment modifies the treatment planning algorithm to incorporate the patient-specific instructions provided by the treatment provider. For example, if the machine-readable instructions specify that a particular tooth should be extracted, the treatment planning algorithm may be adjusted to exclude that tooth from movement calculations and to plan for closure of the resulting space. The adjustment may involve modifying algorithm parameters, adding conditional logic, replacing algorithm components, or otherwise configuring the algorithm to implement the treatment provider's patient-specific instructions. Operations of block 628 may share one or more features with operations of block 610 of FIG. 6A, including performing validation of the machine-readable instructions prior to adjustment, applying the instructions to modify specific algorithm parameters, or generating a patient-specific variant of the treatment planning algorithm.

At block 630, process logic optionally obtains dental data of the dental patient. The dental data may include a three-dimensional model of the patient's dentition, which may be generated from intraoral scan data captured using an intraoral scanner or other dental arch data capturing equipment. The three-dimensional model may represent the current state of the patient's teeth, including tooth positions, orientations, shapes, and relationships between teeth. The dental data may additionally or alternatively include a three-dimensional model predicting the patient's dentition after one or more stages of orthodontic treatment, enabling visualization and planning of intermediate treatment stages. The dental data may also include predictions of the patient's dentition after an upcoming dental treatment, such as a target final dentition (e.g., a 3D model of the patient's dentition at the end of treatment), extraction of one or more teeth, placement of pontics to address missing teeth, or other dental procedures that will affect the patient's dental configuration. Other predictive dental data may include simulations of tooth movement, predictions of treatment outcomes, or models of expected final tooth positions. Process logic further optionally processes the dental data using the treatment planning algorithm that was adjusted at block 628. The adjusted treatment planning algorithm applies the treatment provider's general preferences as encoded in the treatment protocol, as well as the patient-specific instructions encoded in the machine-readable instructions, to the patient's dental data. Utilizing the treatment planning algorithm to process the dental data may generate a treatment plan for the dental patient. The treatment plan may include staging information specifying intermediate tooth positions at each stage of treatment, final tooth positions representing the target outcome, specifications for treatment appliances to be used at each stage, and other treatment-related data necessary to implement the planned treatment.

At block 632, process logic optionally causes one or more treatment appliances to be manufactured based on the treatment plan generated at block 630. The treatment appliances may include orthodontic aligners, retainers, or other dental appliances designed to implement the treatment plan by repositioning the patient's teeth according to the planned staging. In some embodiments, treatment planning data generated as part of the treatment plan may include data facilitating manufacture of one or more treatment appliances, such as three-dimensional models of appliance geometries, material specifications, or manufacturing instructions. Treatment planning data may be stored in a data store to be provided for future manufacturing of appliances, enabling appliances to be manufactured on demand as the patient progresses through treatment stages. One or more appliances may be manufactured in accordance with the treatment planning data included in the treatment plan using direct fabrication techniques such as three-dimensional printing, stereolithography, or other additive manufacturing processes. The manufactured appliances implement the treatment provider's clinical preferences and patient-specific instructions as translated through the AI model and incorporated into the treatment planning algorithm, thereby completing the workflow from natural language input to physical treatment appliances.

FIG. 6C is a flow diagram of a method 600C for updating a set of machine-readable instructions based on natural language input, according to some embodiments. At block 636, process logic optionally obtains natural language instructions encoding treatment provider treatment preferences. The natural language instructions encoding treatment provider preferences may include one or more differences between treatment provider preferences and a default treatment protocol. Process logic may optionally provide the natural language instructions to an AI model. Process logic may obtain machine-readable instructions translating the treatment provider preferences to a treatment planning algorithm.

At block 638, process logic optionally generates first instructions based on output from the AI model. The first instructions may be in a machine-readable format.

At block 640, process logic obtains first instructions in a first machine-readable format configured to generate a treatment plan based on input data. The input data may be patient data. The input data may be patient dental data, e.g., one or more three-dimensional models of patient dentition. The first instructions may include the preferences of the treatment provider.

At block 642, process logic obtains second instructions in natural language. The second instructions correspond to target adjustments to the first instructions. In some embodiments, the first instructions may include a treatment protocol encoding treatment provider preferences, and the second instructions include differences between the treatment protocol and a target treatment plan for a particular patient.

At block 644, process logic provides the second instructions to an AI model. The operations of block 644 may share one or more features with operations of block 604 of FIG. 6A.

At block 646, process logic obtains output from the AI model. The output may include third instructions. The third instructions may be expressed in a second machine-readable format, which may be different than the first machine-readable format. Operations of block 646 may share one or more features with operations of block 606 of FIG. 6A.

At block 648, process logic updates the first instructions based on the third instructions. In some embodiments, operations of block 648 may share one or more features with operations of block 610 of FIG. 6A.

At block 650, process logic optionally causes one or more treatment appliances to be manufactured based on the updated instructions. Operations of block 650 may share one or more features with operations of block 632 of FIG. 6B.

FIG. 6D is a flow diagram of a method 600D for generating machine-readable instructions from natural language instructions associated with dental treatment, according to some embodiments. At block 654, process logic obtains treatment provider instructions in natural language. The treatment provider instructions are indicative of one or more differences between target dental treatment for a target dental patient and a set of treatment preferences associated with the treatment provider. The set of treatment preferences may be encoded in a treatment protocol. The set of treatment preferences may be encoded in a treatment planning algorithm, which may be adjusted during operations of method 600D.

At block 656, process logic obtains a prompt. The prompt may be recovered from memory. The prompt may include multiple parts or pieces, which may be modular, e.g., different parts may be incorporated together to generate the prompt. The prompt includes a description of a set of categories of instructions associated with dental treatment and a set of examples of machine-readable instructions corresponding to natural language instructions within the categories of instructions.

At block 658, process logic provides the treatment provider instructions and the prompt as input to an AI model. Operations of block 658 may share one or more features with operations of block 604 of FIG. 6A.

At block 660, process logic obtains output from the AI model. The output may be generated responsive to providing the prompt and the treatment provider instructions as input to the AI model. The output optionally includes machine-readable instructions corresponding to the first treatment provider instructions, such as in a domain-specific format for orthodontic treatment.

At block 662, process logic optionally performs instruction validation. Instruction validation may include performing one or more tests or checks on machine-readable instructions output by the AI model. Instruction validation optionally includes determining that the machine-readable instructions do not violate one or more treatment heuristics. Instruction validation may include determining that the machine-readable instructions correspond with a set of treatment best practices. Process logic may further optionally update a treatment algorithm or plan responsive to instruction validation.

At block 664, process logic generates a treatment algorithm based on output from the AI model. Process logic further optionally obtains patient data of the target dental patient, optionally including a three-dimensional model of dentition of the patient.

At block 666, process logic generates a treatment algorithm based on the output from the AI model. Generating the treatment algorithm may optionally be performed by adjusting a standard treatment algorithm, which may be based on the treatment preferences. Process logic may optionally generate a treatment plan by applying the treatment algorithm to the patient data. The standard treatment algorithm may be a global default algorithm, an algorithm encoding the preferences of the treatment provider, or the like.

At block 668, shown with dashed lines indicating an optional step, process logic optionally causes one or more treatment appliances to be manufactured based on the updated instructions. Operations of block 668 may share one or more features with operations of block 632 of FIG. 6B.

FIG. 6E is a flow diagram of a method 600E for indexing ordered objects in a machine-readable format, based on natural language input, according to some embodiments. At block 670, process logic obtains first natural language instructions for treating dentition of a patient. The first natural language instructions comprise a first reference to a plurality of ordered teeth according to a first indexing scheme for at least one of teeth or inter-tooth intervals. In some embodiments the teeth may be ordered based on tooth identity, geometrical relationships between the teeth, or the like. The references to the teeth may include identity-based references (e.g., UR3), location-based references (e.g., mesial to another tooth), etc. The natural language instructions may include one or more references to subsets of the ordered teeth, spaces between the teeth, etc. For example, the first natural language instructions may comprise a reference to a first space between two teeth of the plurality of ordered teeth, a reference to a subset of the plurality of ordered teeth, and so on. The natural language instructions may include references in accordance with a first indexing scheme. For example, dentition may be described in accordance with several common indexing schemes, including Palmer, UNS, FDI, etc. In some embodiments, a second reference to the plurality of ordered teeth according to a second indexing scheme may be included in the natural language instructions. In some embodiments, the first natural language instructions comprise an update to a dental treatment protocol with respect to a dental patient, and the plurality of ordered objects comprise teeth of the dental patient. While method 600E is described with reference to ordered teeth, it may also be applied to other sets or subsets of ordered objects and/or spaces between objects.

At block 672, process logic provides the natural language instructions and an accompanying prompt as input to an AI model. The prompt may comprise a description of the first indexing scheme. In some embodiments, the first indexing scheme comprises: a definition of individual tooth identifiers supporting multiple numbering systems; a definition of tooth groups representing anatomical categories; a definition of tooth intervals representing inclusive ranges between two teeth; a definition of relative tooth positions based on mesial or distal direction; and exclusion logic representing a group of exclusions using nested references to include and exclude sets of teeth. In some embodiments, the instructions may include one or more updates to a treatment protocol, such as updates to a treatment provider's treatment preferences, changes to be made to a standard protocol based on a particular patient, or the like. The prompt may further comprise a description of a second indexing scheme. The prompt may further comprise an indication of a preferred indexing scheme associated with the plurality of ordered teeth. In some embodiments, providing the natural language instructions and the accompanying prompt to the AI model comprises transmitting at least one of the natural language instructions or the accompanying prompt to a remote computing device that executes the AI model.

At block 674, process logic obtains, as output from the AI model, first machine-readable instructions for treating the dentition of the patient. The AI model may be a large language model (LLM). The first machine-readable instructions comprise instructions associated with the plurality of ordered objects. The first machine-readable instructions comprise a second reference to the plurality of ordered objects (e.g., ordered teeth) according to a first machine-readable indexing scheme. In some embodiments, obtaining the first machine-readable instructions comprises receiving the first machine-readable instructions from a remote computing device that executes the AI model.

At block 676, process logic optionally performs validation of the first machine-readable instructions. The validation may comprise determining that the first machine-readable instructions do not violate a set of heuristics associated with the first machine-readable instructions. Operations of block 676 may share one or more features with operations of block 608 of FIG. 6A.

At block 678, process logic optionally associates indices of the first machine-readable indexing scheme to a three-dimensional model of the plurality of ordered teeth, wherein the ordered teeth of the three-dimensional model are indexed according to a second machine-readable indexing scheme, different than the first. For example, a three dimensional model of patient dentition may be indexed in accordance with identity of the teeth. This may be less useful in machine-readable contexts where relative positions are referred to in instructions, in particular in cases with unusual dental ordering, missing teeth, extra teeth, or other situations with non-standard geometric relationships between teeth of a dental arch. Mapping references from natural language input, to machine-readable instructions, and then mapping the references in the machine-readable instructions to patient data, may enable machine-generated instructions to perform treatment planning procedures based on geometrically-dependent natural language input by a treatment provider.

In some embodiments, block 678 includes a sequence of operations, set forth in blocks 678A-E.

At block 678A, process logic may segment the three-dimensional model to identify teeth in the three-dimensional model. The segmentation process may identify individual teeth within the three-dimensional model for subsequent processing and mapping operations. Segmentation of the three-dimensional model may be performed using various techniques, including instance segmentation and semantic segmentation. Instance segmentation involves identifying and delineating each individual tooth as a separate object instance within the three-dimensional model, enabling the system to distinguish between different teeth even when they belong to the same tooth type (e.g., distinguishing between a first molar and a second molar). Semantic segmentation involves classifying each point or region of the three-dimensional model according to a predefined set of categories, such as tooth tissue, gingival tissue, or background, without necessarily distinguishing between individual instances of the same category. In some embodiments, a combination of instance segmentation and semantic segmentation may be employed, where semantic segmentation first identifies regions corresponding to dental structures, and instance segmentation subsequently separates individual teeth within those regions. The segmentation may be performed using machine learning models trained on labeled dental scan data, including convolutional neural networks, point cloud processing networks, or other architectures suitable for three-dimensional data analysis. The segmentation output may include mesh segments, point cloud clusters, or voxel labels corresponding to each identified tooth, which are then used for subsequent indexing and mapping operations.

At block 678B, process logic may sort the identified teeth based on geometric position along a jaw arch. In some embodiments, an upper jaw is sorted from right to left along the arch and a lower jaw is sorted from left to right along the arch. The sorted teeth may be cached for reuse across multiple instructions within a same orthodontic case. In some embodiments, the three-dimensional model includes one or more of an unerupted tooth, a supernumerary tooth, or a pontic tooth, and sorting the teeth based on geometric position accounts for the one or more of the unerupted tooth, the supernumerary tooth, or the pontic tooth.

At block 678C, process logic may generate an array of teeth corresponding to the machine-readable instructions for treating the dentition of the patient. The array of teeth may be ordered according to the first machine-readable indexing scheme, such that each element of the array corresponds to a tooth identified in the machine-readable instructions. The ordering of teeth in the array may follow a sequential arrangement based on tooth numbering conventions, such as progressing from one side of the dental arch to the other, or may follow an anatomical ordering based on tooth type and position within the jaw. The array of teeth may include entries for each tooth referenced in the machine-readable instructions, with each entry containing tooth identification information, associated treatment parameters, or other data relevant to the treatment operations specified in the machine-readable instructions. In some embodiments, the array of teeth may be structured as an ordered list or data structure that preserves the positional relationships between teeth as specified in the indexing scheme, enabling subsequent mapping operations to correctly associate treatment instructions with corresponding teeth in the three-dimensional model of the patient's dentition.

At block 678D, process logic may map teeth of the array of teeth to the sorted teeth of the three-dimensional model. In some embodiments, mapping the representation of the one or more teeth to the three-dimensional model comprises applying a matching predicate based on a type of representation. For individual teeth, a tooth may be selected if its identifier matches an entry in the machine-readable instructions. For tooth groups, a tooth may be selected if it belongs to a specified anatomical group, filtered by jaw and side. For intervals, a tooth may be selected if its position lies between begin and end identifiers, inclusive. For relative positions, a tooth may be selected if it is immediately mesial or distal to a referenced tooth based on arch geometry. For exclusion logic, a matching set may be computed by subtracting excluded teeth from an included group.

At block 678E, process logic may determine an array of interteeth intervals from the mapped teeth of the array of teeth. In some embodiments, process logic determines that the natural language instructions include a reference to an interproximal space between teeth. Process logic may partition the mapped teeth into jaw-specific subarrays, such as a first subarray for an upper jaw and a second subarray for a lower jaw. Process logic may construct interteeth intervals between each pair of adjacent teeth within each jaw-specific subarray. The constructed intervals may be applied to generate a treatment plan addressing the interproximal space.

In some embodiments, the prompt is extensible to allow addition of new examples and rules as clinical language evolves, and the LLM produces output that conforms to a predefined schema and captures semantic intent of the natural language treatment instructions. In some embodiments, process logic determines that the machine-readable instructions include an unsupported instruction type or an ambiguous reference, and automatically flags the orthodontic case for manual review responsive to the determining.

FIG. 6F is a flow diagram of a method 600F for adjusting a treatment planning algorithm, according to some embodiments. At block 680, process logic obtains first treatment provider instructions in natural language. The treatment provider instructions may be associated with treatment of a dental patient. The treatment provider instructions may include a first reference to one or more teeth of the dental patient. The reference to the teeth of the dental patient may be expressed in accordance with a first indexing scheme. In some cases, a dental practitioner may include multiple references in accordance with multiple different indexing schemes.

At block 682, process logic provides the natural language instructions and an accompanying prompt as input to an AI model. The prompt may include a description of the first indexing scheme. The prompt may include a description of the second indexing scheme. The prompt may further include instructions to enable the AI model (e.g., an LLM) to generate machine-readable instructions related to the natural language instructions.

At block 684, process logic obtains, from the AI model, first machine-readable instructions related to the treatment provider instructions. The first machine-readable instructions may include a first reference to a first tooth in accordance with a first machine-readable indexing scheme.

At block 686, process logic optionally performs validation of the first machine-readable instructions. Validation may include validating that any indexing of teeth is reasonable, accurate, corresponds to the natural language input, etc. Validation of block 686 may share one or more features with operations of block 608 of FIG. 6A.

At block 688, process logic adjusts a treatment planning algorithm based on the first machine-readable instructions. Operations of block 688 may share one or more features with operations of block 610 of FIG. 6A.

At block 690, process logic optionally obtains dental data of the dental patient. The dental data may include a three-dimensional model of patient dentition. The dental data may include a predictive model of dentition after one or more stages of treatment, such as pontic treatment or extraction of one or more teeth. Process logic further optionally maps a first instruction associated with a first tooth to a second machine-readable indexing scheme of the dental data. The dental data may include a target final arrangement of the patient's teeth (e.g., represented as a 3D model such as a 3D mesh). Process logic further optionally processes the dental data using the treatment planning algorithm to generate a treatment plan for the patient.

At block 692, process logic optionally causes one or more appliances to be manufactured based on the treatment plan. Operations of block 692 may share one or more features with operations of block 632 of FIG. 6B.

FIG. 6G is a flow diagram of a method 600G for translating natural language indexing to machine-readable indexing for updating a treatment planning algorithm, according to some embodiments. The method 600G enables conversion of treatment provider instructions that reference teeth using various natural language indexing schemes into machine-readable formats suitable for automated treatment planning operations.

At block 693, process logic obtains treatment provider instructions in natural language. The treatment provider instructions may be indicative of one or more differences between target dental treatment for a target dental patient and a set of treatment preferences associated with the treatment provider. The treatment provider instructions may include a first reference to one or more teeth of the dental patient expressed in accordance with a first indexing scheme. For example, a treatment provider may provide instructions such as “add optimized attachment to UL2” using the Palmer numbering system, or “place rotation attachment on tooth 1.3” using the FDI numbering system, or “extract tooth 14” using the Universal numbering system. The treatment provider instructions may also reference teeth using descriptive terminology such as “upper left lateral incisor” or “lower right first molar.” The first indexing scheme may include any of the Palmer, FDI, Universal, or descriptive naming conventions commonly used by dental practitioners.

At block 694, process logic obtains a prompt. The prompt may include a description of the first indexing scheme to enable the AI model to recognize and interpret tooth references in the treatment provider instructions. For example, the prompt may describe that in the Palmer system, teeth are identified with letters indicating jaw position (U for upper, L for lower) and side (R for right, L for left) followed by a number from 1 to 8. The prompt may include a description of a set of categories of instructions associated with dental treatment, such as categories for attachments, extractions, interproximal reduction, and/or tooth movement restrictions. The prompt may include a set of examples of machine-readable instructions corresponding to natural language instructions within the categories of instructions. For example, the prompt may include an example showing that “add MDRC to UR3” translates to a JSON object specifying action “add,” attachment type “mesial_distal_root_control,” and tooth identifier in a standardized format. The prompt may include a description of a second indexing scheme, enabling the AI model to handle instructions that reference teeth using multiple numbering conventions within the same set of instructions.

At step 695, process logic provides the treatment provider instructions and the prompt as input to an AI model. The AI model may be an LLM such as a large language model configured to process natural language input and generate structured output. The AI model may be configured via prompt engineering to generate machine-readable instructions from natural language instructions. For example, the AI model may receive the instruction “place G4 attachments on 1.2, 1.3, and 1.4” along with a prompt describing the FDI numbering system and the G4 protocol requirements, enabling the model to generate appropriate machine-readable output that accounts for protocol-specific conditions such as tooth type requirements.

At step 696, process logic obtains output from the AI model. The output may include a second reference to the one or more teeth expressed in accordance with a third indexing scheme. The third indexing scheme may be a machine-readable indexing scheme suitable for processing by treatment planning software. The third indexing scheme may have a geometric component, such as referencing teeth based on their position along the dental arch. The third indexing scheme may include referential indexing, such as indexing a tooth in relation to its position compared to one or more other teeth. For example, the AI model may convert “UL2” from the Palmer system to a Universal system identifier “10” or to a geometric position identifier indicating the second tooth from the midline on the upper left quadrant. The output may include structured JSON data specifying the tooth identifiers in a standardized format along with the associated treatment actions and parameters.

At step 697, process logic optionally performs treatment instruction validation of the output from the AI model. Treatment instruction validation may include verifying that the machine-readable instructions conform to a predefined schema, checking that referenced teeth exist and are valid for the specified treatment actions, and confirming that the instructions do not violate clinical safety heuristics. For example, validation may verify that an instruction to place an extrusion attachment specifies a valid tooth identifier and that the attachment type is appropriate for the identified tooth type. Treatment instruction validation may share one or more features with operations of block 686 of FIG. 6F, operations of block 608 of FIG. 6A, or other validation operations described herein. If validation fails, the instructions may be directed to a manual treatment planning pipeline for review by a trained technician.

At step 698, process logic optionally obtains dental data of the dental patient. The dental data may include a three-dimensional model of patient dentition generated from intraoral scans. Process logic may map a first instruction associated with a first tooth to a fourth machine-readable indexing scheme of the dental data. The fourth indexing scheme may be related to tooth identity as determined from the three-dimensional model, such as unique identifiers assigned to segmented teeth within the model. Mapping the third indexing scheme onto the fourth indexing scheme may include incorporating geometric position information based on the three-dimensional model, enabling application of instructions to teeth based on their actual geometric positions within the patient's dentition. For example, if the AI model output references “tooth 10” in the Universal system, process logic may map this reference to a specific tooth object within the three-dimensional model by matching the geometric position of the tooth along the upper arch. Process logic may further process the dental data using the treatment planning algorithm to generate a treatment plan for the patient, applying the validated machine-readable instructions to the patient-specific dental model to produce staging information, attachment placements, or other treatment plan components.

FIG. 7A is a flow diagram of a method 700A for producing a machine- or model-generated prompt for an LLM, according to some embodiments. The method 700A enables automated generation of prompts that can be used in downstream pipelines for automated text understanding, including dental treatment planning applications. At block 702, process logic obtains a large language model (LLM) base prompt. The base prompt is associated with a first target task for a first LLM. The base prompt may be configured to cause, potentially along with additional input, the first LLM to perform the first task. The first task may include assigning portions of input text to a first set of categories. The first task may include assigning portions of input treatment instructions to a set of categories. The first task may include assigning input to a set of pre-generated categories, e.g., categories delineated in the base prompt. In some embodiments, the base prompt may be configured to cause an LLM to convert natural language dental treatment instructions into a structured, machine-readable format.

At block 704, process logic provides a first prompt generation request as input to a first LLM. The first prompt generation request may include the LLM base prompt, a task description for a second target task different from the first task, a set of design principles associated with the first task, and/or one or more examples. The task description specifies the intended task for which the prompt is being generated. In dental treatment applications, the task description may specify a target type of dental treatment instructions to be processed, such as case-specific treatment instructions for a particular dental patient, treatment modification instructions requesting changes to an existing dental treatment plan, orthodontic finishing instructions specifying final tooth positioning requirements, or clinical protocol instructions specifying treatment preferences applicable to multiple patients.

The set of design principles comprises a description of target properties for the model-generated prompt, specifying structural and behavioral requirements that output from an LLM based on the model-generated prompt is to adhere to. The design principles may include a target output schema for dental treatment data. In some embodiments, the set of design principles comprises one or more of: a required output format for dental treatment data; dental treatment categories to be extracted including orthodontic treatment parameters; expected robustness to variation in dental terminology and tooth numbering systems; extensibility requirements for accommodating new dental treatment instruction types; or mandatory inclusion of representative dental treatment instruction examples. For example, when the first target task comprises interpreting orthodontic instructions using a base prompt such as a specialized instruction conversion prompt, the design principles may specify that the model-generated prompt for the second target task should maintain compatibility with the output schema of the first target task while extending coverage to additional instruction categories.

The one or more examples comprise situations that the prompt generated based on the first prompt generation request will be expected to enable an LLM or other AI model to manage. In some embodiments, the first prompt generation request further comprises a set of unannotated, real-world examples that serve as semantic anchors defining a scope and nature of the second target task without prescribing specific outputs. For dental treatment applications, the examples may comprise unannotated dental treatment instructions from treatment providers, illustrating inputs the model-generated prompt is expected to handle. The examples may include examples of natural language input that may be provided that is to be categorized into a category that the first set of categories does not comprise. For instance, the examples may include doctor-provided modification instructions that request changes to an existing treatment plan, where such modification instructions may not be fully addressed by the categories defined in the base prompt associated with the first target task.

The second target task may include generating machine-readable instructions, categorizing instructions, generating additional categories to expand upon categories of the first task, and/or developing a second set of categories based on additional input text where the second set of categories shares one or more categories with the first set of categories. The second target task may include operations to support generation of a treatment plan for a dental patient. The second target task may include generating machine-implementable instructions (e.g., to update one or more selections of a GUI-based treatment management application) or machine-readable instructions (e.g., code) for performing treatment planning, for generating or amending a treatment protocol, etc. In some embodiments, the second target task comprises generation of a second set of categories to augment the first set of categories, where the natural language input comprises a set of instructions related to healthcare treatment of a patient, such as dental treatment instructions. As a specific example, the second target task may comprise categorizing doctor-provided modification instructions into structured categories, enabling accurate extraction and classification of treatment modification requests. As another example, the second target task may comprise creating a comprehensive extension prompt that interprets and structures instructions that are not currently supported by a treatment planning engine, ensuring that all clinical input is preserved and made machine-readable even when the instructions fall outside the scope of the first target task.

In some embodiments, the first target task and the second target task may be compared and contrasted as follows. The first target task may be a well-established, static, task-specific operation associated with the base prompt. For example, the first target task may comprise converting natural language orthodontic treatment instructions into a structured, machine-readable format using a carefully engineered prompt that covers a defined range of instruction types. The first target task may have proven effective for its intended use case but may be limited in scope to predefined categories and instruction types. In contrast, the second target task may extend, augment, or adapt the first target task to handle new scenarios, additional categories, or cases not covered by the first target task. For example, the second target task may comprise generating a comprehensive prompt capable of interpreting and structuring instructions that are not yet supported by the treatment planning engine, or categorizing modification instructions that fall outside the predefined categories of the base prompt. The second target task may share one or more categories with the first target task while adding new categories to address edge cases, unsupported instruction types, or ambiguous inputs. In some cases, the first target task and the second target task may be the same or similar tasks, where the model-generated prompt is created to alleviate deficiencies of the base prompt, such as improving handling of specific instruction patterns or reducing false positive classifications.

In some embodiments, providing the first prompt generation request to the first LLM comprises transmitting the first prompt generation request to a remote computing device that executes the first LLM. Obtaining the model-generated prompt may comprise receiving the model-generated prompt from the remote computing device. Similarly, providing the model-generated prompt and the input associated with the specific instance of the second target task to the second LLM may comprise transmitting the model-generated prompt and the input to the remote computing device or a second remote computing device that executes the second LLM, and obtaining the output from the second LLM may comprise receiving the second output from the remote computing device or the second remote computing device.

At block 706, process logic obtains, as first output from the first LLM, a model-generated prompt for the second target task. The model-generated prompt may be based on the prompt generation request. The model-generated prompt may be configured to cause an LLM to perform the second target task, e.g., when provided along with additional input to the LLM. In some cases, the first target task and the second target task may be the same task or similar tasks; for example, a model-generated prompt may be created to alleviate deficiencies of the base prompt. Deficiencies of the base prompt may be described in the prompt generation request.

The model-generated prompt may include: a detailed task description; a list of categories and subcategories to be extracted; interpretation rules for linguistic patterns and edge cases; output formatting requirements including adherence to a schema; one or more examples, and/or guidance for handling ambiguous or compound instructions. For dental treatment applications, the model-generated prompt may comprise: a detailed task description for converting dental treatment instructions to machine-readable format; a list of dental treatment categories and subcategories to be extracted, the categories including one or more of tooth movement instructions, attachment instructions, interproximal reduction instructions, and/or staging instructions; interpretation rules for dental terminology and clinical language patterns; output formatting requirements including adherence to a schema for dental treatment data; input and output format specifications with dental treatment instruction examples; examples, and/or guidance for handling ambiguous or compound dental treatment instructions.

In some embodiments, process logic obtains an initial schema as output from the first LLM, the initial schema comprising a structured representation of dental treatment categories, treatment parameters, and formatting rules. The schema may be easier to review, both by an automatic review system or by a subject matter expert, than a full prompt.

At block 708, process logic optionally performs review and refinement analysis of a preliminary model-generated prompt to refine the preliminary model-generated prompt and generate the model-generated prompt. In some embodiments, process logic obtains second output from the first LLM prior to obtaining the first output, the second output comprising the preliminary model-generated prompt. The prompt generation request may be reviewed, automatically (e.g., by the processing device) and/or manually (e.g., by a subject matter expert). In some cases, schema described in the model-generated prompt may be reviewed.

The review and refinement analysis may be performed iteratively across multiple cycles, wherein each cycle processes a focused subset of representative data and key insights are explicitly carried forward between cycles to overcome context window limitations of the first LLM. In some embodiments, the one or more examples comprise a plurality of examples.

In some embodiments, the review and refinement analysis comprises at block 708A generating a schema by the first LLM based on processing of the first prompt generation request or a subsequent prompt generation request. In some embodiments, the review and refinement analysis comprises at block 708B evaluating the schema by the first LLM by processing the plurality of examples in view of the schema. In some embodiments, the review and refinement analysis comprises at block 708C determining, based on a result of the evaluating, a) whether the schema captures semantics of the first prompt generation request or the subsequent prompt generation request, b) whether any use cases are not covered or are misrepresented, c) whether any inconsistencies are present, and/or d) whether any deficiencies and/or weaknesses exist.

For dental treatment applications, performing iterative review and refinement may comprise passing a batch of the unannotated dental treatment instructions through the initial schema. Performing iterative review and refinement may further comprise analyzing outputs to assess how well the schema captures semantics of the unannotated dental treatment instructions. Performing iterative review and refinement may further comprise identifying dental treatment instruction types that are not covered or are misinterpreted. Performing iterative review and refinement may further comprise identifying ambiguities or inconsistencies in dental terminology interpretation. Performing iterative review and refinement may further comprise refining the schema based on the analysis. The first LLM may be used to identify weaknesses in the schema related to dental treatment instruction interpretation and to suggest improvements to the schema. The improvements may comprise one or more of refining dental treatment categories, clarifying edge cases involving dental terminology, and/or adding missing rules and examples for dental treatment instructions.

In some embodiments, one or more first examples of the plurality of examples are evaluated using a first instance of the first LLM in parallel with one or more second examples of the plurality of examples being evaluated using a second instance of the first LLM. This parallel processing enables efficient evaluation of large numbers of examples. In some embodiments, additional instances of the first LLM may be instantiated to evaluate third, fourth, fifth, or additional subsets of examples concurrently. For example, a plurality of LLM instances may be deployed across multiple computing nodes, where each instance processes a distinct subset of the examples simultaneously. The number of parallel instances may be scaled based on available computational resources, the total number of examples to be evaluated, or latency requirements for prompt generation. In some embodiments, the examples may be partitioned across the parallel instances using load balancing techniques to ensure approximately equal processing time across instances. The results from each parallel evaluation may be aggregated upon completion to provide a comprehensive assessment of the draft prompt across all examples. This parallelization approach may reduce the total time required for prompt refinement and validation, particularly when evaluating prompts against large datasets of treatment provider instructions or when iterating through multiple refinement cycles. In some embodiments, different categories of examples may be assigned to different LLM instances, enabling specialized evaluation of how the prompt handles distinct types of natural language instructions associated with dental treatment planning.

At block 708D, process logic may determine whether stopping criteria are satisfied. The stopping criteria may include one or more conditions that indicate the model-generated prompt has reached a sufficient level of quality or refinement. Example stopping criteria may include determining that the schema produced by the LLM correctly handles all provided examples without errors or misclassifications. Stopping criteria may include determining that no additional edge cases, inconsistencies, or deficiencies have been identified during the most recent evaluation iteration. Stopping criteria may include determining that a threshold number of refinement iterations have been completed, such as a maximum iteration count to prevent indefinite refinement loops. Stopping criteria may include determining that the output of the model-generated prompt conforms to a target schema or format specification for a predetermined percentage of test cases. Stopping criteria may include determining that successive iterations produce no substantive changes to the prompt, indicating convergence of the refinement process. Stopping criteria may include determining that the model-generated prompt achieves a target accuracy metric when applied to a validation dataset of natural language instructions and corresponding expected machine-readable outputs. Stopping criteria may include determining that the prompt satisfies design principles specified in the prompt generation request, such as coverage of all instruction categories or adherence to structural requirements for the output format.

If the stopping criteria are not satisfied, the method proceeds to block 708E, where process logic identifies one or more weaknesses of the schema and suggests one or more improvements to the schema to address the one or more weaknesses. The one or more improvements may comprise at least one of: a clarification of one or more edge cases; addition of an additional rule; or addition of an additional example. The review and refinement analysis may be repeated in an iterative manner until the schema satisfies one or more criteria, wherein the schema is refined with each iteration of the review and refinement analysis. If the stopping criteria are satisfied, the method proceeds to block 710.

In some embodiments, process logic validates the model-generated prompt by applying the model-generated prompt to at least some of the one or more examples and confirming that an output has a structure that conforms to a schema defined in the base prompt. For dental treatment applications, validating the model-generated prompt may comprise applying the model-generated prompt to test dental treatment instructions and confirming that output conforms to the refined schema. Providing the model-generated prompt to the second LLM may be performed based on the validation.

In some embodiments, the model-generated prompt is stored in a prompt registry. The model-generated prompt may be used in one or more downstream pipelines for automated text understanding. For dental treatment applications, the validated model-generated prompt may be stored in a prompt registry and provided for use in one or more dental treatment planning pipelines to convert treatment provider instructions into machine-readable treatment planning instructions.

At block 710, process logic provides the model-generated prompt and input associated with a specific instance of the second target task to a second LLM. The second LLM may be configured to perform the second target task. The second LLM may be the same or different than the first LLM. The specific instance may be for treatment of a particular patient.

At block 712, process logic obtains second output from the second LLM based on the model-generated prompt and the second target task. The output may include a set of categories that input may be categorized into. The output may include categorizations of the input, such as assignment of portions of the first input to the second set of categories. The output may include machine-readable instructions. The output may include an analysis of categorizations of input, e.g., an analysis of frequency of occurrence of target types of complaints, changes, updates, or the like. In some embodiments, the second target task comprises generating machine-readable instructions, and the method further comprises executing the output from the second LLM comprising the machine-readable instructions.

At block 714, process logic optionally generates manufacturing data for one or more dental treatment appliances based on the output from the second LLM. In some embodiments, categorized instructions may be provided to further systems to generate actionable recommendations, such as updates to treatment protocols or treatment plans. In some embodiments, machine-readable instructions may be utilized in generating treatment plans. Where the second target task comprises generating machine-readable treatment planning instructions for a dental treatment, executing the output from the second LLM may comprise generating a treatment plan for a dental patient. Process logic may further cause the dental treatment appliances to be manufactured, e.g., by providing the manufacturing data to manufacturing equipment. The method may further comprise generating manufacturing data for one or more dental treatment appliances associated with the treatment plan and manufacturing the one or more dental treatment appliances in accordance with the manufacturing data.

FIG. 7B is a flow diagram of a method 700B for producing a model-generated prompt, according to some embodiments. At block 716, process logic obtains a base prompt configured to cause a first LLM to assign portions of natural language input to a first set of categories. The categories may be elucidated within the base prompt. The natural language input may be or include instructions. The natural language input may be instructions to update one or more stages of dental or orthodontic treatment planning, such as updating a treatment plan or a treatment protocol.

At block 718, process logic provides a first prompt generation request as input to a second LLM. The first prompt generation request may include the base prompt. The first prompt generation request may include a description of a target task. The first prompt generation request may include a description of target properties of the model-generated prompt. The first prompt generation request may include one or more examples of input that is to be categorized. In some embodiments, the target task may include generation of additional categories to augment the first set of categories.

At block 720, process logic obtains a model-generated prompt from the second LLM. Further review, refinement, validation, etc., of the model-generated prompt may also be performed. Operations of block 720 may share one or more features with operations of block 708 of FIG. 7A.

At block 722, process logic provides the model-generated prompt and first input including natural language input associated with the target task to a third LLM. The natural language input optionally includes a set of instructions related to dental or orthodontic treatment of a patient.

At block 724, process logic obtains output from the third LLM based on the model-generated prompt including assignment of portions of the first input to the second set of categories. The second set of categories may be an extension of the categories included in the base prompt.

At block 728, process logic optionally provides instructions identified as belonging to a target category by the third LLM to a fourth LLM. The fourth LLM may be configured to generate machine-readable instructions from natural language instructions. The fourth LLM may be configured to generate machine-readable instructions from natural language instructions of the target category. In some cases, several LLMs may be used for converting natural language instructions of different categories to machine-readable instructions. In some embodiments, process logic further executes the machine readable instructions, which may include updating a treatment protocol or plan, generating a treatment protocol or plan, or the like.

At block 730, process logic optionally generates manufacturing data for one or more dental treatment appliances. Process logic may further cause the one or more dental treatment appliances to be manufactured. Operations of block 730 may share one or more features with operations of block 714 of FIG. 7A.

FIG. 7C is a flow diagram of a method 700C for generating machine-readable instructions from a target category of natural language instructions, according to some embodiments. At block 734, process logic may obtain, by a processing device, a set of treatment provider instructions for a dental treatment of a patient. The treatment provider instructions may be first treatment instructions for a target dental treatment of a patient. In some embodiments, the treatment provider instructions may include instructions that are not based on natural language entry, such as selections made in a treatment planning GUI. In some embodiments, the treatment provider instructions may include natural language, e.g., entered in a comment section of a treatment planning application or program. In some embodiments, the treatment provider instructions may comprise treatment modification comments from a treatment provider associated with a dental treatment, the treatment modification comments expressed in natural language.

At block 735, process logic determines that the treatment instructions comprise one or more manual comments in a natural language format. The manual comments may be associated with the dental treatment. In some embodiments, a comment analyzer (e.g., which may be an LLM in some embodiments) may determine that the treatment provider instructions include natural language instructions associated with a target treatment. The determination that the treatment instructions include manual comments in natural language format may trigger subsequent classification and processing operations.

At block 736, process logic processes the treatment instructions using a classification model. This may include transmitting the instructions to a remote computing device comprising a classification model for processing. The classification model categorizes the treatment instructions into one or more dental treatment categories. The classification model may be an AI model. The classification model may be an LLM configured (e.g., via prompting strategies) to classify instructions into various categories of interest in some embodiments. In some embodiments, the classification model may be a first large language model (LLM) configured to categorize natural language instructions. In some embodiments, the classification model is a neural network, such as a convolutional neural network.

The classification model may be configured to categorize natural language instructions into a plurality of treatment categories. The dental treatment categories may include categories such as dental features, attachments, passive aligners, overcorrection, treatment length, interproximal reduction, precision cuts, bite ramps, and other treatment-related categories. The treatment categories may include one or more treatment plan parameters for the target dental treatment. The treatment plan parameters may be organized hierarchically, with a first category comprising dental appliance features for one or more dental appliances to be used for the target dental treatment. The dental appliance features may include attachments, bite ramps, or modeled appliance features. The dental appliance features may further include mandibular advancement features selected from buccal blocks or occlusal blocks. A second category of treatment plan parameters may comprise one or more planning targets. The planning targets may include intended final positions for one or more teeth, tooth velocities for one or more teeth, target treatment outcome, number of treatment stages, amount of overcorrection, and/or whether to apply passive aligners. In some embodiments, the classification model may determine that the treatment modification comments include instructions belonging to a dental features category and do not include instructions belonging to categories that require manual processing.

At block 740, process logic determines whether each of the one or more dental treatment categories belongs to a specified subset of dental treatment categories. The determination may be made based on output of the classification model. If each of the one or more dental treatment categories belongs to the specified subset (YES branch), process logic proceeds to provide the treatment instructions to an LLM for generating machine-readable instructions. If one or more of the dental treatment categories does not belong to the specified subset (NO branch), process logic may provide the treatment instructions for manual processing. Process logic may determine, using the classification model, that a first category of instructions is not present in the first natural language instructions associated with dental treatment. Process logic may optionally determine that the first natural language instructions include instructions belonging to a second category of instructions. Instructions of the second category may be instructions that are to be processed by an LLM. The second category of instructions may comprise instructions associated with dental features in one embodiment. Process logic may optionally determine that the first natural language instructions include instructions of a third category. The third category may be or include instructions which are not to be included in the target treatment, such as instructions that are not of clinical significance, instructions that are merely polite expressions, or the like. The third category of instructions may comprise instructions that are not applicable to the treatment plan. In some embodiments, process logic may determine, using the classification model, that a third category of instructions is present in the first natural language instructions, and may exclude instructions belonging to the third category of instructions from the portion of the first natural language instructions provided to the LLM. In some embodiments, process logic may determine, using the classification model, that the first treatment instructions comprise one or more instructions belonging to a treatment category not included in the specified subset of dental treatment categories, wherein the one or more instructions are not used in generating the machine-readable instructions.

At block 742, responsive to determining that at least a portion (e.g., each) of the one or more dental treatment categories belongs to the specified subset of dental treatment categories, process logic provides at least the portion of the first treatment instructions and a prompt to a large language model (LLM) to cause the LLM to generate machine-readable instructions corresponding to the specified subset of dental treatment categories. The prompt is configured to cause the LLM to generate machine-readable instructions corresponding to natural language instructions. The prompt may be configured to cause the LLM to generate machine-readable instructions corresponding to natural language instructions of a particular category, e.g., the second category of instructions. The LLM may be configured to accept instructions belonging to the specified subset of dental treatment categories as input. In some embodiments, the at least a portion of the first natural language instructions comprise instructions belonging to the second category of instructions. In some embodiments, providing the at least the portion of the first treatment instructions and the prompt to the LLM comprises transmitting at least one of the portion of the first treatment instructions or the prompt to a remote computing device that executes the LLM. In some embodiments, the treatment modification comments may be provided to a specialized interpretation model configured to extract attachment-related parameters from natural language instructions. The specialized interpretation model may be the LLM or a separate model configured to process instructions belonging to the specified subset of dental treatment categories.

At block 746, process logic obtains machine-readable instructions corresponding to the treatment instructions from the LLM. In some embodiments, obtaining the machine-readable instructions comprises receiving the machine-readable instructions from a remote computing device. The machine-readable instructions may be related to updates to dental features of a dental treatment (e.g., attachments for dental treatment appliances). The machine-readable instructions may comprise instructions for placement of attachments to one or more teeth of a patient for the target dental treatment of the patient. The LLM may extract teeth identifiers of one or more teeth to receive attachments, one or more treatment stages at which to place the attachments, one or more treatment actions to be performed, and attachment types for the attachments. The machine-readable instructions may comprise a text-based schema, such as a JSON format. In some embodiments, the machine-readable instructions may comprise one or more of teeth identifiers, treatment stages, attachment actions, and/or attachment types. The attachment actions may comprise one or more of add, remove, keep, replace, or modify. The machine-readable instructions may comprise updates to one or more treatment plan parameters for the target dental treatment. The one or more treatment plan parameters may comprise dental appliance features for one or more dental appliances to be used for the target dental treatment. The dental appliance features may comprise at least one of attachments, bite ramps, and/or modeled appliance features. The dental appliance features may comprise mandibular advancement features selected from at least one of buccal blocks or occlusal blocks. The one or more treatment plan parameters may comprise one or more planning targets. The one or more planning targets may comprise at least one of intended final positions for one or more teeth, tooth velocities for one or more teeth, target treatment outcome, number of treatment stages, amount of overcorrection, and/or whether to apply passive aligners. In some embodiments, the machine-readable instructions include stage information represented in one or more formats comprising: a first stage indicator representing a beginning of treatment, a last stage indicator representing an end of treatment, a literal stage number, an offset value representing a number of stages relative to a reference stage, or a time indicator specifying whether an action is to be applied before or after a specified stage.

In some embodiments, process logic may determine that the first treatment instructions include terminology variations or misspellings of attachment-related terms. Process logic may normalize the terminology variations or misspellings to standard attachment terminology prior to generating the machine-readable instructions. For example, doctors may write “attachments” in different ways such as “att”, “attach”, “attaches”, or make mistakes when writing such as “atachemens”, and the system may normalize these variations to standard terminology. In some embodiments, process logic may determine that the treatment modification comments are expressed in a non-English language and may translate the treatment modification comments to English in the machine-readable instructions.

In some embodiments, process logic may perform post-processing validation on the machine-readable instructions to confirm compatibility with a treatment planning system. The post-processing validation may include format checking, schema validation, and verification that the machine-readable instructions conform to required structures and constraints. The post-processing validation may confirm that the machine-readable instructions are ready to be applied to update the dental treatment.

At block 748, process logic optionally performs treatment planning operations for the target treatment based on the machine-readable instructions. Process logic may integrate the machine-readable instructions into treatment planning operations. Process logic may apply the machine-readable instructions to update the dental treatment. Process logic may update a treatment plan to generate an updated treatment plan associated with the target dental treatment based on the machine-readable instructions. Process logic may provide a representation of the updated treatment plan for treatment provider review. The representation of the updated treatment plan may comprise one or more of: a natural language description of the updated treatment plan; a medical algorithm depicting the updated treatment plan; or a visualization of a three-dimensional model depicting predicted properties of dentition associated with the updated treatment plan. Process logic may further generate manufacturing data for one or more treatment appliances based on the treatment planning operations. Process logic may further cause the one or more treatment appliances to be manufactured based on the manufacturing data. In some embodiments, process logic may obtain an indication that the updated treatment plan has been accepted, generate manufacturing data for one or more treatment appliances in view of the updated treatment plan being accepted, and cause the one or more treatment appliances to be manufactured.

At block 750, process logic optionally obtains second treatment instructions. The second treatment instructions may include different instructions than the first treatment instructions, and may be identified by the comment analyzer. Process logic may further determine, using the classification model, that the second treatment instructions comprise instructions belonging to a dental treatment category other than the specified subset of dental treatment categories. Process logic may determine, using the classification model, that the second natural language instructions comprise instructions belonging to the first category of instructions. Process logic may further provide the second treatment instructions for manual processing. In some embodiments, a first portion of a set of instructions provided may be processed in a manual processing pipeline (e.g., instructions belonging to the first category or categories outside the specified subset), and a second portion of a set of instructions provided may be processed in an LLM-based pipeline (e.g., a portion of the input instructions which belong to the second category or the specified subset of dental treatment categories).

FIG. 7D is a flow diagram of a method 700D for obtaining machine-readable instructions corresponding to a target category of natural language instructions in relation to a dental treatment, according to some embodiments. At block 752, process logic obtains first natural language instructions from a treatment provider. The first natural language instructions may include target updates to a target dental treatment.

At block 754, process logic provides the first natural language instructions to a first LLM configured to categorize the natural language instructions. Categories into which the instructions are to be categorized may be included in a prompt also provided to the first LLM. In some embodiments, instructions may be categorized into only categories of relevance to a particular system, e.g., non-clinical instructions, instructions belonging to a category for which LLM processing has been developed and is reliable, and other instructions. Further actions may be taken based on which categories the natural language instructions are assigned to.

At block 756, process logic determines that a first category of instructions is not present in the first natural language instructions. Process logic may optionally determine that a second category and/or further categories of instructions are or are not present in the natural language instructions. Operations of block 756 may share one or more features with operations of block 740 of FIG. 7C.

At block 758, process logic provides at least a portion of the first natural language instructions to a second LLM. The portion may include instructions of a target category. The LLM may further be provided with a prompt configured to cause the second LLM to generate machine-readable instructions corresponding to the natural language instructions.

At block 760, process logic obtains machine-readable instructions corresponding to the first natural language instructions from the LLM. The machine-readable instructions may include updates to dental features of a dental treatment.

In some embodiments, further operations may be performed, such as executing treatment operations based on treatment planning, processing further (e.g., second) natural language instructions, etc. Processes similar to those described in connection with blocks 748 and 750 of FIG. 7C may be performed in connection with operations of method 700D.

FIG. 7E is a flow diagram of a method 700E for updating a treatment plan based on a target category of natural language instructions, according to some embodiments. At block 770, process logic obtains first natural language instructions from a treatment provider. The first instructions may be provided via a program, application, webpage, or the like associated with a target dental treatment.

At block 772, process logic provides the first natural language instructions to a classification model.

At block 774, process logic determines that a first category of instructions is not present in the first natural language instructions. In some embodiments, the first category of instructions may be a category of instructions that is to be processed manually. In some embodiments, the first category of instructions may include any instructions for which LLM processing is not to be performed. In some embodiments, the first category of instructions may be a specific category for which LLM processing is not to be performed. Process logic further determines that a second category of instructions is present in the natural language instruction. Process logic may further determine that a third category of instructions not associated with updating the target dental treatment (e.g., a polite expression) is included in the natural language instruction.

At block 776, process logic provides at least a portion of the first natural language instructions to a second LLM. The portion may include instructions of the second category. The second category may include instructions related to dental features, instructions related to attachments, etc. A prompt may also be provided to the second LLM, configured to enable the second LLM to generate machine-readable instructions. Natural language instructions belonging to the third category may be excluded from instructions provided to the second LLM.

At block 778, process logic obtains machine-readable instructions corresponding to the first natural language instructions from the second LLM. The machine-readable instructions may include instructions for updating dental features of a dental treatment.

At block 780, process logic updates a treatment plan to generate an updated treatment plan based on the machine-readable instructions.

At block 782, process logic provides a representation of the updated treatment plan for treatment provider review. The representation may be provided via a treatment planning application, a treatment planning GUI, a web browser displaying a treatment planning webpage, or the like. The representation may be or include a natural language description of the updated treatment plan (e.g., a description of the recommended update). The representation may include a medical algorithm depicting the updated treatment plan. The representation may include a visualization of a three-dimensional model depicting predicted properties of dentition treated in accordance with the updated treatment plan.

At block 784, process logic optionally obtains an indication that the updated treatment plan has been reviewed, accepted, etc. In some embodiments, further operations may be performed, such as executing treatment operations based on treatment planning, processing further (e.g., second) natural language instructions, etc. Processes similar to those described in connection with blocks 748 and 750 of FIG. 7C may be performed in connection with operations of method 700E.

FIG. 8A is a flow diagram of a method 800A for updating a treatment plan or protocol utilizing an AI assistant, according to some embodiments. At block 801, processing logic provides a chat interface associated with a treatment protocol or a treatment plan for a dental treatment. The chat interface may be provided via a GUI, such as a treatment planning GUI. The chat interface may comprise a conversational assistant interface that enables a treatment provider to interact with a dental treatment planning system using natural language. In some embodiments, the chat interface is configured to operate at a plurality of protocol levels. The protocol levels may comprise a prescription level for modifications specific to a single patient, a global clinical protocol level for modifications applicable to all patients of the treatment provider, and a personal protocol level for customized treatment preferences of the treatment provider in some embodiments. The chat interface may include a free text entry element for providing instructions associated with updates to a treatment plan.

At block 802, processing logic obtains, via the chat interface, first natural language comments associated with an update to one of the treatment protocol or the treatment plan. In some embodiments, the comments may be provided via a text entry field of a GUI, such as a treatment planning GUI. The first natural language comments may be associated with an update to a dental treatment protocol or a dental treatment plan. The first natural language comments may comprise a natural language request from the treatment provider associated with modifying one or more treatment parameters. In some embodiments, the first natural language comments may be associated with updating a target treatment plan via the text entry field.

At block 804, processing logic provides the first natural language comments to a model, which converts the first natural language comments into one or more proposed structured, valid protocol changes for the treatment protocol or the dental treatment. The converting may be performed by a large language model (LLM) in embodiments. In some embodiments, providing the first natural language instructions to the LLM comprises transmitting the first natural language instructions to a remote computing device that executes the LLM. Processing logic may provide additional contextual data or other additional information to the LLM. The additional information may include a library of machine-implementable instructions. Processing logic may provide a library of machine-implementable instructions to the LLM, wherein the LLM generates the machine-readable instructions by performing retrieval augmented generation in view of the library. The additional information may include a database of medical explanations. The additional information may include a history of treatment provider comments or updates. The additional information may be provided as one or more documents accessible to the LLM, e.g., in a RAG-based system. Processing logic may provide documentation comprising a library of machine-implementable instructions and associated explanations to the LLM. In some embodiments, the LLM may be configured to limit output or to limit some types of output based on the additional data. For example, the LLM may be configured to only provide machine-readable instructions composed of functions included in a library of instructions. The LLM may be configured to limit machine-implementable instructions output by the LLM to instructions in the documentation. The LLM may be configured to only answer medical questions with verified responses included in a database of medical explanations. In some embodiments, generating the one or more proposed modifications comprises constraining output of the LLM to only include modifications that are valid within a predefined set of protocol commands. In some embodiments, processing logic determines a protocol level at which the modification is to be applied, the protocol level comprising one of a patient-specific prescription level, a global clinical protocol level applicable to multiple patients, or a personal protocol level customized to the treatment provider. In some embodiments, generating the one or more proposed modifications comprises recognizing, by the LLM, a start of a command sequence in the natural language request, and triggering a predefined command sequence to complete the modification, wherein the predefined command sequence comprises a series of GUI manipulations associated with the recognized command. Processing logic may convert the first natural language instructions into structured, valid protocol comments for a dental treatment protocol and process the first natural language instructions to generate machine-readable instructions comprising updates to the target treatment plan corresponding to the first natural language instructions.

At block 805, processing logic may generate a preview and human-readable explanations of the one or more proposed structured, valid protocol changes. The generating may be performed by a large language model (LLM). In some embodiments, the one or more proposed structured, valid protocol changes are maintained in a draft state prior to receiving confirmation.

At block 806, processing logic may provide, via a graphical user interface (GUI), the preview of the one or more proposed modifications in a draft state. Processing logic may output the preview and the human-readable explanations of the one or more proposed structured, valid protocol changes. In some embodiments, processing logic provides an indication of the machine-readable instructions for review via the GUI. The one or more proposed structured, valid protocol changes may comprise adjustments to one or more fields that correspond deterministically to machine-readable code. The one or more proposed structured, valid protocol changes may comprise adjustments to machine-readable code to be applied to the treatment protocol or treatment plan.

At block 807, processing logic optionally obtains additional clarification or information associated with the first natural language comments. In some embodiments, the first natural language comments may be provided via an AI chat function of the GUI. Responsive to natural language comments exhibiting one or more target characteristics (e.g., confidence in recommending updates to a treatment plan by the LLM being below a target threshold), the AI chat function may provide a prompt to a user to provide additional clarifying input, additional information, or the like. In some embodiments, processing logic obtains third natural language comments prior to receiving the first natural language instructions, provides, via the graphical user interface, a prompt to provide additional information, and obtains the first natural language instructions in view of the prompt. In some embodiments, processing logic obtains second natural language instructions via the free text entry element, provides the second natural language instructions to the LLM, obtains, from the LLM, a clarifying inquiry associated with the second natural language instructions, provides the clarifying inquiry, and responsive to providing the clarifying inquiry, obtains via the free text entry element the first natural language instructions. In some embodiments, if no actionable instructions are detected in the first natural language comments, the AI chat function may be configured to provide a prompt to input instructions to update the treatment protocol or treatment plan. In some embodiments, processing logic obtains a natural language inquiry associated with the dental treatment protocol or dental treatment plan, provides the natural language inquiry to the LLM, obtains, from the LLM, an explanation based on the natural language inquiry, and provides the explanation for review, wherein the first natural language comments are provided in view of the explanation. The explanation based on the natural language inquiry may be generated based at least in part on accessed documentation in some embodiments. In some embodiments, processing logic obtains a query from the treatment provider regarding a treatment option or clinical terminology, accesses, by the LLM, a library of clinical explanations including tool tips from the treatment planning system, and provides, via the conversational assistant interface, a natural language explanation responsive to the query using terminology specific to the treatment planning system.

Processing logic may obtain, via the GUI, confirmation or rejection of each of the one or more proposed modifications. In some embodiments, implementing the machine-readable instructions is performed based on obtaining confirmation responsive to providing the indication of the machine-readable instructions for review. In some embodiments, processing logic determines that a treatment protocol may be updated based on the first natural language instructions or the machine-readable instructions, provides a prompt comprising a proposed update of the treatment protocol, obtains a response to the prompt via the free text entry element, determines, using the LLM, that the response to the prompt comprises confirmation of the proposed update, and performs the proposed update responsive to obtaining the response.

At block 812, responsive to receiving confirmation to implement the one or more proposed structured, valid protocol changes, processing logic updates the treatment protocol or the dental treatment. Processing logic may publish the one or more proposed structured, valid protocol changes to an active protocol state responsive to receiving confirmation. Responsive to obtaining confirmation, processing logic publishes the confirmed modifications to an active protocol state at the determined protocol level in embodiments. The machine-implementable instructions may include automating selection of one or more treatment options. The machine-implementable instructions may include machine-readable code, e.g., for updating a treatment protocol or treatment plan. In some embodiments, machine-implementable instructions may not be generated, e.g., an alert may be provided to a user indicating recommended actions that the user could perform, such as updating one or more options or selections in treatment planning software.

Processing logic may cause dental treatment planning operations to be performed based on the machine-implementable instructions. Processing logic may implement the machine-readable instructions by performing treatment planning operations. In some embodiments, processing logic causes the conversational assistant to automatically manipulate one or more graphical user interface elements to effect the one or more proposed structured, valid protocol changes, wherein the manipulation is visible to the treatment provider. The manipulation may comprise opening dropdown menus, selecting options, and/or clicking on interface elements visible to the treatment provider. In some embodiments, processing logic performs verification operations on the machine-implementable instructions, comprising determining that the machine-implementable instructions adhere to existing safety heuristics, wherein performing the dental treatment planning operations is further based on the verification operations.

In some embodiments, the first natural language comments are associated with an update to the treatment protocol, and processing logic obtains second natural language comments associated with an update to the treatment plan that is a specific application of the treatment protocol to a patient. Processing logic may convert the second natural language comments into one or more additional structured, valid protocol changes for the dental treatment. Processing logic may generate a second preview of the one or more additional proposed structured, valid protocol changes. Processing logic may output the second preview and human-readable explanations of the one or more additional proposed structured, valid protocol changes. Responsive to receiving confirmation to implement the one or more additional proposed structured, valid protocol changes, processing logic may update the dental treatment plan. In some embodiments, obtaining the first natural language comments and obtaining the second natural language comments is performed via a text entry field of a graphical user interface. In some embodiments, at least a portion of the first natural language comments are provided to a large language model (LLM) as additional context with the second natural language comments.

In some embodiments, processing logic maintains session context data associated with the treatment provider. The session context data may include a history of prior natural language requests and corresponding modifications. In some embodiments, processing logic detects that the treatment provider has requested a same or similar change across a plurality of patients. Processing logic may generate a recommendation to apply the change to a global treatment protocol applicable to future patients. Responsive to obtaining confirmation from the treatment provider, processing logic may update the global treatment protocol.

In some embodiments, processing logic detects that the natural language request corresponds to a modification at the patient-specific prescription level, determines that the modification is applicable to multiple patients, generates a recommendation to promote the modification to the global clinical protocol level, and responsive to obtaining confirmation of the recommendation, updates the global clinical protocol to include the modification.

At block 814, processing logic optionally generates manufacturing data of one or more treatment appliances based on the machine-implementable instructions. Processing logic further optionally causes the one or more treatment appliances to be manufactured. Processing logic may manufacture one or more dental treatment appliances based on the dental treatment planning operations.

FIG. 8B is a flow diagram of a method 800B for performing dental treatment planning utilizing an LLM, according to some embodiments. At block 820, process logic optionally obtains a natural language inquiry associated with a dental treatment protocol or dental treatment plan. Process logic may provide the natural language inquiry to an LLM. Process logic may obtain form the LLM an explanation based on the natural language inquiry. For example, an inquiry may be provided regarding an aspect of treatment or a particular disorder. The LLM may provide an explanation responsive to receiving the inquiry. The explanation may then be provided (e.g., via an AI chat element of a treatment planning GUI) for review by a user (e.g., treatment provider).

At block 822, process logic obtains first natural language instructions associated with an update to one of a dental treatment protocol or a dental treatment plan. The natural language instructions may be obtained via a text entry GUI element associated with an AI chat function, AI assistant function, or the like.

At block 824, process logic provides the first natural language instructions to an LLM. Optionally, process logic may provide additional context to the LLM, such as discussed in connection with block 804 of FIG. 8A.

At block 826, process logic obtains, from the LLM, output including machine-implementable instructions corresponding to the first natural language instructions. Operations of block 826 may share one or more features with operations of block 808 of FIG. 8A.

At block 828, process logic optionally performs verification operations on the machine-implementable instructions. Verification operations may include verifying that machine-implementable instructions are executable, e.g., verifying syntactic accuracy, verifying correct usage of one or more functions, etc. Verification operations may include determining that the machine-implementable instructions adhere to one or more rules, such as safety rules for dental treatment (e.g., maximum tooth repositioning speed, minimum tooth spacing, etc.). Verification may include checking generated instructions to ensure they adhere to and/or comply with existing heuristics, such as safety heuristics. Dental treatment planning operations may be performed responsive to treatment verification.

At block 830, process logic performs dental treatment planning operations based on the machine-implementable instructions.

At block 832, process logic optionally causes one or more dental treatment appliances to be manufactured based on the dental treatment planning operations.

FIG. 8C is a flow diagram of a method 800C for using an LLM via a GUI element to perform treatment planning, according to some embodiments. At block 840, process logic optionally obtains a preliminary natural language instruction. The preliminary natural language instruction is obtained via a GUI free text entry element, e.g., an AI chat function. Process logic further provides the preliminary instruction to an LLM. Process logic further obtains an associated clarifying inquiry. Responsive to providing the clarifying inquiry, process logic obtains first natural language instructions via the free text entry element. In some embodiments, both the initial input and the clarifying input may be utilized by the LLM in generating output.

At block 842, process logic provides, via a GUI, a free text entry element for providing instructions associated with updates to a treatment plan. In some embodiments, this may be the same free text entry element from block 840.

At block 844, process logic obtains first natural language instructions associated with updating a target treatment plan. Operations of block 844 may share one or more features with operations of block 802 of FIG. 8A.

At block 846, process logic provides the natural language instructions to an LLM. Operations of block 846 may share one or more features with operations of block 804 of FIG. 8A.

At block 848, process logic obtains machine-readable instructions including updates to the target treatment plan corresponding to the first natural language instructions. The machine readable instructions may be obtained as output from the LLM, responsive to providing the natural language instructions as input to the LLM.

At block 850, process logic optionally determines that a treatment protocol may be updated based on the first natural language instructions or the machine-readable instructions. For example, if a practitioner has requested a particular adjustment or change multiple times (e.g., satisfying a threshold condition), in a particular portion of cases, in a particular portion of cases exhibiting some target disorder, behavior, or other similarity, or the like, process logic may determine that the requested adjustment to the treatment plan may be applicable to many treatment plans, and may be integrated into a treatment protocol. Process logic may further provide a prompt (e.g., via the AI chat function of the GUI) indicating the proposed treatment protocol update. Process logic may obtain a response to the prompt via the free text entry element, e.g., a doctor acceptance of the treatment protocol update. In some embodiments, a different free text entry element, e.g., associated with protocol generation may be utilized to provide this prompt. Process logic may, based on the response, determine that the proposed update is to be enacted. Process logic may obtain confirmation of the proposed update from a user, practitioner, etc. Process logic may further perform the proposed update responsive to obtaining the response.

At block 852, process logic optionally provides an indication of the machine-readable instructions for review via the GUI. Operations of block 852 may share one or more features with operations of block 810 of FIG. 8A.

At block 854, process logic implements the machine-readable instructions by performing treatment planning operations. Further operations may include generating manufacturing data for treatment appliances, storing the treatment planning data, causing the treatment appliances to be manufactured, etc. operations of block 854 may share one or more features with operations of block 812 of FIG. 8A.

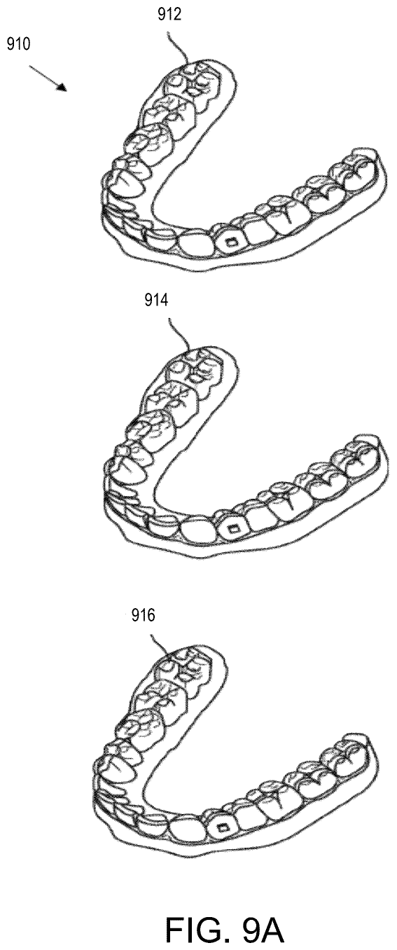

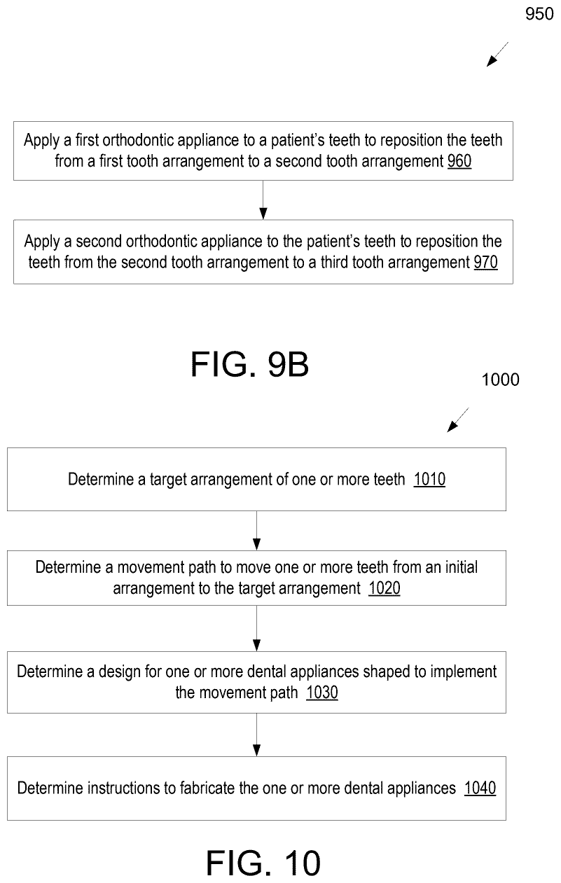

FIG. 9A illustrates a tooth repositioning system 910 including a plurality of appliances 912, 914, 916. The appliances 912, 914, 916 can be designed based on generation of a sequence of 3D models of dental arches, which may be generated according to the techniques discussed herein above. For example, treatment plans may be generated that include a sequence of 3D models. The treatment plans may be generated based on practitioner preferences, as codified in a set of treatment protocols generated based on natural language inputs and/or selection of preferences from a set of treatment options in accordance with aspects of the present disclosure.

Any of the appliances described herein can be designed and/or provided as part of a set of a plurality of appliances used in a tooth repositioning system, and may be designed in accordance with an orthodontic treatment plan generated in accordance with embodiments of the present disclosure. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 910 can include a first appliance 912 corresponding to an initial tooth arrangement, one or more intermediate appliances 914 corresponding to one or more intermediate arrangements, and a final appliance 916 corresponding to a target arrangement. A target tooth arrangement can be a planned final tooth arrangement selected for the patient's teeth at the end of all planned orthodontic treatment, as optionally output using a trained machine learning model. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

In some embodiments, the appliances 912, 914, 916 (or portions thereof) can be produced using indirect fabrication techniques, such as by thermoforming over a positive or negative mold. Indirect fabrication of an orthodontic appliance can involve producing a positive or negative mold of the patient's dentition in a target arrangement (e.g., by rapid prototyping, milling, etc.) and thermoforming one or more sheets of material over the mold in order to generate an appliance shell.

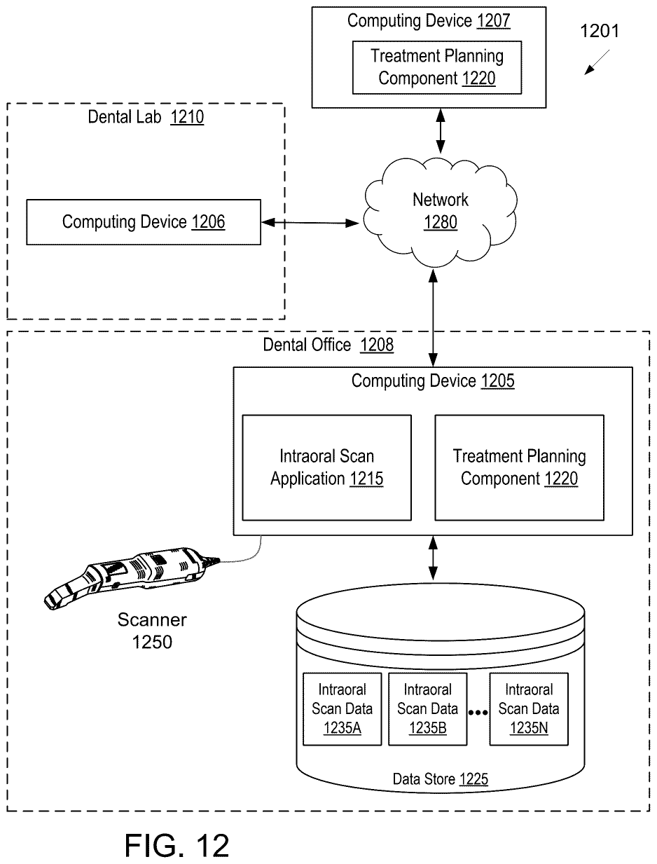

In an example of indirect fabrication, a mold of a patient's dental arch may be fabricated from a digital model of the dental arch generated by a trained machine learning model as described above, and a shell may be formed over the mold (e.g., by thermoforming a polymeric sheet over the mold of the dental arch and then trimming the thermoformed polymeric sheet). The fabrication of the mold may be performed by a rapid prototyping machine (e.g., a stereolithography (SLA) 3D printer). The rapid prototyping machine may receive digital models of molds of dental arches and/or digital models of the appliances 912, 914, 916 after the digital models of the appliances 912, 914, 916 have been processed by processing logic of a computing device, such as the computing device in FIG. 12. The processing logic may include hardware (e.g., circuitry, dedicated logic, programmable logic, microcode, etc.), software (e.g., instructions executed by a processing device), firmware, or a combination thereof. For example, one or more operations may be performed by a processing device executing treatment plan generator 268 of FIG. 2.

To manufacture the molds, a shape of a dental arch for a patient at a treatment stage is determined based on a treatment plan. In the example of orthodontics, the treatment plan may be generated based on an intraoral scan of a dental arch to be modeled. The intraoral scan of the patient's dental arch may be performed to generate a three dimensional (3D) virtual model of the patient's dental arch (mold). For example, a full scan of the mandibular and/or maxillary arches of a patient may be performed to generate 3D virtual models thereof. The intraoral scan may be performed by creating multiple overlapping intraoral images from different scanning stations and then stitching together the intraoral images or scans to provide a composite 3D virtual model. In other applications, virtual 3D models may also be generated based on scans of an object to be modeled or based on use of computer aided drafting techniques (e.g., to design the virtual 3D mold). Alternatively, an initial negative mold may be generated from an actual object to be modeled (e.g., a dental impression or the like). The negative mold may then be scanned to determine a shape of a positive mold that will be produced.

Once the virtual 3D model of the patient's dental arch is generated, a dental practitioner may determine a desired treatment outcome, which includes final positions and orientations for the patient's teeth. In one embodiment, treatment plan generator 268 outputs a desired treatment outcome based on processing the virtual 3D model of the patient's dental arch (or other dental arch data associated with the virtual 3D model). Processing logic may then determine a number of treatment stages to cause the teeth to progress from starting positions and orientations to the target final positions and orientations. The shape of the final virtual 3D model and each intermediate virtual 3D model may be determined by computing the progression of tooth movement throughout orthodontic treatment from initial tooth placement and orientation to final corrected tooth placement and orientation. For each treatment stage, a separate virtual 3D model of the patient's dental arch at that treatment stage may be generated. In one embodiment, for each treatment stage treatment plan generator 268 outputs a different 3D model of the dental arch. The shape of each virtual 3D model will be different. The original virtual 3D model, the final virtual 3D model and each intermediate virtual 3D model is unique and customized to the patient.

Accordingly, multiple different virtual 3D models (digital designs) of a dental arch may be generated for a single patient. A first virtual 3D model may be a unique model of a patient's dental arch and/or teeth as they presently exist, and a final virtual 3D model may be a model of the patient's dental arch and/or teeth after correction of one or more teeth and/or a jaw. Multiple intermediate virtual 3D models may be modeled, each of which may be incrementally different from previous virtual 3D models.

Each virtual 3D model of a patient's dental arch may be used to generate a unique customized physical mold of the dental arch at a particular stage of treatment. The shape of the mold may be at least in part based on the shape of the virtual 3D model for that treatment stage. The virtual 3D model may be represented in a file such as a computer aided drafting (CAD) file or a 3D printable file such as a stereolithography (STL) file. The virtual 3D model for the mold may be sent to a third party (e.g., clinician office, laboratory, manufacturing facility or other entity). The virtual 3D model may include instructions that will control a fabrication system or device in order to produce the mold with specified geometries.

A clinician office, laboratory, manufacturing facility or other entity may receive the virtual 3D model of the mold, the digital model having been created as set forth above. The entity may input the digital model into a 3D printer. 3D printing includes any layer-based additive manufacturing processes. 3D printing may be achieved using an additive process, where successive layers of material are formed in proscribed shapes. 3D printing may be performed using extrusion deposition, granular materials binding, lamination, photopolymerization, continuous liquid interface production (CLIP), or other techniques. 3D printing may also be achieved using a subtractive process, such as milling.

In some instances, stereolithography (SLA), also known as optical fabrication solid imaging, is used to fabricate an SLA mold. In SLA, the mold is fabricated by successively printing thin layers of a photo-curable material (e.g., a polymeric resin) on top of one another. A platform rests in a bath of a liquid photopolymer or resin just below a surface of the bath. A light source (e.g., an ultraviolet laser) traces a pattern over the platform, curing the photopolymer where the light source is directed, to form a first layer of the mold. The platform is lowered incrementally, and the light source traces a new pattern over the platform to form another layer of the mold at each increment. This process repeats until the mold is completely fabricated. Once all of the layers of the mold are formed, the mold may be cleaned and cured.

Materials such as a polyester, a co-polyester, a polycarbonate, a polycarbonate, a thermopolymeric polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, a polytrimethylene terephthalate, a styrenic block copolymer (SBC), a silicone rubber, an elastomeric alloy, a thermopolymeric elastomer (TPE), a thermopolymeric vulcanizate (TPV) elastomer, a polyurethane elastomer, a block copolymer elastomer, a polyolefin blend elastomer, a thermopolymeric co-polyester elastomer, a thermopolymeric polyamide elastomer, or combinations thereof, may be used to directly form the mold. The materials used for fabrication of the mold can be provided in an uncured form (e.g., as a liquid, resin, powder, etc.) and can be cured (e.g., by photopolymerization, light curing, gas curing, laser curing, crosslinking, etc.). The properties of the material before curing may differ from the properties of the material after curing.

Appliances may be formed from each mold and when applied to the teeth of the patient, may provide forces to move the patient's teeth as dictated by the treatment plan. The shape of each appliance is unique and customized for a particular patient and a particular treatment stage. In an example, the appliances 912, 914, 916 can be pressure formed or thermoformed over the molds. Each mold may be used to fabricate an appliance that will apply forces to the patient's teeth at a particular stage of the orthodontic treatment. The appliances 912, 914, 916 each have teeth-receiving cavities that receive and resiliently reposition the teeth in accordance with a particular treatment stage.

In one embodiment, a sheet of material is pressure formed or thermoformed over the mold. The sheet may be, for example, a sheet of polymeric (e.g., an elastic thermopolymeric, a sheet of polymeric material, etc.). To thermoform the shell over the mold, the sheet of material may be heated to a temperature at which the sheet becomes pliable. Pressure may concurrently be applied to the sheet to form the now pliable sheet around the mold. Once the sheet cools, it will have a shape that conforms to the mold. In one embodiment, a release agent (e.g., a non-stick material) is applied to the mold before forming the shell. This may facilitate later removal of the mold from the shell. Forces may be applied to lift the appliance from the mold. In some instances, a breakage, warpage, or deformation may result from the removal forces. Accordingly, embodiments disclosed herein may determine where the probable point or points of damage may occur in a digital design of the appliance prior to manufacturing and may perform a corrective action.

Additional information may be added to the appliance. The additional information may be any information that pertains to the appliance. Examples of such additional information includes a part number identifier, patient name, a patient identifier, a case number, a sequence identifier (e.g., indicating which appliance a particular liner is in a treatment sequence), a date of manufacture, a clinician name, a logo and so forth. For example, after determining there is a probable point of damage in a digital design of an appliance, an indicator may be inserted into the digital design of the appliance. The indicator may represent a recommended place to begin removing the polymeric appliance to prevent the point of damage from manifesting during removal in some embodiments. In embodiments, the additional information may be automatically added to a generated 3D model by treatment plan generator 268 in generation of the 3D model.

After an appliance is formed over a mold for a treatment stage, the appliance is removed from the mold (e.g., automated removal of the appliance from the mold), and the appliance is subsequently trimmed along a cutline (also referred to as a trim line). The processing logic may determine a cutline for the appliance. In one embodiment, treatment plan generator 268 outputs a cutline for an appliance associated with a 3D model output by the dental arch generator 268. The determination of the cutline(s) may be made based on the virtual 3D model of the dental arch at a particular treatment stage, based on a virtual 3D model of the appliance to be formed over the dental arch, or a combination of a virtual 3D model of the dental arch and a virtual 3D model of the appliance. The location and shape of the cutline can be important to the functionality of the appliance (e.g., an ability of the appliance to apply desired forces to a patient's teeth) as well as the fit and comfort of the appliance. For shells such as orthodontic appliances, orthodontic retainers and orthodontic splints, the trimming of the shell may play a role in the efficacy of the shell for its intended purpose (e.g., aligning, retaining or positioning one or more teeth of a patient) as well as the fit of the shell on a patient's dental arch. For example, if too much of the shell is trimmed, then the shell may lose rigidity and an ability of the shell to exert force on a patient's teeth may be compromised. When too much of the shell is trimmed, the shell may become weaker at that location and may be a point of damage when a patient removes the shell from their teeth or when the shell is removed from the mold. In some embodiments, the cut line may be modified in the digital design of the appliance as one of the corrective actions taken when a probable point of damage is determined to exist in the digital design of the appliance.

On the other hand, if too little of the shell is trimmed, then portions of the shell may impinge on a patient's gums and cause discomfort, swelling, and/or other dental issues. Additionally, if too little of the shell is trimmed at a location, then the shell may be too rigid at that location. In some embodiments, the cutline may be a straight line across the appliance at the gingival line, below the gingival line, or above the gingival line. In some embodiments, the cutline may be a gingival cutline that represents an interface between an appliance and a patient's gingiva. In such embodiments, the cutline controls a distance between an edge of the appliance and a gum line or gingival surface of a patient.

Each patient has a unique dental arch with unique gingiva. Accordingly, the shape and position of the cutline may be unique and customized for each patient and for each stage of treatment. For instance, the cutline is customized to follow along the gum line (also referred to as the gingival line). In some embodiments, the cutline may be away from the gum line in some regions and on the gum line in other regions. For example, it may be desirable in some instances for the cutline to be away from the gum line (e.g., not touching the gum) where the shell will touch a tooth and on the gum line (e.g., touching the gum) in the interproximal regions between teeth. Accordingly, it is important that the shell be trimmed along a predetermined cutline.

FIG. 9B illustrates a method 950 of orthodontic treatment using a plurality of appliances, in accordance with embodiments. The method 950 can be practiced using any of the appliances or appliance sets described herein. The method 950 can be applied based on treatment plans developed in accordance with treatment protocols based on selection from a set of treatment options and/or natural language input, in accordance with aspects of the present disclosure. The method 950 can be applied based on treatment plans that are updated with machine-readable instructions produced by an LLM or other AI model based on natural language treatment provider input, in accordance with aspects of the present disclosure.

In block 960, a first orthodontic appliance is applied to a patient's teeth in order to reposition the teeth from a first tooth arrangement to a second tooth arrangement. In block 970, a second orthodontic appliance is applied to the patient's teeth in order to reposition the teeth from the second tooth arrangement to a third tooth arrangement. The method 950 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

FIG. 10 illustrates a method 1000 for designing an orthodontic appliance to be produced by direct or indirect fabrication, in accordance with embodiments. The method 1000 can be applied to any embodiment of the orthodontic appliances described herein, and may be performed using one or more trained machine learning models in embodiments. Some or all of the blocks of the method 1000 can be performed by any suitable data processing system or device, e.g., one or more processors configured with suitable instructions. Method 1000 may be based on treatment protocols generated based on user selection of treatment options from a set, and/or natural language instructions, in accordance with aspects of the present disclosure.

At block 1010 a target arrangement of one or more teeth of a patient may be determined. The target arrangement of the teeth (e.g., a desired and intended end result of orthodontic treatment) can be received from a clinician in the form of a prescription, can be calculated from basic orthodontic principles, can be extrapolated computationally from a clinical prescription, and/or can be generated by a trained machine learning model such as treatment plan generator 268 of FIG. 2. With a specification of the desired final positions of the teeth and a digital representation of the teeth themselves, the final position and surface geometry of each tooth can be specified to form a complete model of the tooth arrangement at the desired end of treatment.

In block 1020, a movement path to move the one or more teeth from an initial arrangement to the target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. An initial arrangement may be estimated by projecting some measurement of the patient's teeth to a latent space, and obtaining from the latent space a representation of the initial arrangement. From the obtained data, a digital data set such as a 3D model of the patient's dental arch or arches can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, optionally including measured or extrapolated hidden surfaces and root structures, as well as surrounding bone and soft tissue.

Having both an initial position and a target position for each tooth, a movement path can be defined for the motion of each tooth. Determining the movement path for one or more teeth may include identifying a plurality of incremental arrangements of the one or more teeth to implement the movement path. In some embodiments, the movement path implements one or more force systems on the one or more teeth (e.g., as described below). In some embodiments, movement paths are determined by a trained machine learning model such as treatment plan generator 276. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In some embodiments, a force system to produce movement of the one or more teeth along the movement path is determined. In one embodiment, the force system is determined by a trained machine learning model. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully-formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be considered. In other embodiments, the treating professional can select an appropriate treatment based on physiological characteristics of the patient. For example, the properties of the palate may also be estimated based on factors such as the patient's age—for example, young juvenile patients will typically require lower forces to expand the suture than older patients, as the suture has not yet fully formed.

In block 1030, a design for one or more dental appliances shaped to implement the movement path is determined. In one embodiment, the one or more dental appliances are shaped to move the one or more teeth toward corresponding incremental arrangements. In some embodiments, results of one or more stages of treatment may be predicted by treatment plan generator 268. Determination of the one or more dental or orthodontic appliances, appliance geometry, material composition, and/or properties can be performed using a treatment or force application simulation environment. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA (Abaqus) software products from Dassault Systemes of Waltham, MA.

In block 1040, instructions for fabrication of the one or more dental appliances are determined or identified. In some embodiments, the instructions identify one or more geometries of the one or more dental appliances. In some embodiments, the instructions identify slices to make layers of the one or more dental appliances with a 3D printer. In some embodiments, the instructions identify one or more geometries of molds usable to indirectly fabricate the one or more dental appliances (e.g., by thermoforming plastic sheets over the 3D printed molds). The dental appliances may include one or more of aligners (e.g., orthodontic aligners), retainers, incremental palatal expanders, attachment templates, and so on.

In one embodiment, instructions for fabrication of the one or more dental appliances are generated by a trained model. In some embodiments, predictions of treatment progression and/or treatment appliances may be performed and/or aided by treatment plan generator 268. The instructions can be configured to control a fabrication system or device in order to produce the orthodontic appliance with the specified orthodontic appliance. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by 3D printing a mold and thermoforming a plastic sheet over the mold.

Method 1000 may comprise additional blocks: 1) The upper arch and palate of the patient is scanned intraorally to generate three dimensional data of the palate and upper arch; 2) The three dimensional shape profile of the appliance is determined to provide a gap and teeth engagement structures as described herein.

Although the above blocks show a method 1000 of designing an orthodontic appliance in accordance with some embodiments, a person of ordinary skill in the art will recognize some variations based on the teaching described herein. Some of the blocks may comprise sub-blocks. Some of the blocks may be repeated as often as desired. One or more blocks of the method 1000 may be performed with any suitable fabrication system or device, such as the embodiments described herein. Some of the blocks may be optional, and the order of the blocks can be varied as desired.

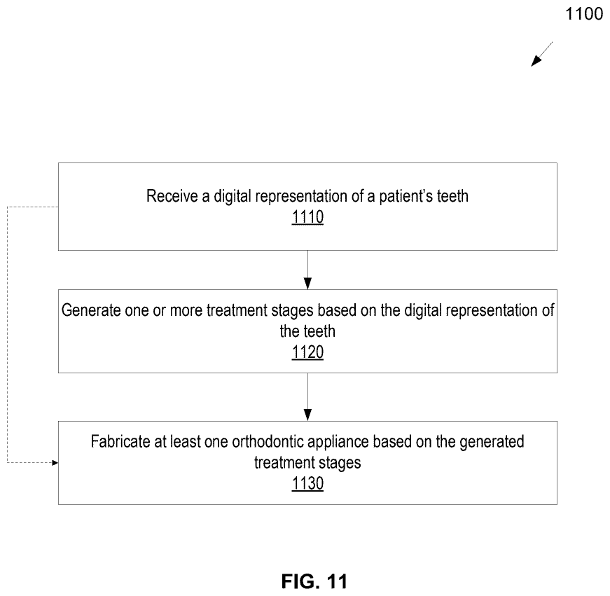

FIG. 11 illustrates a method 1100 for digitally planning an orthodontic treatment and/or design or fabrication of an appliance, in accordance with embodiments. The method 1100 can be applied to any of the treatment procedures described herein and can be performed by any suitable data processing system. The method 1100 may be based on treatment protocols generated in accordance with practitioner selection of treatment preferences from a set of provided options, and/or natural language practitioner input, as described herein.

In block 1110, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In block 1120, one or more treatment stages are generated based on the digital representation of the teeth. In some embodiments, the one or more treatment stages are generated based on processing of input dental arch data by a trained machine learning model such as treatment plan generator 268. Each treatment stage may include a generated 3D model of a dental arch at that treatment stage. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In block 1130, at least one orthodontic appliance is fabricated based on the generated treatment stages. For example, a set of appliances can be fabricated, each shaped according to a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired. The fabrication of the appliance may include automated removal of the appliance from a mold (e.g., automated removal of an untrimmed shell from mold a using a shell removal device).

In some instances, staging of various arrangements or treatment stages may not be necessary for design and/or fabrication of an appliance. As illustrated by the dashed line in FIG. 11, design and/or fabrication of an orthodontic appliance, and perhaps a particular orthodontic treatment, may include use of a representation of the patient's teeth (e.g., receive a digital representation of the patient's teeth at block 1110), followed by design and/or fabrication of an orthodontic appliance based on a representation of the patient's teeth in the arrangement represented by the received representation.

FIG. 12 illustrates one embodiment of a system 1201 for performing intraoral scanning, generating a three-dimensional (3D) surface and/or a virtual three-dimensional model of a dental site, and/or generating a treatment plan. System 1201 includes a dental office 1208 and optionally one or more dental lab 1210. The dental office 1208 and the dental lab 1210 each include a computing device 1205, 1206. Additionally, one or more server computing device 1207 may additionally be provided. The computing devices 1205, 1206, 207 may be connected to one another via a network 1280. The network 1280 may be a local area network (LAN), a public wide area network (WAN) (e.g., the Internet), a private WAN (e.g., an intranet), or a combination thereof.

Computing device 1205 may be coupled to one or more intraoral scanner 1250 (also referred to as a scanner) and/or a data store 1225 via a wired or wireless connection. In one embodiment, multiple scanners 1250 in dental office 1208 wirelessly connect to computing device 1205. In one embodiment, scanner 1250 is wirelessly connected to computing device 1205 via a direct wireless connection. In one embodiment, scanner 1250 is wirelessly connected to computing device 1205 via a wireless network. In one embodiment, the wireless network is a Wi-Fi network. In one embodiment, the wireless network is a Bluetooth network, a Zigbee network, or some other wireless network. In one embodiment, the wireless network is a wireless mesh network, examples of which include a Wi-Fi mesh network, a Zigbee mesh network, and so on. In an example, computing device 1205 may be physically connected to one or more wireless access points and/or wireless routers (e.g., Wi-Fi access points/routers). Intraoral scanner 1250 may include a wireless module such as a Wi-Fi module, and via the wireless module may join the wireless network via the wireless access point/router.

Computing devices 1206, 1207 may also be connected to a data store (not shown). The data stores may be local data stores and/or remote data stores. Computing device 1205 and computing devices 1206, 1207 may each include one or more processing devices, memory, secondary storage, one or more input devices (e.g., such as a keyboard, mouse, tablet, touchscreen, microphone, camera, and so on), one or more output devices (e.g., a display, printer, touchscreen, speakers, etc.), and/or other hardware components.

In embodiments, scanner 1250 includes an inertial measurement unit (IMU). The IMU may include an accelerometer, a gyroscope, a magnetometer, a pressure sensor and/or other sensor. For example, scanner 1250 may include one or more micro-electromechanical system (MEMS) IMU. The IMU may generate inertial measurement data (also referred to as movement data), including acceleration data, rotation data, and so on.

Computing device 1205 and/or data store 1225 may be located at dental office 1208 (as shown), at dental lab 1210, or at one or more other locations such as a server farm that provides a cloud computing service. Computing device 1205 and/or data store 1225 may connect to components that are at a same or a different location from computing device 1205 (e.g., components at a second location that is remote from the dental office 1208, such as a server farm that provides a cloud computing service). For example, computing device 1205 may be connected to a remote server, where some operations of intraoral scan application 1215 are performed on computing device 1205 and some operations of intraoral scan application 1215 are performed on the remote server.

Some additional computing devices may be physically connected to the computing device 1205 via a wired connection. Some additional computing devices may be wirelessly connected to computing device 1205 via a wireless connection, which may be a direct wireless connection or a wireless connection via a wireless network. In embodiments, one or more additional computing devices may be mobile computing devices such as laptops, notebook computers, tablet computers, mobile phones, portable game consoles, and so on. In embodiments, one or more additional computing devices may be traditionally stationary computing devices, such as desktop computers, set top boxes, game consoles, and so on. The additional computing devices may act as thin clients to the computing device 1205. In one embodiment, the additional computing devices access computing device 1205 using remote desktop protocol (RDP). In one embodiment, the additional computing devices access computing device 1205 using virtual network control (VNC). Some additional computing devices may be passive clients that do not have control over computing device 1205 and that receive a visualization of a user interface of intraoral scan application 1215. In one embodiment, one or more additional computing devices may operate in a master mode and computing device 1205 may operate in a slave mode.

Intraoral scanner 1250 may include a probe (e.g., a hand held probe) for optically capturing three-dimensional structures. The intraoral scanner 1250 may be used to perform an intraoral scan of a patient's oral cavity. An intraoral scan application 1215 running on computing device 1205 may communicate with the scanner 1250 to effectuate the intraoral scan. A result of the intraoral scan may be intraoral scan data 1235A, 1235B through 1235N that may include one or more sets of intraoral scans and/or sets of intraoral 2D images. Each intraoral scan may include a 3D image or point cloud that may include depth information of a portion of a dental site. In embodiments, intraoral scans include x, y and z information.

Intraoral scan data 1235A-N may also include color 2D images and/or images of particular wavelengths (e.g., near-infrared (NIRI) images, infrared images, ultraviolet images, images generated using fluorescent imaging, etc.) of a dental site in embodiments. In embodiments, intraoral scanner 1250 alternates between generation of 3D intraoral scans and one or more types of 2D intraoral images (e.g., color images, NIRI images, fluorescent imaging images, etc.) during scanning. For example, one or more 2D color images may be generated between generation of a fourth and fifth intraoral scan by outputting white light and capturing reflections of the white light using multiple cameras.

Intraoral scanner 1250 may be a confocal imaging scanner that generates height maps in some embodiments. In one embodiment, intraoral scanner 1250 corresponds to the iTero® intraoral scanner manufactured by Align Technology®. In some embodiments, intraoral scanner 1250 includes multiple different cameras (e.g., each of which may include one or more image sensors) that generate images (e.g., 2D images) of different regions of a patient's dental arch concurrently during structured light projection. A correspondence algorithm may be solved between projected pattern features and captured pattern features of the structured light pattern to generate an intraoral scan (e.g., a 3D point cloud) from the multiple concurrently generated images. In one embodiment, intraoral scanner 1250 corresponds to the iTero Lumina™ intraoral scanner manufactured by Align Technology®.

The scanner 1250 may transmit the intraoral scan data 1235A, 1235B through 1235N to the computing device 1205. Computing device 1205 may store the intraoral scan data 1235A-135N in data store 1225.

According to an example, a user (e.g., a practitioner) may subject a patient to intraoral scanning. In doing so, the user may apply scanner 1250 to one or more patient intraoral locations. The scanning may be divided into one or more segments (also referred to as roles). As an example, the segments may include a lower dental arch of the patient, an upper dental arch of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the scanner 1250 may provide intraoral scan data 1235A-N to computing device 1205. The intraoral scan data 1235A-N may be provided in the form of intraoral scan data sets, each of which may include 2D intraoral images (e.g., color 2D images) and/or 3D intraoral scans of particular teeth and/or regions of an dental site. In one embodiment, separate intraoral scan data sets are created for the maxillary arch, for the mandibular arch, for a patient bite, and/or for each preparation tooth. Alternatively, a single large intraoral scan data set is generated (e.g., for a mandibular and/or maxillary arch). Intraoral scans may be provided from the scanner 1250 to the computing device 1205 in the form of one or more points (e.g., one or more pixels and/or groups of pixels). For instance, the scanner 1250 may provide an intraoral scan as one or more point clouds. The intraoral scans may each comprise height information (e.g., a height map that indicates a depth for each pixel).

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Alternatively, full scans of upper and/or lower dental arches may be performed if a bridge is to be created. Some dental treatments may call for scanning of a patient's palate, while other dental treatments do not call for scanning of the patient's palate. For example, palatal expansion treatment that uses a sequence of manufactured palatal expanders may call for scanning of the palate, while orthodontic treatment may not call for scanning of the palate. Dental practitioners may not be used to scanning certain soft tissue such as the upper palate or gingiva of patients, and thus may not capture such soft tissue (e.g., palate) at all or may capture an insufficient amount of the soft tissue during intraoral scanning. Embodiments provide feedback mechanisms to notify dental practitioners when capture of additional soft tissue is recommended as well as to indicate to the dental practitioner where such areas are in the patient's oral cavity. In some embodiments, intraoral scan application 1215 includes a missing soft tissue determiner 1272 that identifies missing soft tissue information (e.g., palatal information and/or gingival information) and outputs indicators of the missing soft tissue information.

By way of non-limiting example, dental procedures may be broadly divided into prosthodontic (restorative) and orthodontic procedures (which may also include palatal expansion), and then further subdivided into specific forms of these procedures. Additionally, dental procedures may include identification and treatment of gum disease, sleep apnea, and intraoral conditions. The term prosthodontic procedure refers, inter alia, to any procedure involving the oral cavity and directed to the design, manufacture or installation of a dental prosthesis at a dental site within the oral cavity (dental site), or a real or virtual model thereof, or directed to the design and preparation of the dental site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture or installation of orthodontic elements at a dental site within the oral cavity, or a real or virtual model thereof, or directed to the design and preparation of the dental site to receive such orthodontic elements. These elements may be appliances including but not limited to brackets and wires, retainers, orthodontic aligners, palatal expanders, or other functional appliances.

In embodiments, intraoral scanning may be performed on a patient's oral cavity during a visitation of dental office 1208. The intraoral scanning may be performed, for example, as part of a semi-annual or annual dental health checkup. The intraoral scanning may also be performed before, during and/or after one or more dental treatments, such as orthodontic treatment, palatal expansion treatment, and/or prosthodontic treatment. The intraoral scanning may be a full or partial scan of the upper and/or lower dental arches, and may be performed in order to gather information for performing dental diagnostics, to generate a treatment plan, to determine progress of a treatment plan, and/or for other purposes. Depending on the type of dental treatment to be performed, the intraoral scanning may also include scanning of the patient's palate. The dental information (intraoral scan data 1235A-N) generated from the intraoral scanning may include 3D scan data, 2D color images, fluorescent imaging images, NIRI and/or infrared images, and/or ultraviolet images, of all or a portion of the upper jaw and/or lower jaw. The intraoral scan data 1235A-N may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

Intraoral scanners may work by moving the scanner 1250 inside a patient's mouth to capture all viewpoints of one or more tooth. During scanning, the scanner 1250 is calculating distances to solid surfaces in some embodiments. These distances may be recorded as images called ‘height maps’ or as point clouds in some embodiments. Each scan (e.g., optionally height map or point cloud) is overlapped algorithmically, or ‘stitched’, with the previous set of scans to generate a growing 3D surface. As such, each scan is associated with a rotation in space, or a projection, to how it fits into the 3D surface.

During intraoral scanning, intraoral scan application 1215 may register and stitch together two or more intraoral scans generated thus far from the intraoral scan session to generate a growing 3D surface. The 3D surface may be a representation of a scanned portion of a dental arch in embodiments. As scanning progresses, the scanned portion of the dental arch may grow, and the 3D surface may be updated (e.g., in real time or near real time) to reflect the latest captured intraoral scan data. In one embodiment, performing registration includes capturing 3D data of various points of a surface in multiple scans, and registering the scans by computing transformations between the scans. One or more 3D surfaces may be generated based on the registered and stitched together intraoral scans during the intraoral scanning. The one or more 3D surfaces may be output to a display so that a doctor or technician can view their scan progress thus far. As each new intraoral scan is captured and registered to previous intraoral scans and/or a 3D surface, the one or more 3D surfaces may be updated, and the updated 3D surface(s) may be output to the display. A view of the 3D surface(s) may be periodically or continuously updated according to one or more viewing modes of the intraoral scan application. In one viewing mode, the 3D surface may be continuously updated such that an orientation of the 3D surface that is displayed aligns with a field of view of the intraoral scanner (e.g., so that a portion of the 3D surface that is based on a most recently generated intraoral scan is approximately centered on the display or on a window of the display) and a user sees what the intraoral scanner sees. In one viewing mode, a position and orientation of the 3D surface is static, and an image of the intraoral scanner is optionally shown to move relative to the stationary 3D surface. Other viewing modes may include zoomed in viewing modes that show magnified views of one or more regions of the 3D surface (e.g., of intraoral areas of interest (AOIs)). Other viewing modes are also possible.

In embodiments, separate 3D surfaces are generated for the upper jaw and the lower jaw. This process may be performed in real time or near-real time to provide an updated view of the captured 3D surfaces during the intraoral scanning process.

Intraoral scan application 1215 may include a graphical user interface (GUI) in which representations of the upper dental arch and/or lower dental arch may be displayed during intraoral scanning. These representations may be 3D surfaces generated by stitching together intraoral scans as described above.

When a scan session or a portion of a scan session associated with a particular scanning role (e.g., upper jaw role, lower jaw role, bite role, etc.) is complete (e.g., all scans for a dental arch or a dental site including a portion of a dental arch to be scanned have been captured), intraoral scan application 1215 may generate a virtual 3D model of one or more scanned dental arches (e.g., of an upper jaw and a lower jaw), or portions thereof. The final 3D model may be a set of 3D points and their connections with each other (i.e. a mesh), a volumetric model (e.g., containing voxels), a point cloud, or other 3D representation. To generate the virtual 3D model, intraoral scan application 1215 may register and stitch together the intraoral scans generated from the intraoral scan session that are associated with a particular scanning role. The registration performed at this stage may be more accurate than the registration performed during the capturing of the intraoral scans, and may take more time to complete than the registration performed during the capturing of the intraoral scans. In one embodiment, performing scan registration includes capturing 3D data of various points of a surface in multiple scans, and registering the scans by computing transformations between the scans. The 3D data may be projected into a 3D space of a 3D model to form a portion of the 3D model. The intraoral scans may be integrated into a common reference frame by applying appropriate transformations to points of each registered scan and projecting each scan into the 3D space.

In one embodiment, registration is performed for adjacent or overlapping intraoral scans (e.g., each successive frame of an intraoral video). Registration algorithms are carried out to register two adjacent or overlapping intraoral scans and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, intraoral scan application 1215 may match points of one scan with the closest points interpolated on the surface of another scan, and iteratively minimize the distance between matched points. Other registration techniques may also be used.

Intraoral scan application 1215 may repeat registration for all intraoral scans of a sequence of intraoral scans to obtain transformations for each intraoral scan, to register each intraoral scan with previous intraoral scan(s) and/or with a common reference frame (e.g., with the 3D model). Intraoral scan application 1215 may integrate intraoral scans into a single virtual 3D model by applying the appropriate determined transformations to each of the intraoral scans. Each transformation may include rotations about one to three axes and translations within one to three planes.

Intraoral scan application 1215 may generate one or more 3D models from intraoral scans, and may display the 3D models to a user (e.g., a doctor) via a graphical user interface (GUI). The 3D models can then be checked visually by the doctor. The doctor can virtually manipulate the 3D models via the user interface with respect to up to six degrees of freedom (i.e., translated and/or rotated with respect to one or more of three mutually orthogonal axes) using suitable user controls (hardware and/or virtual) to enable viewing of the 3D model from any desired direction. In some embodiments, a trajectory of a virtual camera imaging the 3D model is automatically computed, and the 3D model is shown according to the determined trajectory. Accordingly, the doctor may review (e.g., visually inspect) the generated 3D model of a dental site and determine whether the 3D model is acceptable (e.g., whether a margin line of a preparation tooth is accurately represented in the 3D model) without manually controlling or manipulating a view of the 3D model. For example, in some embodiments, the intraoral scan application 1215 automatically generates a sequence of views of the 3D model and cycles through the views in the generated sequence. This may include zooming in, zooming out, panning, rotating, and so on.

Once a 3D model of a dental arch is approved by a treatment provider, the 3D model may be applied to a treatment protocol or treatment algorithm generated from natural language instructions to generate a treatment plan. In some embodiments, treatment planning component 1220 automatically generates one or more treatment plans using one or more 3D models of patient dentition (e.g., of dental arches) and a generate treatment protocol or treatment algorithm. In some embodiments, treatment planning component 1220 additionally generates the treatment protocol or treatment algorithm as described above. In some embodiments, treatment planning component 1220 is a local application that executes on computing device 1205. In some embodiments, treatment planning component 1220 is a server-based application that executes on computing device 1207. In some embodiments, some logic of treatment planning component 1220 executes on computing device 1205, and some logic of treatment planning component 1220 executes on computing device 1207.

Once a treatment plan has been generated, the treatment plan may be sent to a dental lab 1210 in some embodiments for verification of the treatment plan.

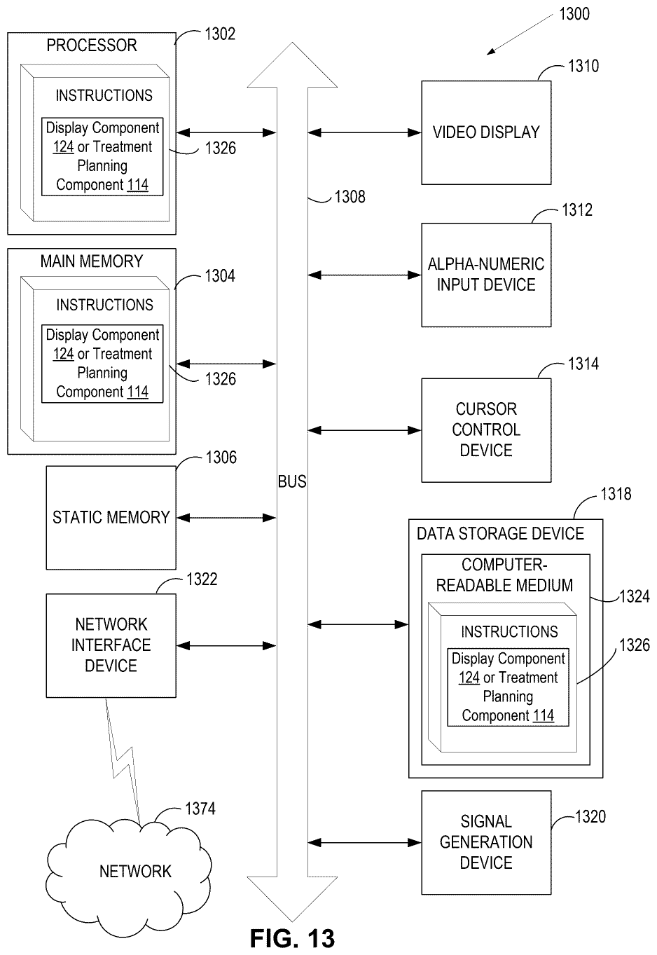

FIG. 13 is a block diagram illustrating a computer system 1300, according to some embodiments. In some embodiments, computer system 1300 may be connected (e.g., via a network, such as a Local Area Network (LAN), an intranet, an extranet, or the Internet) to other computer systems. Computer system 1300 may operate in the capacity of a server or a client computer in a client-server environment, or as a peer computer in a peer-to-peer or distributed network environment. Computer system 1300 may be provided by a personal computer (PC), a tablet PC, a Set-Top Box (STB), a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” shall include any collection of computers that individually or jointly execute a set (or multiple sets) of instructions to perform any one or more of the methods described herein.

In a further aspect, the computer system 1300 may include a processing device 1302, a volatile memory 1304 (e.g., Random Access Memory (RAM)), a non-volatile memory 1306 (e.g., Read-Only Memory (ROM) or Electrically-Erasable Programmable ROM (EEPROM)), and a data storage device 1318, which may communicate with each other via a bus 1308.

Processing device 1302 may be provided by one or more processors such as a general purpose processor (such as, for example, a Complex Instruction Set Computing (CISC) microprocessor, a Reduced Instruction Set Computing (RISC) microprocessor, a Very Long Instruction Word (VLIW) microprocessor, a microprocessor implementing other types of instruction sets, or a microprocessor implementing a combination of types of instruction sets) or a specialized processor (such as, for example, an Application Specific Integrated Circuit (ASIC), a Field Programmable Gate Array (FPGA), a Digital Signal Processor (DSP), or a network processor.

Computer system 1300 may further include a network interface device 1322 (e.g., coupled to network 1374). Computer system 1300 also may include a video display unit 1310 (e.g., an LCD), an alphanumeric input device 1312 (e.g., a keyboard), a cursor control device 1314 (e.g., a mouse), and a signal generation device 1320.

In some embodiments, data storage device 1318 may include a non-transitory computer-readable storage medium 1324 (e.g., non-transitory machine-readable medium) on which may store instructions 1326 encoding any one or more of the methods or functions described herein, including instructions encoding components of FIG. 1 (e.g., treatment planning component 114, display component 124, model 190, etc.) and for implementing methods described herein.

Instructions 1326 may also reside, completely or partially, within volatile memory 1304 and/or within processing device 1302 during execution thereof by computer system 1300, hence, volatile memory 1304 and processing device 1302 may also constitute machine-readable storage media.

While computer-readable storage medium 1324 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein. The term “computer-readable storage medium” shall include, but not be limited to, solid-state memories, optical media, and magnetic media.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and computer program components, or in computer programs.

Unless specifically stated otherwise, terms such as “receiving,” “performing,” “providing,” “obtaining,” “causing,” “accessing,” “determining,” “adding,” “using,” “training,” “reducing,” “generating,” “correcting,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. Also, the terms “first,” “second,” “third,” “fourth,” etc. as used herein are meant as labels to distinguish among different elements and may not have an ordinal meaning according to their numerical designation.

Examples described herein also relate to an apparatus for performing the methods described herein. This apparatus may be specially constructed for performing the methods described herein, or it may include a general purpose computer system selectively programmed by a computer program stored in the computer system. Such a computer program may be stored in a computer-readable tangible storage medium.

The methods and illustrative examples described herein are not inherently related to any particular computer or other apparatus. Various general purpose systems may be used in accordance with the teachings described herein, or it may prove convenient to construct more specialized apparatus to perform methods described herein and/or each of their individual functions, routines, subroutines, or operations. Examples of the structure for a variety of these systems are set forth in the description above.

Example implementations are set forth below.

In a first example implementation, a method includes obtaining, by a processing device, treatment provider instructions associated with a target dental treatment, expressed in a natural language format; providing first input comprising the treatment provided instructions to one or more trained artificial intelligence (AI) models; obtaining from the one or more trained AI models a first treatment protocol in association with the target dental treatment; and providing an alert to the treatment provider comprising the first treatment protocol.

A second example implementation may extend the first example implementation. In the second example implementation, providing the first input to the one or more trained AI models comprises transmitting the first input to a remote computing device that executes the one or more trained AI models; and obtaining the first treatment protocol comprises receiving the first treatment protocol from the remote computing device.

A third example implementation may extend any of the first through second example implementations. In the third example implementation, the one or more trained AI models perform a safety check to determine whether the first treatment protocol is in accordance with one or more clinical threshold conditions associated with the target dental treatment.

A fourth example implementation may extend any of the first through third example implementations. In the fourth example implementation, operations of the one or more trained AI models comprise separating the treatment provider instructions into a plurality of sections, one of the plurality of sections corresponding to the target dental treatment.

A fifth example implementation may extend any of the first through fourth example implementations. In the fifth example implementation, operations of the one or more trained AI models comprise transforming one or more natural language statements of the treatment provider instructions into machine readable statements associated with at least a portion of the target dental treatment.

A sixth example implementation may extend the fifth example implementation. In the sixth example implementation, operations of the one or more trained AI models comprise discarding one or more natural language statements of the treatment provider instructions which do not correspond to machine-readable instructions for the target dental treatment.

A seventh example implementation may extend any of the fifth through sixth example implementations. In the seventh example implementation, transforming the one or more natural language statements of the treatment provider instructions into machine readable statements comprises one or more of: determining that a plurality of natural language statements corresponds to one machine readable statement; or determining that one natural language statement corresponds to a plurality of machine readable statements.

An eighth example implementation may extend any of the first through seventh example implementations. In the eighth example implementation, the one or more trained AI models comprise a large language model.

A ninth example implementation may extend the eighth example implementation. In the ninth example implementation, the first input provided to the one or more trained AI models further comprises a prompt preface, the prompt preface comprising instructions to the one or more trained AI models to perform one or more operations in connection with the treatment provider instructions to generate the first treatment protocol.

A tenth example implementation may extend any of the eighth through ninth example implementations. In the tenth example implementation, the first input is provided to the one or more trained AI models to generate first output, and wherein second input comprising the first output is provided to the trained machine learning model to generate second output, wherein the first treatment protocol is based at least in part on the second output.

An eleventh example implementation may extend any of the first through tenth example implementations. In the eleventh example implementation, the method further includes providing, via a graphical user interface (GUI), a prompt for a treatment provider to provide the treatment provider instructions; obtaining, via the GUI, the treatment provider instructions; and providing, via the GUI, a natural language validation prompt associated with the first treatment protocol, wherein the first treatment protocol is generated responsive to obtaining validation from the treatment provider, and wherein generating the natural language validation prompt comprises: obtaining machine-readable instructions associated with first treatment protocol; and processing the first treatment protocol using a response generator model to generate a natural language description of operations of the machine-readable instructions; and outputting the natural language description.

A twelfth example implementation may extend the eleventh example implementation. In the twelfth example implementation, the response generator model comprises a deterministic or rule-based model.

A thirteenth example implementation may extend any of the first through twelfth example implementations. In the thirteenth example implementation, the method further includes generating a request for one or more treatment details; outputting the request; and receiving additional provider instructions comprising the one or more treatment details in the natural language format; wherein the additional provider instructions are processed by the one or more trained AI models to generate the first treatment protocol.

A fourteenth example implementation may extend the thirteenth example implementation. In the fourteenth example implementation, the method further includes determining that the one or more treatment details are missing in the treatment provider instructions.

A fifteenth example implementation may extend any of the first through fourteenth example implementations. In the fifteenth example implementation, the method further includes performing the following by the one or more trained AI models: updating a formatting of the first input to generate second input; separating the second input into a plurality of sections; determining a malocclusion type for each of the plurality of sections; separately processing each section of the plurality of sections to generate a portion of the first treatment protocol; combining, for each of the plurality of sections, the associated portion of the first treatment protocol to generate the first treatment protocol.

A sixteenth example implementation may extend the fifteenth example implementation. In the sixteenth example implementation, a separate model is used to process sections for each malocclusion type.

A seventeenth example implementation may extend any of the first through sixteenth example implementations. In the seventeenth example implementation, the treatment protocol is a general treatment protocol usable as a template to generate prescriptions for a plurality of patients.

An eighteenth example implementation may extend any of the first through seventeenth example implementations. In the eighteenth example implementation, the treatment protocol is a machine readable prescription for the target dental treatment.

A nineteenth example implementation may extend any of the first through eighteenth example implementations. In the nineteenth example implementation, the first input comprises: an instruction to convert the treatment provider instructions from the natural language format into the treatment protocol; a detailed description of each treatment option that is possible for the first treatment protocol; one or more examples of each treatment option; and instructions on how to process unclear, ambiguous, or unsupported statements.

In a twentieth example implementation, a method includes obtaining a plurality of treatment provider instructions associated with dental treatments; obtaining a plurality of machine-readable instructions corresponding to the treatment provider instructions; and training a machine learning model to generate a trained machine learning model by providing the plurality of treatment provider instructions as training input and the plurality of machine-readable instructions as target output.

A twenty-first example implementation may extend the twentieth example implementation. In the twenty-first example implementation, training the machine learning model comprises adjusting one or more parameters of a large language model.

A twenty-second example implementation may extend the twenty-first example implementation. In the twenty-second example implementation, training the machine learning model comprises performing parameter-efficient fine-tuning operations.

A twenty-third example implementation may extend the twenty-second example implementation. In the twenty-third example implementation, training the machine learning model comprises performing low-rank adaptation operations.

A twenty-fourth example implementation may extend any of the twenty-first through twenty-third example implementations. In the twenty-fourth example implementation, training the machine learning model comprises introducing adapter layers between existing layers of the machine learning model, and adjusting parameters of the adapter layers.

A twenty-fifth example implementation may extend any of the twentieth through twenty-fourth example implementations. In the twenty-fifth example implementation, the method further includes obtaining feedback data associated with operation of the trained machine learning model; and retraining the trained machine learning model based on the feedback data.

A twenty-sixth example implementation may extend the twenty-fifth example implementation. In the twenty-sixth example implementation, the feedback data comprises evaluation data submitted by a user.

A twenty-seventh example implementation may extend any of the twenty-fifth through twenty-sixth example implementations. In the twenty-seventh example implementation, the feedback data comprises indirect feedback data related to one or more of time spent on a dental treatment, number of updates performed on a treatment protocol associated with the dental treatment, or level of confidence of results generated by the trained machine learning model.

In a twenty-eighth example implementation, a method includes obtaining, by a processing device, a data model comprising a plurality of fields of a dental treatment protocol or prescription to be filled, the plurality of fields associated with a dental treatment; generating a first prompt in natural language associated with one or more first fields of the plurality of fields; presenting, via a graphical user interface, the first prompt; obtaining a response to the first prompt; providing the response to a trained machine learning model, wherein the trained machine learning model generates a machine-readable output; and filling the one or more first fields of the dental treatment protocol or prescription using the machine-readable output of the trained machine learning model.

A twenty-ninth example implementation may extend the twenty-eighth example implementation. In the twenty-ninth example implementation, the method further includes providing a second prompt to verify that the output of the trained machine learning model accurately applies to a dental treatment associated with the first one or more fields, wherein filling the fields is performed responsive to receiving verification based on the second prompt.

A thirtieth example implementation may extend any of the twenty-eighth through twenty-ninth example implementations. In the thirtieth example implementation, the trained machine learning model comprises a large language model.

A thirty-first example implementation may extend any of the twenty-eighth through thirtieth example implementations. In the thirty-first example implementation, the method further includes generating a second prompt associated with one or more second fields of the plurality of fields; presenting, via the graphical user interface, the second prompt; obtaining a response to the second prompt; providing the response to the second prompt to the trained machine learning model or a second trained machine learning model, wherein the trained machine learning model or the second trained machine learning model generates a second machine-readable output; and filling the one or more second fields of the dental treatment protocol or prescription using the second machine-readable output.

A thirty-second example implementation includes a non-transitory computer readable medium comprising instructions that, when executed by a processing device, cause the processing device to perform the method of any of the first through thirty-first example implementations.

A thirty-third example implementation includes a system comprising one or more computing device each comprising a memory and one or more processors, wherein the one or more computing devices are configured to perform the method of any of the first through thirty-first example implementations.

In a thirty-fourth example implementation, a method includes providing, by a processing device, a first set of options related to treatment preferences for a first dental condition; obtaining a first selection of one of the first set of options; processing the first selection using a model to generate machine-readable code in a domain-specific language for dental treatment (e.g., such as orthodontic treatment, a palatal expansion treatment, and/or a prosthodontic treatment), wherein the machine-readable code constitutes a clinical protocol for generating orthodontic treatment plans; and outputting the machine-readable code.

A thirty-fifth example implementation may extend the thirty-fourth example implementation. In the thirty-fifth example implementation, the method further includes executing the machine-readable code in association with a dental patient to generate a treatment plan for the first dental condition.

A thirty-sixth example implementation may extend the thirty-fifth example implementation. In the thirty-sixth example implementation, generating the treatment plan comprises obtaining a three-dimensional intraoral scan of the dental patient, obtaining a second selection of one or more treatment goals, and executing the machine-readable code in view of the intraoral scan and the one or more treatment goals.

A thirty-seventh example implementation may extend any of the thirty-fifth through thirty-sixth example implementations. In the thirty-seventh example implementation, generating the treatment plan includes generating designs for one or more treatment appliances, and wherein the method further comprises providing the designs for manufacturing of the appliances.

A thirty-eighth example implementation may extend any of the thirty-fourth through thirty-seventh example implementations. In the thirty-eighth example implementation, the first set of options are provided via a graphical user interface (GUI), and wherein the first selection is obtained via the GUI.

A thirty-ninth example implementation may extend the thirty-eighth example implementation. In the thirty-ninth example implementation, the method further includes providing for inspection via the GUI a three-dimensional model depicting example dentition indicating one or more aspects of a default selection or the first selection.

A fortieth example implementation may extend any of the thirty-eighth through thirty-ninth example implementations. In the fortieth example implementation, the method further includes providing, via the GUI, an indication of an expected outcome with respect to the first selection.

A forty-first example implementation may extend any of the thirty-fourth through fortieth example implementations. In the forty-first example implementation, the method further includes providing a second set of options related to treatment preferences for a second dental condition; obtaining a second selection of one of the second set of options; and further processing the second selection using the model or an additional model to generate the machine-readable code in the domain-specific language for orthodontic treatment, wherein the machine-readable code is associated with the first dental condition and the second dental condition.

A forty-second example implementation may extend any of the thirty-fourth through forty-first example implementations. In the forty-second example implementation, the first dental condition comprises one of: a malocclusion; anterior leveling; or an overbite.

A forty-third example implementation may extend any of the thirty-fourth through forty-second example implementations. In the forty-third example implementation, the method further includes providing a second set of options related to treatment operations applicable to multiple dental treatments, wherein the treatment operations comprise one or more of: interproximal reduction; placing pontics; or extracting teeth.

A forty-fourth example implementation may extend any of the thirty-fourth through forty-third example implementations. In the forty-fourth example implementation, the first set of options comprises a hierarchical tree structure, and wherein obtaining the first selection causes one or more additional options to be displayed or hidden based on the first selection.

A forty-fifth example implementation may extend any of the thirty-fourth through forty-fourth example implementations. In the forty-fifth example implementation, the method further includes generating a version identifier associated with the machine-readable code; storing the machine-readable code in association with the version identifier; and maintaining a history of prior versions of the machine-readable code.

In a forty-sixth example implementation, a method includes providing, by a processing device via a graphical user interface (GUI), a plurality of treatment parameter fields organized into treatment categories, each treatment parameter field associated with one or more selectable options for orthodontic treatment; obtaining, via the GUI, a selection for each of one or more of the plurality of treatment parameter fields; deterministically generating, based on the selections, machine-readable code in a domain-specific language for orthodontic treatment planning, wherein each selection maps to a corresponding code segment such that identical selections produce identical machine-readable code; validating the machine-readable code to confirm syntactic and semantic correctness; and storing the machine-readable code as a treatment protocol version associated with a treatment provider.

A forty-seventh example implementation may extend the forty-sixth example implementation. In the forty-seventh example implementation, the plurality of treatment parameter fields are organized into a hierarchical tree structure, and wherein obtaining a selection for a first treatment parameter field causes one or more additional treatment parameter fields to be displayed or hidden based on the selection.

A forty-eighth example implementation may extend any of the forty-sixth through forty-seventh example implementations. In the forty-eighth example implementation, the method further includes maintaining a version history comprising a plurality of treatment protocol versions; associating each treatment protocol version with a version identifier; and providing, via the GUI, an interface for viewing or reverting to a prior treatment protocol version.

A forty-ninth example implementation may extend any of the forty-sixth through forty-eighth example implementations. In the forty-ninth example implementation, the treatment categories comprise one or more of: interproximal reduction parameters including timing and location; attachment parameters including size and delay stages; precision cut parameters including placement and prioritization; bite ramp parameters including placement locations; or overcorrection parameters including type and arch selection.

A fiftieth example implementation may extend any of the forty-sixth through forty-ninth example implementations. In the fiftieth example implementation, the method further includes providing, via the GUI, a division of treatment options based on patient demographics, wherein a first set of options is associated with adult patients and a second set of options is associated with teen patients; and generating the machine-readable code to include conditional logic based on patient demographic classification.

A fifty-first example implementation may extend any of the forty-sixth through fiftieth example implementations. In the fifty-first example implementation, the method further includes providing, via the GUI, a default treatment option indicator for each of the plurality of treatment parameter fields; and displaying a three-dimensional model depicting example dentition corresponding to the default treatment option.

A fifty-second example implementation may extend any of the forty-sixth through fifty-first example implementations. In the fifty-second example implementation, the method further includes applying the treatment protocol version to generate treatment plans for a plurality of patients; determining that a treatment requirement for a particular patient is outside a scope of the plurality of treatment parameter fields; and providing the treatment requirement to an artificial intelligence model to generate supplemental machine-readable code for the particular patient.

In a fifty-third example implementation, a method includes providing, by a processing device, a first set of treatment options related to a first treatment goal in association with a first dental condition, and a second set of treatment options related to a second treatment goal in association with the first dental condition; obtaining, by the processing device, a first selection from the first set of treatment options and a second selection from the second set of treatment options; generating a treatment protocol in a machine-readable format comprising the first selection and the second selection; obtaining an indication that the first treatment goal is to be applied to a patient in association with the first dental condition; and generating a treatment plan for the patient corresponding to the first treatment goal based on the treatment protocol.

A fifty-fourth example implementation may extend the fifty-third example implementation. In the fifty-fourth example implementation, the method further includes providing a third set of treatment options related to a second dental condition; and obtaining a third selection from the third set of treatment options, wherein the treatment protocol further comprises the third selection.

A fifty-fifth example implementation may extend any of the fifty-third through fifty-fourth example implementations. In the fifty-fifth example implementation, generating the treatment protocol comprises providing the first selection and the second selection to a model that generates machine-readable treatment protocols based on the first selection and the second selection.

A fifty-sixth example implementation may extend any of the fifty-third through fifty-fifth example implementations. In the fifty-sixth example implementation, generating the treatment plan comprises utilizing a three-dimensional model of dentition of the patient to generate one or more treatment appliance designs.

A fifty-seventh example implementation may extend the fifty-sixth example implementation. In the fifty-seventh example implementation, the method further includes providing the one or more treatment appliance designs for manufacturing.

A fifty-eighth example implementation may extend any of the fifty-third through fifty-seventh example implementations. In the fifty-eighth example implementation, the first set of treatment options is provided via a graphical user interface (GUI), and wherein the first selection is obtained via the GUI.

A fifty-ninth example implementation may extend any of the fifty-third through fifty-eighth example implementations. In the fifty-ninth example implementation, the first dental condition comprises: tooth crowding; tooth spacing; shifted midline; anterior level; anterior-posterior relationship; posterior crossbite; or overbite.

In a sixtieth example implementation, a method includes providing, via a graphical user interface (GUI), a set of treatment categories in association with operations of a dental treatment; providing, for each of the set of treatment categories, a corresponding set of treatment options; obtaining, for a first of the set of treatment categories, a selection from the set of treatment options via the GUI; providing the selection to a model that processes the selection to generate a treatment protocol in a machine-readable format comprising a domain-specific language for orthodontic treatment; and displaying the treatment protocol via the GUI.

A sixty-first example implementation may extend the sixtieth example implementation. In the sixty-first example implementation, providing the selection to the model comprises transmitting the first selection to a remote computing device that executes the model, the method further comprising: receiving the treatment protocol from the remote computing device.

A sixty-second example implementation may extend any of the sixtieth through sixty-first example implementations. In the sixty-second example implementation, the method further includes executing the treatment protocol in connection with a dental patient to generate a treatment plan for the patient.

A sixty-third example implementation may extend the sixty-second example implementation. In the sixty-third example implementation, the method further includes generating one or more dental treatment appliance designs corresponding to the treatment plan; and providing the one or more dental treatment appliance designs for manufacturing.

A sixty-fourth example implementation may extend any of the sixtieth through sixty-third example implementations. In the sixty-fourth example implementation, the set of treatment categories comprises a set of dental conditions, comprising at least one of: tooth crowding; tooth spacing; shifted midline; anterior level; anterior-posterior relationship; posterior crossbite; or overbite.

A sixty-fifth example implementation may extend any of the sixtieth through sixty-fourth example implementations. In the sixty-fifth example implementation, the set of treatment categories comprises treatment options, comprising one or more of: interproximal reduction; placing pontics; or extracting teeth.

A sixty-sixth example implementation may extend any of the sixtieth through sixty-fifth example implementations. In the sixty-sixth example implementation, the method further includes providing, for display by the GUI, a model of example dentition, the model comprising indications of treatment areas of interest in association with one of the set of treatment categories.

A sixty-seventh example implementation may extend any of the sixtieth through sixty-sixth example implementations. In the sixty-seventh example implementation, the method further includes obtaining, for each of the set of treatment categories, either a selection from an associated set of treatment options or a default selection from the associated set of treatment options, wherein the treatment protocol comprises machine-readable instructions for each of the treatment categories.

A sixty-eighth example implementation may extend any of the sixtieth through sixty-seventh example implementations. In the sixty-eighth example implementation, the method further includes obtaining a natural language instruction associated with a dental treatment that is outside a scope of the set of treatment categories; providing the natural language instruction to an artificial intelligence (AI) model; obtaining from the AI model an update to the treatment protocol; and updating the treatment protocol based on the update.

A sixty-ninth example implementation may extend any of the sixtieth through sixty-eighth example implementations. In the sixty-ninth example implementation, the method further includes obtaining dental data of a patient; determining that one or more aspects of dentition of the patient is outside a scope of the set of treatment categories based on the dental data; obtaining a natural language instruction associated with treatment of the patient; providing the natural language instruction to an artificial intelligence (AI) model; obtaining output from the AI model comprising either additional treatment protocol data or first treatment plan data; and generating a treatment plan for the patient based on the treatment protocol and either the additional treatment protocol data or the first treatment plan data.

A seventieth example implementation includes a non-transitory computer readable medium comprising instructions that, when executed by a processing device, cause the processing device to perform the method of any of the thirty-fourth through sixty-ninth example implementations.

A seventy-first example implementation includes a system comprising one or more computing device each comprising a memory and one or more processors, wherein the one or more computing devices are configured to perform the method of any of the thirty-fourth through sixty-ninth example implementations.

In a seventy-second example implementation, a method includes obtaining first treatment provider instructions in natural language, the first treatment provider instructions associated with treatment of a dental patient; providing the first treatment provider instructions and a prompt comprising rules for a domain-specific format for dental treatment plans (e.g., for orthodontic treatment plans, prosthodontic treatment plans, palatal expansion treatment plans, etc.) to a trained artificial intelligence (AI) model; obtaining, as output from the trained AI model, first machine-readable instructions related to the treatment provider instructions structured according to the domain-specific format; and causing a treatment planning algorithm to be adjusted based on the first machine-readable instructions.

A seventy-third example implementation may extend the seventy-second example implementation. In the seventy-third example implementation, providing the first treatment provider instructions and the prompt to the trained AI model comprises transmitting at least one of the first treatment provider instructions or the prompt to a remote computing device that executes the trained AI model; and obtaining the first machine-readable instructions comprises receiving the first machine-readable instructions from the remote computing device.

A seventy-fourth example implementation may extend the seventy-third example implementation. In the seventy-fourth example implementation, causing the treatment planning algorithm to be adjusted comprises: receiving confirmation to apply the first machine-readable instructions; and transmitting the first machine-readable instructions to the remote computing device or a second remote computing device executing an orthodontic treatment planning application.

A seventy-fifth example implementation may extend any of the seventy-second through seventy-fourth example implementations. In the seventy-fifth example implementation, the method further includes obtaining second machine-readable instructions encoding treatment preferences of the treatment provider; and generating the treatment planning algorithm based on the second machine-readable instructions.

A seventy-sixth example implementation may extend the seventy-fifth example implementation. In the seventy-sixth example implementation, the method further includes obtaining treatment provider preferences in natural language; and generating the second machine-readable instructions based on the treatment provider preferences.

A seventy-seventh example implementation may extend any of the seventy-fifth through seventy-sixth example implementations. In the seventy-seventh example implementation, the method further includes obtaining a generic treatment planning algorithm, wherein generating the treatment planning algorithm comprises adjusting the generic treatment planning algorithm based on the second machine-readable instructions.

A seventy-eighth example implementation may extend any of the seventy-second through seventy-seventh example implementations. In the seventy-eighth example implementation, the prompt comprises a set of classifications of treatment instruction categories, and one or more examples of machine-readable instructions.

A seventy-ninth example implementation may extend any of the seventy-second through seventy-eighth example implementations. In the seventy-ninth example implementation, the method further includes performing treatment instruction validation on the first machine-readable instructions, wherein treatment instruction validation comprises determining that the first machine-readable instructions do not violate a set of treatment instruction heuristics.

An eightieth example implementation may extend any of the seventy-second through seventy-ninth example implementations. In the eightieth example implementation, the method further includes obtaining second treatment provider instructions in natural language; providing the second treatment provider instructions to the trained AI model; obtaining, as output from the trained AI model, second machine-readable instructions; performing treatment instruction validation on the second machine-readable instructions; and responsive to determining that the second machine-readable instructions violate one or more of a set of treatment instruction heuristics, provide the second treatment provider instructions to a manual treatment planning pipeline.

An eighty-first example implementation may extend any of the seventy-second through eightieth example implementations. In the eighty-first example implementation, the method further includes obtaining dental data of the dental patient; and processing the dental data using the adjusted treatment planning algorithm to generate a treatment plan for the dental patient.

An eighty-second example implementation may extend the eighty-first example implementation. In the eighty-second example implementation, the method further includes causing one or more treatment appliances to be manufactured based on the treatment plan.

An eighty-third example implementation may extend any of the seventy-second through eighty-second example implementations. In the eighty-third example implementation, the first machine-readable instructions are structured according to a language-independent data-interchange format schema that organizes the treatment provider instructions into distinct categories, each category representing a specific aspect of treatment planning.

An eighty-fourth example implementation may extend any of the seventy-second through eighty-third example implementations. In the eighty-fourth example implementation, the method further includes obtaining second machine-readable instructions encoding general treatment preferences of the treatment provider; determining a priority relationship between the first machine-readable instructions and the second machine-readable instructions; and adjusting the treatment planning algorithm such that the first machine-readable instructions override corresponding portions of the second machine-readable instructions.

In an eighty-fifth example implementation, a method includes obtaining first instructions in a first machine-readable format configured to represent parameters for a treatment plan based on input data; obtaining second instructions in natural language corresponding to target adjustments to the first instructions; providing the second instructions to a trained artificial intelligence (AI) model; obtaining output from the trained AI model based on the second instructions, the output comprising third instructions in a second machine-readable format corresponding to the second instructions; and updating the first instructions based on the third instructions.

An eighty-sixth example implementation may extend the eighty-fifth example implementation. In the eighty-sixth example implementation, the treatment plan comprises a dental treatment plan.

An eighty-seventh example implementation may extend any of the eighty-fifth through eighty-sixth example implementations. In the eighty-seventh example implementation, the target adjustments to the first instructions are based on a target dental patient.

An eighty-eighth example implementation may extend any of the eighty-fifth through eighty-seventh example implementations. In the eighty-eighth example implementation, the first instructions encode preferences of a treatment provider, and wherein the second instructions comprise one or more deviations from the preferences of the treatment provider.

An eighty-ninth example implementation may extend any of the eighty-fifth through eighty-eighth example implementations. In the eighty-ninth example implementation, the method further includes obtaining fourth instructions in a third machine-readable format associated with treatment preferences of a treatment provider, wherein the first instructions are based on the fourth instructions.

A ninetieth example implementation may extend the eighty-ninth example implementation. In the ninetieth example implementation, the method further includes obtaining fifth instructions in natural language, wherein the fifth instructions comprise the treatment preferences of the treatment provider; providing the fifth instructions to the trained AI model; and obtaining, as output from the trained AI model, the fourth instructions.

In a ninety-first example implementation, a method includes obtaining, by a processing device, free-text treatment instructions from a treatment provider associated with a specific dental patient, the free-text treatment instructions expressed in natural language; providing the free-text treatment instructions and a prompt to a large language model (LLM), wherein the prompt includes a description of instruction categories and examples of machine-readable instructions corresponding to natural language instructions within each category; obtaining, as output from the LLM, case-specific machine-readable instructions in a structured format, the case-specific machine-readable instructions corresponding to the free-text treatment instructions; performing validation of the case-specific machine-readable instructions to determine compatibility with a treatment planning engine; responsive to determining that the case-specific machine-readable instructions are compatible with the treatment planning engine, providing the case-specific machine-readable instructions and patient data to the treatment planning engine; and generating, by the treatment planning engine, a treatment plan for the specific dental patient based on the case-specific machine-readable instructions.

A ninety-second example implementation may extend the ninety-first example implementation. In the ninety-second example implementation, the method further includes obtaining general treatment protocol instructions associated with the treatment provider, the general treatment protocol instructions applicable to multiple patients; and combining the case-specific machine-readable instructions with the general treatment protocol instructions, wherein the case-specific machine-readable instructions have a higher priority than the general treatment protocol instructions such that the case-specific machine-readable instructions override corresponding portions of the general treatment protocol instructions.

A ninety-third example implementation may extend any of the ninety-first through ninety-second example implementations. In the ninety-third example implementation, performing validation of the case-specific machine-readable instructions comprises: determining whether each instruction type in the case-specific machine-readable instructions is supported by the treatment planning engine; determining whether parameter values in the case-specific machine-readable instructions fall within predefined acceptable ranges; and responsive to determining that an instruction type is unsupported or a parameter value falls outside an acceptable range, directing the free-text treatment instructions to a manual treatment planning process.

A ninety-fourth example implementation may extend any of the ninety-first through ninety-third example implementations. In the ninety-fourth example implementation, the structured format comprises a language-independent data-interchange format schema that organizes the case-specific machine-readable instructions into distinct categories, each category representing a specific aspect of treatment planning, and wherein the schema includes a default category for instructions that do not fit into predefined instruction categories.

A ninety-fifth example implementation may extend any of the ninety-first through ninety-fourth example implementations. In the ninety-fifth example implementation, the free-text treatment instructions include a reference to one or more teeth expressed according to a first indexing scheme, and wherein the method further comprises: obtaining a three-dimensional model of dentition of the specific dental patient; sorting teeth of the three-dimensional model based on geometric position along a jaw arc; and mapping the reference to the one or more teeth to corresponding teeth in the sorted three-dimensional model based on the geometric position.

A ninety-sixth example implementation may extend the ninety-fifth example implementation. In the ninety-sixth example implementation, the reference to the one or more teeth comprises one or more of: an interval specification identifying teeth between two specified teeth; a group specification identifying teeth belonging to an anatomical category; a relative position specification identifying teeth based on position relative to a reference tooth; or an exclusion specification identifying teeth by excluding specified teeth from a group.

A ninety-seventh example implementation may extend any of the ninety-fifth through ninety-sixth example implementations. In the ninety-seventh example implementation, the three-dimensional model includes one or more of a supernumerary tooth or a missing tooth, and wherein mapping the reference to the one or more teeth accounts for the supernumerary tooth or the missing tooth based on the geometric position along the jaw arc.

In a ninety-eighth example implementation, a method includes obtaining treatment provider instructions in natural language, wherein the treatment provider instructions are indicative of one or more differences between target dental treatment for a target dental patient and a set of treatment preferences associated with the treatment provider; obtaining a prompt comprising: a description of a plurality of categories of instructions associated with dental treatment, and a plurality of examples of machine-readable instructions corresponding to natural language instructions within the categories of instructions; providing the treatment provider instructions and the prompt as input to a trained artificial intelligence (AI) model; obtaining output from the trained AI model, the output comprising a machine-readable format that is a domain-specific format for orthodontic treatment; and generating a treatment plan based on the output from the trained AI model.

A ninety-ninth example implementation may extend the ninety-eighth example implementation. In the ninety-ninth example implementation, the method further includes generating a treatment algorithm based on output from the trained AI model; and obtaining patient data of the target dental patient, wherein generating the treatment plan comprises applying the treatment algorithm to the patient data.

A one hundredth example implementation may extend the ninety-ninth example implementation. In the one hundredth example implementation, the method further includes obtaining a standard treatment algorithm, wherein generating the treatment algorithm comprises adjusting the standard treatment algorithm.

A one hundred first example implementation may extend the one hundredth example implementation. In the one hundred first example implementation, the standard treatment algorithm is associated with the treatment preferences.

A one hundred second example implementation may extend any of the ninety-ninth through one hundred first example implementations. In the one hundred second example implementation, the patient data comprises a three-dimensional model of dentition of the patient.

A one hundred third example implementation may extend any of the ninety-eighth through one hundred second example implementations. In the one hundred third example implementation, the output from the trained AI model comprises machine-readable instructions associated with the first treatment provider instructions, and wherein the method further comprises: performing instruction validation; and responsive to instruction validation, updating a treatment algorithm based on the output from the trained AI model.

A one hundred fourth example implementation may extend the one hundred third example implementation. In the one hundred fourth example implementation, instruction validation comprises determining that the machine-readable instructions to not violate one or more of a set of treatment instruction heuristics.

A one hundred fifth example implementation may extend any of the ninety-eighth through one hundred fourth example implementations. In the one hundred fifth example implementation, the method further includes causing one or more treatment appliances to be manufactured based on the treatment plan.

A one hundred sixth example implementation includes a non-transitory computer readable medium comprising instructions that, when executed by a processing device, cause the processing device to perform the method of any of the seventy-second through one hundred fifth example implementations.

A one hundred seventh example implementation includes a system comprising one or more computing device each comprising a memory and one or more processors, wherein the one or more computing devices are configured to perform the method of any of the seventy-second through one hundred fifth example implementations.

In a one hundred eighth example implementation, a method includes obtaining, by a processing device, first natural language instructions for treating dentition of a patient, the first natural language instructions comprising a first reference to a plurality of ordered teeth (and/or other ordered objects) according to a first indexing scheme for at least one of teeth or inter-tooth intervals (and/or for other ordered objects); providing the natural language instructions and an accompanying prompt as input to an artificial intelligence (AI) model, wherein the prompt comprises a description of the first indexing scheme; and obtaining, as output from the AI model, first machine-readable instructions for treating the dentition of the patient, wherein the first machine-readable instructions comprise instructions associated with the plurality of ordered teeth, and wherein the first machine-readable instructions comprise a second reference to the plurality of ordered teeth according to a first machine-readable indexing scheme.

A one hundred ninth example implementation may extend the one hundred eighth example implementation. In the one hundred ninth example implementation, providing the natural language instructions and the accompanying prompt to the AI model comprises transmitting at least one of the natural language instructions or the accompanying prompt to a remote computing device that executes the AI model; and obtaining the first machine-readable instructions comprises receiving the first machine-readable instructions from the remote computing device.

A one hundred tenth example implementation may extend any of the one hundred eighth through one hundred ninth example implementations. In the one hundred tenth example implementation, the first indexing scheme comprises: a definition of individual tooth identifiers supporting multiple numbering systems; a definition of tooth groups representing anatomical categories; a definition of tooth intervals representing inclusive ranges between two teeth; a definition of relative tooth positions based on mesial or distal direction; and exclusion logic representing a group of exclusions using nested references to include and exclude sets of teeth.

A one hundred eleventh example implementation may extend any of the one hundred eighth through one hundred tenth example implementations. In the one hundred eleventh example implementation, the prompt further comprises a description of a second indexing scheme.

A one hundred twelfth example implementation may extend the one hundred eleventh example implementation. In the one hundred twelfth example implementation, the first natural language instructions further comprise a third reference to the plurality of ordered teeth according to the second indexing scheme.

A one hundred thirteenth example implementation may extend any of the one hundred eighth through one hundred twelfth example implementations. In the one hundred thirteenth example implementation, the prompt further comprises an indication of a preferred indexing scheme associated with the plurality of ordered teeth.

A one hundred fourteenth example implementation may extend any of the one hundred eighth through one hundred thirteenth example implementations. In the one hundred fourteenth example implementation, the method further includes associating indices of the first machine-readable indexing scheme to a three-dimensional model of the plurality of ordered teeth, wherein the ordered teeth of the three-dimensional model are indexed according to a second machine-readable indexing scheme, different than the first.

A one hundred fifteenth example implementation may extend the one hundred fourteenth example implementation. In the one hundred fifteenth example implementation, the method further includes segmenting the three-dimensional model to identify teeth in the three-dimensional model; sorting the identified teeth based on geometric position along a jaw arch; generating an array of teeth corresponding to the machine-readable instructions for treating the dentition of the patient; and mapping teeth of the array of teeth to the sorted teeth of the three-dimensional model.

A one hundred sixteenth example implementation may extend the one hundred fifteenth example implementation. In the one hundred sixteenth example implementation, the method further includes determining an array of interteeth intervals from the mapped teeth of the array of teeth.

A one hundred seventeenth example implementation may extend any of the one hundred eighth through one hundred sixteenth example implementations. In the one hundred seventeenth example implementation, the first natural language instructions further comprise a fourth reference to a first space between two teeth of the plurality of ordered teeth, and wherein the first machine-readable instructions comprise a fifth reference to the first space according to the first machine-readable indexing scheme.

A one hundred eighteenth example implementation may extend any of the one hundred eighth through one hundred seventeenth example implementations. In the one hundred eighteenth example implementation, the first natural language instructions further comprise a sixth reference to a subset of the plurality of ordered teeth, and wherein the first machine-readable instructions comprise a seventh reference to the subset of the plurality of ordered teeth according to the first machine-readable indexing scheme.

A one hundred nineteenth example implementation may extend any of the one hundred eighth through one hundred eighteenth example implementations. In the one hundred nineteenth example implementation, the AI model comprises a large language model.

A one hundred twentieth example implementation may extend any of the one hundred eighth through one hundred nineteenth example implementations. In the one hundred twentieth example implementation, the method further includes performing validation of the first machine-readable instructions, wherein the validation comprises determining that the first machine-readable instructions do not violate a set of heuristics associated with the first machine-readable instructions.

A one hundred twenty-first example implementation may extend any of the one hundred eighth through one hundred twentieth example implementations. In the one hundred twenty-first example implementation, the first natural language instructions comprise an update to a dental treatment protocol with respect to a dental patient, and the plurality of ordered teeth comprise teeth of the dental patient.

A one hundred twenty-second example implementation may extend any of the one hundred eighth through one hundred twenty-first example implementations. In the one hundred twenty-second example implementation, the method further includes determining that the natural language instructions include a reference to an interproximal space between teeth; partitioning the mapped teeth into jaw-specific subarrays; and constructing interteeth intervals between each pair of adjacent teeth within each jaw-specific subarray.

In a one hundred twenty-third example implementation, a method includes obtaining, by a processing device, natural language treatment instructions from treatment provider, the natural language treatment instructions including a reference to one or more teeth expressed according to a first indexing scheme; obtaining an indication of a preferred numbering system associated with the treatment provider; providing the natural language treatment instructions, the indication of the preferred numbering system, and a prompt to a large language model (LLM), wherein the prompt comprises: a definition of individual tooth identifiers supporting a plurality of numbering systems, a definition of tooth groups representing anatomical categories, a definition of tooth intervals representing inclusive ranges between specified teeth, a definition of relative tooth positions, and exclusion logic representing a group of exclusions using nested references to include and exclude sets of teeth; obtaining, as output from the LLM, machine-readable instructions in a structured format, the machine-readable instructions including a representation of the one or more teeth; and mapping the representation of the one or more teeth to a three-dimensional model of dentition of a dental patient.

A one hundred twenty-fourth example implementation may extend the one hundred twenty-third example implementation. In the one hundred twenty-fourth example implementation, mapping the representation of the one or more teeth to the three-dimensional model comprises applying a matching predicate based on a type of representation, wherein: for individual teeth, a tooth is selected if its identifier matches an entry in the machine-readable instructions; for tooth groups, a tooth is selected if it belongs to a specified anatomical group, filtered by jaw and side; for intervals, a tooth is selected if its position lies between begin and end identifiers, inclusive; for relative positions, a tooth is selected if it is immediately mesial or distal to a referenced tooth based on arch geometry; and for exclusion logic, a matching set is computed by subtracting excluded teeth from an included group.

A one hundred twenty-fifth example implementation may extend any of the one hundred twenty-third through one hundred twenty-fourth example implementations. In the one hundred twenty-fifth example implementation, the natural language treatment instructions include a reference to an interproximal space, and wherein the method further comprises: partitioning mapped teeth into a first subarray for an upper jaw and a second subarray for a lower jaw; for each subarray, iterating through teeth in order and constructing intervals between each pair of adjacent teeth; and applying the constructed intervals to generate a treatment plan addressing the interproximal space.

A one hundred twenty-sixth example implementation may extend any of the one hundred twenty-third through one hundred twenty-fifth example implementations. In the one hundred twenty-sixth example implementation, the three-dimensional model includes one or more of an unerupted tooth, a supernumerary tooth, or a pontic tooth, and wherein sorting the teeth based on geometric position accounts for the one or more of the unerupted tooth, the supernumerary tooth, or the pontic tooth.

A one hundred twenty-seventh example implementation may extend any of the one hundred twenty-third through one hundred twenty-sixth example implementations. In the one hundred twenty-seventh example implementation, the prompt is extensible to allow addition of new examples and rules as clinical language evolves, and wherein the LLM produces output that conforms to a predefined schema and captures semantic intent of the natural language treatment instructions.

A one hundred twenty-eighth example implementation may extend any of the one hundred twenty-third through one hundred twenty-seventh example implementations. In the one hundred twenty-eighth example implementation, the method further includes determining that the machine-readable instructions include an unsupported instruction type or an ambiguous reference; and automatically flagging the orthodontic case for manual review responsive to the determining.

A one hundred twenty-ninth example implementation may extend any of the one hundred twenty-third through one hundred twenty-eighth example implementations. In the one hundred twenty-ninth example implementation, mapping the representation of the one or more teeth to the three-dimensional model comprises: sorting teeth of the three-dimensional model based on geometric position along a jaw arch, wherein an upper jaw is sorted from right to left along the arch and a lower jaw is sorted from left to right along the arch; caching the sorted teeth; and reusing the cached sorted teeth across multiple instructions within a same orthodontic case.

In a one hundred thirtieth example implementation, a method includes obtaining first treatment provider instructions comprising first natural language instructions associated with treatment of a dental patient, the first natural language instructions comprising a first reference to one or more teeth of the dental patient expressed in accordance with a first indexing scheme for at least one of teeth or inter-tooth intervals; providing the first natural language instructions and an accompanying prompt as input to an artificial intelligence (AI) model, wherein the prompt comprises a description of the first indexing scheme; obtaining, as output from the AI model, first machine-readable instructions related to the treatment provider instructions comprising a first reference to a first tooth in accordance with a first machine-readable indexing scheme; and adjusting a treatment planning algorithm based on the first machine-readable instructions.

A one hundred thirty-first example implementation may extend the one hundred thirtieth example implementation. In the one hundred thirty-first example implementation, the prompt further comprises a description of a second indexing scheme, and wherein the first natural language instructions further comprise a second reference to one or more teeth of the dental patient expressed in accordance with the second indexing scheme.

A one hundred thirty-second example implementation may extend any of the one hundred thirtieth through one hundred thirty-first example implementations. In the one hundred thirty-second example implementation, the method further includes performing treatment instruction validation on the first machine-readable instructions, wherein treatment instruction validation comprises determining that the first machine-readable instructions do not violate a set of treatment instruction heuristics.

A one hundred thirty-third example implementation may extend any of the one hundred thirtieth through one hundred thirty-second example implementations. In the one hundred thirty-third example implementation, the method further includes obtaining second treatment provider instructions in natural language; providing the second treatment provider instructions to the trained AI model; obtaining, as output from the trained AI model, third machine-readable instructions; performing treatment instruction validation on the third machine-readable instructions; and responsive to determining that the third machine-readable instructions violate one or more of a set of treatment instruction heuristics, provide the second treatment provider instructions to a manual treatment planning pipeline.

A one hundred thirty-fourth example implementation may extend any of the one hundred thirtieth through one hundred thirty-third example implementations. In the one hundred thirty-fourth example implementation, the method further includes obtaining dental data of the dental patient; mapping a first instruction associated with the first tooth to a second machine-readable indexing scheme associated with the dental data; and processing the dental data using the treatment planning algorithm in view of mapping the first instruction to the second machine-readable indexing scheme to generate a treatment plan for the dental patient.

A one hundred thirty-fifth example implementation may extend the one hundred thirty-fourth example implementation. In the one hundred thirty-fifth example implementation, the method further includes causing one or more treatment appliances to be manufactured based on the treatment plan.

In a one hundred thirty-sixth example implementation, a method includes obtaining treatment provider instructions in natural language indicative of one or more differences between target dental treatment for a target dental patient and a set of treatment preferences associated with the treatment provider, wherein the treatment provider instructions comprise a first reference to one or more teeth of the dental patient expressed in accordance with a first indexing scheme; obtaining a prompt comprising: a description of the first indexing scheme, a description of a plurality of categories of instructions associated with dental treatment, and a plurality of examples of machine-readable instructions corresponding to natural language instructions within the categories of instructions; providing the treatment provider instructions and the prompt as input to an artificial intelligence (AI) model; obtaining output from the AI model comprising a second reference to the one or more teeth expressed in accordance with a second indexing scheme; and generating a treatment plan based on the output from the AI model.

A one hundred thirty-seventh example implementation may extend the one hundred thirty-sixth example implementation. In the one hundred thirty-seventh example implementation, the prompt further comprises a description of a second indexing scheme, and wherein the treatment provider instructions further comprise a second reference to one or more teeth of the dental patient expressed in accordance with the second indexing scheme.

A one hundred thirty-eighth example implementation may extend any of the one hundred thirty-sixth through one hundred thirty-seventh example implementations. In the one hundred thirty-eighth example implementation, the method further includes generating a treatment algorithm based on output from the trained AI model; and obtaining patient data of the target dental patient, wherein generating the treatment plan comprises applying the treatment algorithm to the patient data.

A one hundred thirty-ninth example implementation may extend the one hundred thirty-eighth example implementation. In the one hundred thirty-ninth example implementation, the patient data comprises a three-dimensional model of the patient's dentition, and wherein the method further comprises mapping the second indexing scheme to a third indexing scheme of the three-dimensional model.

A one hundred fortieth example implementation may extend the one hundred thirty-ninth example implementation. In the one hundred fortieth example implementation, the third indexing scheme is based on tooth identification, and wherein the second indexing scheme is based on jaw geometry of the dental patient.

A one hundred forty-first example implementation may extend any of the one hundred thirty-sixth through one hundred fortieth example implementations. In the one hundred forty-first example implementation, the method further includes performing treatment instruction validation on the output from the AI model, wherein treatment instruction validation comprises determining that the output from the AI model does not violate a set of treatment instruction heuristics.

A one hundred forty-second example implementation includes a non-transitory computer readable medium comprising instructions that, when executed by a processing device, cause the processing device to perform the method of any of the one hundred eighth through one hundred forty-first example implementations.

A one hundred forty-third example implementation includes a system comprising one or more computing device each comprising a memory and one or more processors, wherein the one or more computing devices are configured to perform the method of any of the one hundred eighth through one hundred forty-first example implementations.

In a one hundred forty-fourth example implementation, a method includes obtaining, by a processing device, a large language model (LLM) base prompt associated with a first target task; providing a first prompt generation request, comprising the LLM base prompt, a task description for a second target task different from the first task, a set of design principles associated with the first task, and one or more examples, as input to a first LLM; obtaining, as first output from the first LLM, a model-generated prompt for the second target task; providing the model-generated prompt and input associated with a specific instance of the second target task to a second LLM; and obtaining second output from the second LLM based on the model-generated prompt and the second target task.

A one hundred forty-fifth example implementation may extend the one hundred forty-fourth example implementation. In the one hundred forty-fifth example implementation, the first prompt generation request further comprises a set of unannotated, real-world examples that serve as semantic anchors defining a scope and nature of the second target task without prescribing specific outputs.

A one hundred forty-sixth example implementation may extend any of the one hundred forty-fourth through one hundred forty-fifth example implementations. In the one hundred forty-sixth example implementation, the method further includes storing the model-generated prompt in a prompt registry; and using the model-generated prompt in one or more downstream pipelines for automated text understanding.

A one hundred forty-seventh example implementation may extend any of the one hundred forty-fourth through one hundred forty-sixth example implementations. In the one hundred forty-seventh example implementation, the model-generated prompt includes: a detailed task description; a list of categories and subcategories to be extracted; interpretation rules for linguistic patterns and edge cases; output formatting requirements including adherence to a schema; and guidance for handling ambiguous or compound instructions.

A one hundred forty-eighth example implementation may extend any of the one hundred forty-fourth through one hundred forty-seventh example implementations. In the one hundred forty-eighth example implementation, providing the first prompt generation request to the first LLM comprises transmitting the first prompt generation request to a remote computing device that executes the first LLM; obtaining the model-generated prompt comprises receiving the model-generated prompt from the remote computing device; providing the model-generated prompt and the input associated with the specific instance of the second target task to the second LLM comprises transmitting the model-generated prompt and the input to the remote computing device or a second remote computing device that executes the second LLM; and obtaining the output from the second LLM comprises receiving the second output from the remote computing device or the second remote computing device.

A one hundred forty-ninth example implementation may extend any of the one hundred forty-fourth through one hundred forty-eighth example implementations. In the one hundred forty-ninth example implementation, the set of design principles comprises a description of target properties for the model-generated prompt.

A one hundred fiftieth example implementation may extend any of the one hundred forty-fourth through one hundred forty-ninth example implementations. In the one hundred fiftieth example implementation, the method further includes obtaining second output from the first LLM prior to obtaining the first output, the second output comprising a preliminary model-generated prompt; performing review and refinement analysis of the preliminary model-generated prompt to refine the preliminary model-generated prompt and generate the model-generated prompt.

A one hundred fifty-first example implementation may extend the one hundred fiftieth example implementation. In the one hundred fifty-first example implementation, the review and refinement analysis is performed iteratively across multiple cycles, wherein each cycle processes a focused subset of representative data and key insights are explicitly carried forward between cycles to overcome context window limitations of the first LLM.

A one hundred fifty-second example implementation may extend the one hundred fifty-first example implementation. In the one hundred fifty-second example implementation, the one or more examples comprise a plurality of examples, and wherein the review and refinement analysis comprises: generating a schema based on processing of the first prompt generation request or an iteration thereof by the first LLM; evaluating the schema by the first LLM by processing the plurality of examples in view of the schema; determining, based on a result of the evaluating: whether the schema captures semantics of the first prompt generation request or the subsequent prompt generation request; whether any use cases are not covered or are misrepresented; and whether any inconsistencies are present.

A one hundred fifty-third example implementation may extend the one hundred fifty-second example implementation. In the one hundred fifty-third example implementation, one or more first examples of the plurality of examples are evaluated using a first instance of the first LLM in parallel with one or more second examples of the plurality of examples being evaluated using a second instance of the first LLM.

A one hundred fifty-fourth example implementation may extend any of the one hundred fifty-second through one hundred fifty-third example implementations. In the one hundred fifty-fourth example implementation, the method further includes identifying one or more weaknesses of the schema; suggesting one or more improvements to the schema to address the one or more weaknesses, wherein the one or more improvements comprise at least one of: a clarification of one or more edge cases; addition of an additional rule; or addition of an additional example.

A one hundred fifty-fifth example implementation may extend any of the one hundred fifty-second through one hundred fifty-fourth example implementations. In the one hundred fifty-fifth example implementation, the method further includes repeating the review and refinement analysis in an iterative manner until the schema satisfies one or more criteria, wherein the schema is refined with each iteration of the review and refinement analysis.

A one hundred fifty-sixth example implementation may extend any of the one hundred forty-fourth through one hundred fifty-fifth example implementations. In the one hundred fifty-sixth example implementation, the method further includes validating the model-generated prompt by applying the model-generated prompt to at least some of the one or more examples and confirming that an output has a structure that conforms to a schema defined in the base prompt.

A one hundred fifty-seventh example implementation may extend any of the one hundred forty-fourth through one hundred fifty-sixth example implementations. In the one hundred fifty-seventh example implementation, the second target task comprises generating machine-readable instructions, and wherein the method further comprises executing the output from the second LLM comprising the machine-readable instructions.

A one hundred fifty-eighth example implementation may extend the one hundred fifty-seventh example implementation. In the one hundred fifty-eighth example implementation, the second target task comprises generating machine-readable treatment planning instructions for a dental treatment, and wherein executing the output from the second LLM comprises generating a treatment plan for a dental patient.

A one hundred fifty-ninth example implementation may extend the one hundred fifty-eighth example implementation. In the one hundred fifty-ninth example implementation, the method further includes generating manufacturing data for one or more dental treatment appliances associated with the treatment plan; and manufacturing the one or more dental treatment appliances in accordance with the manufacturing data.

A one hundred sixtieth example implementation may extend any of the one hundred forty-fourth through one hundred fifty-ninth example implementations. In the one hundred sixtieth example implementation, the first target task comprises assigning portions of input text to a first set of categories, and wherein the second target task comprises developing a second set of categories based on additional input text, wherein the second set of categories shares one or more categories with the first set of categories.

In a one hundred sixty-first example implementation, a method includes obtaining, by a processing device, a base prompt configured to cause a large language model (LLM) to assign portions of natural language input to a first set of categories; providing a first prompt generation request, comprising the base prompt and a description of a target task comprising generation of a second set of categories to augment the first set of categories, as input to a first LLM; obtaining, as output from the first LLM, a model-generated prompt; providing the model-generated prompt and first input comprising natural language associated with the target task to a second LLM; and obtaining output from the second LLM based on the model-generated prompt, wherein the output comprises assignment of portions of the first input to the second set of categories.

A one hundred sixty-second example implementation may extend the one hundred sixty-first example implementation. In the one hundred sixty-second example implementation, the natural language input comprises a set of instructions, and wherein the first set of categories comprise categories that the instructions may belong to.

A one hundred sixty-third example implementation may extend the one hundred sixty-second example implementation. In the one hundred sixty-third example implementation, the natural language input comprises instructions related to healthcare treatment of a patient.

A one hundred sixty-fourth example implementation may extend the one hundred sixty-third example implementation. In the one hundred sixty-fourth example implementation, the natural language input comprises instructions related to dental treatment of the patient.

A one hundred sixty-fifth example implementation may extend any of the one hundred sixty-first through one hundred sixty-fourth example implementations. In the one hundred sixty-fifth example implementation, the prompt generation request further comprises a description of target properties of the model-generated prompt.

A one hundred sixty-sixth example implementation may extend any of the one hundred sixty-first through one hundred sixty-fifth example implementations. In the one hundred sixty-sixth example implementation, the prompt generation request further comprises one or more examples of natural language input that may be provided that is to be categorized into a category that the first set of categories does not comprise.

A one hundred sixty-seventh example implementation may extend any of the one hundred sixty-first through one hundred sixty-sixth example implementations. In the one hundred sixty-seventh example implementation, the method further includes obtaining second output from the first LLM based on a second prompt generation request; providing the second output, comprising a preliminary model-generated prompt, and the second prompt generation request for review and refinement analysis; and obtaining the first prompt generation request based on the review and refinement analysis of the second output.

A one hundred sixty-eighth example implementation may extend any of the one hundred sixty-first through one hundred sixty-seventh example implementations. In the one hundred sixty-eighth example implementation, the method further includes providing the model-generated prompt for validation, wherein providing the model-generated prompt to the second LLM is performed based on the validation.

A one hundred sixty-ninth example implementation may extend any of the one hundred sixty-first through one hundred sixty-eighth example implementations. In the one hundred sixty-ninth example implementation, the method further includes providing instructions assigned to one or more categories by the second LLM to a third LLM, and obtaining output from the third LLM comprising machine-readable instructions associated with the natural language instructions.

A one hundred seventieth example implementation may extend the one hundred sixty-ninth example implementation. In the one hundred seventieth example implementation, the method further includes executing the machine-readable instructions.

A one hundred seventy-first example implementation may extend the one hundred seventieth example implementation. In the one hundred seventy-first example implementation, the machine-readable instructions comprise instructions for performing orthodontic treatment planning, and wherein the method further comprises: generating, based on the machine-readable instructions, manufacturing data for one or more orthodontic appliances; and manufacturing the one or more orthodontic appliances.

In a one hundred seventy-second example implementation, a method includes obtaining, by a processing device, a base prompt configured to cause a large language model (LLM) to convert natural language dental treatment instructions into a structured, machine-readable format; obtaining a task description for a dental text processing task, the task description specifying a target type of dental treatment instructions to be processed; obtaining a set of design principles specifying structural and behavioral requirements for a model-generated prompt, the design principles including a target output schema for dental treatment data; obtaining a set of unannotated dental treatment instructions from treatment providers, the unannotated dental treatment instructions illustrating inputs the model-generated prompt is expected to handle; providing the base prompt, the task description, the set of design principles, and the set of unannotated dental treatment instructions as input to a first LLM; obtaining, as output from the first LLM, an initial schema comprising a structured representation of dental treatment categories, treatment parameters, and formatting rules; performing iterative review and refinement of the initial schema using the first LLM, wherein each iteration processes a subset of the unannotated dental treatment instructions and carries forward key insights to subsequent iterations; generating, based on the refined schema, the model-generated prompt for the dental text processing task; and validating the model-generated prompt by applying the model-generated prompt to test dental treatment instructions and confirming that output conforms to the refined schema.

A one hundred seventy-third example implementation may extend the one hundred seventy-second example implementation. In the one hundred seventy-third example implementation, performing iterative review and refinement comprises: passing a batch of the unannotated dental treatment instructions through the initial schema; analyzing outputs to assess how well the schema captures semantics of the unannotated dental treatment instructions; identifying dental treatment instruction types that are not covered or are misinterpreted; identifying ambiguities or inconsistencies in dental terminology interpretation; and refining the schema based on the analysis.

A one hundred seventy-fourth example implementation may extend any of the one hundred seventy-second through one hundred seventy-third example implementations. In the one hundred seventy-fourth example implementation, performing iterative review and refinement further comprises: using the first LLM to identify weaknesses in the schema related to dental treatment instruction interpretation; and using the first LLM to suggest improvements to the schema, wherein the improvements comprise one or more of refining dental treatment categories, clarifying edge cases involving dental terminology, or adding missing rules and examples for dental treatment instructions.

A one hundred seventy-fifth example implementation may extend any of the one hundred seventy-second through one hundred seventy-fourth example implementations. In the one hundred seventy-fifth example implementation, the model-generated prompt comprises: a detailed task description for converting dental treatment instructions to machine-readable format; a list of dental treatment categories and subcategories to be extracted, the categories including one or more of tooth movement instructions, attachment instructions, interproximal reduction instructions, or staging instructions; interpretation rules for dental terminology and clinical language patterns; output formatting requirements including adherence to a schema for dental treatment data; input and output format specifications with dental treatment instruction examples; and guidance for handling ambiguous or compound dental treatment instructions.

A one hundred seventy-sixth example implementation may extend any of the one hundred seventy-second through one hundred seventy-fifth example implementations. In the one hundred seventy-sixth example implementation, the method further includes storing the validated model-generated prompt in a prompt registry; and providing the model-generated prompt for use in one or more dental treatment planning pipelines to convert treatment provider instructions into machine-readable treatment planning instructions.

A one hundred seventy-seventh example implementation may extend any of the one hundred seventy-second through one hundred seventy-sixth example implementations. In the one hundred seventy-seventh example implementation, the set of design principles comprises one or more of: a required output format for dental treatment data; dental treatment categories to be extracted including orthodontic treatment parameters; expected robustness to variation in dental terminology and tooth numbering systems; extensibility requirements for accommodating new dental treatment instruction types; or mandatory inclusion of representative dental treatment instruction examples.

A one hundred seventy-eighth example implementation may extend any of the one hundred seventy-second through one hundred seventy-seventh example implementations. In the one hundred seventy-eighth example implementation, the target type of dental treatment instructions comprises one or more of: case-specific treatment instructions for a particular dental patient; treatment modification instructions requesting changes to an existing dental treatment plan; orthodontic finishing instructions specifying final tooth positioning requirements; or clinical protocol instructions specifying treatment preferences applicable to multiple patients.

A one hundred seventy-ninth example implementation includes a non-transitory computer readable medium comprising instructions that, when executed by a processing device, cause the processing device to perform the method of any of the one hundred forty-fourth through one hundred seventy-eighth example implementations.

A one hundred eightieth example implementation includes a system comprising one or more computing device each comprising a memory and one or more processors, wherein the one or more computing devices are configured to perform the method of any of the one hundred forty-fourth through one hundred seventy-eighth example implementations.

In a one hundred eighty-first example implementation, a method includes obtaining, by a processing device, first treatment instructions for a target dental treatment of a patient; determining that the treatment instructions comprise one or more manual comments in a natural language format; processing the treatment instructions using a classification model, wherein the classification model categorizes the treatment instructions into one or more dental treatment categories; determining that each of the one or more dental treatment categories belongs to a specified subset of dental treatment categories; responsive to determining that each of the one or more dental treatment categories belongs to the specified subset of dental treatment categories, providing at least a portion of the first treatment instructions and a prompt to a large language model (LLM) to cause the LLM to generate machine-readable instructions corresponding to the specified subset of dental treatment categories; and obtaining the machine-readable instructions corresponding to the treatment instructions from the LLM.

A one hundred eighty-second example implementation may extend the one hundred eighty-first example implementation. In the one hundred eighty-second example implementation, providing the at least the portion of the first treatment instructions and the prompt to the LLM comprises transmitting at least one of the portion of the first treatment instructions or the prompt to a remote computing device that executes the LLM; and obtaining the machine-readable instructions comprises receiving the machine-readable instructions from the remote computing device.

A one hundred eighty-third example implementation may extend any of the one hundred eighty-first through one hundred eighty-second example implementations. In the one hundred eighty-third example implementation, the method further includes performing treatment planning operations for the target dental treatment based on the machine-readable instructions.

A one hundred eighty-fourth example implementation may extend the one hundred eighty-third example implementation. In the one hundred eighty-fourth example implementation, the method further includes generating manufacturing data for one or more treatment appliances based on the treatment planning operations; and causing the one or more treatment appliances to be manufactured based on the manufacturing data.

A one hundred eighty-fifth example implementation may extend any of the one hundred eighty-first through one hundred eighty-fourth example implementations. In the one hundred eighty-fifth example implementation, the method further includes obtaining second treatment instructions; determining, using the classification model, that the second treatment instructions comprise instructions belonging to a dental treatment category other than the specified subset of dental treatment categories; and providing the second treatment instructions for manual processing.

A one hundred eighty-sixth example implementation may extend any of the one hundred eighty-first through one hundred eighty-fifth example implementations. In the one hundred eighty-sixth example implementation, the LLM is configured to accept instructions belonging to the specified subset of dental treatment categories as input.

A one hundred eighty-seventh example implementation may extend any of the one hundred eighty-first through one hundred eighty-sixth example implementations. In the one hundred eighty-seventh example implementation, the method further includes determining, using the classification model, that the first treatment instructions comprise one or more instructions belonging to a treatment category not included in the specified subset of dental treatment categories, wherein the one or more instructions are not used in generating the machine-readable instructions.

A one hundred eighty-eighth example implementation may extend any of the one hundred eighty-first through one hundred eighty-seventh example implementations. In the one hundred eighty-eighth example implementation, the machine-readable instructions comprise instructions for placement of attachments to one or more teeth of a patient for the target dental treatment of the patient.

A one hundred eighty-ninth example implementation may extend any of the one hundred eighty-first through one hundred eighty-eighth example implementations. In the one hundred eighty-ninth example implementation, the LLM extracts teeth identifiers of one or more teeth to receive attachments, one or more treatment stages at which to place the attachments, one or more treatment actions to be performed, and attachment types for the attachments.

A one hundred ninetieth example implementation may extend any of the one hundred eighty-first through one hundred eighty-ninth example implementations. In the one hundred ninetieth example implementation, the machine-readable instructions comprise a text-based schema.

A one hundred ninety-first example implementation may extend any of the one hundred eighty-first through one hundred ninetieth example implementations. In the one hundred ninety-first example implementation, the method further includes determining that the first treatment instructions include terminology variations or misspellings of attachment-related terms; and normalizing the terminology variations or misspellings to standard attachment terminology prior to generating the machine-readable instructions.

A one hundred ninety-second example implementation may extend any of the one hundred eighty-first through one hundred ninety-first example implementations. In the one hundred ninety-second example implementation, the machine-readable instructions include stage information represented in one or more formats comprising: a first stage indicator representing a beginning of treatment, a last stage indicator representing an end of treatment, a literal stage number, an offset value representing a number of stages relative to a reference stage, or a time indicator specifying whether an action is to be applied before or after a specified stage.

In a one hundred ninety-third example implementation, a method includes obtaining, by a processing device, first natural language instructions from a treatment provider comprising target updates to a target dental treatment; providing the first natural language instructions to a first large language model (LLM), wherein the first large language model is configured to categorize natural language instructions; determining, using the first LLM, that a first category of instructions is not present in the first natural language instructions associated with dental treatment; providing at least a portion of the first natural language instructions and a prompt configured to cause a second LLM to generate machine-readable instructions corresponding to natural language instructions to the second LLM; and obtaining machine-readable instructions corresponding to the first natural language instructions from the second LLM.

A one hundred ninety-fourth example implementation may extend the one hundred ninety-third example implementation. In the one hundred ninety-fourth example implementation, the method further includes integrating the machine-readable instructions into treatment planning operations.

A one hundred ninety-fifth example implementation may extend the one hundred ninety-fourth example implementation. In the one hundred ninety-fifth example implementation, the method further includes causing one or more treatment appliances to be manufactured based on the treatment planning operations.

A one hundred ninety-sixth example implementation may extend any of the one hundred ninety-third through one hundred ninety-fifth example implementations. In the one hundred ninety-sixth example implementation, the method further includes obtaining second natural language instructions; determining, using the first LLM, that the second natural language instructions comprise instructions belonging to the first category of instructions; and providing the second natural language instructions for manual processing.

A one hundred ninety-seventh example implementation may extend any of the one hundred ninety-third through one hundred ninety-sixth example implementations. In the one hundred ninety-seventh example implementation, the method further includes determining, using the first LLM, that the first natural language instructions comprise instructions belonging to a second category of instructions, wherein the second LLM is configured to accept instructions belonging to the second category of instructions as input.

A one hundred ninety-eighth example implementation may extend any of the one hundred ninety-third through one hundred ninety-seventh example implementations. In the one hundred ninety-eighth example implementation, the method further includes determining, using the first LLM, that the first natural language instructions comprise instructions belonging to a third category of instructions, wherein the third category of instructions comprises instructions that are not to be included in the target treatment.

A one hundred ninety-ninth example implementation may extend any of the one hundred ninety-third through one hundred ninety-eighth example implementations. In the one hundred ninety-ninth example implementation, the machine-readable instructions comprise updates to one or more treatment plan parameters for the target dental treatment.

A two hundredth example implementation may extend the one hundred ninety-ninth example implementation. In the two hundredth example implementation, the one or more treatment plan parameters comprise dental appliance features for one or more dental appliances to be used for the target dental treatment.

A two hundred first example implementation may extend the two hundredth example implementation. In the two hundred first example implementation, the dental appliance features comprise at least one of attachments, bite ramps, or modeled appliance features.

A two hundred second example implementation may extend any of the two hundredth through two hundred first example implementations. In the two hundred second example implementation, the dental appliance features comprise mandibular advancement features selected from at least one of buccal blocks or occlusal blocks.

A two hundred third example implementation may extend any of the one hundred ninety-ninth through two hundred second example implementations. In the two hundred third example implementation, the one or more treatment plan parameters comprise one or more planning targets.

A two hundred fourth example implementation may extend the two hundred third example implementation. In the two hundred fourth example implementation, the one or more planning targets comprise at least one of intended final positions for one or more teeth, tooth velocities for one or more teeth, target treatment outcome, number of treatment stages, amount of overcorrection, and whether to apply passive aligners.

In a two hundred fifth example implementation, a method includes obtaining, by a processing device, first natural language instructions from a treatment provider associated with a target dental treatment; providing the first natural language instructions to a classification model; determining, using the classification model, that a first category of instructions is not present in the first natural language instructions, and that a second category of instructions is present in the first natural language instructions; providing at least a portion of the first natural language instructions and a prompt configured to cause a large language model (LLM) to generate machine-readable instructions corresponding to natural language instructions to the LLM; obtaining machine-readable instructions corresponding to the first natural language instructions from the LLM; updating a treatment plan to generate an updated treatment plan associated with the target dental treatment based on the machine-readable instructions; and providing a representation of the updated treatment plan for treatment provider review.

A two hundred sixth example implementation may extend the two hundred fifth example implementation. In the two hundred sixth example implementation, the representation of the updated treatment plan comprises one or more of: a natural language description of the updated treatment plan; a medical algorithm depicting the updated treatment plan; or a visualization of a three-dimensional model depicting predicted properties of dentition associated with the updated treatment plan.

A two hundred seventh example implementation may extend any of the two hundred fifth through two hundred sixth example implementations. In the two hundred seventh example implementation, the method further includes obtaining an indication that the updated treatment plan has been accepted; generating manufacturing data for one or more treatment appliances in view of the updated treatment plan being accepted; and causing the one or more treatment appliances to be manufactured.

A two hundred eighth example implementation may extend any of the two hundred fifth through two hundred seventh example implementations. In the two hundred eighth example implementation, the at least a portion of the first natural language instructions comprise instructions belonging to the second category of instructions.

A two hundred ninth example implementation may extend the two hundred eighth example implementation. In the two hundred ninth example implementation, the second category of instructions comprises instructions associated with dental features.

A two hundred tenth example implementation may extend any of the two hundred eighth through two hundred ninth example implementations. In the two hundred tenth example implementation, the method further includes determining, using the classification model, that a third category of instructions is present in the first natural language instructions, wherein the third category of instructions comprises instructions that are not applicable to the treatment plan; and excluding instructions belonging to the third category of instructions from the at least a portion of the first natural language instructions provided to the LLM.

A two hundred eleventh example implementation may extend any of the two hundred fifth through two hundred tenth example implementations. In the two hundred eleventh example implementation, the method further includes obtaining second natural language instructions; determining, using the classification model, that the second natural language instructions comprise instructions belonging to the first category of instructions; and providing the second natural language instructions for manual processing.

In a two hundred twelfth example implementation, a method includes obtaining, by a processing device, treatment modification comments from a treatment provider associated with a dental treatment, the treatment modification comments expressed in natural language; providing the treatment modification comments to a classification model configured to categorize natural language instructions into a plurality of treatment categories; determining, using the classification model, that the treatment modification comments include instructions belonging to a dental features category and do not include instructions belonging to categories that require manual processing; providing the treatment modification comments to a specialized interpretation model configured to extract attachment-related parameters from natural language instructions; obtaining, from the specialized interpretation model, machine-readable instructions comprising one or more of teeth identifiers, treatment stages, attachment actions, or attachment types; performing post-processing validation on the machine-readable instructions to confirm compatibility with a treatment planning system; and applying the machine-readable instructions to update the dental treatment.

A two hundred thirteenth example implementation may extend the two hundred twelfth example implementation. In the two hundred thirteenth example implementation, the method further includes determining that the treatment modification comments include terminology variations or misspellings of attachment-related terms; and normalizing the terminology variations or misspellings to standard attachment terminology; wherein the attachment actions comprise one or more of add, remove, keep, replace, or modify.

A two hundred fourteenth example implementation may extend any of the two hundred twelfth through two hundred thirteenth example implementations. In the two hundred fourteenth example implementation, the method further includes determining that the treatment modification comments are expressed in a non-English language; and translating the treatment modification comments to English in the machine-readable instructions.

A two hundred fifteenth example implementation includes a non-transitory computer readable medium comprising instructions that, when executed by a processing device, cause the processing device to perform the method of any of the one hundred eighty-first through two hundred fourteenth example implementations.

A two hundred sixteenth example implementation includes a system comprising one or more computing device each comprising a memory and one or more processors, wherein the one or more computing devices are configured to perform the method of any of the one hundred eighty-first through two hundred fourteenth example implementations.

In a two hundred seventeenth example implementation, a method includes providing a chat interface associated with a treatment protocol or a treatment plan for a dental treatment; obtaining, via the chat interface, first natural language comments associated with an update to one of the treatment protocol or the treatment plan; converting the first natural language comments into one or more proposed structured, valid protocol changes for the treatment protocol or the dental treatment; generating a preview and human-readable explanations of the one or more proposed structured, valid protocol changes; outputting the preview and the human-readable explanations of the one or more proposed structured, valid protocol changes; and responsive to receiving confirmation to implement the one or more proposed structured, valid protocol changes, updating the treatment protocol or the dental treatment.

A two hundred eighteenth example implementation may extend the two hundred seventeenth example implementation. In the two hundred eighteenth example implementation, the one or more proposed structured, valid protocol changes comprise adjustments to one or more fields that correspond deterministically to machine-readable code.

A two hundred nineteenth example implementation may extend any of the two hundred seventeenth through two hundred eighteenth example implementations. In the two hundred nineteenth example implementation, the converting and the generating are performed by a large language model (LLM).

A two hundred twentieth example implementation may extend the two hundred nineteenth example implementation. In the two hundred twentieth example implementation, the method further includes providing a library of machine-implementable instructions to the LLM.

A two hundred twenty-first example implementation may extend any of the two hundred seventeenth through two hundred twentieth example implementations. In the two hundred twenty-first example implementation, the one or more proposed structured, valid protocol changes comprise adjustments to machine-readable code to be applied to the treatment protocol or treatment plan.

A two hundred twenty-second example implementation may extend any of the two hundred seventeenth through two hundred twenty-first example implementations. In the two hundred twenty-second example implementation, the first natural language comments are associated with an update to the treatment protocol, and wherein the method further comprises: obtaining, by the processing device, second natural language comments associated with an update to the treatment plan that is a specific application of the treatment protocol to a patient; converting the second natural language comments into one or more additional structured, valid protocol changes for the dental treatment; generating a second preview of the one or more additional proposed structured, valid protocol changes; outputting the second preview and human-readable explanations of the one or more additional proposed structured, valid protocol changes; and responsive to receiving confirmation to implement the one or more additional proposed structured, valid protocol changes, updating the dental treatment.

A two hundred twenty-third example implementation may extend the two hundred twenty-second example implementation. In the two hundred twenty-third example implementation, obtaining the first natural language comments and obtaining the second natural language comments is performed via a text entry field of a graphical user interface.

A two hundred twenty-fourth example implementation may extend the two hundred twenty-third example implementation. In the two hundred twenty-fourth example implementation, the method further includes obtaining third natural language comments prior to receiving the first natural language instructions; providing, via the graphical user interface, a prompt to provide additional information; and obtaining the first natural language instructions in view of the prompt.

A two hundred twenty-fifth example implementation may extend any of the two hundred twenty-second through two hundred twenty-fourth example implementations. In the two hundred twenty-fifth example implementation, at least a portion of the first natural language comments are provided to a large language model (LLM) as additional context with the second natural language comments.

A two hundred twenty-sixth example implementation may extend any of the two hundred seventeenth through two hundred twenty-fifth example implementations. In the two hundred twenty-sixth example implementation, the chat interface is configured to operate at a plurality of protocol levels comprising: a prescription level for modifications specific to a single patient; a global clinical protocol level for modifications applicable to all patients of the treatment provider; and a personal protocol level for customized treatment preferences of the treatment provider.

A two hundred twenty-seventh example implementation may extend any of the two hundred seventeenth through two hundred twenty-sixth example implementations. In the two hundred twenty-seventh example implementation, the method further includes causing the conversational assistant to automatically manipulate one or more graphical user interface elements to effect the one or more proposed structured, valid protocol changes, wherein the manipulation is visible to the treatment provider.

A two hundred twenty-eighth example implementation may extend any of the two hundred seventeenth through two hundred twenty-seventh example implementations. In the two hundred twenty-eighth example implementation, the method further includes detecting that the treatment provider has requested a same or similar change across a plurality of patients; generating a recommendation to apply the change to a global treatment protocol applicable to future patients; and responsive to obtaining confirmation from the treatment provider, updating the global treatment protocol.

A two hundred twenty-ninth example implementation may extend any of the two hundred seventeenth through two hundred twenty-eighth example implementations. In the two hundred twenty-ninth example implementation, the method further includes maintaining the one or more proposed structured, valid protocol changes in a draft state prior to receiving confirmation; and publishing the one or more proposed structured, valid protocol changes to an active protocol state responsive to receiving confirmation.

In a two hundred thirtieth example implementation, a method includes obtaining, by a processing device, first natural language instructions associated with an update to one of a dental treatment protocol or a dental treatment plan; providing the first natural language instructions to a large language model (LLM); obtaining, from the LLM, machine-implementable instructions corresponding to the first natural language instructions; and causing dental treatment planning operations to be performed based on the machine-implementable instructions.

A two hundred thirty-first example implementation may extend the two hundred thirtieth example implementation. In the two hundred thirty-first example implementation, providing the first natural language instructions to the LLM comprises transmitting the first natural language instructions to a remote computing device that executes the LLM; and obtaining the machine-implementable instructions comprises receiving the machine-implementable instructions from the remote computing device.

A two hundred thirty-second example implementation may extend any of the two hundred thirtieth through two hundred thirty-first example implementations. In the two hundred thirty-second example implementation, the method further includes obtaining a natural language inquiry associated with the dental treatment protocol or dental treatment plan; providing the natural language inquiry to the LLM; obtaining, from the LLM, an explanation based on the natural language inquiry; providing the explanation for review, wherein the first natural language instructions are provided in view of the explanation.

A two hundred thirty-third example implementation may extend the two hundred thirty-second example implementation. In the two hundred thirty-third example implementation, the method further includes providing documentation comprising a library of machine-implementable instructions and associated explanations to the LLM, wherein the explanation based on the natural language inquiry is generated based on the documentation.

A two hundred thirty-fourth example implementation may extend the two hundred thirty-third example implementation. In the two hundred thirty-fourth example implementation, the LLM is configured to limit machine-implementable instructions output by the LLM to instructions in the documentation.

A two hundred thirty-fifth example implementation may extend any of the two hundred thirtieth through two hundred thirty-fourth example implementations. In the two hundred thirty-fifth example implementation, the method further includes performing verification operations on the machine-implementable instructions, comprising determining that the machine-implementable instructions adhere to existing safety heuristics, wherein performing the dental treatment planning operations is further based on the verification operations.

A two hundred thirty-sixth example implementation may extend any of the two hundred thirtieth through two hundred thirty-fifth example implementations. In the two hundred thirty-sixth example implementation, the method further includes manufacturing one or more dental treatment appliances based on the dental treatment planning operations.

In a two hundred thirty-seventh example implementation, a method includes providing, via a graphical user interface (GUI), a free text entry element for providing instructions associated with updates to a treatment plan; obtaining first natural language instructions associated with updating a target treatment plan via the free text entry element; converting the first natural language instructions into structured, valid protocol comments for a dental treatment protocol; providing the first natural language instructions to a large language model (LLM) to generate machine-readable instructions comprising updates to the target treatment plan corresponding to the first natural language instructions; and implementing the machine-readable instructions by performing treatment planning operations.

A two hundred thirty-eighth example implementation may extend the two hundred thirty-seventh example implementation. In the two hundred thirty-eighth example implementation, the method further includes obtaining second natural language instructions via the free text entry element; providing the second natural language instructions to the LLM; obtaining, from the LLM, a clarifying inquiry associated with the second natural language instructions; providing the clarifying inquiry; and responsive to providing the clarifying inquiry, obtaining via the free text entry element the first natural language instructions.

A two hundred thirty-ninth example implementation may extend any of the two hundred thirty-seventh through two hundred thirty-eighth example implementations. In the two hundred thirty-ninth example implementation, the method further includes determining that a treatment protocol may be updated based on the first natural language instructions or the machine-readable instructions; providing a prompt comprising a proposed update of the treatment protocol; obtaining a response to the prompt via the free text entry element; determining, using the LLM, that the response to the prompt comprises confirmation of the proposed update; and performing the proposed update responsive to obtaining the response.

A two hundred fortieth example implementation may extend any of the two hundred thirty-seventh through two hundred thirty-ninth example implementations. In the two hundred fortieth example implementation, the method further includes providing an indication of the machine-readable instructions for review via the GUI, wherein implementing the machine-readable instructions is performed based on obtaining confirmation responsive to providing the indication of the machine-readable instruction for review.

A two hundred forty-first example implementation may extend any of the two hundred thirty-seventh through two hundred fortieth example implementations. In the two hundred forty-first example implementation, the method further includes providing a library of machine-implementable instructions to the LLM, wherein the LLM generates the machine-readable instructions by performing retrieval augmented generation in view of the library.

A two hundred forty-second example implementation may extend any of the two hundred thirty-seventh through two hundred forty-first example implementations. In the two hundred forty-second example implementation, the method further includes manufacturing one or more treatment appliances based on the treatment planning operations.

In a two hundred forty-third example implementation, a method includes providing, by a processing device, a conversational assistant interface for a treatment provider to interact with a dental treatment planning system using natural language; obtaining, via the conversational assistant interface, a natural language request from the treatment provider associated with modifying one or more treatment parameters; determining a protocol level at which the modification is to be applied, the protocol level comprising one of a patient-specific prescription level, a global clinical protocol level applicable to multiple patients, or a personal protocol level customized to the treatment provider; generating, using a large language model (LLM) constrained to operate within predefined protocol rules, one or more proposed modifications corresponding to the natural language request; providing, via a graphical user interface (GUI), a preview of the one or more proposed modifications in a draft state; obtaining, via the GUI, confirmation or rejection of each of the one or more proposed modifications; and responsive to obtaining confirmation, publishing the confirmed modifications to an active protocol state at the determined protocol level.

A two hundred forty-fourth example implementation may extend the two hundred forty-third example implementation. In the two hundred forty-fourth example implementation, generating the one or more proposed modifications comprises: constraining output of the LLM to only include modifications that are valid within a predefined set of protocol commands; and providing the LLM with access to a library of protocol documentation including available treatment options and protocol constraints.

A two hundred forty-fifth example implementation may extend any of the two hundred forty-third through two hundred forty-fourth example implementations. In the two hundred forty-fifth example implementation, the method further includes causing the conversational assistant to automatically manipulate one or more GUI elements to effect the confirmed modifications, wherein the manipulation comprises opening dropdown menus, selecting options, and clicking on interface elements visible to the treatment provider.

A two hundred forty-sixth example implementation may extend any of the two hundred forty-third through two hundred forty-fifth example implementations. In the two hundred forty-sixth example implementation, the method further includes detecting that the natural language request corresponds to a modification at the patient-specific prescription level; determining that the modification is applicable to multiple patients; generating a recommendation to promote the modification to the global clinical protocol level; and responsive to obtaining confirmation of the recommendation, updating the global clinical protocol to include the modification.

A two hundred forty-seventh example implementation may extend any of the two hundred forty-third through two hundred forty-sixth example implementations. In the two hundred forty-seventh example implementation, the method further includes maintaining session context data associated with the treatment provider, the session context data comprising a history of prior natural language requests and corresponding modifications; detecting that the treatment provider has requested a same or similar modification across a plurality of patient treatments; and generating a recommendation to apply the modification to the global clinical protocol level based on the detected pattern.

A two hundred forty-eighth example implementation may extend any of the two hundred forty-third through two hundred forty-seventh example implementations. In the two hundred forty-eighth example implementation, generating the one or more proposed modifications comprises: recognizing, by the LLM, a start of a command sequence in the natural language request; and triggering a predefined command sequence to complete the modification, wherein the predefined command sequence comprises a series of GUI manipulations associated with the recognized command.

A two hundred forty-ninth example implementation may extend any of the two hundred forty-third through two hundred forty-eighth example implementations. In the two hundred forty-ninth example implementation, the method further includes obtaining a query from the treatment provider regarding a treatment option or clinical terminology; accessing, by the LLM, a library of clinical explanations including tool tips from the treatment planning system; and providing, via the conversational assistant interface, a natural language explanation responsive to the query using terminology specific to the treatment planning system.

A two hundred fiftieth example implementation includes a non-transitory computer readable medium comprising instructions that, when executed by a processing device, cause the processing device to perform the method of any of the two hundred seventeenth through two hundred forty-ninth example implementations.

A two hundred fifty-first example implementation includes a system comprising one or more computing device each comprising a memory and one or more processors, wherein the one or more computing devices are configured to perform the method of any of the two hundred seventeenth through two hundred forty-ninth example implementations.

In a two hundred fifty-second example implementation, a method includes obtaining, by a processing device, a base prompt associated with a first target task for a first large language model (LLM); providing a first prompt generation request, comprising the base prompt, as input to a second LLM; obtaining, as first output from the second LLM, a model-generated prompt; providing the model-generated prompt and input associated with a second target task, different than the first target task, to a third LLM; and obtaining output from the third LLM based on the model-generated prompt and the second target task.

A two hundred fifty-third example implementation may extend the two hundred fifty-second example implementation. In the two hundred fifty-third example implementation, the prompt generation request further comprises a description of the second target task.

A two hundred fifty-fourth example implementation may extend any of the two hundred fifty-second through two hundred fifty-third example implementations. In the two hundred fifty-fourth example implementation, the prompt generation request further comprises a description of target properties the model-generated prompt, and one or more examples of types of input the second LLM is to be provided with along with the model-generated prompt.

A two hundred fifty-fifth example implementation may extend any of the two hundred fifty-second through two hundred fifty-fourth example implementations. In the two hundred fifty-fifth example implementation, the method further includes obtaining second output from the second LLM based on a second prompt generation request; providing the second output, comprising a preliminary model-generated prompt, and the second prompt generation request for review and refinement analysis; and obtaining the first prompt generation request based on the first prompt generation request and the review and refinement analysis.

A two hundred fifty-sixth example implementation may extend any of the two hundred fifty-second through two hundred fifty-fifth example implementations. In the two hundred fifty-sixth example implementation, the method further includes providing the model-generated prompt for validation, wherein providing the model-generated prompt to the third LLM is performed based on the validation.

A two hundred fifty-seventh example implementation may extend any of the two hundred fifty-second through two hundred fifty-sixth example implementations. In the two hundred fifty-seventh example implementation, the second target task comprises generating machine-readable instructions, and wherein the method further comprises executing the output from the third LLM comprising the machine-readable instructions.

A two hundred fifty-eighth example implementation may extend the two hundred fifty-seventh example implementation. In the two hundred fifty-eighth example implementation, the second target task comprises generating machine-readable treatment planning instructions for a dental treatment, and wherein executing the output from the third LLM comprises generating a treatment plan for a dental patient.

A two hundred fifty-ninth example implementation may extend the two hundred fifty-eighth example implementation. In the two hundred fifty-ninth example implementation, the method further includes generating manufacturing data for one or more dental treatment appliances associated with the treatment plan; and manufacturing the one or more dental treatment appliances in accordance with the manufacturing data.

A two hundred sixtieth example implementation may extend any of the two hundred fifty-second through two hundred fifty-ninth example implementations. In the two hundred sixtieth example implementation, the first target task comprises assigning portions of input text to a first set of categories, and wherein the second target task comprises developing a second set of categories based on additional input text, wherein the second set of categories shares one or more categories with the first set of categories.

In a two hundred sixty-first example implementation, a method includes obtaining, by a processing device, first natural language instructions from a treatment provider associated with a target treatment; providing the first natural language instructions to a classification model; determining, using the classification model, that a first category of instructions is not present in the first natural language instructions; providing at least a portion of the first natural language instructions and a prompt configured to cause a large language model (LLM) to generate machine-readable instructions corresponding to natural language instructions to the LLM; and obtaining machine-readable instructions corresponding to the first natural language instructions from the LLM.

A two hundred sixty-second example implementation may extend the two hundred sixty-first example implementation. In the two hundred sixty-second example implementation, the method further includes performing treatment planning operations for the target treatment based on the machine-readable instructions.

A two hundred sixty-third example implementation may extend the two hundred sixty-second example implementation. In the two hundred sixty-third example implementation, the method further includes generating manufacturing data for one or more treatment appliances based on the treatment planning operations, and causing the one or more treatment appliances to be manufactured based on the manufacturing data.

A two hundred sixty-fourth example implementation may extend any of the two hundred sixty-first through two hundred sixty-third example implementations. In the two hundred sixty-fourth example implementation, the method further includes providing a set of treatment provider instructions to a comment analyzer; determining, by the comment analyzer, that the treatment provider instructions comprise the first natural language instructions; and providing the first natural language instructions to the processing device responsive to determining that the treatment provider instructions comprise the first natural language instructions.

A two hundred sixty-fifth example implementation may extend any of the two hundred sixty-first through two hundred sixty-fourth example implementations. In the two hundred sixty-fifth example implementation, the method further includes obtaining second natural language instructions; determining, using the classification model, that the second natural language instructions comprise instructions belonging to the first category of instructions; and providing the second natural language instructions for manual processing.

A two hundred sixty-sixth example implementation may extend any of the two hundred sixty-first through two hundred sixty-fifth example implementations. In the two hundred sixty-sixth example implementation, the method further includes determining, using the classification model, that the first natural language instructions comprise instructions belonging to a second category of instructions, wherein the LLM is configured to accept instructions belonging to the second category of instructions as input.

A two hundred sixty-seventh example implementation may extend the two hundred sixty-sixth example implementation. In the two hundred sixty-seventh example implementation, the method further includes determining, using the classification model, that the first natural language instructions comprise instructions belonging to a third category of instructions, wherein the third category of instructions comprises instructions that are not to be included in the target treatment.

A two hundred sixty-eighth example implementation may extend the two hundred sixty-seventh example implementation. In the two hundred sixty-eighth example implementation, the at least a portion of the first natural language instructions comprise instructions belonging to the second category of instructions, and do not comprise instructions belonging to the third category of instructions.

A two hundred sixty-ninth example implementation may extend any of the two hundred sixty-first through two hundred sixty-eighth example implementations. In the two hundred sixty-ninth example implementation, the machine-readable instructions comprise updates to attachments for a dental treatment.

In a two hundred seventieth example implementation, a method includes obtaining, by a processing device, first natural language comments associated with an update to one of a treatment protocol or a treatment plan; providing the first natural language comments to a large language model (LLM); obtaining, from the LLM, machine-implementable instructions corresponding to the first natural language comments; and executing the machine-implementable instructions.

A two hundred seventy-first example implementation may extend the two hundred seventieth example implementation. In the two hundred seventy-first example implementation, the machine-implementable instructions comprise adjustments to one or more fields that corresponds deterministically to machine-readable code.

A two hundred seventy-second example implementation may extend the two hundred seventy-first example implementation. In the two hundred seventy-second example implementation, the method further includes providing indications of the adjustments to the one or more fields, via a graphical user interface, for treatment provider review.

A two hundred seventy-third example implementation may extend any of the two hundred seventieth through two hundred seventy-second example implementations. In the two hundred seventy-third example implementation, the machine-implementable instructions comprise adjustments to machine-readable code to be applied to the treatment protocol or treatment plan.

A two hundred seventy-fourth example implementation may extend any of the two hundred seventieth through two hundred seventy-third example implementations. In the two hundred seventy-fourth example implementation, the first natural language comments are associated with an update to a treatment protocol, and wherein the method further comprises: obtaining, by the processing device, second natural language comments associated with an update to a treatment plan; providing the second natural language comments to the LLM; obtaining, from the LLM, machine-implementable instructions corresponding to the second natural language comments; and executing the machine-implementable instructions.

A two hundred seventy-fifth example implementation may extend the two hundred seventy-fourth example implementation. In the two hundred seventy-fifth example implementation, obtaining the first natural language comments and obtaining the second natural language comments is performed via a text entry field of a graphical user interface.

A two hundred seventy-sixth example implementation may extend the two hundred seventy-fifth example implementation. In the two hundred seventy-sixth example implementation, the method further includes obtaining third natural language comments; providing, via the graphical user interface, a prompt to provide additional information; and obtaining the first natural language instructions in view of the prompt.

A two hundred seventy-seventh example implementation may extend any of the two hundred seventy-fourth through two hundred seventy-sixth example implementations. In the two hundred seventy-seventh example implementation, at least a portion of the first natural language comments are provided to the LLM as additional context with the second natural language comments.

A two hundred seventy-eighth example implementation may extend any of the two hundred seventieth through two hundred seventy-seventh example implementations. In the two hundred seventy-eighth example implementation, the method further includes providing a library of machine-implementable instructions to the LLM.

A two hundred seventy-ninth example implementation may extend any of the two hundred seventieth through two hundred seventy-eighth example implementations. In the two hundred seventy-ninth example implementation, executing the machine-implementable instructions comprises updating the treatment protocol or the treatment plan, and wherein the method further comprises: generating manufacturing data for one or more treatment appliances based on the machine-implementable instructions; and causing the one or more treatment appliances to be manufactured.

The above description is intended to be illustrative, and not restrictive. Although the present disclosure has been described with references to specific illustrative examples and embodiments, it will be recognized that the present disclosure is not limited to the examples and embodiments described. The scope of the disclosure should be determined with reference to the following claims, along with the full scope of equivalents to which the claims are entitled.

What is claimed is:

1. A method, comprising:
obtaining first treatment provider instructions in natural language, the first treatment provider instructions associated with treatment of a dental patient;
providing the first treatment provider instructions and a prompt comprising rules for a domain-specific format for dental treatment plans to a trained artificial intelligence (AI) model;
obtaining, as output from the trained AI model, first machine-readable instructions related to the treatment provider instructions structured according to the domain-specific format; and
causing a treatment planning algorithm to be adjusted based on the first machine-readable instructions.

2. The method of claim 1, wherein:
providing the first treatment provider instructions and the prompt to the trained AI model comprises transmitting at least one of the first treatment provider instructions or the prompt to a remote computing device that executes the trained AI model; and
obtaining the first machine-readable instructions comprises receiving the first machine-readable instructions from the remote computing device.

3. The method of claim 2, wherein causing the treatment planning algorithm to be adjusted comprises:
receiving confirmation to apply the first machine-readable instructions; and
transmitting the first machine-readable instructions to the remote computing device or a second remote computing device executing an orthodontic treatment planning application.

4. The method of claim 1, further comprising:
obtaining treatment provider preferences in natural language;
generating second machine-readable instructions based on the treatment provider preferences;
obtaining the second machine-readable instructions encoding treatment preferences of the treatment provider;
obtaining a generic treatment planning algorithm; and
generating the treatment planning algorithm based on the second machine-readable instructions by adjusting the generic treatment planning algorithm.

5. The method of claim 1, further comprising performing treatment instruction validation on the first machine-readable instructions, wherein treatment instruction validation comprises determining that the first machine-readable instructions do not violate a set of treatment instruction heuristics.

6. The method of claim 1, further comprising:
obtaining second treatment provider instructions in natural language;
providing the second treatment provider instructions to the trained AI model;
obtaining, as output from the trained AI model, second machine-readable instructions;
performing treatment instruction validation on the second machine-readable instructions; and
responsive to determining that the second machine-readable instructions violate one or more of a set of treatment instruction heuristics, provide the second treatment provider instructions to a manual treatment planning pipeline.

7. The method of claim 1, further comprising:
obtaining dental data of the dental patient;
processing the dental data using the adjusted treatment planning algorithm to generate a treatment plan for the dental patient; and
causing one or more treatment appliances to be manufactured based on the treatment plan.

8. The method of claim 1, wherein the first machine-readable instructions are structured according to a language-independent data-interchange format schema that organizes the treatment provider instructions into distinct categories, each category representing a specific aspect of treatment planning.

9. The method of claim 1, further comprising:
obtaining second machine-readable instructions encoding general treatment preferences of the treatment provider;
determining a priority relationship between the first machine-readable instructions and the second machine-readable instructions; and
adjusting the treatment planning algorithm such that the first machine-readable instructions override corresponding portions of the second machine-readable instructions.

10. A non-transitory computer-readable medium comprising instructions that, when executed by a processing device, cause the processing device to perform operations comprising:
obtaining, by a processing device, free-text treatment instructions from a treatment provider associated with a specific dental patient, the free-text treatment instructions expressed in natural language;
providing the free-text treatment instructions and a prompt to a language model, wherein the prompt includes a description of instruction categories and examples of machine-readable instructions corresponding to natural language instructions within each category;
obtaining, as output from the language model, case-specific machine-readable instructions in a structured format, the case-specific machine-readable instructions corresponding to the free-text treatment instructions;
performing validation of the case-specific machine-readable instructions to determine compatibility with a treatment planning engine;
responsive to determining that the case-specific machine-readable instructions are compatible with the treatment planning engine, providing the case-specific machine-readable instructions and patient data to the treatment planning engine; and
generating, by the treatment planning engine, a treatment plan for the specific dental patient based on the case-specific machine-readable instructions.

11. The non-transitory computer-readable medium of claim 10, wherein the operations further comprise:
obtaining general treatment protocol instructions associated with the treatment provider, the general treatment protocol instructions applicable to multiple patients; and
combining the case-specific machine-readable instructions with the general treatment protocol instructions, wherein the case-specific machine-readable instructions have a higher priority than the general treatment protocol instructions such that the case-specific machine-readable instructions override corresponding portions of the general treatment protocol instructions.

12. The non-transitory computer-readable medium of claim 10, wherein performing validation of the case-specific machine-readable instructions comprises:
determining whether each instruction type in the case-specific machine-readable instructions is supported by the treatment planning engine;
determining whether parameter values in the case-specific machine-readable instructions fall within predefined acceptable ranges; and
responsive to determining that an instruction type is unsupported or a parameter value falls outside an acceptable range, directing the free-text treatment instructions to a manual treatment planning process.

13. The non-transitory computer-readable medium of claim 10, wherein the structured format comprises a language-independent data-interchange format schema that organizes the case-specific machine-readable instructions into distinct categories, each category representing a specific aspect of treatment planning, and wherein the schema includes a default category for instructions that do not fit into predefined instruction categories.

14. The non-transitory computer-readable medium of claim 10, wherein the free-text treatment instructions include a reference to one or more teeth expressed according to a first indexing scheme, and wherein the operations further comprise:
obtaining a three-dimensional model of dentition of the specific dental patient;
sorting teeth of the three-dimensional model based on geometric position along a jaw arc; and
mapping the reference to the one or more teeth to corresponding teeth in the sorted three-dimensional model based on the geometric position.

15. The non-transitory computer-readable medium of claim 14, wherein the reference to the one or more teeth comprises
one or more of:
an interval specification identifying teeth between two specified teeth;
a group specification identifying teeth belonging to an anatomical category;
a relative position specification identifying teeth based on position relative to a reference tooth; or
an exclusion specification identifying teeth by excluding specified teeth from a group.

16. The non-transitory computer-readable medium of claim 14, wherein the three-dimensional model includes one or more of a supernumerary tooth or a missing tooth, and wherein mapping the reference to the one or more teeth accounts for the supernumerary tooth or the missing tooth based on the geometric position along the jaw arc.

17. The non-transitory computer-readable medium of claim 10, wherein the language model comprises a large language model, a small language model, or a specialized language model.

18. A system comprising one or more computing devices each comprising a memory and one or more processors, wherein the one or more computing devices are configured to:
obtain treatment provider instructions in natural language, wherein the treatment provider instructions are indicative of one or more differences between target dental treatment for a target dental patient and a set of treatment preferences associated with the treatment provider;
obtain a prompt comprising:
a description of a plurality of categories of instructions associated with dental treatment, and
a plurality of examples of machine-readable instructions corresponding to natural language instructions within the categories of instructions;
provide the treatment provider instructions and the prompt as input to a trained artificial intelligence (AI) model;
obtain output from the trained AI model, the output comprising a machine-readable format that is a domain-specific format for orthodontic treatment; and
generate a treatment plan based on the output from the trained AI model.

19. The system of claim 18, wherein the one or more computing devices are further configured to:
generate a treatment algorithm based on output from the trained AI model; and
obtain patient data of the target dental patient, wherein generating the treatment plan comprises applying the treatment algorithm to the patient data.

20. The system of claim 18, wherein the output from the trained AI model comprises machine-readable instructions associated with the first treatment provider instructions, and wherein the one or more computing devices are further configured to:
perform instruction validation comprising determining that the machine-readable instructions to not violate one or more of a set of treatment instruction heuristics; and
responsive to instruction validation, update a treatment algorithm based on the output from the trained AI model.
